(19) United States
(12) Patent Application Publication
(10) Pub. No.: US 2015/0017186 A1
(43) Pub. Date: Jan. 15, 2015

(54) Compositions and combinations of organophosphorus bioscavengers and hyaluronan-degrading enzymes, and methods of use

(71) Applicants: John K. Troyer, Towson, MD (US); Elizabeth Leffel, Berryville, VA (US)

(72) Inventors: John K. Troyer, Towson, MD (US); Elizabeth Leffel, Berryville, VA (US)

(21) Appl. No.: 14/121,452

(22) Filed: Sep. 8, 2014

Related U.S. Application Data
(62) Division of application No. 13/573,472, filed on Sep. 14, 2012.
(60) Provisional application No. 61/627,142, filed on Sep. 16, 2011.

(51) Int. Cl. A61K 38/54; A61K 45/06; A61K 38/47; A61K 38/46; A61K 47/48

(52) U.S. Cl. CPC A61K 38/54; A61K 38/465; A61K 47/48215; A61K 38/47; C12Y 301/01008; C12Y 302/01035; A61K 47/4843; A61K 45/06
USPC 424/178.1; 424/94.2

(57) ABSTRACT

Provided are compositions and combinations containing an organophosphorus bioscavenger and a hyaluronan-degrading enzyme. The provided compositions and combinations can be used to treat or prevent organophosphorus poisoning, including nerve agent poisoning and pesticide poisoning.

### RELATED APPLICATIONS

This application is a divisional of co-pending U.S. application Ser. No. 13/573,472, entitled “COMPOSITIONS AND COMBINATIONS OF ORGANOPHOSPHORUS BIOSCAVENGERS AND HYALURONAN-DEGRADING ENZYMES, AND METHODS OF USE,” filed Sep. 14, 2012, which claims priority to U.S. Provisional Application Ser. No. 61/627,142 to John K. Troyer and Elizabeth K. Leffel, entitled “COMPOSITIONS AND COMBINATIONS OF ORGANOPHOSPHORUS BIOSCAVENGERS AND HYALURONAN-DEGRADING ENZYMES, AND METHODS OF USE,” filed Sep. 16, 2011. The subject matter of the above-referenced applications is incorporated by reference in its entirety.

This application is related to International PCT Patent Application No. PCT/US2012/055638, filed Sep. 14, 2012, entitled “COMPOSITIONS AND COMBINATIONS OF ORGANOPHOSPHORUS BIOSCAVENGERS AND HYALURONAN-DEGRADING ENZYMES, AND USES THEREOF,” which claims priority to U.S. Provisional Application Ser. No. 61/627,142.

The subject matter of the above-noted related applications is incorporated by reference in its entirety.

### GOVERNMENT LICENSE RIGHTS

Work described herein was made with government support under Grant Number U01 NS058207 awarded by the CounterACT Program, Office of the Director, National Institutes of Health (OD) and the National Institute of Neurological Disorders and Stroke (NINDS). The United States Government has certain rights in such subject matter.

### INCORPORATION BY REFERENCE OF SEQUENCE LISTING PROVIDED ON COMPACT DISCS

An electronic version on compact disc (CD-R) of the Sequence Listing is filed herewith in duplicate (labeled Copy 1 and Copy 2), the contents of which are incorporated by reference in their entirety. The computer-readable file on each of the aforementioned compact discs, created on Sep. 4, 2014, is identical, 1,285 kilobytes in size, and titled 3102Bseq.001.txt.

### FIELD OF INVENTION

Provided herein are compositions and combinations of organophosphate bioscavengers and hyaluronan-degrading enzymes. Such compositions and combinations can be used in methods of treating organophosphorus poisoning, including nerve agent poisoning and organophosphorus pesticide poisoning.

### BACKGROUND

Use of organophosphorus and related compounds as pesticides and in warfare over the last several decades has resulted in a rising number of cases of acute and delayed intoxication, causing damage to the peripheral and central nervous systems and resulting in myopathy, psychosis, general paralysis, and death. Such noxious agents act by inhibiting cholinesterase enzymes and thereby preventing the breakdown of neurotransmitters, such as acetylcholine, causing hyperactivity of the nervous system. For example, build-up of acetylcholine causes continued stimulation of the muscarinic receptor sites (exocrine glands and smooth muscles) and the nicotinic receptor sites (skeletal muscles). In addition, exposure to cholinesterase-inhibiting substances can cause symptoms ranging from mild (e.g., twitching, trembling) to severe (e.g., paralyzed breathing, convulsions), and in extreme cases, death, depending on the type and amount of cholinesterase-inhibiting substances involved.

Organophosphorus poisoning can be treated by intravenous or intramuscular administration of combinations of drugs, including carbamates (e.g., pyridostigmine), anti-muscarinics (e.g., atropine), cholinesterase reactivators (ChE-reactivators), such as pralidoxime chloride (2-PAM, Protopam) and anti-convulsives. Alternatively, organophosphorus poisoning can be treated by administration of organophosphorus bioscavengers that bind to or hydrolyze organophosphorus compounds before they reach their physiological targets and exert toxic effects on the subject. Improved compositions for treatment of organophosphorus poisoning are needed.

### SUMMARY

Provided herein are compositions containing an organophosphorus (OP) bioscavenger and a hyaluronan-degrading enzyme. In some examples, the compositions provided herein are formulated for single dosage administration. Also provided herein are combinations containing a first composition containing an organophosphorus bioscavenger and a second composition containing a hyaluronan-degrading enzyme. Also provided herein are containers containing two compartments wherein a first compartment contains a therapeutically effective amount of an organophosphorus bioscavenger, wherein the amount is for single dosage or a plurality of dosages and a single dosage is effective for prevention of organophosphorus poisoning and a second compartment contains a therapeutically effective amount of hyaluronan-degrading enzyme, wherein the therapeutically effective amount of hyaluronan-degrading enzyme is effective to increase bioavailability or absorption of the organophosphorus bioscavenger. Provided herein are methods for preventing organophosphorus poisoning by administering a composition or combination provided herein. Also provided herein are methods for treating organophosphorus poisoning by administering a composition or combination provided herein. Provided herein are methods for preventing organophosphorus poisoning wherein the method involves administering to a subject an organophosphorus bioscavenger and a hyaluronan-degrading enzyme. Also provided herein are methods for treating organophosphorus poisoning, wherein the method involves administering an organophosphorus bioscavenger and a hyaluronan-degrading enzyme. Any of the methods provided herein can further contain a step of administering another pharmaceutical agent selected from among carbamates, anti-muscarinics, cholinesterase reactivators and anti-convulsives. Also provided are uses and compositions containing an organophosphorus bioscavenger and a hyaluronan-degrading enzyme for treating or preventing organophosphorus poisoning.

Provided herein are compositions containing an organophosphorus (OP) bioscavenger and a hyaluronan-degrading enzyme. In some examples, the compositions provided herein are formulated for single dosage administration. Also provided herein are combinations containing a first composition containing an organophosphorus bioscavenger and a second composition containing a hyaluronan-degrading enzyme.

In some examples, the provided compositions and containers contain an organophosphorus bioscavenger that is present in the composition in an amount between or between about 1 μg to 100 mg, 1 μg to 50 mg, 1 μg to 10 mg, 1 μg to 1 mg, 1 μg to 500 μg, 1 μg to 250 μg, 1 μg to 100 μg, 50 μg to 50 mg, 50 μg to 25 mg, 50 μg to 10 mg, 50 μg to 1 mg, 50 μg to 500 μg, 50 μg to 250 μg, 100 μg to 50 mg, 100 μg to 10 mg, 100 μg to 1 mg, 100 μg to 500 μg, 100 μg to 250 μg, 250 μg to 50 mg, 250 μg to 25 mg, 250 μg to 10 mg, 250 μg to 1 mg, 250 μg to 500 μg, 500 μg to 100 mg, 500 to 50 mg, 500 μg to 25 mg, 500 μg to 10 mg, 500 μg to 1 mg, 1 mg to 500 mg, 1 mg to 250 mg, 1 mg to 100 mg, 50 mg to 1000 mg, 250 mg to 1000 mg, 250 mg to 750 mg, 250 mg to 500 mg, 500 mg to 1000 mg, 500 mg to 750 mg, or is at least or is about at least 1 μg, 10 μg, 50 μg, 100 μg, 250 μg, 500 μg, 1 mg, 10 mg, 50 mg, 100 mg, 250 mg, 500 mg, 750 mg or 1000 mg when a single dosage is administered.

In other examples, the provided compositions and containers contain an OP bioscavenger that is present in the composition in an amount between 1 to 1000 μg/mL, 1 to 500 μg/mL, 1 to 250 μg/mL, 1 to 100 mg/mL, 50 to 1000 μg/mL, 50 to 750 μg/mL, 50 to 500 μg/mL, 50 to 250 μg/mL, 100 to 1000 μg/mL, 100 to 500 μg/mL, 100 to 250 μg/mL, 250 to 1000 μg/mL, 250 to 750 μg/mL, 250 to 500 μg/mL, 500 to 1000 μg/mL, 0.5 to 50 mg/mL, 0.5 to 10 mg/mL, 0.5 to 1 mg/mL, 1 to 100 mg/mL, 1 to 50 mg/mL, 1 to 25 mg/mL, 1 to 1000 mg/mL, 1 to 500 mg/mL, 1 to 250 mg/mL, 10 to 500 mg/mL, 10 to 250 mg/mL, 10 to 150 mg/mL, 10 to 100 mg/mL, 10 to 50 mg/mL, 50 to 1500 mg/mL, 50 to 1000 mg/mL, 50 to 750 mg/mL, 50 to 500 mg/mL, 50 to 250 mg/mL, 100 to 1500 mg/mL, 100 to 1000 mg/mL, 100 to 750 mg/mL, 100 to 500 mg/mL, 100 to 7500 mg/mL, 100 to 5000 mg/mL, 100 to 2500 mg/mL, 500 to 7500 mg/mL, 500 to 5000 mg/mL, 500 to 2500 mg/mL, or 500 to 1000 mg/mL.

The hyaluronan-degrading enzyme in the provided compositions and combinations can be present in the composition an amount between 0.5 mg/mL to 100 mg/mL, 0.5 mg/mL to 50 mg/mL, 0.5 mg/mL to 10 mg/mL, 1 mg/mL to 250 mg/mL, 1 mg/mL to 100 mg/mL, 1 mg/mL to 50 mg/mL, 50 mg/mL to 500 mg/mL, 50 mg/mL to 250 mg/mL, 50 mg/mL to 100 mg/mL, 100 mg/mL to 500 mg/mL, 100 mg/mL to 250 mg/mL or 250 mg/mL to 500 mg/mL. In some examples, the hyaluronan-degrading enzyme is present in the compositions and combinations provided herein in an amount between or about between 10 U/mL to 5000 U/mL, 50 U/mL to 4000 U/mL, 100 U/mL to 2000 U/mL, 300 U/mL to 2000 U/mL, 600 U/mL to 2000 U/mL, or 100 U/mL to 1000 U/mL. In other examples, the compositions and combinations provided herein contain a hyaluronan-degrading enzyme in an amount between or about between at least or is about or is 30 U/mL, 35 U/mL, 40 U/mL, 50 U/mL, 100 U/mL, 150 U/mL, 200 U/mL, 250 U/mL, 300 U/mL, 350 U/mL, 400 U/mL, 450 U/mL, 500 U/mL, 600 U/mL, 700 U/mL, 800 U/mL, 900 U/mL, 1000 U/mL or 2000 U/mL.

In the provided compositions and combinations, the volume of the composition can be between or between about 0.5 mL to 15 mL, 1 mL to 10 mL, 2 mL to 8 mL, 5 mL to 7 mL or 4 mL to 6 mL. In some examples, the organophosphorus bioscavenger is present in the composition in an amount between or about between 50 mg/mL to 200 mg/mL and the volume of the composition is between or between about 5 mL to 7 mL. In other examples, the organophosphorus bioscavenger is present in the composition in an amount between or about between 50 mg/mL to 200 mg/mL and the volume of the composition is between or between about 4 mL to 6 mL.

Also provided herein are containers containing two compartments wherein a first compartment contains a therapeutically effective amount of an organophosphorus bioscavenger, wherein the amount is for single dosage or a plurality of dosages and a single dosage is effective for prevention of organophosphorus poisoning and a second compartment contains a therapeutically effective amount of hyaluronan-degrading enzyme, wherein the therapeutically effective amount of hyaluronan-degrading enzyme is effective to increase bioavailability or absorption of the organophosphorus bioscavenger. The organophosphorus poisoning can be organophosphorus pesticide poisoning or nerve agent poisoning. In some examples, the containers further include a mixing compartment. The containers can be a tube or bottle, or alternatively, a syringe. In some examples, the container includes a needle for injection.

In some examples of the containers provided herein, the organophosphorus bioscavenger and hyaluronan-degrading enzyme are provided in an amount for multiple dosage administration. In other examples, the organophosphorus bioscavenger and hyaluronan-degrading enzyme are provided in a single unit dosage. For example, the organophosphorus bioscavenger in the container is for single dosage administration and is present in an amount between or between about 1 μg to 100 mg, 1 μg to 50 mg, 1 μg to 10 mg, 1 μg to 1 mg, 1 μg to 500 μg, 1 μg to 250 μg, 1 μg to 100 μg, 50 μg to 50 mg, 50 μg to 25 mg, 50 μg to 10 mg, 50 μg to 1 mg, 50 μg to 500 μg, 50 μg to 250 μg, 100 μg to 50 mg, 100 μg to 10 mg, 100 μg to 1 mg, 100 μg to 500 μg, 100 μg to 250 μg, 250 μg to 50 mg, 250 μg to 25 mg, 250 μg to 10 mg, 250 μg to 1 mg, 250 μg to 500 μg, 500 μg to 100 mg, 500 μg to 50 mg, 500 μg to 25 mg, 500 μg to 10 mg, 500 μg to 1 mg, 1 mg to 500 mg, 1 mg to 250 mg, 1 mg to 100 mg, 50 mg to 1000 mg, 250 mg to 1000 mg, 250 mg to 750 mg, 250 mg to 500 mg, 500 mg to 1000 mg, 500 mg to 750 mg, or is at least or is about at least 1 μg, 10 μg, 50 μg, 100 μg, 250 μg, 500 μg, 1 mg, 10 mg, 50 mg, 100 mg, 250 mg, 500 mg, 750 mg or 1000 mg when a single dosage is administered. In another example, the organophosphorus bioscavenger is present in an amount between or about between 10 to 500 mg/mL, 10 to 250 mg/mL, 10 to 150 mg/mL, 10 to 100 mg/mL, 10 to 50 mg/mL, 50 to 1500 mg/mL, 50 to 1000 mg/mL, 50 to 750 mg/mL, 50 to 500 mg/mL, 50 to 250 mg/mL, 100 to 1500 mg/mL, 100 to 1000 mg/mL, 100 to 750 mg/mL, 100 to 500 mg/mL, 100 to 7500 mg/mL, 100 to 5000 mg/mL, 100 to 2500 mg/mL, 500 to 7500 mg/mL, 500 to 5000 mg/mL, 500 to 2500 mg/mL, or 500 to 1000 mg/mL. In the containers provided herein, the total volume of the liquid in the container can be from or be from about 0.1 to 10 mL, 0.1 to 5 mL, 0.1 to 3 mL, 0.1 to 1 mL, 1 to 10 mL, 3 to 10 mL, 5 to 10 mL, 5 to 7 mL, 4 to 6 mL, 1 to 5 mL, 1 to 3 mL or 3 to 5 mL.

In some examples of a container provided herein, the hyaluronan-degrading enzyme is present in an amount between or about between 10 U/mL to 5000 U/mL, 50 U/mL to 4000 U/mL, 100 U/mL to 2000 U/mL, 300 U/mL to 2000 U/mL, 600 U/mL to 2000 U/mL, or 100 U/mL to 1000 U/mL. In other examples, the hyaluronan-degrading enzyme is present in an amount between or about between at least or is about or is 30 U/mL, 35 U/mL, 40 U/mL, 50 U/mL, 100 U/mL, 150 U/mL, 200 U/mL, 250 U/mL, 300 U/mL, 350 U/mL, 400 U/mL, 450 U/mL, 500 U/mL, 600 U/mL, 700 U/mL, 800 U/mL, 900 U/mL, 1000 U/mL or 2000 U/mL.

Also provided herein is a container containing two compartments, wherein a first compartment contains a therapeutically effective amount of a butyrylcholinesterase, wherein the amount is effective for preventing organophosphorus poisoning and a second compartment contains a sufficient amount of a hyaluronan-degrading enzyme to increase bioavailability or absorption of the organophosphorus bioscavenger. The organophosphorus poisoning can be organophosphorus pesticide poisoning or nerve agent poisoning. In some examples, the butyrylcholinesterase has a sequence of amino acids set forth in SEQ ID NO:236, or is an allelic or species variant or any other variant of SEQ ID NO:236. The butyrylcholinesterase can be present in the provided container in an amount between or between about 250 mg to 1000 mg, 250 mg to 750 mg, 250 mg to 500 mg, 500 mg to 1000 mg, 500 mg to 750 mg, or is or is about 250 mg, 500 mg, 750 mg or 1000 mg; or is present at 10 to 500 mg/mL, 10 to 250 mg/mL, 10 to 150 mg/mL, 10 to 100 mg/mL, 10 to 50 mg/mL, 50 to 1500 mg/mL, 50 to 1000 mg/mL, 50 to 750 mg/mL, 50 to 500 mg/mL, 50 to 250 mg/mL, 100 to 1500 mg/mL, 100 to 1000 mg/mL, 100 to 750 mg/mL, 100 to 500 mg/mL, 100 to 7500 mg/mL, 100 to 5000 mg/mL, 100 to 2500 mg/mL, 500 to 7500 mg/mL, 500 to 5000 mg/mL, 500 to 2500 mg/mL, or 500 to 1000 mg/mL.

In some examples of the containers provided herein, the organophosphorus bioscavenger can be in solution or suspension. In other examples of the containers provided herein, the hyaluronan-degrading enzyme can be in solution or suspension. In yet other examples of the containers provided herein, both the OP bioscavenger and the hyaluronan-degrading enzyme are in solution or suspension. In the containers provided herein, the volume of the solution or suspension in the container can be between or between about 0.5 mL to 15 mL, 1 mL to 10 mL, 2 mL to 8 mL, 5 mL to 7 mL or 4 mL to 6 mL. In some examples, the organophosphorus bioscavenger is present in solution or suspension in an amount between or about between 50 mg/mL to 200 mg/mL and the volume of the solution or suspension is between or between about 5 mL to 7 mL. In other examples, the organophosphorus bioscavenger is present in solution or in suspension in an amount between or about between 50 mg/mL to 200 mg/mL and the volume of the solution or suspension is between or between about 4 mL to 6 mL. In some examples of the containers provided herein, the organophosphorus bioscavenger is lyophilized. In other examples, the hyaluronan-degrading enzyme is lyophilized. In yet other examples, both the OP bioscavenger and the hyaluronan-degrading enzyme are lyophilized.

The OP bioscavenger in the compositions, combinations and containers provided herein can be an esterase, cholinesterase, paraoxonase, aryldialkylphosphatase or diisopropylfluorophosphatase. For example, the OP bioscavenger is selected from among acetylcholinesterase (AChE), butyrylcholinesterase (BChE), prolidase, organophosphate acid anhydrolase (OPAA), phosphotriesterase, aryldialkylphosphatase, organophosphorus hydrolase (OPH), parathion hydrolase, diisopropylfluorophosphatase (DFPase), organophosphorus acid anhydrase, sarinase and paraoxonase (PON). In one embodiment, the OP bioscavenger is an active portion or a variant of an acetylcholinesterase (AChE), butyrylcholinesterase (BChE), prolidase, organophosphate acid anhydrolase (OPAA), phosphotriesterase, aryldialkylphosphatase, organophosphorus hydrolase (OPH), parathion hydrolase, diisopropylfluorophosphatase (DFPase), organophosphorus acid anhydrase, sarinase or paraoxonase (PON) that exhibits at least 80%, 85%, 90%, 95%, or more OP binding or inactivating activity.

In some examples provided herein, the organophosphorus bioscavenger has a sequence of amino acids set forth in any of SEQ ID NOS: 214-256 and 258-301. In other examples, the OP bioscavenger is an active portion thereof or a variant thereof that exhibits at least 80%, 85%, 90%, 95%, or more sequence identity to any of SEQ ID NOS: 214-256 and 258-301. In such examples, the active portion or variant exhibits OP binding or inactivating activity. For example, the active portion or variant can exhibit at least 40%, 50%, 60%, 70%, 80%, 90%, or more activity compared to the corresponding organophosphorus bioscavenger set forth in any of SEQ ID NOS: 214-256 and 258-301.

In some examples, the OP bioscavenger in the compositions, combinations and containers provided herein is a variant that has a sequence of amino acids that contains an amino acid modification compared to organophosphorus bioscavenger that has a sequence of amino acids set forth in any of SEQ ID NOS: 214-256 and 258-301. The amino acid modification can be an amino acid replacement or substitution, deletion or addition. In some examples, the variant exhibits increased catalytic activity or other inhibitory activity compared to the organophosphorus bioscavenger not containing the amino acid modification.

In some embodiments, the compositions, combinations and containers provided herein contain an organophosphorus bioscavenger that is a cholinesterase. For example, the OP bioscavenger is a cholinesterase that is an acetylcholinesterase or butyrylcholinesterase. The cholinesterase can be a monomer, dimer or a tetramer. In an exemplary embodiment, the organophosphorus bioscavenger is butyrylcholinesterase. For example, the OP bioscavenger is a butyrylcholinesterase that has a sequence of amino acids set forth in SEQ ID NO:236, or is an active portion thereof or is a variant thereof that exhibits at least 85% sequence identity to a sequence of amino acids set forth in SEQ ID NO:236. For example, the butyrylcholinesterase has at least 86%, 87%, 88%, 89%, 90%, 91%, 92%, 93%, 94%, 95%, 96%, 97%, 98% or 99% sequence identity to a sequence of amino acids set forth in SEQ ID NO:236 and exhibits OP binding or inactivating activity. In some examples provided herein, the organophosphorus bioscavenger is a cholinesterase and the active portion is a truncated fragment lacking a tryptophan amphiphilic tetramerization (WAT) domain.

In some examples, the OP bioscavenger in the provided compositions, combinations and containers exhibits a half-life of at least 30 hours, 40 hours, 50 hours, 60 hours, 70 hours, 80 hours, 90 hours or 100 hours; or between or about between 30 hours to 130 hours, 40 hours to 100 hours, 40 hours to 80 hours, 40 hours to 60 hours.

The organophosphorus bioscavenger in the compositions, combinations and containers provided can be modified with a polymer. For example, the OP bioscavenger can be modified with a polymer that is a polyethylene glycol (PEG). In other examples, the OP bioscavenger can be linked directly or indirectly via a linker to an immunoglobulin, immunoglobulin domain, albumin, transferrin, or transferrin receptor protein. The linker can be a chemical linker or a polypeptide linker, for example, a peptide, polypeptide or an amino acid.

The hyaluronan-degrading enzyme in the compositions, combinations and containers provided herein can be a hyaluronidase or a chondroitinase, or a variant or a truncated form thereof that exhibits hyaluronan-degrading activity. In some examples, the hyaluronan-degrading enzyme is a hyaluronidase that is active at neutral pH. For example, the hyaluronan-degrading enzyme is a Hyal1, Hyal2, Hyal4 or PH20 hyaluronidase, or a variant or a truncated form thereof that exhibits hyaluronidase activity. In a specific example, the hyaluronan-degrading enzyme is a hyaluronidase that is a PH20 or a variant or a truncated form thereof that exhibits hyaluronidase activity. For example, the hyaluronidase is a PH20 that is a non-human or a human PH20 or variant or a truncated form thereof that exhibits hyaluronidase activity. Such PH20 hyaluronidases include human, monkey, bovine, ovine, rat, mouse or guinea pig PH20, or a variant or a truncated form thereof that exhibits hyaluronidase activity.

In some examples of the provided compositions, combinations and containers, the hyaluronan-degrading enzyme lacks all or a portion of a glycophosphatidylinositol (GPI) anchor or is not membrane-associated when expressed from a cell. For example, the hyaluronan-degrading enzyme contains C-terminal truncations of one or more amino acid residues to remove all or part of a GPI anchor. In a specific example, the hyaluronan-degrading enzyme is a truncated human PH20 that has a sequence of amino acids set forth in SEQ ID NO:1 that contains a C-terminal truncation after amino acid position 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 of the sequence of amino acids set forth in SEQ ID NO:1, or is a variant thereof that exhibits at least 85% sequence identity to a sequence of amino acids that contains a C-terminal truncation after amino acid position 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 of the sequence of amino acids set forth in SEQ ID NO:1 and exhibits hyaluronidase activity.

In other examples provide herein, the PH20 in the provided compositions, combinations and containers has at least 86%, 87%, 88%, 89%, 90%, 91%, 92%, 93%, 94%, 95%, 96%, 97%, 98% or 99% sequence identity to a sequence of amino acids that contains that contains a C-terminal truncation after amino acid position 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 of the sequence of amino acids set forth in SEQ ID NO:1 and exhibits hyaluronidase activity. In a specific example, the PH20 has a sequence of amino acids that contains at least amino acids 36-464 of SEQ ID NO:1, or has a sequence of amino acids that has at least 85% sequence identity to a sequence of amino acids that contains at least amino acids 36-464 of SEQ ID NO:1 and exhibits hyaluronidase activity. In other examples, the PH20 has at least 86%, 87%, 88%, 89%, 90%, 91%, 92%, 93%, 94%, 95%, 96%, 97%, 98% or 99% sequence identity to a sequence of amino acids that contains at least amino acids 36-464 of SEQ ID NO:1 and exhibits hyaluronidase activity. In one embodiment, the PH20 is truncated at an amino acid corresponding to 477, 478, 479, 480, 481, 482, 483, 484, 485, 486, 487, 488, 489, 490, 491, 492, 494, 494, 495, 496, 497, 498, 499 or 500 of the sequence of amino acids set forth in SEQ ID NO:1 and exhibits hyaluronidase activity.

In another example, hyaluronan-degrading enzyme in the provided compositions, combinations and containers is a C-terminal truncated PH20 that contains a sequence of amino set forth in any of SEQ ID NOS: 4-9. In some instances, the hyaluronan-degrading enzyme is modified with a polymer, such as a polyethylene glycol (PEG).

Also provided herein are kits containing a composition provided herein and optionally instructions for use. Also provided herein are kits containing a combination provided herein and optionally instructions for use.

Also provided herein are combinations containing any container provided herein and one or more additional containers containing another pharmacologically effective agent selected from among carbamates, anti-muscarinics, cholinesterase reactivators and anti-convulsives. In some examples, the pharmacologically effective agents are provided in separate containers. In one example, the additional container contains at least two pharmacologically effective agents and the agents are provided as separate compositions separated from each other. In another example, the pharmacologically effective agents are provided in the same container in the same composition. In specific examples, the additional container contains a carbamate, anti-muscarinic and cholinesterase reactivator. In some examples, the additional container(s) is a syringe. In some combinations provided herein, the additional agents are in solution or suspension and the total volume of solution or suspension in the additional container(s) is from or is from about 0.1 to 10 mL, 0.1 to 5 mL, 0.1 to 3 mL, 0.1 to 1 mL, 1 to 10 mL, 3 to 10 mL, 5 to 10 mL, 1 to 5 mL, 1 to 3 mL or 3 to 5 mL.

Also provided herein are kits containing any container provided herein, a device for administration and, optionally instructions for administration. Also provided herein are kits containing any combination provided herein, a device for administration and, optionally instructions for administration.

Provided herein are methods for preventing organophosphorus poisoning by administering a composition or combination provided herein. Also provided herein are methods for treating organophosphorus poisoning by administering a composition or combination provided herein. Provided herein are methods for preventing organophosphorus poisoning wherein the method involves administering to a subject an organophosphorus bioscavenger and a hyaluronan-degrading enzyme. Also provided herein are methods for treating organophosphorus poisoning, wherein the method involves administering an organophosphorus bioscavenger and a hyaluronan-degrading enzyme.

In some examples of the methods provided herein, the organophosphorus bioscavenger and hyaluronan-degrading enzyme are administered parenterally. In other examples of the methods provided herein, the organophosphorus bioscavenger and hyaluronan-degrading enzyme are administered by subcutaneous administration, intramuscular administration, intralesional administration or intradermal administration. In an exemplary embodiment, the organophosphorus bioscavenger and hyaluronan-degrading enzyme are administered by intramuscular administration.

In some examples of the methods provided herein, the organophosphorus bioscavenger is administered in an amount between or about between 1 μg to 100 mg, 1 μg to 50 mg, 1 μg to 10 mg, 1 μg to 1 mg, 1 μg to 500 μg, 1 μg to 250 μg, 1 μg to 100 μg, 50 μg to 50 mg, 50 μg to 25 mg, 50 μg to 10 mg, 50 μg to 1 mg, 50 μg to 500 μg, 50 μg to 250 μg, 100 μg to 50 mg, 100 μg to 10 mg, 100 μg to 1 mg, 100 μg to 500 μg, 100 μg to 250 μg, 250 μg to 50 mg, 250 μg to 25 mg, 250 μg to 10 mg, 250 μg to 1 mg, 250 μg to 500 μg, 500 μg to 100 mg, 500 μg to 50 mg, 500 μg to 25 mg, 500 μg to 10 mg, 500 μg to 1 mg, 1 mg to 500 mg, 1 mg to 250 mg, 1 mg to 100 mg, 50 mg to 1000 mg, 250 mg to 1000 mg, 250 mg to 750 mg, 250 mg to 500 mg, 500 mg to 1000 mg, 500 mg to 750 mg, or is at least or is about at least 1 μg, 10 μg, 50 μg, 100 μg, 250 μg, 500 μg, 1 mg, 10 mg, 50 mg, 100 mg, 250 mg, 500 mg, 750 mg or 1000 mg when a single dosage is administered.

In other examples of the methods provided herein, the hyaluronan-degrading enzyme is administered at a dosage between or about between 10,000 Units to 6,000,000 Units, for example, between 10,000 U to 150,000 U, 10,000 U to 100,000 U, 10,000 U to 50,000 U, 50,000 U to 200,000 U, 50,000 U to 150,000 U, 50,000 U to 100,000 U, 10,000 U to 1,000,000 U, 50,000 U to 1,000,000 U, 500,000 U to 6,000,000 U, 500,000 U to 4,000,000 U, 500,000 U to 2,000,000 U, 500,000 U to 1,000,000 U, 1,000,000 U to 6,000,000 U, 1,000,000 U to 5,000,000 U, 1,000,000 U to 4,000,000 U, 1,000,000 U to 3,000,000 U, 1,000,000 U to 2,000,000 U, 2,000,000 U to 6,000,000 U, 2,000,000 U to 5,000,000 U, 2,000,000 U to 4,000,000 U, 2,000,000 U to 3,000,000 U, 3,000,000 U to 6,000,000 U, 4,000,000 U to 6,000,000 U, 5,000,000 U to 6,000,000 U, or is at least or least about or is 10,000 U, 20,000 U, 30,000 U, 40,000 U, 50,000 U, 60,000 U, 70,000 U, 80,000 U, 90,000 U, 100,000 U, 110,000 U, 120,000 U, 130,000 U, 140,000 U, 150,000 U, 160,000 U, 170,000 U, 180,000 U, 190,000 U, 200,000 U, 300,000 U, 400,000 U, 500,000 U, 600,000 U, 700,000 U, 800,000 U, 900,000 U, 1,000,000 U, 1,500,000 U, 2,000,000 U, 2,500,000 U, 3,000,000 U, 3,500,000 U, 4,000,000 U, 5,000,000 U, 6,000,000 U or more, per single dosage.

In some examples of the methods provided herein, the composition is administered between at or about 6 to 48 hours, 6 to 36 hours, 6 to 24 hours, 12 to 48 hours, 12 to 36 hours, 12 to 24 hours, 24 to 48 hours, 24 to 36 hours, or at or about 6, 7, 8, 9, 10, 11, 12, 13, 14, 15, 16, 17, 18, 24, 30, 36, 42 or 48 hours before exposure to the organophosphorus compound.

Any of the methods provided herein can further contain a step of administering another pharmaceutical agent selected from among carbamates, anti-muscarinics, cholinesterase reactivators and anti-convulsives. In any of the methods provided herein, the compositions can be administered by percutaneous administration. For example, the compositions are administered by subcutaneous or intramuscular administration. In an exemplary embodiment, the compositions are administered by intramuscular administration.

Provided herein are uses and pharmaceutical compositions containing a provided composition or combination for use in treating or preventing organophosphorus poisoning. Also provided herein are uses or pharmaceutical compositions containing an organophosphorus bioscavenger and a hyaluronan-degrading enzyme for use in treating or preventing organophosphorus poisoning. Provided herein are uses containing a provided combination or composition for formulation of a medicament for use in treating or preventing organophosphorus poisoning.

In such uses and compositions, the compositions and combinations of an organophosphorus bioscavenger and a hyaluronan-degrading enzyme can be formulated for percutaneous administration. For example, the uses or compositions are formulated for subcutaneous administration, intramuscular administration, intralesional administration or intradermal administration. In one example, the uses or compositions are formulated to subcutaneous or intramuscular administration. In an exemplary embodiment, the uses or compositions are formulated for intramuscular administration. In some examples of the uses and compositions provided herein, the hyaluronan-degrading enzyme is formulated in the same composition as the organophosphorus bioscavenger or in a separate composition.

In any of the uses or compositions provided herein, the organophosphorus bioscavenger can be present in the composition an amount between or between about 1 μg to 100 mg, 1 μg to 50 mg, 1 μg to 10 mg, 1 μg to 1 mg, 1 μg to 500 μg, 1 μg to 250 μg, 1 μg to 100 μg, 50 μg to 50 mg, 50 μg to 25 mg, 50 μg to 10 mg, 50 μs to 1 mg, 50 μg to 500 μg, 50 μg to 250 μg, 100 μg to 50 mg, 100 μg to 10 mg, 100 μg to 1 mg, 100 μg to 500 μg, 100 μg to 250 μg, 250 μg to 50 mg, 250 μg to 25 mg, 250 μg to 10 mg, 250 μg to 1 mg, 250 μg to 500 μg, 500 μg to 100 mg, 500 μg to 50 mg, 500 μg to 25 mg, 500 μg to 10 mg, 500 μg to 1 mg, 1 mg to 500 mg, 1 mg to 250 mg, 1 mg to 100 mg, 50 mg to 1000 mg, 250 mg to 1000 mg, 250 mg to 750 mg, 250 mg to 500 mg, 500 mg to 1000 mg, 500 mg to 750 mg, or is at least or is about at least 1 μg, 10 μg, 50 μg, 100 μg, 250 μg, 500 μg, 1 mg, 10 mg, 50 mg, 100 mg, 250 mg, 500 mg, 750 mg or 1000 mg per single dosage.

In any of the uses or compositions provided herein, the hyaluronan-degrading enzyme can be present in the composition in an amount between or about between 10,000 Units to 6,000,000 Units, for example, between 10,000 U to 150,000 U, 10,000 U to 100,000 U, 10,000 U to 50,000 U, 50,000 U to 200,000 U, 50,000 U to 150,000 U, 50,000 U to 100,000 U, 10,000 U to 1,000,000 U, 50,000 U to 1,000,000 U, 500,000 U to 6,000,000 U, 500,000 U to 4,000,000 U, 500,000 U to 2,000,000 U, 500,000 U to 1,000,000 U, 1,000,000 U to 6,000,000 U, 1,000,000 U to 5,000,000 U, 1,000,000 U to 4,000,000 U, 1,000,000 U to 3,000,000 U, 1,000,000 U to 2,000,000 U, 2,000,000 U to 6,000,000 U, 2,000,000 U to 5,000,000 U, 2,000,000 U to 4,000,000 U, 2,000,000 U to 3,000,000 U, 3,000,000 U to 6,000,000 U, 4,000,000 U to 6,000,000 U, 5,000,000 U to 6,000,000 U, or is at least or least about or is 10,000 U, 20,000 U, 30,000 U, 40,000 U, 50,000 U, 60,000 U, 70,000 U, 80,000 U, 90,000 U, 100,000 U, 110,000 U, 120,000 U, 130,000 U, 140,000 U, 150,000 U, 160,000 U, 170,000 U, 180,000 U, 190,000 U, 200,000 U, 300,000 U, 400,000 U, 500,000 U, 600,000 U, 700,000 U, 800,000 U, 900,000 U, 1,000,000 U, 1,500,000 U, 2,000,000 U, 2,500,000 U, 3,000,000 U, 3,500,000 U, 4,000,000 U, 5,000,000 U, 6,000,000 U or more, per single dosage.

In the methods and uses and compositions provided herein, the OP bioscavenger can be an esterase, cholinesterase, paraoxonase, aryldialkylphosphatase or diisopropylfluorophosphatase. For example, the OP bioscavenger is selected from among acetylcholinesterase (AChE), butyrylcholinesterase (BChE), prolidase, organophosphate acid anhydrolase (OPAA), phosphotriesterase, aryldialkylphosphatase, organophosphorus hydrolase (OPH), parathion hydrolase, diisopropylfluorophosphatase (DFPase), organophosphorus acid anhydrase, sarinase and paraoxonase (PON). In one embodiment, the OP bioscavenger is an active portion or a variant of an acetylcholinesterase (AChE), butyrylcholinesterase (BChE), prolidase, organophosphate acid anhydrolase (OPAA), phosphotriesterase, aryldialkylphosphatase, organophosphorus hydrolase (OPH), parathion hydrolase, diisopropylfluorophosphatase (DFPase), organophosphorus acid anhydrase, sarinase or paraoxonase (PON) that exhibits at least 80%, 85%, 90%, 95%, or more OP binding or inactivating activity.

In some examples of the methods and uses and compositions provided herein, the organophosphorus bioscavenger has a sequence of amino acids set forth in any of SEQ ID NOS: 214-256 and 258-301. In other examples, the OP bioscavenger is an active portion thereof or a variant thereof that exhibits at least 80%, 85%, 90%, 95%, or more sequence identity to any of SEQ ID NOS: 214-256 and 258-301. In such examples, the active portion or variant exhibits OP binding or inactivating activity. For example, the active portion or variant can exhibit at least 40%, 50%, 60%, 70%, 80%, 90%, or more activity compared to the corresponding organophosphorus bioscavenger set forth in any of SEQ ID NOS: 214-256 and 258-301. In some examples of the methods provided herein, the active portion of an OP bioscavenger is a truncated fragment lacking a tryptophan amphiphilic tetramerization (WAT) domain.

In some examples of the methods and uses and compositions, the OP bioscavenger is a variant that has a sequence of amino acids that contains an amino acid modification compared to organophosphorus bioscavenger that has a sequence of amino acids set forth in any of SEQ ID NOS: 214-256 and 258-301. The amino acid modification can be an amino acid replacement or substitution, deletion or addition. In some examples, the variant exhibits increased catalytic activity or other inhibitory activity compared to the organophosphorus bioscavenger not containing the amino acid modification.

In some embodiments, the organophosphorus bioscavenger in the provided methods and uses and compositions is a cholinesterase. For example, the OP bioscavenger is a cholinesterase that is an acetylcholinesterase or butyrylcholinesterase. The cholinesterase can be a monomer, dimer or a tetramer. In an exemplary embodiment, the organophosphorus bioscavenger is butyrylcholinesterase. For example, the OP bioscavenger is a butyrylcholinesterase that has a sequence of amino acids set forth in SEQ ID NO:236, or is an active portion thereof or is a variant thereof that exhibits at least 85% sequence identity to a sequence of amino acids set forth in SEQ ID NO:236. For example, the butyrylcholinesterase has at least 86%, 87%, 88%, 89%, 90%, 91%, 92%, 93%, 94%, 95%, 96%, 97%, 98% or 99% sequence identity to a sequence of amino acids set forth in SEQ ID NO:236 and exhibits OP binding or inactivating activity. In some examples provided herein, the organophosphorus bioscavenger is a cholinesterase and the active portion is a truncated fragment lacking a tryptophan amphiphilic tetramerization (WAT) domain.

In some examples of the methods and uses or compositions provided herein, the OP bioscavenger exhibits a half-life of at least 30 hours, 40 hours, 50 hours, 60 hours, 70 hours, 80 hours, 90 hours or 100 hours; or between or about between 30 hours to 130 hours, 40 hours to 100 hours, 40 hours to 80 hours, 40 hours to 60 hours.

In some examples of the provided methods and uses or compositions, the organophosphorus bioscavenger can be modified with a polymer. For example, the OP bioscavenger can be modified with a polymer that is a polyethylene glycol (PEG). In other examples, the OP bioscavenger can be linked directly or indirectly via a linker to an immunoglobulin, immunoglobulin domain, albumin, transferrin, or transferrin receptor protein. The linker can be a chemical linker or a polypeptide linker, for example, a peptide, polypeptide or an amino acid.

In the methods and uses or compositions provided herein, the hyaluronan-degrading enzyme can be a hyaluronidase or a chondroitinase, or a variant or a truncated form thereof that exhibits hyaluronan-degrading activity. In some examples, the hyaluronan-degrading enzyme is a hyaluronidase that is active at neutral pH. For example, the hyaluronan-degrading enzyme is a Hyal1, Hyal2, Hyal4 or PH20 hyaluronidase, or a variant or a truncated form thereof that exhibits hyaluronidase activity. In a specific example, the hyaluronan-degrading enzyme is a hyaluronidase that is a PH20 or a variant or a truncated form thereof that exhibits hyaluronidase activity. For example, the hyaluronidase is a PH20 that is a non-human or a human PH20 or variant or a truncated form thereof that exhibits hyaluronidase activity. Such PH20 hyaluronidases include human, monkey, bovine, ovine, rat, mouse or guinea pig PH20, or a variant or a truncated form thereof that exhibits hyaluronidase activity.

In some examples of the provided methods and uses or compositions, the hyaluronan-degrading enzyme lacks all or a portion of a glycophosphatidylinositol (GPI) anchor or is not membrane-associated when expressed from a cell. For example, the hyaluronan-degrading enzyme contains C-terminal truncations of one or more amino acid residues to remove all or part of a GPI anchor. In a specific example, the hyaluronan-degrading enzyme is a truncated human PH20 that has a sequence of amino acids set forth in SEQ ID NO:1 that contains a C-terminal truncation after amino acid position 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 of the sequence of amino acids set forth in SEQ ID NO:1, or is a variant thereof that exhibits at least 85% sequence identity to a sequence of amino acids that contains a C-terminal truncation after amino acid position 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 of the sequence of amino acids set forth in SEQ ID NO:1 and exhibits hyaluronidase activity.

In other examples of the methods and uses or compositions provided herein, the PH20 has at least 86%, 87%, 88%, 89%, 90%, 91%, 92%, 93%, 94%, 95%, 96%, 97%, 98% or 99% sequence identity to a sequence of amino acids that contains that contains a C-terminal truncation after amino acid position 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 of the sequence of amino acids set forth in SEQ ID NO:1 and exhibits hyaluronidase activity. In a specific example, the PH20 has a sequence of amino acids that contains at least amino acids 36-464 of SEQ ID NO:1, or has a sequence of amino acids that has at least 85% sequence identity to a sequence of amino acids that contains at least amino acids 36-464 of SEQ ID NO:1 and exhibits hyaluronidase activity. In other examples, the PH20 has at least 86%, 87%, 88%, 89%, 90%, 91%, 92%, 93%, 94%, 95%, 96%, 97%, 98% or 99% sequence identity to a sequence of amino acids that contains at least amino acids 36-464 of SEQ ID NO:1 and exhibits hyaluronidase activity. In one embodiment, the PH20 is truncated at an amino acid corresponding to 477, 478, 479, 480, 481, 482, 483, 484, 485, 486, 487, 488, 489, 490, 491, 492, 494, 494, 495, 496, 497, 498, 499 or 500 of the sequence of amino acids set forth in SEQ ID NO:1 and exhibits hyaluronidase activity.

In another example of the methods and uses or compositions provided herein, hyaluronan-degrading enzyme in the provided compositions, combinations and containers is a C-terminal truncated PH20 that contains a sequence of amino set forth in any of SEQ ID NOS: 4-9. In some instances, the hyaluronan-degrading enzyme is modified with a polymer, such as a polyethylene glycol (PEG).

### DETAILED DESCRIPTION

A. Definitions

B. COMPOSITIONS AND COMBINATIONS OF ORGANOPHOSPHORUS BIOSCAVENGERS AND HYALURONAN-DEGRADING ENZYMES
1. Nerve Agent Poisoning
2. Treatments for Nerve Agent Poisoning
3. Cotherapy with Hyaluronan-Degrading Enzyme

C. ORGANOPHOSPHORUS BIOSCAVENGERS
1. Cholinesterases
   a. Acetylcholinesterases
   b. Butyrylcholinesterases
      rBChE
      PEG-RBChE
2. Other Organophosphorus Bioscavengers
   a. Aryldialkylphosphatases
      i. Paraoxonases
      ii. Organophosphorus hydrolases
      iii. Parathion hydrolases
   b. Dfisopropyl fluorophosphatases
      i. Organophosphate acid anhydrolases
3. Modified Organophosphorus Bioscavengers
   a. Polymer modified organophosphorus bioscavengers
      PEGylated organophosphorus bioscavengers
   b. Other modifications

D. HYALURONAN-DEGRADING ENZYMES
1. Hyaluronidases
   a. Mammalian-type hyaluronidases
      PH20
   b. Bacterial hyaluronidases
   c. Hyaluronidases from leeches, other parasites and crustaceans
2. Other hyaluronan-degrading enzymes
3. Soluble hyaluronan-degrading enzymes
   a. Soluble Human PH20
   b. rHuPH20
4. Glycosylation of hyaluronan-degrading enzymes
5. Modified (Polymer-Conjugated) Hyaluronan-degrading enzymes
   PEGylated soluble hyaluronan-degrading enzymes

E. PHARMACEUTICAL COMPOSITIONS AND FORMULATIONS, DOSAGES AND ADMINISTRATION
1. Dosages and Administration
   a. Organophosphorus Bioscavengers
   b. Hyaluronan-degrading Enzymes
2. Injectables, solutions and emulsions
   Lyophilized powders
3. Topical administration
4. Compositions for other routes of administration

F. METHODS OF PRODUCING NUCLEIC ACIDS ENCODING AN ORGANOPHOSPHORUS BIOSCAVENGER OR HYALURONAN-DEGRADING ENZYME AND POLYPEPTIDES THEREOF
1. Vectors and Cells
2. Expression
   a. Prokaryotic Cells
   b. Yeast Cells
   c. Insect Cells
   d. Mammalian Cells
      i. Generation of Transgenic Animals
   e. Plants
3. Purification Techniques
4. PEGylation

G. METHODS OF ASSESSING ACTIVITY
Pharmacokinetics and tolerability
Assays to assess hyaluronan activity
   a. Assays to assess the activity of a Hyaluronan-degrading Enzyme

H. THERAPEUTIC USES

I. COMBINATION THERAPIES

J. ARTICLES OF MANUFACTURE AND KITS

K. EXAMPLES

A. Definitions

Unless defined otherwise, all technical and scientific terms used herein have the same meaning as is commonly understood by one of skill in the art to which the invention(s) belong. All Patents, Pat. applications, published applications and publications, GENBANK sequences, websites and other published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it is understood that such identifiers can change and particular information on the interne can come and go, but equivalent information is known and can be readily accessed, such as by searching the interne and/or appropriate databases. Reference thereto evidences the availability and public dissemination of such information.

As used herein, “organophosphorus poisoning” or “OP poisoning” refers to deleterious or undesirable effects to a living creature exposed to an organophosphorus compound, such as an organophosphorus nerve agent or an organophosphorus pesticide.

As used herein, “organophosphorus compound,” “organophosphate compound” or “OP compound,” which are used interchangeably herein, refer to chemical compounds that contain a phosphoryl center, and further contain two or three ester linkages. In some aspects, the type of phosphoester bond and/or additional covalent bond at the phosphoryl center classifies an organophosphorus compound. In embodiments wherein the phosphorus is linked to an oxygen by a double bond (PdbdO), the OP compound is known as an “oxon OP compound” or “oxon organophosphorus compound.” In embodiments wherein the phosphorus is linked to a sulfur by a double bond (PdbdS), the OP compound is known as a “thion OP compound” or “thion organophosphorus compound.” Organophosphorus compounds include organophosphorus pesticides and organophosphorus nerve agents.

As used herein, “organophosphorus pesticide,” “organophosphate pesticide” or “OP pesticide” refers to an organophosphorus compound that can be used a pesticide or insecticide to destroy pests and insects. An organophosphorus pesticide can be any organophosphorus pesticide, including, but not limited to, acephate, azinphos-methyl, bensulide, cadusafos, chlorethoxyfos, chlorpyrifos, chlorpyrifos methyl, chlorthiophos, coumaphos, dialiflor, diazinon, diehlorvos (DDVP), dierotophos, dimethoate, dioxathion, disulfoton, ethion, ethoprop, ethyl parathion, fenamiphos, fenitrothion, fenthion, fonofos, isazophos methyl, isofenphos, malathion, methamidophos, methidathion, methyl parathion, mevinphos, monocrotophos, naled, oxydemeton methyl, phorate, phosalone, phosmet, phosphamidon, phostebupirim, pirimiphos methyl, profenofos, propetamphos, sulfotepp, sulprofos, temephos, terbufos, tetraehlorvinphos, tribufos (JDEF) and trichlorfon.

As used herein, “nerve agent” refers to a chemical compound that disrupts the functioning of the nervous system of an organism, such as by inhibiting the actions of the enzyme acetylcholinesterase. Nerve agents are generally prepared by chemical synthesis or extraction from natural sources, and can cause deleterious or undesirable effects to a living creature if inhaled, absorbed, ingested, or otherwise encountered. A nerve agent can be any organophosphorus nerve agent, including, but not limited to G-type nerve agents and V-type nerve agents. Exemplary organophosphorus nerve agents include tabun (GA), sarin (GB), soman (GD), cyclosarin (GF), VX, Russian VX (VR), classified non-traditional nerve agents (NTAs) and diisopropylfluorophosphate (DFP).

As used herein, “cholinergic toxicity” refers to toxicity achieved by nerve agent inhibition of acetylcholinesterase, accumulation of the neurotransmitter acetylcholine, and concomitant affects on the parasympathetic, sympathetic, motor, and central nervous systems. Cholinergic toxicity can result in myopathy, psychosis, general paralysis and death. Symptoms of exposure include twitching, trembling, hypersecretion, paralyzed breathing, convulsions, and ultimately death. Cholinergic toxicity can be monitored by measuring circulating cholinesterase levels in the plasma. Generally lethality occurs only when cholinesterase activity falls below 20% of normal levels due to binding by nerve agents.

As used herein, “organophosphorus exposure associated damage” refers to short term (e.g., minutes to several hours post-exposure) and long term (e.g., one week up to several years post-exposure) damage, for example, due to cholinergic toxicity, to physiological function (e.g., motor and cognitive functions). Organophosphorus exposure associated damage can be manifested by the following clinical symptoms including, but not limited to, headache, diffuse muscle cramping, weakness, excessive secretions, nausea, vomiting and diarrhea. The condition can progress to seizure, coma, paralysis, respiratory failure, delayed neuropathy, muscle weakness, tremor, convulsions, permanent brain dysmorphology, social/behavioral deficits and general cholinergic crisis (which can be manifested for instance by exacerbated inflammation and low blood cell count). Extreme cases can lead to death of the poisoned subjects.

As used herein, an “organophosphorus bioscavenger” or “organophosphate bioscavenger” or “OP bioscavenger” is an enzyme capable of binding to or hydrolyzing an organophosphorus compound, including organophosphorus pesticides and organophosphorus nerve agents. Organophosphorus bioscavengers include, but are not limited to, cholinesterases, aryldialkylphosphatases, organophosphate hydrolases (OPH), carboxylesterases, triesterases, phosphotriesterases, arylesterases, paraoxonases (PON), organophosphate acid anhydrases and diisopropylfluorophosphatases (DFPases). Organophosphorus bioscavengers can be stoichiometric organophosphorus bioscavengers or catalytic organophosphorus bioscavengers.

As used herein, a “stoichiometric organophosphorus bioscavenger” or “stoichiometric OP bioscavenger” refers to an enzyme that binds to an organophosphorus compound in a stoichiometric 1:1 ratio. Stoichiometric OP bioscavengers include, but are not limited, to cholinesterases, such as acetylcholinesterases and butyrylcholinesterases.

As used herein, a “catalytic organophosphorus bioscavenger” or “catalytic OP bioscavenger” refers to an enzyme that hydrolyzes an organophosphorus compound. Catalytic OP bioscavengers include, but are not limited to, aryldialkylphosphatases, organophosphate hydrolases (OPH), carboxylesterases, triesterases, phosphotriesterases, arylesterases, paraoxonases (PON), organophosphate acid anhydrases and diisopropylfluorophosphatases (DFPases).

As used herein, “organophosphorus inactivating activity” or “OP inactivating activity” refers to the ability of an organophosphorus bioscavenger to inactivate an OP compound. OP bioscavengers can inactivate an OP compound by stoichiometrically binding to the OP compound or by hydrolyzing the OP compound. Hence, inactivating activity can be a function of the binding activity or the hydrolytic activity of the OP bioscavenger to the OP compound. Thus inactivating activity means that the organophosphorus bioscavenger exhibits binding activity and/or hydrolytic activity for an organophosphorus compound. An activity of an organophosphorus compound is inactivated if the activity is reduced in the presence of an OP bioscavenger compared to its absence by at least 20%, 30%, 40%, 50%, 60%, 70%, 80%, 90% or more.

As used herein, “binding activity” with reference to an OP bioscavenger for an OP compound refers to the ability of the OP bioscavenger to bind to the OP compound. For example, OP bioscavengers covalently bind to an OP compound by attack of the active site serine on the phosphorus of the OP compound.

As used herein, “hydrolytic activity” refers to the ability of an OP bioscavenger to catalyze the hydrolysis of an OP compound. For example, the OP bioscavenger hydrolyzes the organophosphorus compound resulting in its breakdown. Assays to assess hydrolytic or enzymatic activity of OP bioscavengers are described herein.

As used herein, a “cholinesterase” refers to a family of enzymes involved in nerve impulse transmissions. Cholinesterases catalyze the hydrolysis of acetylcholine at cholinergic synapses. Cholinesterases include but are not limited to acetylcholinesterases and butyrylcholinesterases. Exemplary cholinesterases are acetylcholinesterases and butyrylcholinesterases.

As used herein, “acetylcholinesterase” or “AChE” refers to enzymes or polypeptides capable of hydrolyzing acetyl esters, including acetylcholine, and whose catalytic activity is inhibited by the chemical inhibitor BW 284C51. Acetylcholinesterases include, but are not limited to, plasma derived or recombinant acetylcholinesterases. Acetylcholinesterases include any of non-human origin including, but not limited to, rat, mouse, cat, chicken, rabbit, cow, pacific electric ray and fruit fly acetylcholinesterases. Exemplary non-human acetylcholinesterase include, acetylcholinesterases from rat (SEQ ID NO:220), mouse (SEQ ID NO:222), cat (SEQ ID NO:228), chicken (SEQ ID NO:226), rabbit (SEQ ID NO:224), cow (bovine; SEQ ID NO:230), pacific electric ray (SEQ ID NO:232) and fruit fly (SEQ ID NO:234). Acetylcholinesterases also include acetylcholinesterase of human origin. Exemplary human acetylcholinesterases include human AChE set forth in SEQ ID NO:215. Acetylcholinesterases can be in any form, including, but not limited to, monomer, dimer and tetramer forms.

Reference to acetylcholinesterases includes precursor acetylcholinesterases and mature acetylcholinesterases (such as those in which a signal sequence has been removed), truncated forms thereof that have activity, and includes allelic variant and species variants, variants encoded by splice variants, and other variants, including polypeptides that have at least 40%, 45%, 50%, 55%, 65%, 70%, 75%, 80%, 85%, 90%, 95%, 96%, 97%, 98%, 99% or more sequence identity to the polypeptides set forth in SEQ ID NOS: 214-234 and 294-296. Variants exhibit OP inactivating activity. Acetylcholinesterases also include those that contain chemical or posttranslational modifications and those that do not contain chemical or posttranslational modifications. Such modifications include, but are not limited to, PEGylation, albumination, glycosylation, farnesylation, carboxylation, hydroxylation, phosphorylation, and other polypeptide modifications known in the art. A truncated acetylcholinesterase is any C-terminal shortened form thereof, particularly forms that are truncated and have OP inactivating activity.

As used herein, “butyrylcholinesterase” or “BChE” refers to enzymes or polypeptides capable of hydrolyzing acetylcholine and butyrylcholine, and whose catalytic activity is inhibited by the chemical inhibitor tetraisopropyl-pyrophosphoramide (iso-OMPA). Butyrylcholinesterase include, but are not limited to, plasma derived or recombinant butyrylcholinesterases. Butyrylcholinesterases include any of non-human origin including, but not limited to, rat, mouse, cat, horse, chicken, pig, rabbit, cow, sheep, rhesus monkey and Bengal tiger butyrylcholinesterases. Exemplary non-human butyrylcholinesterase include, butyrylcholinesterases from rat (SEQ ID NO:240), mouse (SEQ ID NO:242), cat (SEQ ID NO:244), horse (SEQ ID NO:245), chicken (SEQ ID NO:247), pig (SEQ ID NO:248), rabbit (SEQ ID NO:250), cow (bovine; SEQ ID NO:252), sheep (Ovis aries; SEQ ID NO:253), rhesus monkey (SEQ ID NO:254) and Bengal tiger (SEQ ID NO:256). Butyrylcholinesterases also include butyrylcholinesterases of human origin. Exemplary human butyrylcholinesterases include human BChE set forth in SEQ ID NO:236. Butyrylcholinesterases can be in any form, including, but not limited to, monomer, dimer and tetramer forms. An exemplary recombinant human BChE is rBChE expressed in the milk of transgenic goats.

Reference to butyrylcholinesterases includes precursor butyrylcholinesterases and mature butyrylcholinesterases (such as those in which a signal sequence has been removed), truncated forms thereof that have activity, and includes allelic variant and species variants, variants encoded by splice variants, and other variants, including polypeptides that have at least 40%, 45%, 50%, 55%, 65%, 70%, 75%, 80%, 85%, 90%, 95%, 96%, 97%, 98%, 99% or more sequence identity to the polypeptides set forth in SEQ ID NOS: 235-256 and 291-293. Variants exhibit OP inactivating activity. Butyrylcholinesterases also include those that contain chemical or posttranslational modifications and those that do not contain chemical or posttranslational modifications. Such modifications include, but are not limited to, PEGylation, albumination, glycosylation, farnesylation, carboxylation, hydroxylation, phosphorylation, and other polypeptide modifications known in the art. An exemplary PEGylated form of rBChE is PEG-rBChE (Protexia®) which is expressed in the milk of transgenic goats and further modified by attachment of polyethylene glycol (PEG) polymers. A truncated butyrylcholinesterase is any C-terminal shortened form thereof, particularly forms that are truncated and have OP inactivating activity.

As used herein, “wild-type” or “native” with reference to OP bioscavengers, such as butyrylcholinesterase, refers to a butyrylcholinesterase polypeptide encoded by a native or naturally occurring butyrylcholinesterase gene, including allelic variants, that is present in an organism, including a plant, in nature.

As used herein, “aryldialkylphosphatase” refers to naturally occurring or recombinant enzymes that inactivate or hydrolyze organophosphorus compounds. Aryldialkylphosphatases (EC 3.1.8.1) are class of metal-dependent OP-hydrolases that are capable of hydrolyzing a broad range of organophosphorus compounds. Aryldialkylphosphatases require a binuclear metal, such as Zn²⁺, Mn²⁺, Co²⁺ or Cd²⁺, at their active site for enzymatic activity. Aryldialkylphosphatases include, but are not limited to, phosphotriesterases or OP hydrolases (PTE or OPH), paraoxon hydrolases or paraoxonases, parathion hydrolases (PH), OpdA, carboxylesterases, triesterases, phosphotriesterases and arylesterases. Aryldialkylphosphatases include, but are not limited to, organophosphorus hydrolases from Pseudomonas diminuata MG, Flavobacterium sp., Plesiomonas sp. strain M6, Streptomyces lividans and Agrobacterium radiobacter; parathion hydrolases from Burkholderia sp. JBA3, Pseudomonas diminuta MG, Brevundiomonas diminuta, Flavobacterium sp. strain ATCC 27552, and Sulfolobus acidocaldarius; and methyl parathion hydrolases (MPH) from Bacillus subtilis WB800 and Plesiomonas sp. strain M6. Exemplary aryldiakylphosphatases include, but are not limited to, aryldialkylphosphatases set forth in any of SEQ ID NOS:277-285, 297-298 and 300.

Reference to aryldialkylphosphatases includes truncated forms thereof that have activity, and includes allelic variant and species variants, variants encoded by splice variants, and other variants, including polypeptides that have at least 40%, 45%, 50%, 55%, 65%, 70%, 75%, 80%, 85%, 90%, 95%, 96%, 97%, 98%, 99% or more sequence identity to the polypeptides set forth in SEQ ID NOS: 277-285, 297-298 and 300. Variants exhibit OP inactivating activity.

As used herein, “paraoxonase” refers to naturally occurring or recombinant enzymes that inactivate or hydrolyze organophosphorus compounds. Paraoxonases include, but are not limited to, native or recombinant paraoxonases. Non-human paraoxonases include, but are not limited to, paraoxonases from rabbit, mouse, rat, pig, cow, chicken, turkey and dog. Exemplary paraoxonases include, but are not limited to, human paraoxonases, including PON1 (SEQ ID NO:258), PON2 (SEQ ID NO:264), and PON3 (SEQ ID NO:271); rabbit paraoxonases, including PON1 (SEQ ID NO:259) and PON3 (SEQ ID NO:274); mouse paraoxonases, including PON1 (SEQ ID NO:260), PON2 (SEQ ID NO:265), and PON3 (SEQ ID NO:272); rat paraoxonases, including PON1 (SEQ ID NO:261), PON2 (SEQ ID NO:270), and PON3 (SEQ ID NO:273); pig paraoxonases, including PON1 (SEQ ID NO:262) and PON3 (SEQ ID NO:276); cow paraoxonases, including PON1 (SEQ ID NO:263), PON2 (SEQ ID NO:269), PON3 (SEQ ID NO:275); chicken PON2 (SEQ ID NO:266); turkey PON2 (SEQ ID NO:267); dog PON2 (SEQ ID NO:268). Paraoxonases also include human paraoxonases, including, but not limited, to PON1 (SEQ ID NO:258), PON2 (SEQ ID NO:264), and PON3 (SEQ ID NO:271). An exemplary human paraoxonase is PON1 set forth in SEQ ID NO:258.

Reference to paraoxonases includes truncated forms thereof that have activity, and includes allelic variant and species variants, variants encoded by splice variants, and other variants, including polypeptides that have at least 40%, 45%, 50%, 55%, 65%, 70%, 75%, 80%, 85%, 90%, 95%, 96%, 97%, 98%, 99% or more sequence identity to the polypeptides set forth in SEQ ID NOS: 258-276. Variants exhibit OP inactivating activity. Paraoxonases also include those that contain chemical or posttranslational modifications and those that do not contain chemical or posttranslational modifications. Such modifications include, but are not limited to, PEGylation, albumination, glycosylation, farnesylation, carboxylation, hydroxylation, phosphorylation, and other polypeptide modifications known in the art

As used herein, “diisopropylfluorophosphatase” refers to naturally occurring or recombinant enzymes that inactivate or hydrolyze organophosphorus compounds. Diisopropylfluorophosphatases include, but are not limited to, diisopropylfluorophosphatases from Loligo vulgaris, Alteromonas sp., Pseudoalteromonas haloplanktis, Marinomonas mediterranea, Aplysia californica, Octopus vulgaris and rat senescence marker protein 30; and organophosphate acid anhydrolases from Mycobacterium sp, Amycolatopsis mediterranei, Streptomyces coelicolor, Streptomyces sp AA4, Streptomyces lividans TK24, Streptomyces sviceus and Streptomyces griseoaurantiacus M045. Exemplary diisopropylfluorophosphatases and organophosphate acid anhydrolases include, but are not limited to, diisopropylfluorophosphatases organophosphate acid anhydrolases set forth in any of SEQ ID NOS: 286-290, 299 and 301.

Reference to diisopropylfluorophosphatases includes truncated forms thereof that have activity, and includes allelic variant and species variants, variants encoded by splice variants, and other variants, including polypeptides that have at least 40%, 45%, 50%, 55%, 65%, 70%, 75%, 80%, 85%, 90%, 95%, 96%, 97%, 98%, 99% or more sequence identity to the polypeptides set forth in SEQ ID NOS: 286-290, 299 and 301. Variants exhibit OP inactivating activity.

As used herein, “carbamate” refers to any compound that is a carbamate inhibitor of cholinesterase. An exemplary carbamate is pyridostigmine bromide.

As used herein, “anti-muscarinic” refers to any compound that is a competitive antagonist to muscarinic receptors, including muscarinic acetylcholine receptors. An exemplary anti-muscarine is atropine.

As used herein, “cholinesterase reactivator” refers to any compound that releases a bound organophosphorus compound from a cholinesterase. Cholinesterase reactivators include choline-reactivating oximes, including, pyridinium and bispyridinium aldoximes, including, but not limited to, pralidoxime, trimedoxime, obidoxime and HI-6, methoxime and diazepam.

As used herein, “anti-convulsive” refers to any compound that protects against or reverses seizures.

As used herein, a “hyaluronan-degrading enzyme” refers to an enzyme that catalyzes the cleavage of a hyaluronan polymer (also referred to as hyaluronic acid or HA) into smaller molecular weight fragments. Exemplary of hyaluronan-degrading enzymes are hyaluronidases, and particular chondroitinases and lyases that have the ability to depolymerize hyaluronan. Exemplary chondroitinases that are hyaluronan-degrading enzymes include, but are not limited to, chondroitin ABC lyase (also known as chondroitinase ABC), chondroitin AC lyase (also known as chondroitin sulfate lyase or chondroitin sulfate eliminase) and chondroitin C lyase. Chondroitin ABC lyase contains two enzymes, chondroitin-sulfate-ABC endolyase (EC 4.2.2.20) and chondroitin-sulfate-ABC exolyase (EC 4.2.2.21). An exemplary chondroitin-sulfate-ABC endolyases and chondroitin-sulfate-ABC exolyases include, but are not limited to, those from Proteus vulgaris and Flavobacterium heparinum (the Proteus vulgaris chondroitin-sulfate-ABC endolyase is set forth in SEQ ID NO:98; Sato et al. (1994) Appl. Microbiol. Biotechnol. 41(1):39-46). Exemplary chondroitin AC lyases from the bacteria include, but are not limited to, those from Flavobacterium heparinum, set forth in SEQ ID NO:99, Victivallis vadensis, set forth in SEQ ID NO:100, and Arthrobacter aurescens (Tkalec et al. (2000) Applied and Environmental Microbiology 66(1):29-35; Ernst et al. (1995) Critical Reviews in Biochemistry and Molecular Biology 30(5):387-444). Exemplary chondroitin C lyases from the bacteria include, but are not limited to, those from Streptococcus and Flavobacterium (Hibi et al. (1989) FEMS-Microbiol-Lett. 48(2): 121-4; Michelacci et al. (1976) J. Biol. Chem. 251:1154-8; Tsuda et al. (1999) Eur. J. Biochem. 262: 127-133).

As used herein, “hyaluronidase” refers to a class of hyaluronan-degrading enzymes. Hyaluronidases include bacterial hyaluronidases (EC 4.2.2.1 or EC 4.2.99.1), hyaluronidases from leeches, other parasites, and crustaceans (EC 3.2.1.36), and mammalian-type hyaluronidases (EC 3.2.1.35). Hyaluronidases include any of non-human origin including, but not limited to, murine, canine, feline, leporine, avian, bovine, ovine, porcine, equine, piscine, ranine, bacterial, and any from leeches, other parasites, and crustaceans. Exemplary non-human hyaluronidases include, hyaluronidases from cows (SEQ ID NOS:10, 11, 64 and BH55 (U.S. Pat. Nos. 5,747,027 and 5,827,721), yellow jacket wasp (SEQ ID NOS:12 and 13), honey bee (SEQ ID NO:14), white-face hornet (SEQ ID NO:15), paper wasp (SEQ ID NO:16), mouse (SEQ ID NOS:17-19, 32), pig (SEQ ID NOS:20-21), rat (SEQ ID NOS:22-24, 31), rabbit (SEQ ID NO:25), sheep (SEQ ID NOS:26, 27, 63 and 65), chimpanzee (SEQ ID NO:101), Rhesus monkey (SEQ ID NO:102), orangutan (SEQ ID NO:28), cynomolgus monkey (SEQ ID NO:29), guinea pig (SEQ ID NO:30), Arthrobacter sp. strain FB24 (SEQ ID NO:67), Bdellovibrio bacteriovorus (SEQ ID NO:68), Propionibacterium acnes (SEQ ID NO:69), Streptococcus agalactiae ((SEQ ID NO:70); 18RS21 (SEQ ID NO:71); serotype Ia (SEQ ID NO:72); serotype HI (SEQ ID NO:73), Staphylococcus aureus (strain COL (SEQ ID NO:74); strain MRSA252 (SEQ ID NOS:75 and 76); strain MSSA476 (SEQ ID NO:77); strain NCTC 8325 (SEQ ID NO:78); strain bovine RF122 (SEQ ID NOS:79 and 80); strain USA300 (SEQ ID NO:81), Streptococcus pneumoniae ((SEQ ID NO:82); strain ATCC BAA-255/R6 (SEQ ID NO:83); serotype 2, strain D39/NCTC 7466 (SEQ ID NO:84), Streptococcus pyogenes (serotype M1) (SEQ ID NO:85); serotype M2, strain MGAS10270 (SEQ ID NO:86); serotype M4, strain MGAS10750 (SEQ ID NO:87); serotype M6 (SEQ ID NO:88); serotype M12, strain MGAS2096 (SEQ ID NOS:89 and 90); serotype M12, strain MGAS9429 (SEQ ID NO:91); serotype M28 (SEQ ID NO:92); Streptococcus suis (SEQ ID NOS:93-95); Vibrio fischeri (strain ATCC 700601/ES114 (SEQ ID NO:96)), and the Streptomyces hyaluronolyticus hyaluronidase enzyme, which is specific for hyaluronic acid and does not cleave chondroitin or chondroitin sulfate (Ohya, T. and Kaneko, Y. (1970) Biochim. Biophys. Acta 198:607). Hyaluronidases also include those of human origin. Exemplary human hyaluronidases include HYAL1 (SEQ ID NO:36), HYAL2 (SEQ ID NO:37), HYAL3 (SEQ ID NO:38), HYAL4 (SEQ ID NO:39), and PH20 (SEQ ID NO:1). Also included amongst hyaluronidases are soluble hyaluronidases, including, ovine and bovine PH20, soluble human PH20 and soluble rHuPH20. Examples of commercially available bovine or ovine soluble hyaluronidases include Vitrase® (ovine hyaluronidase), Amphadase® (bovine hyaluronidase) and Hydase™ (bovine hyaluronidase).

As used herein, “purified bovine testicular hyaluronidase” refers to a bovine hyaluronidase purified from bovine testicular extracts (see U.S. Pat. Nos. 2,488,564, 2,488,565, 2,806,815, 2,808,362, 2,676,139, 2,795,529, 5,747,027 and 5,827,721). Examples of commercially available purified bovine testicular hyaluronidases include Amphadase® and Hydase™, and bovine hyaluronidases, including, but not limited to, those available from Sigma Aldrich, Abnova, EMD Chemicals, GenWay Biotech, Inc., Raybiotech, Inc., and Calzyme. Also included are recombinantly produced bovine hyaluronidases, such as but not limited to, those generated by expression of a nucleic acid molecule set forth in any of SEQ ID NOS:190-192.

As used herein, “purified ovine testicular hyaluronidase” refers to an ovine hyaluronidase purified from ovine testicular extracts (see U.S. Pat. Nos. 2,488,564, 2,488,565 and 2,806,815 and International PCT Publication No. WO2005/118799). Examples of commercially available purified ovine testicular extract include Vitrase®, and ovine hyaluronidases, including, but not limited to, those available from Sigma Aldrich, Cell Sciences, EMD Chemicals, GenWay Biotech, Inc., Mybiosource.com and Raybiotech, Inc. Also included are recombinantly produced ovine hyaluronidases, such as, but not limited to, those generated by expression of a nucleic acid molecule set forth in any of SEQ ID NOS:66 and 193-194.

As used herein, “PH20” refers to a type of hyaluronidase that occurs in sperm and is neutral-active. PH-20 occurs on the sperm surface, and in the lysosome-derived acrosome, where it is bound to the inner acrosomal membrane. PH20 includes those of any origin including, but not limited to, human, chimpanzee, Cynomolgus monkey, Rhesus monkey, murine, bovine, ovine, guinea pig, rabbit and rat origin. Exemplary PH20 polypeptides include those from human (SEQ ID NO:1), chimpanzee (SEQ ID NO:101), Rhesus monkey (SEQ ID NO:102), Cynomolgus monkey (SEQ ID NO:29), cow (e.g., SEQ ID NOS:11 and 64), mouse (SEQ ID NO:32), rat (SEQ ID NO:31), rabbit (SEQ ID NO:25), sheep (SEQ ID NOS:27, 63 and 65) and guinea pig (SEQ ID NO:30).

Reference to hyaluronan-degrading enzymes includes precursor hyaluronan-degrading enzyme polypeptides and mature hyaluronan-degrading enzyme polypeptides (such as those in which a signal sequence has been removed), truncated forms thereof that have activity, and includes allelic variants and species variants, variants encoded by splice variants, and other variants, including polypeptides that have at least 40%, 45%, 50%, 55%, 65%, 70%, 75%, 80%, 85%, 90%, 95%, 96%, 97%, 98%, 99% or more sequence identity to the precursor polypeptides set forth in SEQ ID NOS: 1 and 10-48, 63-65, 67-102, or the mature forms thereof (lacking the signal sequence). Variants exhibit hyaluronidase activity. For example, reference to hyaluronan-degrading enzyme also includes the human PH20 precursor polypeptide variants set forth in SEQ ID NOS:50-51. Hyaluronan-degrading enzymes also include those that contain chemical or posttranslational modifications and those that do not contain chemical or posttranslational modifications. Such modifications include, but are not limited to, PEGylation, albumination, glycosylation, farnesylation, carboxylation, hydroxylation, phosphorylation, and other polypeptide modifications known in the art. A truncated PH20 hyaluronidase is any C-terminal shortened form thereof, particularly forms that are truncated and neutral active when N-glycosylated.

As used herein, a “soluble PH20” refers to any form of PH20 that is soluble under physiologic conditions. A soluble PH20 can be identified, for example, by its partitioning into the aqueous phase of a Triton® X-114 solution at 37° C. (Bordier et al., (1981) J. Biol. Chem., 256:1604-7). Membrane-anchored PH20, such as lipid-anchored PH20, including GPI-anchored PH20, will partition into the detergent-rich phase, but will partition into the detergent-poor or aqueous phase following treatment with Phospholipase-C. Included among soluble PH20 are membrane-anchored PH20 in which one or more regions associated with anchoring of the PH20 to the membrane has been removed or modified, for example by truncation of C-terminal amino acid residues that for the GPI-anchor attachment signal sequence, where the soluble form retains hyaluronidase activity. Soluble PH20 also include recombinant soluble PH20 and those contained in or purified from natural sources, such as, for example, testes extracts from sheep or cows. Exemplary of such soluble PH20 is soluble human PH20.

As used herein, soluble human PH20 or sHuPH20 includes PH20 polypeptides lacking all or a portion of the glycosylphosphatidylinositol (GPI) anchor sequence at the C-terminus such that upon expression, the polypeptides are secreted into the medium. The secreted polypeptides are soluble under physiological conditions. Solubility can be assessed by any suitable method that demonstrates solubility under physiologic conditions. Exemplary of such methods is the Triton® X-114 assay, that assesses partitioning into the aqueous phase and that is described above and in the examples. In addition, a soluble human PH20 polypeptide is, if produced in CHO cells, such as CHO—S cells, a polypeptide that is expressed and is secreted into the cell culture medium. Soluble human PH20 polypeptides, however, are not limited to those produced in CHO cells, but can be produced in any cell or by any method, including recombinant expression and polypeptide synthesis. Reference to secretion in CHO cells is definitional. Hence, if a polypeptide could be expressed and secreted in CHO cells and is soluble, i.e. partitions into the aqueous phase when extracted with Triton® X-114, it is a soluble PH20 polypeptide whether or not it is so-produced. The precursor polypeptides for sHuPH20 polypeptides can include a signal sequence, such as a heterologous or non-heterologous (i.e. native) signal sequence. Exemplary of the precursors are those that include a signal sequence, such as the native 35 amino acid signal sequence at amino acid positions 1-35 (see, e.g., amino acids 1-35 of SEQ ID NO:1). It is understood that recombinantly expressed PH20 polypeptides and compositions thereof, including esPH20 and other forms, can include a plurality of species whose C-terminus exhibits heterogeneity. For example, compositions of recombinantly expressed esPH20 produced by expression of the polypeptide of SEQ ID NO:107, which encodes an esPH20 that has amino acids 36-497, can include forms with fewer amino acids, such as 36-496, 36-495.

As used herein, an “extended soluble PH20” or “esPH20” includes soluble PH20 polypeptides that contain residues up to the GPI anchor-attachment signal sequence and one or more contiguous residues from the GPI-anchor attachment signal sequence such that the esPH20 is soluble under physiological conditions. Solubility under physiological conditions can be determined by any method known to those of skill in the art. For example, it can be assessed by the Triton® X-114 assay described above and in the examples. In addition, as discussed above, a soluble PH20 is, if produced in CHO cells, such as CHO—S cells, a polypeptide that is expressed and is secreted into the cell culture medium. Soluble human PH20 polypeptides, however, are not limited to those produced in CHO cells, but can be produced in any cell or by any method, including recombinant expression and polypeptide synthesis. Reference to secretion in CHO cells is definitional. Hence, if a polypeptide could be expressed and secreted in CHO cells and is soluble, i.e. partitions into the aqueous phase when extracted with Triton® X-114, it is a soluble PH20 polypeptide whether or not it is so-produced. Human soluble esPH20 polypeptides include, in addition to residues 36-490, one or more contiguous amino acids from amino acid residue position 491 of SEQ ID NO:1, inclusive, such that the resulting polypeptide is soluble. Exemplary human esPH20 soluble polypeptides are those that have amino acids residues corresponding to amino acids 36-491, 36-492, 36-493, 36-494, 36-495, 36-496 and 36-497 of SEQ ID NO:1. Exemplary of these are those with an amino acid sequence set forth in any of SEQ ID NOS:151-154 and 185-187. Also included are allelic variants and other variants, such as any with 40%, 45%, 50%, 55%, 65%, 70%, 75%, 80%, 85%, 90%, 91%, 92%, 93%, 94%, 95%, 96%, 97%, 98%, 99% or greater sequence identity with the corresponding polypeptides of SEQ ID NOS:151-154 and 185-187 that retain neutral activity and are soluble. Reference to sequence identity can refer to variants with amino acid substitutions. Reference to “esPH20s” also include those that contain chemical or posttranslational modifications and those that do not contain chemical or posttranslational modifications. Such modifications include, but are not limited to, PEGylation, albumination, glycosylation, farnesylation, carboxylation, hydroxylation, phosphorylation, and other polypeptide modifications known in the art.

As used herein, “recombinant human PH20 (rHuPH20)” refers to a composition containing soluble form of human PH20 as recombinantly expressed and secreted in Chinese Hamster Ovary (CHO) cells. rHuPH20 is encoded by a nucleic acid molecule that includes the signal sequence and is set forth in SEQ ID NO:49. The nucleic acid encoding soluble rHuPH20 is expressed in CHO cells which secrete the mature polypeptide. As produced in the culture medium, there is heterogeneity at the C-terminus so that the product includes a mixture of species that can include any one or more of SEQ ID NO:4 to SEQ ID NO:9 in various abundance.

As used herein, an “N-linked moiety” refers to an asparagine (N) amino acid residue of a polypeptide that is capable of being glycosylated by post-translational modification. Exemplary N-linked moieties of human PH20 include amino acids N82, N166, N235, N254, N368 and N393 of human PH20 set forth in SEQ ID NO:1. As used herein, an “N-glycosylated polypeptide” refers to a PH20 polypeptide or truncated form thereto containing oligosaccharide linkage of at least three N-linked amino acid residues, for example, N-linked moieties corresponding to amino acid residues N235, N368 and N393 of SEQ ID NO:1. An N-glycosylated polypeptide can include a polypeptide where three, four, five and up to all of the N-linked moieties are linked to an oligosaccharide. The N-linked oligosaccharides can include oligomannose, complex, hybrid or sulfated oligosaccharides, or other oligosaccharides and monosaccharides.

As used herein, an “N-partially glycosylated polypeptide” refers to a polypeptide that minimally contains an N-acetylglucosamine glycan linked to at least three N-linked moieties. A partially glycosylated polypeptide can include various glycan forms, including monosaccharides, oligosaccharides, and branched sugar forms, including those formed by treatment of a polypeptide with EndoH, EndoF1, EndoF2 and/or EndoF3.

As used herein, a “deglycosylated PH20 polypeptide” refers to a PH20 polypeptide in which fewer than all possible glycosylation sites are glycosylated. Deglycosylation can be effected, for example, by removing glycosylation, by preventing it, or by modifying the polypeptide to eliminate a glycosylation site. Particular N-glycosylation sites are not required for activity, whereas others are.

As used herein, a hyaluronidase substrate refers to a substrate (e.g. protein or polysaccharide) that is cleaved and/or depolymerized by a hyaluronidase enzyme. Generally, a hyaluronidase substrate is a glycosaminoglycan. An exemplary hyaluronidase substrate is hyaluronan (HA).

As used herein, specific activity refers to Units of activity per mg protein. The milligrams of hyaluronidase is defined by the absorption of a solution of at 280 nm assuming a molar extinction coefficient of approximately 1.7, in units of M⁻¹cm⁻¹.

As used herein, “activity” refers to a functional activity or activities of a polypeptide or portion thereof associated with a full-length (complete) protein. For example, active fragments of a polypeptide can, exhibit an activity of a full-length protein. Functional activities include, but are not limited to, biological activity, catalytic or enzymatic activity, antigenicity (ability to bind or compete with a polypeptide for binding to an anti-polypeptide antibody), immunogenicity, ability to form multimers, and the ability to specifically bind to a receptor or ligand for the polypeptide.

As used herein, “hyaluronidase activity” or “hyaluronan-degrading activity” refers to the ability to enzymatically catalyze the cleavage of hyaluronic acid. The United States Pharmacopeia (USP) XXII assay for hyaluronidase determines hyaluronidase activity indirectly by measuring the amount of higher molecular weight hyaluronic acid, or hyaluronan, (HA) substrate remaining after the enzyme is allowed to react with the HA for 30 min at 37° C. (USP XXII-NF XVII (1990) 644-645 United States Pharmacopeia Convention, Inc, Rockville, Md.). A Reference Standard solution can be used in an assay to ascertain the relative activity, in units, of any hyaluronidase. In vitro assays to determine the hyaluronidase activity of hyaluronidases, such as PH20, including soluble PH20 and esPH20, are known in the art and described herein. Exemplary assays include the microturbidity assay that measures cleavage of hyaluronic acid by hyaluronidase indirectly by detecting the insoluble precipitate formed when the uncleaved hyaluronic acid binds with serum albumin and the biotinylated-hyaluronic acid assay that measures the cleavage of hyaluronic acid indirectly by detecting the remaining biotinylated-hyaluronic acid non-covalently bound to microtiter plate wells with a streptavidin-horseradish peroxidase conjugate and a chromogenic substrate. Reference Standards can be used, for example, to generate a standard curve to determine the activity in Units of the hyaluronidase being tested.

As used herein, “neutral active” refers to the ability of a PH20 polypeptide to enzymatically catalyze the cleavage of hyaluronic acid at neutral pH (e.g. at or about pH 7.0). Generally, a neutral active and soluble PH20, e.g., C-terminally truncated or N-partially glycosylated PH20, has or has about at least or 30%, 40%, 50%, 60%, 70%, 80%, 90%, 91%, 92%, 93%, 94%, 95%, 96%, 97%, 98%, 99%, 100%, 110%, 120%, 130%, 140%, 150%, 200%, 300%, 400%, 500%, 1000% or more activity compared to the hyaluronidase activity of a corresponding neutral active PH20 that is not C-terminally truncated or N-partially glycosylated.

As used herein, a “GPI-anchor attachment signal sequence” is a C-terminal sequence of amino acids that directs addition of a preformed GPI-anchor to the polypeptide within the lumen of the ER. GPI-anchor attachment signal sequences are present in the precursor polypeptides of GPI-anchored polypeptides, such as GPI-anchored PH20 polypeptides. The C-terminal GPI-anchor attachment signal sequence typically contains a predominantly hydrophobic region of 8-20 amino acids, preceded by a hydrophilic spacer region of 8-12 amino acids, immediately downstream of the ω-site, or site of GPI-anchor attachment. GPI-anchor attachment signal sequences can be identified using methods well known in the art. These include, but are not limited to, in silico methods and algorithms (see, e.g. Udenfriend et al. (1995) Methods Enzymol. 250:571-582, Eisenhaber et al., (1999) J. Biol. Chem. 292: 741-758, Fankhauser et al., (2005) Bioinformatics 21:1846-1852, Omaetxebarria et al., (2007) Proteomics 7:1951-1960, Pierleoni et al., (2008) BMC Bioinformatics 9:392), including those that are readily available on bioinformatic websites, such as the ExPASy Proteomics tools site (e.g., the WorldWideWeb site expasy.ch/tools/).

As used herein, a “polymer” refers to any high molecular weight natural or synthetic moiety that is conjugated to, i.e. stably linked directly or indirectly via a linker, to a polypeptide. Such polymers, typically increase serum half-life, and include, but are not limited to sialic moieties, PEGylation moieties, dextran, and sugar and other moieties, such as for glycosylation. For example, cholinesterases, such as AChE and BChE, and hyaluronidases, such as a soluble PH20 or rHuPH20, can be conjugated to a polymer.

As used herein, “PEGylated” refers to covalent or other stable attachment of polymeric molecules, such as polyethylene glycol (PEGylation moiety PEG) to proteins, including organophosphorus bioscavengers, such as cholinesterases, and hyaluronan-degrading enzymes, such as hyaluronidases. The addition of a PEGylation moiety can increase serum half-life of the protein. As used herein, a “conjugate” refers to a polypeptide linked directly or indirectly to one or more other polypeptides or chemical moieties. Such conjugates include fusion proteins, those produced by chemical conjugates and those produced by any other methods. For example, a conjugate refers to organophosphorus bioscavengers and hyaluronan-degrading enzymes linked directly or indirectly to one or more other polypeptides or chemical moieties, whereby at least one organophosphorus bioscavenger or hyaluronan-degrading enzyme is linked, directly or indirectly to another polypeptide or chemical moiety so long as the conjugate retains hyaluronidase activity.

As used herein, a “fusion” protein refers to a polypeptide encoded by a nucleic acid sequence containing a coding sequence from one nucleic acid molecule and the coding sequence from another nucleic acid molecule in which the coding sequences are in the same reading frame such that when the fusion construct is transcribed and translated in a host cell, the protein is produced containing the two proteins. The two molecules can be adjacent in the construct or separated by a linker polypeptide that contains, 1, 2, 3, or more, but typically fewer than 10, 9, 8, 7, or 6 amino acids. The protein product encoded by a fusion construct is referred to as a fusion polypeptide.

As used herein, “half-life” or “half-life of elimination” or “t½” refers to the time required for any specified property to decrease by half. For example, half-life refers to the time it takes a substance (e.g. an organophosphorus bioscavenger or a hyaluronan-degrading enzyme) to lose half of its activity or its original level. Hence, half-life can be determined by measuring the activity of a substance in plasma, or it can be determined by measuring the plasma level of the substance in the plasma. For example, half-life can be determined as the time necessary for the drug to be reduced to half of its original level in the body through various bodily processes. The longer the half-life, the longer it will take the substance or drug to be purged from the body. Units for half-life are generally units of time such as hour, minute or day.

As used herein, “absorption” refers to the movement of a drug into the bloodstream.

As used herein, “bioavailability” refers to the fraction of an administered dose of drug that reaches the systemic circulation. Bioavailability is a function of the absorption of the drug into the bloodstream.

As used herein, “dose” refers to the quantity or amount of drug that is administered to a subject for therapeutic or prophylactic purposes.

As used herein, “nucleic acids” include DNA, RNA and analogs thereof, including peptide nucleic acids (PNA) and mixtures thereof. Nucleic acids can be single or double-stranded. When referring to probes or primers, which are optionally labeled, such as with a detectable label, such as a fluorescent or radiolabel, single-stranded molecules are contemplated. Such molecules are typically of a length such that their target is statistically unique or of low copy number (typically less than 5, generally less than 3) for probing or priming a library. Generally a probe or primer contains at least 14, 16 or 30 contiguous nucleotides of sequence complementary to or identical to a gene of interest. Probes and primers can be 10, 20, 30, 50, 100 or more nucleic acids long.

As used herein, a peptide refers to a polypeptide that is greater than or equal to 2 amino acids in length, and less than or equal to 40 amino acids in length.

As used herein, the amino acids which occur in the various sequences of amino acids provided herein are identified according to their known, three-letter or one-letter abbreviations (Table 1). The nucleotides which occur in the various nucleic acid fragments are designated with the standard single-letter designations used routinely in the art.

As used herein, an “amino acid” is an organic compound containing an amino group and a carboxylic acid group. A polypeptide contains two or more amino acids. For purposes herein, amino acids include the twenty naturally-occurring amino acids, non-natural amino acids and amino acid analogs (i.e., amino acids wherein the α-carbon has a side chain).

As used herein, “amino acid residue” refers to an amino acid formed upon chemical digestion (hydrolysis) of a polypeptide at its peptide linkages. The amino acid residues described herein are presumed to be in the “L” isomeric form. Residues in the “D” isomeric form, which are so designated, can be substituted for any L-amino acid residue as long as the desired functional property is retained by the polypeptide. NH₂ refers to the free amino group present at the amino terminus of a polypeptide. COOH refers to the free carboxy group present at the carboxyl terminus of a polypeptide. In keeping with standard polypeptide nomenclature described in J. Biol. Chem., 243: 3557-3559 (1968), and adopted 37 C.F.R. §§1.821-1.822, abbreviations for amino acid residues are shown in Table 1:

TABLE 1
Table of Correspondence

| SYMBOL | | |
|---|---|---|
| 1-Letter | 3-Letter | AMINO ACID |
| Y | Tyr | Tyrosine |
| G | Gly | Glycine |
| F | Phe | Phenylalanine |
| M | Met | Methionine |
| A | Ala | Alanine |
| S | Ser | Serine |
| I | Ile | Isoleucine |
| L | Leu | Leucine |
| T | Thr | Threonine |
| V | Val | Valine |
| P | Pro | Proline |
| K | Lys | Lysine |
| H | His | Histidine |
| Q | Gln | Glutamine |
| E | Glu | Glutamic acid |
| Z | Glx | Glu and/or Gln |
| W | Trp | Tryptophan |
| R | Arg | Arginine |
| D | Asp | Aspartic acid |
| N | Asn | Asparagine |
| B | Asx | Asn and/or Asp |
| C | Cys | Cysteine |
| X | Xaa | Unknown or other |

All amino acid residue sequences represented herein by formulae have a left to right orientation in the conventional direction of amino-terminus to carboxyl-terminus. In addition, the phrase “amino acid residue” is defined to include the amino acids listed in the Table of Correspondence (Table 1) and modified and unusual amino acids, such as those referred to in 37 C.F.R. §§1.821-1.822, and incorporated herein by reference. Furthermore, it should be noted that a dash at the beginning or end of an amino acid residue sequence indicates a peptide bond to a further sequence of one or more amino acid residues, to an amino-terminal group such as NH₂ or to a carboxyl-terminal group such as COOH.

As used herein, the “naturally occurring α-amino acids” are the residues of those 20 α-amino acids found in nature which are incorporated into protein by the specific recognition of the charged tRNA molecule with its cognate mRNA codon in humans. Non-naturally occurring amino acids thus include, for example, amino acids or analogs of amino acids other than the 20 naturally-occurring amino acids and include, but are not limited to, the D-stereoisomer of amino acids. Exemplary non-natural amino acids are described herein and are known to those of skill in the art.

As used herein, a “non-native” amino acid refers to an amino acid that is not normally located at a position or an amino acid or amino acid analog that has been chemically modified to allow conjugation with a polymer such as polyethylene glycol.

As used herein, a DNA construct is a single- or double-stranded, linear or circular DNA molecule that contains segments of DNA combined and juxtaposed in a manner not found in nature. DNA constructs exist as a result of human manipulation, and include clones and other copies of manipulated molecules.

As used herein, a DNA segment is a portion of a larger DNA molecule having specified attributes. For example, a DNA segment encoding a specified polypeptide is a portion of a longer DNA molecule, such as a plasmid or plasmid fragment, which, when read from the 5′ to 3′ direction, encodes the sequence of amino acids of the specified polypeptide.

As used herein, the term polynucleotide means a single- or double-stranded polymer of deoxyribonucleotides or ribonucleotide bases read from the 5′ to the 3′ end. Polynucleotides include RNA and DNA, and can be isolated from natural sources, synthesized in vitro, or prepared from a combination of natural and synthetic molecules. The length of a polynucleotide molecule is given herein in terms of nucleotides (abbreviated “nt”) or base pairs (abbreviated “bp”). The term nucleotides is used for single- and double-stranded molecules where the context permits. When the term is applied to double-stranded molecules it is used to denote overall length and will be understood to be equivalent to the term base pairs. It will be recognized by those skilled in the art that the two strands of a double-stranded polynucleotide can differ slightly in length and that the ends thereof can be staggered; thus all nucleotides within a double-stranded polynucleotide molecule may not be paired. Such unpaired ends will, in general, not exceed 20 nucleotides in length.

As used herein, “similarity” between two proteins or nucleic acids refers to the relatedness between the sequence of amino acids of the proteins or the nucleotide sequences of the nucleic acids. Similarity can be based on the degree of identity and/or homology of sequences of residues and the residues contained therein. Methods for assessing the degree of similarity between proteins or nucleic acids are known to those of skill in the art. For example, in one method of assessing sequence similarity, two amino acid or nucleotide sequences are aligned in a manner that yields a maximal level of identity between the sequences. “Identity” refers to the extent to which the amino acid or nucleotide sequences are invariant. Alignment of amino acid sequences, and to some extent nucleotide sequences, also can take into account conservative differences and/or frequent substitutions in amino acids (or nucleotides). Conservative differences are those that preserve the physico-chemical properties of the residues involved. Alignments can be global (alignment of the compared sequences over the entire length of the sequences and including all residues) or local (the alignment of a portion of the sequences that includes only the most similar region or regions).

“Identity” per se has an art-recognized meaning and can be calculated using published techniques. (See, e.g. Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exists a number of methods to measure identity between two polynucleotide or polypeptides, the term “identity” is well known to skilled artisans (Carrillo, H. & Lipman, D., SIAM J Applied Math 48:1073 (1988)).

As used herein, homologous (with respect to nucleic acid and/or amino acid sequences) means about greater than or equal to 25% sequence homology, typically greater than or equal to 25%, 40%, 50%, 60%, 70%, 80%, 85%, 90% or 95% sequence homology; the precise percentage can be specified if necessary. For purposes herein the terms “homology” and “identity” are often used interchangeably, unless otherwise indicated. In general, for determination of the percentage homology or identity, sequences are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48:1073). By sequence homology, the number of conserved amino acids is determined by standard alignment algorithms programs, and can be used with default gap penalties established by each supplier. Substantially homologous nucleic acid molecules would hybridize typically at moderate stringency or at high stringency all along the length of the nucleic acid of interest. Also contemplated are nucleic acid molecules that contain degenerate codons in place of codons in the hybridizing nucleic acid molecule.

Whether any two molecules have nucleotide sequences or amino acid sequences that are at least 60%, 70%, 80%, 85%, 90%, 95%, 96%, 97%, 98% or 99% “identical” or “homologous” can be determined using known computer algorithms such as the “FASTA” program, using for example, the default parameters as in Pearson et al. (1988) Proc. Natl. Acad. Sci. USA 85:2444 (other programs include the GCG program package (Devereux, J., et al., Nucleic Acids Research 12(I):387 (1984)), BLASTP, BLASTN, FASTA (Altschul, S. F., et al., J Mol Biol 215:403 (1990)); Guide to Huge Computers, Martin J. Bishop, ed., Academic Press, San Diego, 1994, and Carrillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.). Percent homology or identity of proteins and/or nucleic acid molecules can be determined, for example, by comparing sequence information using a GAP computer program (e.g., Needleman et al. (1970) J. Mol. Biol. 48:443, as revised by Smith and Waterman ((1981) Adv. Appl. Math. 2:482). Briefly, the GAP program defines simi-laity as the number of aligned symbols (i.e., nucleotides or amino acids), which are similar, divided by the total number of symbols in the shorter of the two sequences. Default parameters for the GAP program can include: (1) a unary comparison matrix (containing a value of 1 for identities and 0 for non-identities) and the weighted comparison matrix of Gribskov et al. (1986) Nucl. Acids Res. 14:6745, as described by Schwartz and Dayhoff, eds., ATLAS OF PROTEIN SEQUENCE AND STRUCTURE, National Biomedical Research Foundation, pp. 353-358 (1979); (2) a penalty of 3.0 for each gap and an additional 0.10 penalty for each symbol in each gap; and (3) no penalty for end gaps.

Therefore, as used herein, the term “identity” or “homology” represents a comparison between a test and a reference polypeptide or polynucleotide. As used herein, the term at least “90% identical to” refers to percent identities from 90 to 99.99 relative to the reference nucleic acid or amino acid sequence of the polypeptide. Identity at a level of 90% or more is indicative of the fact that, assuming for exemplification purposes a test and reference polypeptide length of 100 amino acids are compared. No more than 10% (i.e., 10 out of 100) of the amino acids in the test polypeptide differ from those of the reference polypeptide. Similar comparisons can be made between test and reference polynucleotides. Such differences can be represented as point mutations randomly distributed over the entire length of a polypeptide or they can be clustered in one or more locations of varying length up to the maximum allowable, e.g. 10/100 amino acid difference (approximately 90% identity). Differences are defined as nucleic acid or amino acid substitutions, insertions or deletions. At the level of homologies or identities above about 85-90%, the result should be independent of the program and gap parameters set; such high levels of identity can be assessed readily, often by manual alignment without relying on software.

As used herein, an aligned sequence refers to the use of homology (similarity and/or identity) to align corresponding positions in a sequence of nucleotides or amino acids. Typically, two or more sequences that are related by 50% or more identity are aligned. An aligned set of sequences refers to 2 or more sequences that are aligned at corresponding positions and can include aligning sequences derived from RNAs, such as ESTs and other cDNAs, aligned with genomic DNA sequence.

As used herein, “primer” refers to a nucleic acid molecule that can act as a point of initiation of template-directed DNA synthesis under appropriate conditions (e.g., in the presence of four different nucleoside triphosphates and a polymerization agent, such as DNA polymerase, RNA polymerase or reverse transcriptase) in an appropriate buffer and at a suitable temperature. It will be appreciated that certain nucleic acid molecules can serve as a “probe” and as a “primer.” A primer, however, has a 3′ hydroxyl group for extension. A primer can be used in a variety of methods, including, for example, polymerase chain reaction (PCR), reverse-transcriptase (RT)-PCR, RNA PCR, LCR, multiplex PCR, panhandle PCR, capture PCR, expression PCR, 3′ and 5′ RACE, in situ PCR, ligation-mediated PCR and other amplification protocols.

As used herein, “primer pair” refers to a set of primers that includes a 5′ (upstream) primer that hybridizes with the 5′ end of a sequence to be amplified (e.g. by PCR) and a 3′ (downstream) primer that hybridizes with the complement of the 3′ end of the sequence to be amplified.

As used herein, “specifically hybridizes” refers to annealing, by complementary base-pairing, of a nucleic acid molecule (e.g. an oligonucleotide) to a target nucleic acid molecule. Those of skill in the art are familiar with in vitro and in vivo parameters that affect specific hybridization, such as length and composition of the particular molecule. Parameters particularly relevant to in vitro hybridization further include annealing and washing temperature, buffer composition and salt concentration. Exemplary washing conditions for removing non-specifically bound nucleic acid molecules at high stringency are 0.1×SSPE, 0.1% SDS, 65° C., and at medium stringency are 0.2×SSPE, 0.1% SDS, 50° C. Equivalent stringency conditions are known in the art. The skilled person can readily adjust these parameters to achieve specific hybridization of a nucleic acid molecule to a target nucleic acid molecule appropriate for a particular application. Complementary, when referring to two nucleotide sequences, means that the two sequences of nucleotides are capable of hybridizing, typically with less than 25%, 15% or 5% mismatches between opposed nucleotides. If necessary, the percentage of complementarity will be specified. Typically the two molecules are selected such that they will hybridize under conditions of high stringency.

As used herein, substantially identical to a product means sufficiently similar so that the property of interest is sufficiently unchanged so that the substantially identical product can be used in place of the product.

As used herein, it also is understood that the terms “substantially identical” or “similar” varies with the context as understood by those skilled in the relevant art.

As used herein, an allelic variant or allelic variation references any of two or more alternative forms of a gene occupying the same chromosomal locus. Allelic variants refer to variations in proteins among members of the same species. Allelic variation arises naturally through mutation, and can result in phenotypic polymorphism within populations. Gene mutations can be silent (no change in the encoded polypeptide) or can encode polypeptides having altered amino acid sequence. The term “allelic variant” also is used herein to denote a protein encoded by an allelic variant of a gene. Typically the reference form of the gene encodes a wildtype form and/or predominant form of a polypeptide from a population or single reference member of a species. Typically, allelic variants, which include variants between and among species typically have at least 80%, 90% or greater amino acid identity with a wildtype and/or predominant form from the same species; the degree of identity depends upon the gene and whether comparison is interspecies or intraspecies. Generally, intraspecies allelic variants have at least about 80%, 85%, 90% or 95% identity or greater with a wildtype and/or predominant form, including 96%, 97%, 98%, 99% or greater identity with a wildtype and/or predominant form of a polypeptide. Reference to an allelic variant herein generally refers to variations n proteins among members of the same species.

As used herein, “allele,” which is used interchangeably herein with “allelic variant” refers to alternative forms of a gene or portions thereof. Alleles occupy the same locus or position on homologous chromosomes. When a subject has two identical alleles of a gene, the subject is said to be homozygous for that gene or allele. When a subject has two different alleles of a gene, the subject is said to be heterozygous for the gene. Alleles of a specific gene can differ from each other in a single nucleotide or several nucleotides, and can include modifications such as substitutions, deletions and insertions of nucleotides. An allele of a gene also can be a form of a gene containing a mutation.

As used herein, species variants refer to variants in polypeptides among different species, including different mammalian species, such as mouse and human. For example for PH20, exemplary of species variants provided herein are primate PH20, such as, but not limited to, human, chimpanzee, macaque and cynomolgous monkey. Generally, species variants have 70%, 75%, 80%, 85%, 90%, 91%, 92%, 93%, 94%, 95%, 96%, 97%, 98% or more sequence identity. Corresponding residues between and among species variants can be determined by comparing and aligning sequences to maximize the number of matching nucleotides or residues, for example, such that identity between the sequences is equal to or greater than 95%, equal to or greater than 96%, equal to or greater than 97%, equal to or greater than 98% or equal to or greater than 99%. The position of interest is then given the number assigned in the reference nucleic acid molecule. Alignment can be effected manually or by eye, particularly, where sequence identity is greater than 80%.

As used herein, a human protein is one encoded by a nucleic acid molecule, such as DNA, present in the genome of a human, including all allelic variants and conservative variations thereof. A variant or modification of a protein is a human protein if the modification is based on the wildtype or prominent sequence of a human protein.

As used herein, a splice variant refers to a variant produced by differential processing of a primary transcript of genomic DNA that results in more than one type of mRNA.

As used herein, modification is in reference to modification of a sequence of amino acids of a polypeptide or a sequence of nucleotides in a nucleic acid molecule and includes deletions, insertions, and replacements (e.g. substitutions) of amino acids and nucleotides, respectively. Exemplary of modifications are amino acid substitutions. Methods of modifying a polypeptide are routine to those of skill in the art, such as by using recombinant DNA methodologies.

As used herein, reference to a “modified” or “variant” polypeptide refers to a polypeptide that has one or more amino acid differences compared to a corresponding unmodified or wild-type polypeptide. The one or more amino acid differences can be amino acid mutations such as one or more amino acid replacements (substitutions), insertions or deletions, or can be insertions or deletions of entire domains, and any combinations thereof. A modified or variant polypeptide, such as an amino-acid substituted polypeptide, can exhibit 65%, 70%, 80%, 85%, 90%, 91%, 92%, 93%, 94%, 95%, 96%, 97%, 98% or more sequence identity to a polypeptide not containing the amino acid substitutions. Amino acid substitutions can be conservative or non-conservative. Generally, any modification to a polypeptide retains an activity of the polypeptide. Any modification is contemplated as long as the resulting polypeptide exhibits activity. For example, a variant OP bioscavenger exhibits OP inactivating activity, OP binding activity and/or OP hydrolytic activity. In another example, a variant hyaluronan-degrading enzyme exhibits hyaluronidase activity. The activity that is exhibited by the variant can be at least or about at least 40%, 50%, 60%, 70%, 80%, 90%, 100%, 110%, 120%, 130%, 140%, 150%, 200%, 300%, 400%, 500% or more of the activity of the corresponding polypeptide not containing the modification.

As used herein, suitable conservative substitutions of amino acids are known to those of skill in this art and can be made generally without altering the biological activity of the resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p. 224). Such substitutions can be made in accordance with those set forth in TABLE 2 as follows:

TABLE 2

| Original residue | Exemplary conservative substitution |
|---|---|
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions also are permissible and can be determined empirically or in accord with known conservative substitutions.

As used herein, the term promoter means a portion of a gene containing DNA sequences that provide for the binding of RNA polymerase and initiation of transcription. Promoter sequences are commonly, but not always, found in the 5′ non-coding region of genes.

As used herein, isolated or purified polypeptide or protein or biologically-active portion thereof is substantially free of cellular material or other contaminating proteins from the cell or tissue from which the protein is derived, or substantially free from chemical precursors or other chemicals when chemically synthesized.

Preparations can be determined to be substantially free if they appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound, however, can be a mixture of stereoisomers. In such instances, further purification might increase the specific activity of the compound.

Hence, reference to a substantially purified polypeptide, such as a substantially purified soluble PH20, refers to preparations of proteins that are substantially free of cellular material includes preparations of proteins in which the protein is separated from cellular components of the cells from which it is isolated or recombinantly-produced. In one embodiment, the term substantially free of cellular material includes preparations of enzyme proteins having less than about 30% (by dry weight) of non-enzyme proteins (also referred to herein as a contaminating protein), generally less than about 20% of non-enzyme proteins or 10% of non-enzyme proteins or less than about 5% of non-enzyme proteins. When the enzyme protein is recombinantly produced, it also is substantially free of culture medium, i.e., culture medium represents less than about or at 20%, 10% or 5% of the volume of the enzyme protein preparation.

As used herein, the term substantially free of chemical precursors or other chemicals includes preparations of enzyme proteins in which the protein is separated from chemical precursors or other chemicals that are involved in the synthesis of the protein. The term includes preparations of enzyme proteins having less than about 30% (by dry weight), 20%, 10%, 5% or less of chemical precursors or non-enzyme chemicals or components.

As used herein, synthetic, with reference to, for example, a synthetic nucleic acid molecule or a synthetic gene or a synthetic peptide refers to a nucleic acid molecule or polypeptide molecule that is produced by recombinant methods and/or by chemical synthesis methods.

As used herein, production by recombinant means or using recombinant DNA methods means the use of the well known methods of molecular biology for expressing proteins encoded by cloned DNA.

As used herein, vector (or plasmid) refers to discrete elements that are used to introduce a heterologous nucleic acid into cells for either expression or replication thereof. The vectors typically remain episomal, but can be designed to effect integration of a gene or portion thereof into a chromosome of the genome. Also contemplated are vectors that are artificial chromosomes, such as yeast artificial chromosomes and mammalian artificial chromosomes. Selection and use of such vehicles are well known to those of skill in the art.

As used herein, an expression vector includes vectors capable of expressing DNA that is operatively linked with regulatory sequences, such as promoter regions, that are capable of effecting expression of such DNA fragments. Such additional segments can include promoter and terminator sequences, and optionally can include one or more origins of replication, one or more selectable markers, an enhancer, a polyadenylation signal. Expression vectors are generally derived from plasmid or viral DNA, or can contain elements of both. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, vector also includes “virus vectors” or “viral vectors.” Viral vectors are engineered viruses that are operatively linked to exogenous genes to transfer (as vehicles or shuttles) the exogenous genes into cells.

As used herein, “operably” or “operatively linked” when referring to DNA segments means that the segments are arranged so that they function in concert for their intended purposes, e.g., transcription initiates downstream of the promoter and upstream of any transcribed sequences. The promoter is usually the domain to which the transcriptional machinery binds to initiate transcription and proceeds through the coding segment to the terminator.

As used herein the term “assessing” is intended to include quantitative and qualitative determination in the sense of obtaining an absolute value for the activity of a protein, such as an enzyme, or a domain thereof, present in the sample, and also of obtaining an index, ratio, percentage, visual or other value indicative of the level of the activity. Assessment can be direct or indirect. For example, the chemical species actually detected need not of course be the enzymatically cleaved product itself but can for example be a derivative thereof or some further substance. For example, detection of a cleavage product can be a detectable moiety such as a fluorescent moiety.

As used herein, biological activity refers to the in vivo activities of a compound or physiological responses that result upon in vivo administration of a compound, composition or other mixture. Biological activity, thus, encompasses therapeutic effects and pharmaceutical activity of such compounds, compositions and mixtures. Biological activities can be observed in in vitro systems designed to test or use such activities. Thus, for purposes herein a biological activity of a hyaluronidase enzyme is its degradation of hyaluronic acid. For purposes herein, a biological activity of an organophosphorus bioscavenger is its binding to, or hydrolyzing, an OP compound.

As used herein equivalent, when referring to two sequences of nucleic acids, means that the two sequences in question encode the same sequence of amino acids or equivalent proteins. When equivalent is used in referring to two proteins or peptides, it means that the two proteins or peptides have substantially the same amino acid sequence with only amino acid substitutions that do not substantially alter the activity or function of the protein or peptide. When equivalent refers to a property, the property does not need to be present to the same extent (e.g., two peptides can exhibit different rates of the same type of enzymatic activity), but the activities are usually substantially the same.

As used herein, “modulate” and “modulation” or “alter” refer to a change of an activity of a molecule, such as a protein. Exemplary activities include, but are not limited to, biological activities, such as signal transduction. Modulation can include an increase in the activity (i.e., up-regulation or agonist activity), a decrease in activity (i.e., down-regulation or inhibition) or any other alteration in an activity (such as a change in periodicity, frequency, duration, kinetics or other parameter). Modulation can be context dependent and typically modulation is compared to a designated state, for example, the wildtype protein, the protein in a constitutive state, or the protein as expressed in a designated cell type or condition.

As used herein, a composition refers to any mixture. It can be a solution, suspension, liquid, powder, paste, aqueous, non-aqueous or any combination thereof.

As used herein, a combination refers to any association between or among two or more items. The combination can be two or more separate items, such as two compositions or two collections, can be a mixture thereof, such as a single mixture of the two or more items, or any variation thereof. The elements of a combination are generally functionally associated or related.

As used herein, “disease or disorder” refers to a pathological condition in an organism resulting from cause or condition including, but not limited to, infections, acquired conditions, genetic conditions, and characterized by identifiable symptoms. Diseases and disorders of interest herein are organophosphorus poisoning, including organophosphorus pesticide poisoning and nerve agent poisoning.

As used herein, “treating” a subject with a disease or condition means that the subject's symptoms are partially or totally alleviated, or remain static following treatment. Hence treatment encompasses prophylaxis, therapy and/or cure. Prophylaxis refers to prevention of a potential disease and/or a prevention of worsening of symptoms or progression of a disease.

As used herein, a pharmaceutically effective agent, includes any therapeutic agent or bioactive agents, including, but not limited to, for example, carbamates, anti-muscarinics, cholinesterase reactivators, anti-convulsives, dispersing agents, conventional therapeutic drugs, including small molecule drugs and therapeutic proteins.

As used herein, treatment means any manner in which the symptoms of a condition, disorder or disease or other indication, are ameliorated or otherwise beneficially altered.

As used herein, therapeutic effect means an effect resulting from treatment of a subject that alters, typically improves or ameliorates the symptoms of a disease or condition or that cures a disease or condition. A therapeutically effective amount refers to the amount of a composition, molecule or compound which results in a therapeutic effect following administration to a subject.

As used herein, the term “subject” refers to an animal, including a mammal, such as a human being.

As used herein, a “subject in need thereof” refers to a human or animal subject who is sensitive to OP toxic effects. Thus, the subject may be exposed or at a risk of exposure to organophosphorus poisoning. Examples include, but are not limited to, civilians contaminated by a terrorist attack at a public event, accidental spills in industry and during transportation, field workers subjected to pesticide or insecticide organophosphorus poisoning, truckers who transport pesticides, pesticide manufacturers, dog groomers who are overexposed to flea dip, pest control workers various domestic and custodial workers who use organophosphorus compounds and military personnel exposed to nerve gases.

As used herein, a patient refers to a human subject exhibiting symptoms of a disease or disorder.

As used herein, about the same means within an amount that one of skill in the art would consider to be the same or to be within an acceptable range of error. For example, typically, for pharmaceutical compositions, within at least 1%, 2%, 3%, 4%, 5% or 10% is considered about the same. Such amount can vary depending upon the tolerance for variation in the particular composition by subjects.

As used herein, dosing regime refers to the amount of agent, for example, the composition containing an organophosphorus bioscavenger, for example, a butyrylcholinesterase or other agent, and a hyaluronan-degrading enzyme, for example a soluble hyaluronidase or other agent, administered, and the frequency of administration. The dosing regime is a function of the disease or condition to be treated, and thus can vary.

As used herein, frequency of administration refers to the time between successive administrations of treatment. For example, frequency can be hours, days, weeks or months. For example, frequency can be more than once weekly, for example, twice a week, three times a week, four times a week, five times a week, six times a week or daily. Frequency also can be one, two, three or four weeks. The particular frequency is a function of the particular disease or condition treated. Generally, frequency is more than once weekly, and generally is twice weekly.

As used herein, a “cycle of administration” refers to the repeated schedule of the dosing regime of administration of the enzyme and/or a second agent that is repeated over successive administrations.

As used herein, when referencing dosage based on mg/kg of the subject, an average human subject is considered to have a mass of about 70 kg-75 kg, such as 70 kg.

As used herein, amelioration of the symptoms of a particular disease or disorder by a treatment, such as by administration of a pharmaceutical composition or other therapeutic, refers to any lessening, whether permanent or temporary, lasting or transient, of the symptoms or, adverse effects of a condition, such as, for example, organophosphorus poisoning.

As used herein, “ameliorating” or “reducing” a side effect or adverse event, or variations thereof, refers to lessening adverse effects or side effects, whether permanent or temporary, lasting or transient. For purposes herein, ameliorating or reducing includes lessening side effects associated with organophosphorus poisoning.

As used herein, prevention or prophylaxis refers to reduction in the risk of developing a disease or condition. For purposes herein, prevention means that the compositions and combinations containing OP bioscavengers effect prophylactic protection.

As used herein, “prophylactic protection” refers to protection against the effects of nerve agent exposure such that cholinergic toxicity of nerve agents is reduced or eliminated. For example, prophylactic protection is achieved when circulating endogenous cholinesterase levels are maintained at a level that is at least 30% of baseline activity after exposure to a nerve agent. Generally, prophylactic protection means that circulating OP bioscavenger agents are present in the plasma at a level that is a least 15 μg/mL, and in particular at a level that is at least 20 μg/mL, 21 μg/mL, 22 μg/mL, 23 μg/mL, 24 μg/mL, 25 μg/mL, 26 μg/mL, 27 μg/mL, 28 μg/mL, 29 μg/mL, 30 μg/mL, 40 μg/mL, 50 μg/mL, 100 μg/mL, 200 μg/mL, 300 μg/mL, 400 μg/mL, 500 μg/mL, 600 μg/mL or more in order to combat the effects of exposure to nerve agents. In particular examples, nerve agent protection is achieved by prophylactic protection within 24 hours of administration of an OP bioscavenger enzyme and for a duration of time of at least 10 days.

As used herein “baseline activity” refers to a measurable value that represents the normal or beginning level of activity a protein or other substance. Baseline activity can be measured in the plasma isolated from a subject prior to treatment or exposure to any agent or substance. Hence, it represents the normal level. The baseline activity can be used as a corrective measure to normalize levels of exogenously administered drugs or substances.

As used herein, a “therapeutically effective amount” or a “therapeutically effective dose” refers to the quantity of an agent, compound, material, or composition containing a compound that is at least sufficient to produce a therapeutic effect. Hence, it is the quantity necessary for preventing, curing, ameliorating, arresting or partially arresting a symptom of a disease or disorder.

As used herein, “percutaneous” administration refers to any medical procedure where access to inner organs or other tissues is effected by passage through the skin, such as by needle puncture of the skin. Percutaneous administration includes, for example, intramuscular injection, subcutaneous injection and intradermal injection.

As used herein, “intramuscular injection” refers to injection into deep muscle tissue. Typically, injection is given in the buttocks, thigh or the upper arm area. Intramuscular injection can be facilitated by use of a needle that is or is about 1.0 to 1.5 inches in length or longer, and generally more than one inch in length or more. For example, intramuscular injection can be made using a 20 to 22 gauge needle.

As used herein, “subcutaneous injection” refers to injection given through the epidermis and dermis to reach the subcutaneous fatty (adipose) tissue. As used herein, “intradermal injection” refers to injection into the dermal layer of the skin.

As used herein, unit dose form refers to physically discrete units suitable for human and animal subjects and packaged individually as is known in the art.

As used herein, a single dosage formulation refers to a formulation as a single dose.

As used herein, formulation for direct administration means that the composition does not require further dilution for administration.

As used herein, an “article of manufacture” is a product that is made and sold. As used throughout this application, the term is intended to encompass anti-hyaluronan agents, for example hyaluronan-degrading enzyme, such as hyaluronidase, and second agent compositions contained in articles of packaging. For example, a second agent is an OP bioscavenger.

As used herein, fluid refers to any composition that can flow. Fluids thus encompass compositions that are in the form of semi-solids, pastes, solutions, aqueous mixtures, gels, lotions, creams and other such compositions.

As used herein a kit refers to a combination of components, such as a combination of the compositions herein and another item for a purpose including, but not limited to, reconstitution, activation, and instruments/devices for delivery, administration, diagnosis, and assessment of a biological activity or property. Kits optionally include instructions for use.

As used herein, a cellular extract or lysate refers to a preparation or fraction which is made from a lysed or disrupted cell.

As used herein, animal includes any animal, such as, but are not limited to primates including humans, gorillas and monkeys; rodents, such as mice and rats; fowl, such as chickens; ruminants, such as goats, cows, deer, sheep; pigs and other animals. Non-human animals exclude humans as the contemplated animal. The hyaluronidases provided herein are from any source, animal, plant, prokaryotic and fungal. Most hyaluronidases are of animal origin, including mammalian origin. Generally hyaluronidases are of human origin.

As used herein, a control refers to a sample that is substantially identical to the test sample, except that it is not treated with a test parameter, or, if it is a plasma sample, it can be from a normal volunteer not affected with the condition of interest. A control also can be an internal control.

As used herein, the singular forms “a,” “an” and “the” include plural referents unless the context clearly dictates otherwise. Thus, for example, reference to a compound comprising or containing “an extracellular domain” includes compounds with one or a plurality of extracellular domains.

As used herein, ranges and amounts can be expressed as “about” a particular value or range. About also includes the exact amount. Hence “about 5 bases” means “about 5 bases” and also “5 bases.”

As used herein, “optional” or “optionally” means that the subsequently described event or circumstance does or does not occur, and that the description includes instances where said event or circumstance occurs and instances where it does not. For example, an optionally substituted group means that the group is unsubstituted or is substituted.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:1726).

### B. Compositions and Combinations of Organophosphorus Bioscavengers and Hyaluronan-Degrading Enzymes

Provided herein are compositions and combinations of an organophosphorus (OP) bioscavenger (such as butyrylcholinesterase) and a hyaluronan-degrading enzyme (such as a hyaluronidase for example a PH20 or truncated form thereof that is active, e.g. rHuPH20). The hyaluronan-degrading enzyme degrades hyaluronan, the extracellular matrix gel-like substance in the skin, and acts as a spreading agent such that co-administration of a composition or combination containing an organophosphorus bioscavenger and a hyaluronidase results in early systemic exposure and increased overall bioavailability of the OP bioscavenger. The formulations, compositions and compositions provided herein can be used as effective therapeutics for the treatment of organophosphorus poisoning, such as poisoning by chemical warfare and pesticide poisoning, without nerve-agent induced impairment and the adverse side effects exhibited by approved therapeutics. Hence, provided herein are methods and uses to prevent, treat or ameliorate nerve agent poisoning by providing a composition or combination of an organophosphorus bioscavenger and hyaluronan-degrading enzyme provided herein.

1. Nerve Agent Poisoning

Nerve agent poisoning by organophosphates and related compounds found in chemical weapons and pesticides remains a constant threat to public safety. For example, three million pesticide poisonings occur each year worldwide, resulting in 220,000 deaths (World Health Organization (1986) Informal Consultation on Planning Strategy for the Prevention of Pesticide Poisoning (WHO, Geneva), WHONBC/86.926; World Health Organization (1990) Public Health Impact of Pesticides Used in Agriculture (WHO, Geneva)).

Organophosphates (OPs) are generally highly lipid soluble and can be absorbed upon exposure by the skin, mucous membranes, gastrointestinal system, and respiratory system. Organophosphates are potent inhibitors of cholinesterases that act by permanently binding to these enzymes. Acetylcholinesterase inhibition causes a buildup of excess neurotransmitters resulting in continued stimulation of muscarinic receptor sites (exocrine glands and smooth muscles) and nicotinic receptor sites (skeletal muscles), and thereby cholinergic toxicity. Organophosphates exert their toxic effects by inhibiting the activity of acetylcholinesterase at nerve endings, leading to the accumulation of the neurotransmitter acetylcholine, and affecting the parasympathetic, sympathetic, motor, and central nervous systems. Thus, exposure to organophosphates causes damage to the peripheral and central nervous systems and results in myopathy, psychosis, general paralysis and death. Symptoms of exposure include twitching, trembling, hypersecretion, paralyzed breathing, convulsions, and ultimately death. When inhaled, signs of organophosphate poisoning can be observed within minutes. When exposure is percutaneous, absorption is slower and later onset and longer duration of symptoms and ailments occurs.

2. Treatments for Nerve Agent Poisoning

Typical treatment of nerve agent poisoning involves intravenous or intramuscular administration of combinations of drugs that serve to antagonize the effects of elevated acetylcholine levels, restore normal acetylcholinesterase activity, and treat symptoms, such a tremors and convulsions. These drugs include carbamates (e.g., pyridostigmine), anti-muscarinics (e.g., atropine), and ChE-reactivators, for example, oximes, such as pralidoxime chloride (pyridinium-2-aldoxime, 2-PAM, Protopam®). Administration of these drugs promotes survival but does not afford complete protection against either nerve agent-induced motor and cognitive defects or neuronal pathology (Lenz et al., (2007) Toxicology 233:31-39). In addition, each of these drugs causes adverse side effects, such as impairment of central nervous system function, dizziness, headaches and increased blood pressure and heart rate (Lenz et al., (2007) Toxicology 233:31-39). Further, the use of oximes to reactivate OP-inhibited acetylcholinesterase is not always effective. For example, the nerve agent Soman (also known as GD) is refractory to reactivation by clinically available oximes (see, e.g., Kassa, J. (2002) J Toxicol Clin Toxicol 40:803-816.) Approximately 10-40% of poisoned patients die, even in developing countries after treatment with “normal” therapeutics (Eyer et al., (2003) Toxicol Rev 22:143-163).

In recent years, it has been demonstrated that enzymes, such as esterases and cholinesterases, can be used as bioscavengers for organophosphates (see, e.g., Broomfield et al., (1991) J Pharmacol Exp Ther 259:633-638; Castro et al., (1994) Neurotox Teratol 16:145-148; Lenz et al., (2001) Nerve agent bioscavengers: protection against high- and low-dose organophosphorus exposure. In: Somani and Romano (Eds), Chemical Warfare Agents: Toxicity at Low Levels, CRC Press, Boca Raton, pp. 245-260; Lenz et al. (2005) Chem Biol Interact 157:205-210; Cerasoli et al. (2005) Chem Biol Interact 157:363-365; and Huang et al., (2008) BMC Biotechnol 8:50.) Bioscavenger enzymes act by binding to and/or hydrolyzing the organophosphate nerve agent which inhibits its ability to bind to acetylcholinesterase. These proteins are advantageous in that they remain in stable circulation over a long period of time and can be used as a prophylactic therapy, allowing prevention of toxic effects as opposed to treatment of toxic effects.

One such bioscavenger that has proven successful, although it is constrained by its limited availability, is human butyrylcholinesterase (Broomfield et al., (1991) J Pharmacol Exp Ther 259:633-638). Recently, exogenous administration of recombinantly produced human butyrylcholinesterase (rBChE), including a PEGylated form thereof, has been proven effective for preventing organophosphate poisoning (see, e.g., Huang et al., (2007) Proc Natl Acad Sci USA 101:13603-13608). The PEGylated rBChE has a half-life that is comparable to the human butyrlcholinesterase (in a guinea pig model) of 40-45 hours compared to only 6-7 hours for non-PEGylated rBChE (Huang et al. (2007)). When PEG-rBChE is administered prior to nerve agent exposure in guinea pigs all animals survived with no signs of cholinergic toxicity.

Although bioscavengers have proven effective as prophylactic therapies for nerve agent poisoning, they do not provide complete therapeutic protection against nerve agent exposure. An effective therapeutic must be able to counteract a broad range of organophosphorus agents, have a quick onset of action, prevent nerve-agent induced motor and cognitive defects, and have limited side effects. Importantly, for successful nerve agent prophylactic protection in humans, a therapeutic should provide prophylactic protection within 24 hours and have a duration of action of at least 10 days. These requirements have not yet been successfully met by any nerve agent bioscavenger.

3. Cotherapy with Hyaluronan-Degrading Enzyme

It is found herein that co-administering (as a combination or composition) an OP bioscavenger with a hyaluronan-degrading enzyme enhances exposure in the circulation to the biotherapeutic agent within 24 hours at levels that provide prophylactic protection. Thus, administering the combinations or compositions provided herein can result in exposure to circulating levels of bioscavenger of at least or about at least 15 μg/mL bioscavenger within 24 hours, such as at least or about at least 20 μg/mL, 21 μg/mL, 22 μg/mL, 23 μg/mL, 24 μg/mL, 25 μg/mL, 26 μg/mL, 27 μg/mL, 28 μg/mL, 29 μg/mL, 30 μg/mL, 40 μg/mL, 50 μg/mL, or more. This means that the OP bioscavenger is available in the blood stream in amounts that can combat the effects of exposure to nerve agents.

Co-administering an OP bioscavenger with a hyaluronan-degrading enzyme also can result in maintenance of circulating levels of bioscavenger at this level or higher for at least 10 days or more by inclusion in the combinations or compositions an OP bioscavenger that has a long duration of action (e.g. due to PEGylation). For example, in some examples of the combinations and compositions provided herein, the duration of action (e.g. half-life) of the OP bioscavenger is at least 24 hours, such as at least 30 hours, 40 hours, 50 hours, 60 hours or more. Hence, the combinations and compositions provided herein result in prophylactic protection sufficient to counteract the effects of exposure to nerve agent poisoning. For example, the combinations and compositions provided herein can provide prophylactic protection within 24 hours and have a duration of action of at least 10 days.

In particular examples, due to the effect on prophylactic protection afforded by co-administration with a hyaluronan-degrading enzyme, the combinations/compositions provided herein can be administered via percutaneous injection. For example, the combinations/compositions provided herein can be administered by subcutaneous injection, intramuscular injection or intradermal injection. In some examples, the product can be provided separate or together in a vial, syringe or auto-injector device (e.g. a pen), which can permit self-administration. The product can be in lyophilized form or in solution. When in lyophilized form, the vial or syringe or other device can contain a solution that can permit admixture immediately prior to use. This ability to percutaneously administer, by IM or SC injection, the provided formulations of cholinesterase and hyaluronan-degrading enzyme, provides ease of use and portability and thus presents a new approach to counteract nerve agent poisoning.

For example, as shown herein, pharmacokinetic analysis of plasma levels of an exemplary cholinesterase, PEG-rBChE, when co-administered with a hyaluronan-degrading enzyme (e.g., rHuPH20), reveals that the addition of the hyaluronan-degrading enzyme results in a quicker onset, e.g., shorter time, to the maximum observed concentration of rBChE and additionally results in increased absolute bioavailability (see Example 7 below). That is, the bioavailability of rBChE in the first 24 hours is greatly increased when co-administered with rHuPH20, as compared to rBChE administered alone. For example, in experiments described herein using guinea pigs, co-administration with rHuPH20 was shown to increase early AUC within the first 24 hours by 50% and 81% when administered by intramuscular (IM) or subcutaneous (SC) injection, respectively, and resulted in an absolute bioavailability of bioscavenger that was increased from 73% (IM) and 53% (SC) to 81% and 66%, respectively.

Further, as shown herein, the co-administration with a hyaluronan-degrading enzyme results in an overall increase in early exposure of the bioscavenger that is greater when the combinations/compositions are administered by IM injection as compared to SC injection. Hence, in particular examples herein, the combinations/compositions provided herein are administered by IM injection. Intramuscular injections can reduce irritations caused by injection into the subcutaneous tissues. Hence, intramuscular injections are generally safe and well tolerated.

Co-therapy with a hyaluronan-degrading enzyme also has other benefits. For example, the inclusion of a hyaluronan-degrading enzyme in a composition can allow for dose sparing such that less OP bioscavenger can be administered while still achieving prophylactic protection in the circulation. For illustration purposes, dose sparing means that an OP bioscavenger (e.g. PEG-rBChE) that is administered at or about at 100 mg/mL in a volume of 5-7 mL can be administered instead in a volume of 4-6 mL, thereby resulting in administration of a lower total dose of OP bioscavenger. This illustration is not intended to be limiting. It is within the level of one of skill in the art to empirically determine the appropriate or recommended amount of OP bioscavenger to administer and in what appropriate volume depending on the particular bioscavenger enzyme, whether it is intended for prophylactic protection or treatment, and/or the route of administration.

In other examples, the inclusion of a hyaluronan-degrading enzyme in a combination or composition provided herein can permit higher or greater amounts of OP bioscavenger to be administered in order to afford a higher level nerve agent protection. For example, the higher level of nerve agent protection means that increased or greater circulating bioscavenger is available in the blood stream earlier and/or for longer in order to combat affects from nerve agent exposure. For example, 800 mg to 2000 mg of bioscavenger can be administered, such as 1000 mg to 1400 mg (e.g. of 200 mg/mL formulation). This means that the frequency of dosing with an OP bioscavenger can be less in order to maintain the circulating levels of bioscavenger in the plasma at levels that are at least 15 μg/mL or more, and generally at least 20-30 μg/mL, such as at least 29 μg/mL. In other examples, this means that prophylactic protection can be achieved in less than 24 hours, such as by or within 6 hours to 24 hours, 8 to 18 hours, 12 to 16 hours, such as within 7 hours, 8 hours, 9 hours, 10 hours, 11 hours, 12 hours, 14 hours, 16 hours or 18 hours.

Any hyaluronan-degrading enzyme, including any truncated or variant thereof form thereof, can be used herein provided the enzyme exhibits hyaluronan-degrading activity. Any suitable bioscavenger, including any variant thereof can be used in the provided formulations, compositions and combinations, provided the bioscavenger binds the organophosphate compound and/or exhibits nerve agent-inactivating activity. For example, the bioscavenger can be a cholinesterase, such as an acetylcholinesterase or butyrylcholinesterase, such as a recombinant butyrylcholinesterase. One or the other or both of the bioscavenger and the hyaluronan-degrading enzyme can be modified by conjugation to a polymer (e.g. PEG) to increase the half-life of the enzyme. Typically, the bioscavenger is modified to increase its half-life, for example, by PEGylation. The increased half-life can increase systemic hyaluronidase and/or cholinesterase activity and sustained duration of action.

The formulations, compositions and combinations of an organophosphorus bioscavenger and hyaluronan-degrading enzyme, provided herein can be provided separately or provided in a single composition. The formulations, compositions and combinations can be provided in vials, syringes or any other suitable container for administration to a subject in need. The hyaluronan-degrading enzyme, such as hyaluronidase, e.g., rHuPH20, can be administered immediately before, immediately after or simultaneously with the organophosphate bioscavenger. The formulations, compositions and combinations can be administered by intravenous bolus (IV), or percutaneously, such as by subcutaneous (SC) or intramuscular (IM) injection. Typically, the formulations, compositions and combinations of an organophosphate bioscavenger and hyaluronan-degrading enzyme provided herein are administered by intramuscular (IM) injection. The formulations, compositions and combinations can be administered in a single dose, or can be administered in multiple doses.

In the methods provided herein, the formulations, compositions and combinations containing an organophosphorus bioscavenger and hyaluronan-degrading enzyme also can be combined and/or co-formulated with another agent useful for the treatment of organophosphorus poisoning. The second agent can be administered with or separate from the provided formulations, compositions and combinations containing an organophosphorus bioscavenger and hyaluronan-degrading enzyme.

In order to sustain the therapeutic effect, cycles of administration can be effected. Hence, the formulations, compositions and combinations can be administered successively over a dosing regime in order to maintain a constant level of OP bioscavenger for any desired length of time. The length of time of the cycle of administration depends on the severity of the organophosphorus poisoning, the particular patient, and other considerations within the level of skill of the treating physician. Over the course of treatment, evidence of toxicity can be monitored.

The following sections describe exemplary formulations, compositions and combinations containing an OP bioscavenger, such as a cholinesterase, and a hyaluronan-degrading enzyme, methods of making them, and using them to treat or prevent organophosphorus poisoning.

### C. Organophosphorus Bioscavengers

Provided herein are compositions and combinations containing an organophosphorus bioscavenger and a hyaluronan-degrading enzyme. The compositions and combinations can be used in methods of treatment and uses for counteracting the effects of poisoning caused by OP compounds and nerve agents, including in prophylactic treatments.

Organophosphate bioscavengers are proteins that act as biological scavengers for organophosphorus compounds (organophosphates). Organophosphates (OP), including organophosphorus pesticides and nerve agents, are phosphorus-containing organic chemicals that inhibit the action of acetylcholinesterase by binding covalently within the active site of the enzyme where acetylcholine undergoes hydrolysis. Acetylcholinesterase terminates the action of the neurotransmitter acetylcholine at postsynaptic membranes and neuromuscular junctions. Thus, OP compounds prevent the breakdown of acetylcholine leading to a state of constant contraction of muscles. Examples of OP compounds are set forth in Table 3. Other organophosphorus compounds include, but are not limited to, the OP compounds set forth in Section H below. Exemplary OP compounds that have been used as chemical weapons or nerve agents include, for example, O-ethyl-N,N-dimethyl phosphoramidocyanidate (tabun or GA); diisopropyl phosphonofluoridate (sarin or GB); pinacolozymethyl-fluorophosphonate (soman or GD); cyclohexylmethyl phosphonofluoridate (cyclosarin or GF) and ethyl-5-diisopropylaminoethyl methylphosphonothiolate (VX).

OP bioscavengers are enzyme agents that act to sequester or catalyze toxic OP compounds in circulation before they reach their physiological targets. OP bioscavengers can react with multiple nerve agents so that they are effective against all or mostly all OP nerve agents. Organophosphorus scavengers include enzymes that react specifically, rapidly and irreversibly to organophosphorus compounds. Moreover, bioscavengers typically should have prolonged circulation, be innocuous in the absence of the organophosphorus compound, and be non-antigenic in humans.

There are at least two types of OP bioscavengers, stoichiometric or catalytic bioscavengers. Stoichiometric organophosphorus bioscavengers act by stoichiometrically binding OPs in a 1:1 mole ratio, thereby both inactivating and sequestering the toxic OP compounds before they can exert effects on the nervous system. Examples of stoichiometric organophosphorus bioscavengers include, for example, cholinesterases and carboxylesterases. Catalytic OP bioscavengers act by catalyzing the hydrolysis, or breakdown, of organophosphorus compounds. Examples of catalytic OP bioscavengers include, for example, hydrolases, anhydrases and paraoxonases.

In the combinations, compositions and methods provided herein, the organophosphorus bioscavenger can be a stoichiometric bioscavenger or a catalytic bioscavenger. It is understood that any organophosphorus bioscavenger can be used in the compositions, combinations and methods provided herein, including any known in the art (see, e.g., U.S. Pat. Nos. 7,754,461, 7,572,764, 6,642,037; U.S. Pat. Pub. Nos. 201000333221, 20090208480, 20070184045, 20070111279, 20060194301, 20040005681, 20030113902, 20040016005, 20040168208 and 20060253913). For example, the organophosphorus bioscavenger can be an isolated or purified naturally occurring protein, a recombinantly generated protein or a synthetically generated protein. Further, the organophosphorus bioscavenger can be a truncated form of a full-length protein that binds to and sequesters an organophosphorus compound, e.g., OP sequestering activity, or hydrolyses an organophosphorus compound, e.g., OP hydrolytic activity. Hence, organophosphorus bioscavengers provided in the compositions, combinations and methods herein include full-length enzymes or active portions thereof that exhibit OP sequestering and/or hydrolytic activity. The activity generally is at least or at least about or 30%, 40%, 50%, 60%, 70%, 80%, 90% or 1.5 more of the activity of the corresponding full-length enzyme.

Exemplary organophosphorus bioscavengers include, but are not limited to, esterases, such as cholinesterases (ChE), including acetylcholinesterase (AChE) and butyrylcholinesterase (BChE), prolidases, such as organophosphate acid anhydrolase (OPAA), phosphotriesterases, such as aryldialkylphosphatases (organophosphorus hydrolases, OPH, OdpA), parathion hydrolases (PH, organophosphorus acid anhydrase), diisopropyl fluorophosphatases (DFPase), and sarinases, and paraoxonases (PON), or any active fragment thereof or any variant thereof that exhibits OP sequestering activity (binds to and sequesters an organophosphorus compound) or hydrolytic activity. The sequences of exemplary organophosphorus bioscavengers are set forth in any of SEQ ID NOS: 214-256 and 258-301. Organophosphorus bioscavengers and the OP compounds they are capable of neutralizing are set forth in Table 3 below.

TABLE 3

| Organophosphorus Bioscavenger | Organophosphorus Compound(s) |
|---|---|
| Acetylcholinesterase | Tabun or GA |
| | Soman or GD |
| | Sarin or GB |
| | VX |
| Butyrlchlinesterase | Tabun or GA |
| | Soman or GD |
| | Sarin or GB |
| | VX |
| | diisopropyl fluorophosphate (DFP) |
| Paraoxonases | Paraoxon |
| | Soman or GD |
| | Sarin or GB |
| | VX |
| | Diazoxon |
| | Chlorpyrifosoxon |
| | Diazonin |
| Organophosphorus hydrolases | Parathion |
| | Soman or GD |
| | Sarin or GB |
| | Cyclosarin or GF |
| | VX |
| | Paraoxon |
| | Diazoxon |
| | Chlorpyrifosoxon |
| | Diazonin |
| | diisopropyl fluorophosphate (DFP) |
| | Coumaphos |
| | Bensulide |
| Parathion hydrolases | Parathion |
| Aryldialkylphosphatases | Sarin or GB |
| | Soman or GD |
| | Cyclosarin or GF |
| | Tabun or GA |
| Diisopropylfluorophosphatases | Sarin or GB |
| | Soman or GD |
| | Cyclosarin or GF |
| | VX |
| | diisopropyl fluorophosphate (DFP) |
| Organophosphate acid anhydrolases | Paraoxon |
| | Tabun or GA |
| | Sarin or GB |
| | Soman or GD |
| | Cyclosarin or GF |
| | diisopropyl fluorophosphate (DFP) |

Organophosphorus bioscavengers provided herein also include variants of any of the above OP bioscavengers. In some examples, variants include variants of a cholinesterase or a phosphotriesterase. For example, the variants can include allelic or species variants or other variants of OP bioscavengers. For example, an organophosphorus bioscavenger can contain one or more variations in its primary sequence, such as amino acid substitutions, additions and/or deletions. A variant of an OP bioscavenger generally exhibits at least or about at least or 60%, 70%, 80%, 90%, 91%, 92%, 93%, 94%, 95%, 96%, 97%, 98%, 99% or more sequence identity compared to the OP bioscavenger not containing the variation, for example, any OP bioscavenger set forth in any of SEQ ID NOS:214-256 and 258-301. Any variation can be included in the organophosphorus bioscavenger for the purposes herein provided the OP bioscavenger exhibits OP inactivating activity, such as OP sequestering or hydrolytic activity, such as at least or about at least or 5%, 10%, 15%, 20%, 25%, 30%, 35%, 40%, 45%, 50%, 55%, 60%, 65%, 70%, 75%, 80%, 85%, 90%, 95% or more of the OP inactivating activity, such as OP sequestering or hydrolytic activity, of an OP bioscavenger not containing the variation (as measured in vitro and/or in vivo assays well known in the art and described herein).

Organophosphorus bioscavengers also include those that contain chemical or posttranslational modifications. Such modifications include, but are not limited to, PEGylation, albumination, glycosylation, farnesylation, carboxylation, hydroxylation, phosphorylation, and other polypeptide modifications known in the art. It is understood that such modified forms exhibit OP inactivating activity, such as OP sequestering or hydrolytic activity, such as at least or about at least or 5%, 10%, 15%, 20%, 25%, 30%, 35%, 40%, 45%, 50%, 55%, 60%, 65%, 70%, 75%, 80%, 85%, 90%, 95% or more of the OP inactivating activity, such as OP sequestering or hydrolytic activity, of an OP bioscavenger not containing the modification (as measured in vitro and/or in vivo assays well known in the art and described herein).

Exemplary of organophosphorus bioscavengers in the compositions, combinations and methods provided herein are butyrylcholinesterases, and modified forms thereof. For example, Protexia® (PharmAthene) is a recombinant version of a PEGylated human butyrylcholinesterase (BChE) produced in transgenic goats, designated PEG-rBChE (see, U.S. Pat. Pub. Nos. 20040016005, 20040168208 and 20060253913, and International Pat. Pub. No. WO2003054182).

A description is provided below of exemplary OP bioscavengers included in the compositions and combinations provided herein, or for use in the methods provided herein.

1. Cholinesterases

Cholinesterases (ChE) are a family of enzymes involved in nerve impulse transmissions. Cholinesterases are classified into two groups based on substrate preference and sensitivity to selective inhibitors. Acetylcholinesterases (AChE) preferentially hydrolyze acetyl esters such as acetylcholine and are sensitive to the chemical inhibitor BW 284C51. Butyrylcholinesterases (BChE) preferentially hydrolyze other types of esters, such as butyrylcholine, and are sensitive to the chemical inhibitor tetraisopropylpyrophosphoramide (iso-OMPA). OP compounds irreversibly inhibit cholinesterases. Inhibition of cholinesterases by OP compounds occurs through nucleophilic attack of the active serine on the phosphorus atom of the OP compound. This forms a covalent bond between the cholinesterase and the OP compound. Titration of organophosphates both in vitro and in vivo demonstrates a 1:1 stoichiometry between cholinesterases and the cumulative dose of the toxic nerve agent due to the formation of a covalent conjugate of the organophosphorus compound with the ChE active site serine.

Cholinesterases exist in several different polymeric forms, including monomers, dimers and tetramers. Native human cholinesterases are tetrameric proteins whereas recombinantly produced cholinesterases typically contain a mixture of monomers, dimers and tetramers.

a. Acetylcholinesterases

Acetylcholinesterases (EC 3.1.1.7) are type B carboxylesterases that rapidly hydrolyze the neurotransmitter acetylcholine at neuromuscular junctions and brain cholinergic synapses. Acetylcholinesterases are also referred to as acetylcholine acetylhydrolases, or true, specific, genuine, erythrocyte, red cell or Type I cholinesterases.

Human AChE is a globular, tetrameric serine esterase, membrane-bound glycoprotein that is found in erythrocytes, nerve endings, lungs, spleen and the gray matter of the brain. The gene encoding human AChE is located on chromosome 7 and several isoforms or splice variants and naturally occurring genetic variants exist.

Acetylcholinesterases of this type include, but are not limited to, cholinesterases from rat (SEQ ID NO:220), mouse (SEQ ID NO:222), cat (SEQ ID NO:228), chicken (SEQ ID NO:226), rabbit (SEQ ID NO:224), cow (bovine; SEQ ID NO:230), pacific electric ray (SEQ ID NO:232) and fruit fly (SEQ ID NO:234). Exemplary of acetylcholinesterases used in the compositions, combinations and methods provided herein are human acetylcholinesterases (set forth in SEQ ID NO:215). An acetylcholinesterase set forth in any of SEQ ID NOS: 214-234 and 294-296, active fragments thereof, or variants thereof can be used in the combinations, compositions or methods provided herein. The acetylcholinesterases can be monomers or can be oligomers, or combinations thereof. For example, compositions containing an acetylcholinesterases in the compositions and combinations herein can contain an acetylcholinesterase that is a monomer, dimer, trimer or tetramer, or combinations thereof in various abundance.

Specifically, the human acetylcholinesterase transcript is normally translated to form a 614 amino acid precursor polypeptide (SEQ ID NO:214) containing a 31 amino acid signal sequence at the N-terminus (amino acid residues 1-31). The mature AChE therefore, is a 583 amino acid polypeptide set forth in SEQ ID NO:215. Human acetylcholinesterase has three N-linked glycans at residues N296, N381 and N495 of SEQ ID NO:214 (corresponding to N265, N350 and N464 or SEQ ID NO:215). Human AChE has eight native cysteines, six of which form three disulfide bonds at residues C100-C127, C288-C303 and C440-0560 of precursor SEQ ID NO:214 (corresponding to C69-C96, C257-C272 and C409-0529 of SEQ ID NO:215). An interchain disulfide bond is formed at residues C611 (corresponding to C580 of SEQ ID NO:215).

The active site of AChE is found at the bottom of a 20 Å deep cavity that is lined with 14 aromatic residues (see Sussman et al., (1991) Science 253(5022):872-879). The active site catalytic triad, which binds the acyl moiety of the substrate acetylcholine, includes amino acids Ser234, Glu365 and His478 of SEQ ID NO:215 (corresponding to Ser203, Glu334 and H is 447 of SEQ ID NO:215).

Human acetylcholinesterases exist in several molecular forms including at least four isoforms produced by alternative slicing, including Isoform T (AChE-S, synaptic; U.S. Pat. No. 5,595,903); Isoform H (AChE-E, erythrocytic); Isoform R (Ache-R, readthrough; U.S. Pat. No. 6,025,183)); and Isoform 4.

Also included are variants of any of SEQ ID NOS:214-234 and 294-296 that have at least or about at least or about or 60%, 70%, 80%, 90%, 91%, 92%, 93%, 94%, 95%, 96%, 97%, 98%, 99% or more sequence identity to any of SEQ ID NOS: 214-234 and 294-296. Amino acid variants include variants that contain conservative and non-conservative mutations. It is understood that residues that are important or otherwise required for the activity of an acetylcholinesterase, such as any described above or known to skill in the art, are generally invariant and cannot be changed. These include, for example, active site residues. Thus, for example, amino acid residues 203, 334 and 447 (corresponding to residues in the mature human AChE set forth in SEQ ID NO:215) of a human acetylcholinesterase are generally invariant and are not altered.

For example, variants of an acetylcholinesterase include allelic variants (SEQ ID NO:294-296), splice variants (U.S. Pat. No. 5,932,780), and post-translational modification variants such as non-glycosylated variants (U.S. Pat. No. 5,284,604),

Acetylcholinesterases in the combinations and compositions provided herein also can include variants that have an amino acid modification and that exhibits an altered, such as improved, activity compared to an acetylcholinesterase not including the modification. Such variants include those that contain an amino acid modification that enhances the catalytic activity of the acetylcholinesterase. For example, the amino acid modification can be an amino acid replacement (substitution), deletion or insertion. Such modified acetylcholinesterases include any acetylcholinesterase described in the art, including, but not limited to, acetylcholinesterases containing mutations or amino acid replacements at amino acid residues corresponding to R3, P104, G121, G122, Y124, D134, E202, A258, E268, F295, V302, H322, F338, D385, F455 or C580 in the sequence of amino acid set forth in SEQ ID NO:215. The mutation or amino acid replacement can be any replacement to any one of the other 19 amino acids at that position, so long as the variant exhibits acetylcholinesterase activity to inactivate an OP compound. Hence, provided in the compositions and combinations herein are modified or variant acetylcholinesterases containing at least one mutation corresponding to R3Q, P104A, G121H, G122H, Y124H, D134H, E202Q, A258T, E268T, F295H, V302E, H322N, F338D, F338E, F338A, D384G, F455L, C580A, F295H/F338D, F295H/F338E, D134H/E202Q, D134H/F338A with respect to the acetylcholinesterase set forth in SEQ ID NO:215 (see, e.g., U.S. Pat. No. 6,001,625; Kucukkilinc et al., (2010) Chem Biol Interact 187:238-240).

b. Butyrylcholinesterases

Butyrylcholinesterases (EC 3.1.1.8) are non-specific cholinesterases that hydrolyze various choline esters. Butyrylcholinesterases are also referred to as pseudocholinesterases, or non-specific, plasma, serum, benzoyl, false or Type II cholinesterases. Although its natural substrate is unknown, butyrylcholinesterases are known to hydrolyze acetylcholine and a variety of different choline esters. An addition, butyrylcholinesterases preferentially use butyrylcholine and benzoylcholine as in vitro substrates. Butyrylcholinesterases are reactive towards a wide variety of organophosphorus compounds (see Table 3 above). Butyrylcholinesterases are stoichiometric scavenger in vivo, that is, they form stable stoichiometric (1:1) covalent conjugates between the organophosphorus compound and the active site serine residue.

Human BChE is a globular, tetrameric serine esterase with a molecular mass of approximately 340 kDa. BChE in human serum has a concentration of 5 mg/L and a half-life of 12 days (Ostergaard et al., (1988) Acta Anaesthesiol Scand 32:266-269). BChE is synthesized in the liver and is present in mammalian blood plasma, liver, pancreas, intestinal mucosa, the white matter of the central nervous system, smooth muscle and heart. The gene encoding human BChE is located on chromosome 3. Exemplary of butyrylcholinesterases used in the compositions, combinations and methods provided herein are human butyrylcholinesterases (set forth in SEQ ID NO:236). Species variants of butyrylcholinesterases type include, but are not limited to cholinesterases from rat (SEQ ID NO:240), mouse (SEQ ID NO:242), cat (SEQ ID NO:244), horse (SEQ ID NO:245), chicken (SEQ ID NO:247), pig (SEQ ID NO:248), rabbit (SEQ ID NO:250), cow (bovine; SEQ ID NO:252), sheep (Ovis aries; SEQ ID NO:253), rhesus monkey (SEQ ID NO:254) and Bengal tiger (SEQ ID NO:256). A butyrylcholinesterase set forth in any of SEQ ID NOS: 235-257 and 291-293, active fragments thereof, or variants thereof can be used in the combinations, compositions or methods provided herein. The butyrylcholinesterase can be monomers or can be oligomers, or combinations thereof. For example, compositions containing a butyrylcholinesterase in the compositions and combinations herein can contain a butyrylcholinesterase that is a monomer, dimer, trimer or other higher ordered oligomer, or combinations thereof in various abundances.

The human butyrylcholinestase transcript is normally translated to form a 602 amino acid precursor polypeptide (SEQ ID NO:235) containing a 28 amino acid signal sequence at the N-terminus (amino acid residues 1-28). The mature BChE therefore, is a 574 amino acid polypeptide set forth in SEQ ID NO:236. Structural analysis reveals BChE is a tetrameric glycoprotein containing four identical subunits, each containing 574 amino acids and nine N-linked glycans and having a molecular weight of 85 kDa. (Lockridge et al., (1987) J Biol Chem 262:549-557). The nine N-linked glycans in human BChE are at residues N45, N85, N134, N269, N284, N369, N483, N509, N513 and N514 of SEQ ID NO:235 (corresponding to amino acids N17, N57, N106, N241, N256, N341, N455, N481, N485 and N486 of SEQ ID NO:236). Human butyrylcholinesterase has eight native cysteines, six of which form three disulfide bonds, at residues C93-C120, C280-C291 and C428-0547 of precursor SEQ ID NO:235 (corresponding to C65-C92, C252-C263 and C400-0519 of SEQ ID NO:236). In addition, an interchain disulfide bond is formed at residue C599 of SEQ ID NO:235 (corresponding to C571 of SEQ ID NO:236). The C-terminus of human BChE (amino acid residues 559-602 of SEQ ID NO:235 corresponding to amino acid residues 531-574 of SEQ ID NO:236) is rich in aromatic amino acids that cause aggregation and is referred to as the tetramerization or aggregation domain. C-terminal amino acids 525-574 of SEQ ID NO:236 are not necessary for BChE biological activity (Blong et al., (1997) Biochem J 327:747-757)

The active site catalytic triad includes residues Ser226, Glu353 and His466 of precursor set forth in SEQ ID NO:235 (corresponding to Ser198, Glu325 and His438 of SEQ ID NO:236). The acyl binding pocket of BChE is lined with aliphatic residues (see, Nicolet et al., (2003) J Biol Chem 278:41141-41147).

Also included in the compositions, combinations or methods provided herein are variants of any of SEQ ID NOS:235-257 and 291-293 that have at least or about at least or about or 60%, 70%, 80%, 90%, 91%, 92%, 93%, 94%, 95%, 96%, 97%, 98%, 99% or more sequence identity to any of SEQ ID NOS: 235-257 and 291-293. Amino acid variants include variants that contain conservative and non-conservative mutations. It is understood that residues that are important or otherwise required for the activity of a butyrylcholinesterase, such as any described above or known to skill in the art, are generally invariant and cannot be changed. These include, for example, active site residues. Thus, for example, amino acid residues 198, 325 and 438 (corresponding to residues in the mature human BChE set forth in SEQ ID NO:236) of a human butyrylcholinesterase are generally invariant and are not altered.

For example, there are at least four predominant allelic forms of human BChE, including Eu (wildtype), Ea (A, atypical, containing the mutation D70G in SEQ ID NO:236 (McGuire et al., (1989) Proc Natl Acad Sci USA 86:953-957)), Ef and Es and over 30 naturally occurring genetic variants and more than eleven isoenzyme variants are known. Additional natural variants include the J variant containing the mutation E497V (Bartels et al. (1992) Am J Hum Genet. 50:1104-1114); K variant containing the mutation A539T (Bartels et al., (1992) Am J Hum Genet. 50:1086-1103); and H variant containing the mutation V142M (all in the sequence of amino acids set forth in SEQ ID NO:236).

Butyrylcholinesterases in the combinations or compositions provided herein also can include variants that have an amino acid modification and that exhibits an altered, such as improved, activity compared to a butyrylcholinesterase not including the modification. Such variants include those that contain an at least one amino acid modification that enhances the catalytic activity of the butyrylcholinesterase. Such modified butyrylcholinesterases include any butyrylcholinesterase described in the art, including, but not limited to, butyrylcholinesterases containing mutations or amino acid replacements at amino acid residues corresponding to D70, W82, W112, G115, G116, G117, Q119, T120, Y128, E197, S198, A199, S224, F227, W231, A277, P285, L286, S287, V288, G291, E325, A328, F329, V331, Y332, C400, Y419, W430, H438, G439, Y440 and E441 in the sequence of amino acid set forth in SEQ ID NO:236. The mutation or amino acid replacement can be any replacement to any one of the other 19 amino acids at that position, so long as the variant exhibits butyrylcholinesterase activity to inactivate an OP compound. For example, modified butyrylcholinesterases that can be included in the combinations or compositions provided herein include any containing at least one mutation corresponding to D70N, G115A, G116F, G116W, G116H, G116S, G117H, G117K, G117C, G117N, Q119Y, Q119H, Q119E, Q119D, T120F, E197Q, E197D, E197G, A199S, S224Y, F227A, F227G, F227P, F227T, F227S, F227C, F227M, F2271, F227L, F227V, A277D, A277E, P285Q, P285S, P285A, P285G, P2851, P285K, L286A, L286H, L286W, L286M, S287G, V288F, V288H, V288W, V288E, G291E, G291D, A328F, A328Y, A328G, A328H, A3281, A328W, F329A, F329D, F329S, V331L, Y332A, Y332S, Y332F, Y332M, Y332G, Y332P, C400S, Y419S, G439A, G439L, E441D. The variant or modified butyrylcholinesterases can include those with more than one mutation such as any having amino acid replacements corresponding to G117H/Q119E, G117H/E197Q, Q119HN288E, Q119DN288H, Q119E/V288H, F227A/A328W, L286H/F329D, F288H/G291E, F288H/G291D, V288H/A277D, V288H/A277E, A328W/F227A, A328W/V331L, A328W/Y332M, A328W/Y332P, A328W/Y332S, A328W/Y332A, A328W/Y332G, N68Y/Q119Y/A277W, Q119Y/V288F/A328Y, A199S/S287G/A328W, A199S/A328W/Y332G, F227A/S287G/A328W, A328W/Y332A/Y419S, A199S/F227A/A328W/Y332G, A199S/S287G/A328W/Y332G, A199S/F227A/S287G/A328W, F227A/S287G/A328W/Y332M, A199S/F227A/S287G/A328W/Y332G, A199S/F227A/S287G/A328W/Y332M, A199S/F227A/S287G/A328W/E441D, A199S/F227A/P285A/S287G/A328W/Y332G, A199S/F227A/P285S/S287G/A328W/Y332G, A199S/F227A/P285Q/S287G/A328W/Y332G, A199S/F228P/S287G/A328W/Y332G, A199S/F227A/P285G/S287G/A328W/Y332G, A199S/F227A/L286M/S287G/A328W/Y332G, A199S/P285Q/S287G/A328W/Y332G, A199S/SP285I/287G/A328W/Y332G, A199S/F227G/S287G/A328W/Y332G, A199S/P285S/S287G/A328W/Y332G, A199S/F227V/S287G/A328W/Y332G, A199S/P285G/S287G/A328W/Y332G, A199S/F2271/S287G/A328W/Y332G, A199S/F227L/S287G/A328W/Y332G, A199S/L286M/S287G/A328W/Y332G, and A199S/F227A/P285K/S287G/A328W/Y332G in SEQ ID NO:236 (see, e.g., U.S. Pat. Nos. 6,001,625, 7,049,121, 7,070,973, 7,892,537 and 7,919,082; U.S. Pat. Pub. No. 2009/0249503; and Xie et al., (1999) Molecular Pharmacology 55:83-91, Vyas et al., (2010) Chem Biol Interact 187:241-245). Additional variants include those with at least one amino acid mutation in one or more regions corresponding to amino acid positions 62-82, 110-121, 194-201, 224-234, 277-289, 327-332, and/or 429-442 of SEQ ID NO:236 (U.S. Pat. No. 7,070,973).

In some instances in the combinations and compositions provided herein, a human butyrylcholinesterase can be truncated to remove all or part of the C-terminus. Such truncation can remove all or part of the aggregation domain. For example, a human butyrylcholinesterase provided herein can be C-terminally truncated by 1, 2, 3, 4, 5, 6, 7, 8, 9, 10, 15, 20, 25, 30, 40, 50, 60 or more amino acids compared to the wild type butyrylcholinesterase, for example a butyrylcholinesterase set forth in SEQ ID NO:236, provided that the C-terminally truncated butyrylcholinesterase exhibits OP inactivating activity, such as OP sequestering and/or hydrolytic activity. In some instances, a butyrylcholinestase provided herein is truncated after amino acid residue 524 of SEQ ID NO:236. In other examples, a butyrylcholinestase provided herein is truncated after amino acid residue 530 of SEQ ID NO:236.

rBChE

Any of the above butyrylcholinesterases can be produced recombinantly. Methods of recombinant DNA techniques are well-known to one of skill in the art. For example, a butyrylcholinesterase, active portion thereof or variant thereof can be produced recombinantly by expression of an encoding nucleic acid in mammalian cells, for example CHO cells. In other examples, a butyrylcholinesterase, active portion thereof or variant thereof also can be produced in other expression systems such as by expression in transgenic animals or plants.

For example, a butyrylcholinesterase set forth in SEQ ID NO:236 (and encoded by a sequence of nucleic acids set forth in SEQ ID NO:257) has been expressed recombinantly from CHO cells. The crystal structure of full-length monomeric recombinant human butyrylcholinesterase produced in CHO cells has been determined (see Ngamelue et al., (2007) Acta Crsytallogr Sect F Structu Biol Cryst Commun 63(Pt 9):723-727). When expressed in CHO cells, recombinant BChE is recovered as a mixture of oligomeric forms, including 15-40% monomers, 50-55% dimers and 10-30% tetramers. Recombinant BChE has also been expressed in E. coli (Masson P. (1992) in Multidisciplinary Approaches to Cholinesterase Functions, eds Shafferman A, Velan B (Plenum, New York) pp 49-52), microinjected Xenopus laevis oocytes; insect cell lines in vitro and larvae in vivo; silkworm Bombyx mori; and mammalian COS cells, 293T cells (Altamirano and Lockridge (1999) Chem Biol Interact 119-120:53-60) and CHO cells (Millard et al (1995) Biochemistry 34:15925-15933).

In addition, human butyrylcholinesterases have been expressed in transgenic mammals, including, but not limited to, mice and goats (see, Huang et al., (2007) Proc Natl Acad Sci USA 104:13603-13608; and U.S. Pat. Pub. Nos. 20040016005 20060253913). Protexia® is a recombinant human butyrylcholinesterase (rBChE) expressed in the milk of transgenic goats (see, Huang et al., (2007) Proc Natl Acad Sci USA 104:13603-13608 and U.S. Pat. Pub. Nos. 20040016005 and 20040168208; and International Pat. Pub. No. WO2003054182). rBChE can be expressed in transgenic goats under the control of a goat β-casein promoter (set forth in SEQ ID NO:195) and can be produced in concentrations of up to 5 g/L of milk. Transgenic recombinant BChE secreted in goat's milk is about 80% dimers and 20% monomers. rBChE produced in this manner is underglycosylated, having more fucose and GalNac and less mannose, galactose, GlcNac and sialic acid. rBChE is expressed mainly in dimeric form, with a half life in human serum of approximately 6.5 hours, if administered intravenously, and 7.1 hours if administered by intramuscular injection.

PEG-rBChE

Any of the above butyrylcholinesterases also can be further modified to increase half-life. This is further described in subsection 3. For example, PEG-rBChE is a known therapeutic agent and contains a sequence of amino acid thiols conjugated to PEG moieties. Each monomeric subunit is conjugated to a single PEG moiety. When PEGylated (see Section 3 below), PEG-rBChE has a half life of 44.2 and 40.7 hours when administered intravenously or by intramuscular injection, respectively. In addition, rBChE was shown to bind to nerve agents soman (GD), sarin (GB), tabun (GX) and VX in a 1:1 molar ratio as demonstrated by in vitro inhibition assays. PEG-rBChE purified from the milk of transgenic goats had a specific activity of approximately 700 U/mg (as measured by the Ellman assay). Non-denaturing PAGE gels stained for activity with butyryl-thiocholine revealed that PEG-rBChE secreted in the milk of transgenic goats contains a mixture of monomer, dimer and tetramer species with dimer being the predominant form. The mixture of these forms was either assembled into tetramers in vitro (approximately 60-70% tetramer content) using poly-proline or subjected to PEGylation using standard techniques (see U.S. Pat. Pub. No. 20090208480).

2. Other Organophosphorus Bioscavengers

Additional organophosphorus bioscavengers that bind to or hydrolyze organophosphorus compounds can be used in the provided compositions, combinations and methods. For example, aryldialkylphosphatases, such as paraoxonases, organophosphorus hydrolases, and parathion hydrolases, and diisopropyl fluorophosphatases, including organophosphate acid anhydrolases, that either bind to, or hydrolyze, organophosphorus compounds can be employed in the compositions, combinations and methods provided herein.

a. Aryldialkylphosphatases

Aryldialkylphosphatases (EC 3.1.8.1) are class of metal-dependent OP-hydrolases that are capable of hydrolyzing a broad range of organophosphorus compounds, including sarins and insecticidal organophosphates (Bird et al. (2008) Toxicology 21:88-92). Aryldialkylphosphatases require a binuclear metal, such as Zn²⁺, Mn²⁺, Co²⁺ or Cd²⁺, at their active site for enzymatic activity. Aryldialkylphosphatases include phosphotriesterases or OP hydrolases (PTE or OPH), paraoxon hydrolases or paraoxonases, parathion hydrolases (PH), and OpdA.

i. Paraoxonases

Serum paraoxonases (PON, EC 3.1.1.2; EC 3.1.8.1) are high-density lipoprotein (HDL)-associated esterases that hydrolyze toxic metabolites of organophosphorus insecticides, pesticides and nerve agents (see, U.S. Pat. Nos. 7,026,140 and 7,211,387). For example, paraoxonases are known to hydrolyze soman and sarin (Gan et al., (1991) Drug Metab Dispos 19:100-106). PON enzymes possess a conserved, hydrophobic leader sequence at their N-terminus of about or approximately 20 amino acids, which is not excised and is part of the mature protein. The leader sequence is directly involved in the binding of PONS to high density lipoproteins (HDL).

Human serum paraoxonase (HuPON1) is synthesized in the liver and secreted into the bloodstream where it is found bound to high-density lipoprotein in the circulation (Primo-Parmo et al., (1996) Genomics 33:498-507; Shih et al., (1998) Nature 394:284-287). HuPON1 hydrolyzes multiple classes of substrates, including aryl esters (including phenyl acetate), lactones, and organophosphorus compounds including paraoxon, diazoxon (DZO), and chlorpyrifosoxon and the chemical warfare agents sarin, soman and VX (Gan et al., (1991) Drug Metabl Dispos 19:100-106; Costa et al., (1999) Chem Biol Interact 119-120:429-438; Davies et al., (1996) Nat Genet. 14:334-336; Broomfield et al., (1991) J Pharmacol Exp Ther 259:633-638, Yeung et al., (2005) FEBS J 272:2225-2230; Stevens et al., (2008) Proc Natl Acad Sci USA 105:12780-12784).

Paraoxonases of this type include, but are not limited to, human paraoxonases, including PON1 (SEQ ID NO:258), PON2 (SEQ ID NO:264), and PON3 (SEQ ID NO:271); rabbit paraoxonases, including PON1 (SEQ ID NO:259) and PON3 (SEQ ID NO:274); mouse paraoxonases, including PON1 (SEQ ID NO:260), PON2 (SEQ ID NO:265), and PON3 (SEQ ID NO:272); rat paraoxonases, including PON1 (SEQ ID NO:261), PON2 (SEQ ID NO:270), and PON3 (SEQ ID NO:273); pig paraoxonases, including PON1 (SEQ ID NO:262) and PON3 (SEQ ID NO:276); cow paraoxonases, including PON1 (SEQ ID NO:263), PON2 (SEQ ID NO:269), PON3 (SEQ ID NO:275); chicken PON2 (SEQ ID NO:266); turkey PON2 (SEQ ID NO:267); dog PON2 (SEQ ID NO:268). Exemplary of paraoxonases used in the combinations, compositions and methods provided herein is human paraoxonase 1 (set forth in SEQ ID NO:258). A paraoxonase set forth in any of SEQ ID NOS:258-276, active fragments thereof, or variants thereof can be used in the combinations, compositions or methods provided herein.

Specifically, human serum paraoxonase 1 is a 43-45 kDa glycosylated protein containing 354 amino acids (PON¹, SEQ ID NO:258). Five PON1 promoter region polymorphisms and two PON1 coding region polymorphisms (including PON¹-55) have been identified (Costa et al. (2003) Annu Rev Med 54:371-392).

Also included in the compositions, combinations or methods provided herein are variants of any of SEQ ID NOS:258-276 that have at least or about at least or about or 60%, 70%, 80%, 90%, 91%, 92%, 93%, 94%, 95%, 96%, 97%, 98%, 99% or more sequence identity to any of SEQ ID NOS: 258-276. Amino acid variants include variants that contain conservative and non-conservative mutations. It is understood that residues that are important or otherwise required for the activity of a paraoxonase, such as any described above or known to skill in the art, are generally invariant and cannot be changed.

Paraoxonases in the combinations and compositions provided herein also can include variants that have an amino acid modification and that exhibits an altered, such as improved, activity compared to a paraoxonase not including the modification. Such variants include those that contain an amino acid modification that enhances the catalytic activity of the paraoxonase. For example, the amino acid modification can be an amino acid replacement (substitution), deletion or insertion. Hence, provided in the compositions and combinations herein are modified or variant paraoxonases containing at least one mutation corresponding to A6E, L10S, L14M, L28Y, N41D, E49N, L55M, L69G, L69I, L69V, L69A, L69S, L69M, K70A, K70S, K70Q, K70N, Y71F, Y71C, Y71A, Y71L, Y71I, V97A, 1102V, H115W, H115L, H115V, H115C, H115Q, A126T, H134Q, H134R, H134N, P135A, K138S, L143V, R160G, F178V, Q192R, Q192K, M196V, M196L, M196F, F222S, F222M, F222C, N227L, K233E, L240S, L240V, H243R, F264L, E276A, C284A, C284S, H285R, M2891, F292S, F292V, F292L, F293S, A301G, N324D, T326S, T332S, T332M, T332C, T332A, and V346A with respect to a paraoxonase set forth in SEQ ID NO:258 (see, U.S. Pat. App No. 20110171197; Int. Pat. Pub. No. WO2011033506; Hassett et al., (1991) Biochemistry 30:10141-10149; Adkins et al., (1993) Am J Hum Genet. 52:598-608; Sorenson et al., (1995) Proc Natl Acad Sci USA 92:7187-7191; Sorenson et al., (1999) Arterioscler Thromb Vasc Biol 19:2214-2225; Harel et al., (2004) Nat Struct Mol Biol 11:412-419; and Marchesani et al., (2003) J Natl Cancer Inst 95:812-818).

ii. Organophosphorus Hydrolases

Organophosphorus hydrolases (EC 3.1.8.1; OPH, also known as phosphotriesterase, PTE) are (β/α)8-barrel enzymes with binuclear metal centers located at the C-terminal end of the barrel that hydrolyze organophosphorus compounds (Vanhooke et al., (1996) Biochemistry 35:6020-6025). The reaction mechanism is proposed to proceed via an SN2-like mechanism in which the metal center enables a hydroxide ion (bridged to the two metal ions) to attack the electrophilic phosphorus of the substrate (Aubert et al., (2004) Biochemistry 43:5707-5715). OPHs are active against a wide range of phosphotriester OP pesticides, including substrates with phosphoryl sulfur (such as parathion) and substrates with methoxy and ethoxy groups.

Organophosphorus hydrolases are encoded by OpdA genes. Bacterial OpdAs that can be used in the provided combinations and compositions include, but are not limited to, those from Brevunduinibas diminuata MG (SEQ ID NO:278; Serdar and Gibson (1985) Bio/Technology 3:567-571); Flavobacterium sp. (Mulbry et al., (1986) Appl Environ Microbiol 51:926-930); Plesiomonas sp. strain M6 (Zhongli et al., (2001) Appl Environ Microbiol 67:4922-4925); Streptomyces lividans (Rowland et al., (1991) Appl Environ Microbiol 57:440-444); Agrobacterium radiobacter (Horne et al. (2002) Appl Environ Microbiol 68:3371-3376, Gresham et al., (2010) Acad Emerg Med 17:736-740); Sulfolobus solfataricus (SEQ ID NO:277); E. coli K12 (SEQ ID NO:282), Drosophila melanogaster (SEQ ID NO:283).

Also included in the compositions, combinations or methods provided herein are variants of any of SEQ ID NOS:277-278 and 282-283 that have at least or about at least or about or 60%, 70%, 80%, 90%, 91%, 92%, 93%, 94%, 95%, 96%, 97%, 98%, 99% or more sequence identity to any of SEQ ID NOS: 277-278 and 282-283. Amino acid variants include variants that contain conservative and non-conservative mutations. It is understood that residues that are important or otherwise required for the activity of an organophosphorus hydrolase, such as any described above or known to skill in the art, are generally invariant and cannot be changed. These include, for example, active site residues.

Organophosphorus hydrolases in the combinations and compositions provided herein also can include variants that have one or more amino acid modifications and that exhibit an altered, such as improved, activity compared to an organophosphorus hydrolase not including the modification. Such variants include those that contain one or more amino acid modifications that enhance the catalytic activity of the organophosphorus hydrolase. For example, the amino acid modification(s) can be an amino acid replacement(s) (substitution(s)), deletion(s) or insertion(s). Hence, provided in the compositions and combinations herein are modified or variant organophosphorus hydrolases containing at least one mutation corresponding to A14T, L17P, R67H, A80V, L87S, V116I, L130M, Q148R, L182S, K185R, A203T, H254R, H257Y, 1274N, V310A, P342S and S365P with respect to the organophosphorus hydrolase set forth in SEQ ID NO:278 (Cho et al., (2002) Appl Environ Microbiol 68:2026-2030; Cho et al. (2006) Protein Eng Des Sel 19:99-105; and Cho et al., (2004) Appl Environ Microbiol 70:4681-4685). The variant or modified organophosphorus hydrolases can include those with more than one mutation such as having any amino acid replacements corresponding to A80V/S365P, L182S1V301A, H257Y/1274N/S365P, L130M/H257Y/1274N, A14T/A80V/K185R/H257Y/1274N, A14T/A80V/K185R/1274N, A14T/R67H/A80V/L87S/Q148R/K185R, A14T/A80V/K185R, A14T/A80V/K185R/H254R/1274N and A14T/L17P/A80VN1161/K185R/A203T/1274N/P342S of the sequence of amino acids set forth in SEQ ID NO:278 (Cho et al., (2002) Appl Environ Microbiol 68:2026-2030; Chi) et al. (2006) Protein Eng Des Sel 19:99-105; and Cho et al., (2004) Appl Environ Microbiol 70:4681-4685).

iii. Parathion hydrolases

Parathion hydrolases are enzymes, typically bacterial enzymes, categorized by their ability to hydrolyze the organophosphorus compound parathion (O,O-diethyl-O-4-nitrophenyl phosphorothioate). Parathion hydrolases that can be included in the compositions, combinations and methods provided herein include, but are not limited to, parathion hydrolase from Burkholderia sp. JBA3 (SEQ ID NO:300; Kim et al., (2007) J Microbiol Biotechnol 17:1890-1893); Pseudomonas diminuta MG or Brevundiomonas diminuta (SEQ ID NO:278) (Serdar et al., (1989) Nature Biotechnology 7:1151-1155); Flavobacterium sp. strain ATCC 27551 (SEQ ID NO:279); Flavobacterium sp. strain MTCC 2495 (SEQ ID NO:298); and Sulfolobus acidocaldarius (SEQ ID NOS:280 and 281); methyl parathion hydrolase (MPH) from Bacillus subtilis WB800 (Zhang et al., (2005) Appl Environ Microbiol 71:4101-4103); and methyl parathion hydrolase from Plesiomonas sp. strain M6 (SEQ ID NO:297, Zhongli et al., (2001) Appl Environ Microbiol 67:4922-4925). A parathion hydrolase set forth in any of SEQ ID NOS:278-281, 297-298 and 300, active fragments thereof, and variants thereof can be used in the combinations, compositions or methods provided herein.

Also included in the compositions, combinations or methods provided herein are variants of any of SEQ ID NOS: 278-281, 297-298 and 300 that have at least or about at least or about or 60%, 70%, 80%, 90%, 91%, 92%, 93%, 94%, 95%, 96%, 97%, 98%, 99% or more sequence identity to any of SEQ ID NOS: 278-281, 297-298 and 300. Amino acid variants include variants that contain conservative and non-conservative mutations. It is understood that residues that are important or otherwise required for the activity of a parathion hydrolase, such as any described above or known to skill in the art, are generally invariant and cannot be changed. These include, for example, active site residues.

b. Diisopropyl fluorophosphatases

Diisopropyl fluorophosphatases (DFPase; EC 3.1.8.2) are divalent cation-dependent phosphotriesterases that are capable of hydrolyzing a variety of organophosphorus compounds including diisopropyl fluorophosphates (DFP) and G-type organophosphorus nerve agents, such as sarin (GB), soman (GD) and cyclosarin (GF) (Blum et al., (2010) Acta Crystallogr Sect F Struct Biol Cryst Commun 66(Pt 4):379-385). DFPases include tabunase, somanase, organophosphorus acid anhydrolase (OPA), organophosphate acid anhydrase (OPA), OPA anhydrase (OPAA), prolidases, diisopropylphosphofluoridase, dialkylfluorophosphatase, diisopropyl phosphorofluoridate hydrolase, isopropylphosphorofluoridase, diisopropyl fluorophosphonate dehalogenase and senescence marker protein 30 (SMP30).

Diisopropyl fluorophosphatases include, but are not limited to, DFPases from Loligo vulgaris (squid; SEQ ID NO:286), Alteromonas sp. (SEQ ID NO:287), Pseudoalteromonas haloplanktis (SEQ ID NO:288), Marinomonas mediterranea (SEQ ID NO:289), Aplysia californica (SEQ ID NO:301; Int. Pat Pub. No. WO2010128116), Octopus vulgaris (SEQ ID NO:299; Int. Pat. Pub. No. WO2010128115), Homo sapiens (SEQ ID NO:285) and rat senescence marker protein 30 (SEQ ID NO:290). Exemplary of a diisopropyl fluorophosphatase used in the combinations, compositions or methods provided herein is diisopropyl fluorophosphatase from Loligo vulgaris (SEQ ID NO:286). A diisopropyl fluorophosphatases set forth in any of SEQ ID NOS:285-290, 299 and 301, active fragments thereof, and variants thereof can be used in the combinations, compositions or methods provided herein

Also included in the compositions, combinations or methods provided herein are variants of any of SEQ ID NOS: 286-290, 299 and 301 that have at least or about at least or about or 60%, 70%, 80%, 90%, 91%, 92%, 93%, 94%, 95%, 96%, 97%, 98%, 99% or more sequence identity to any of SEQ ID NOS: 285-290, 299 and 301. Amino acid variants include variants that contain conservative and non-conservative mutations. It is understood that residues that are important or otherwise required for the activity of a diisopropyl fluorophosphatase, such as any described above or known to skill in the art, are generally invariant and cannot be changed. These include, for example, active site residues. Thus, for example, amino acid residues His287 (corresponding to residues in the diisopropyl fluorophosphatase Loligo vulgaris set forth in SEQ ID NO:286) of a diisopropyl fluorophosphatase is generally invariant and is not altered.

Diisopropyl fluorophosphatases in the combinations and compositions provided herein also can include variants that have one or more amino acid modifications and that exhibit an altered, such as improved, activity compared to an diisopropyl fluorophosphatase not including the modification. Such variants include those that contain one or more amino acid modifications that enhance the catalytic activity of the diisopropyl fluorophosphatase. For example, the amino acid modification(s) can be an amino acid replacement(s) (substitution(s)), deletion(s) or insertion(s). Hence, provided in the compositions and combinations herein are modified or variant diisopropyl fluorophosphatases containing at least one mutation corresponding to Q77Y, Q77W, Y144S, R146S, M148A, F173L, F173V, F173W, H181N, T195L, T195V, H219N, H224N, D232S, N237S, W244Y, W244H, H248N, S271A, H287F, H287L, H287W, Q304F, Q304W and F314A with respect to the diisopropyl fluorophosphatase set forth in SEQ ID NO:286 (Katsemi et al., (2005) Biochemistry 44:9022-9033; Hartleib and Rueterjans (2001) Biochim Biophys Acta 1546:312-324; and Scharff et al., (2001) Structure 9:493-502).

i. Organophosphate acid anhydrolases

Organophosphate acid anhydrolases (OPAA) are members of a class of bimetalloenzymes that hydrolyze a variety of toxic acetylcholinesterase-inhibiting organophosphorus compounds, including fluorine-containing chemical nerve agents. It also belongs to a family of prolidases, with significant activity against various Xaa-Pro dipeptides. The X-ray structure of native OPAA (58 kDa mass) from Alteromonas sp. strain JD6.5 reveals the OPAA structure is composed of two domains, amino and carboxy domains, with the latter exhibiting a “pita bread” architecture and harboring the active site with the binuclear Mn²⁺ ions (Vyas et al., (2010) Biochemistry 49:547-549).

Organophosphate acid anhydrolases include, but are not limited to, OPAA from Mycobacterium sp.; Amycolatopsis mediterranei; Streptomyces coelicolor; Streptomyces sp AA4; Streptomyces lividans TK24; Streptomyces sviceus ATCC29083; and Streptomyces griseoaurantiacus M045 and active fragments thereof, and variants thereof can be used in the combinations, compositions or methods provided herein. Amino acid variants include variants that contain conservative and non-conservative mutations. It is understood that residues that are important or otherwise required for the activity of an organophosphate acid anhydrolase, such as any described above or known to skill in the art, are generally invariant and cannot be changed. These include, for example, active site residues.

3. Modified Organophosphorus Bioscavengers

The organophosphorus bioscavengers for use in the compositions, combinations and methods provided herein can be modified, such as by, conjugation to polymeric molecules or by fusion or attachment to other proteins. For example, the provided compositions, combinations and methods contain organophosphorus bioscavengers can be modified by conjugation to one or more polymeric molecules (e.g., polymer). In some examples, conjugation to a polymer increases the half-life of the organophosphorus bioscavenger, for example, to promote prolonged/sustained treatment effects in a subject. In another example, the organophosphorus bioscavengers used in the provided compositions, combinations and methods can be modified by fusion or attachment to proteins, such as immunoglobulins or immunoglobulin domains, albumins, transferrins, and transferrin receptor proteins, which also can effect an increase in stability and/or serum half-life.

a. Polymer Modified Organophosphorus Bioscavengers

Any OP bioscavenger in the provided compositions, combinations and methods can be modified by attachment to polymers. Covalent or other stable attachment (conjugation) of polymeric molecules, such as polyethylene glycol (PEGylation moiety (PEG)), to the OP bioscavengers, such as cholinesterases, impart beneficial properties to the resulting OP bioscavenger-polymer composition. Such properties include improved biocompatibility, extension of protein (and enzymatic activity) half-life in the serum within a subject, effective shielding of the protein from proteases and hydrolysis, improved biodistribution, enhanced pharmacokinetics and/or pharmacodynamics, and increased water solubility.

Exemplary polymers that can be conjugated to the organophosphorus bioscavenger, such as a butyrylcholinesterase, in the provided compositions and combinations include natural and synthetic homopolymers, such as polyols (i.e. poly-OH), polyamines (i.e. poly-NH₂) and polycarboxyl acids (i.e. poly-COOH), and further heteropolymers i.e. polymers containing one or more different coupling groups e.g. a hydroxyl groups and amine groups. Examples of suitable polymeric molecules include polymeric molecules selected from among polyalkylene oxides (PAO), such as polyalkylene glycols (PAG), including polyethylene glycols (PEG), methoxypolyethylene glycols (mPEG) and polypropylene glycols, PEG-glycidyl ethers (Epox-PEG), PEG-oxycarbonylimidazole (CDI-PEG), branched polyethylene glycols (PEGs), polyvinyl alcohol (PVA), polycarboxylates, polyvinylpyrrolidone, poly-D,L-amino acids, polyethylene-co-maleic acid anhydride, polystyrene-co-maleic acid anhydride, dextrans including carboxymethyl-dextrans, heparin, homologous albumin, celluloses, including methylcellulose, carboxymethylcellulose, ethylcellulose, hydroxyethylcellulose carboxyethylcellulose and hydroxypropylcellulose, hydrolysates of chitosan, starches such as hydroxyethyl-starches and hydroxypropyl-starches, glycogen, agaroses and derivatives thereof, guar gum, pullulan, inulin, xanthan gum, carrageenan, pectin, alginic acid hydrolysates and bio-polymers.

Typically, the polymers are polyalkylene oxides (PAO), such as polyethylene oxides, such as PEG, typically mPEG, which, in comparison to polysaccharides such as dextran and pullulan, have few reactive groups capable of cross-linking. Typically, the polymers are non-toxic polymeric molecules such as (m)polyethylene glycol (mPEG) which can be covalently conjugated to the organophosphorus bioscavenger, such as a butyrylcholinesterase (e.g. to attachment groups on the protein's surface) using a relatively simple chemistry.

PEGylation of therapeutics has been reported to increase resistance to proteolysis, increase plasma half-life, and decrease antigenicity and immunogenicity. Examples of PEGylation methodologies are known in the art (see for example, Lu and Felix, Int. J. Peptide Protein Res., 43:127-138, 1994; Lu and Felix, Peptide Res., 6:140-6, 1993; Felix et al., Int. J. Peptide Res., 46:253-64, 1995; Benhar et al., J. Biol. Chem., 269: 13398-404, 1994; Brumeanu et al., J. Immunol., 154:3088-95, 1995; see also, Caliceti et al. (2003) Adv. Drug Deliv. Rev. 55(10):1261-77 and Molineux (2003) Pharmacotherapy 23 (8 Pt 2):3S-8S). PEGylation also can be used in the delivery of nucleic acid molecules in vivo. For example, PEGylation of adenovirus can increase stability and gene transfer (see, e.g., Cheng et al. (2003) Pharm. Res. 20(9):1444-51).

Suitable polymeric molecules for attachment to the organophosphorus bioscavengers, include, but are not limited to, polyethylene glycol (PEG) and PEG derivatives such as methoxy-polyethylene glycols (mPEG), PEG-glycidyl ethers (Epox-PEG), PEG-oxycarbonylimidazole (CDI-PEG), branched PEGs, and polyethylene oxide (PEO) (see e.g. Roberts et al., Advanced Drug Delivery Review (2002) 54: 459-476; Harris and Zalipsky, S (eds.) “Poly(ethylene glycol), Chemistry and Biological Applications” ACS Symposium Series 680, 1997; Mehvar et al., J. Pharm. Pharmaceut. Sci., 3(1):125-136, 2000; Harris, (2003) Nature Reviews Drug Discovery 2:214-221; and Tsubery, (2004) J Biol. Chem. 279(37):38118-24). The polymeric molecule can be of a molecular weight typically ranging from about 3 kDa to about 60 kDa. In some embodiments the polymeric molecule that is conjugated to an OP bioscavenger has a molecular weight of at least or about at least or 5, 10, 15, 20, 25, 30, 35, 40, 45, 50, 55, 60 or more, such as more than 60 kDa.

PEGylated Organophosphorus Bioscavengers

The organophosphorus bioscavenger used in the provided compositions, combinations and methods can be a PEGylated organophosphorus bioscavenger, such as a PEGylated acetylcholinesterase or PEGylated butyrylcholinesterase, e.g., PEG-rBChE (Protexia®). Various methods of modifying polypeptides by covalently attaching (conjugating) a PEG or PEG derivative (“PEGylation”) are known in the art (see, e.g., U.S. Pat. Publication Nos. 20110135623, 20090249503, 20090208480, and 20040235734; U.S. Pat. Nos. 7,572,764; 5,672,662, 6,737,505; and Huang et al., (2007) Proc Nall Acad Sci USA 104:13603-13608; Kronman et al., (2010) Chem Biol Interact 187:253-258). Techniques for PEGylation include, but are not limited to, specialized linkers and coupling chemistries (see e.g., Roberts, Adv. Drug Deliv. Rev. 54:459-476, 2002), attachment of multiple PEG moieties to a single conjugation site (such as via use of branched PEGs; see e.g., Guiotto et al., Bioorg. Med. Chem. Lett. 12:177-180, 2002), site-specific PEGylation and/or mono-PEGylation (see e.g., Chapman et al., Nature Biotech. 17:780-783, 1999), and site-directed enzymatic PEGylation (see e.g., Sato, Adv. Drug Deliv. Rev., 54:487-504, 2002). Methods and techniques described in the art can produce proteins having 1, 2, 3, 4, 5, 6, 7, 8, 9, 10 or more than 10 PEGs or PEG derivatives attached to a single protein molecule (see e.g., U.S. 2009/0249503).

Numerous reagents for PEGylation have been described in the art. Such reagents include, but are not limited to, N-hydroxysuccinimidyl (NHS) activated PEG, succinimidyl mPEG, mPEG₂-N-hydroxysuccinimide, mPEG succinimidyl alpha-methylbutanoate, mPEG succinimidyl propionate, mPEG succinimidyl butanoate, mPEG carboxymethyl 3-hydroxybutanoic acid succinimidyl ester, homobifunctional PEG-succinimidyl propionate, homobifunctional PEG propionaldehyde, homobifunctional PEG butyraldehyde, PEG maleimide, PEG hydrazide, p-nitrophenyl-carbonate PEG, mPEG-benzotriazole carbonate, propionaldehyde PEG, mPEG butryaldehyde, branched mPEG₂ butyraldehyde, mPEG acetyl, mPEG piperidone, mPEG methylketone, mPEG “linkerless” maleimide, mPEG vinyl sulfone, mPEG thiol, mPEG orthopyridylthioester, mPEG orthopyridyl disulfide, Fmoc-PEG-NHS, Boc-PEG-NHS, vinylsulfone PEG-NHS, acrylate PEG-NHS, fluorescein PEG-NHS, and biotin PEG-NHS (see e.g., Monfardini et al., Bioconjugate Chem. 6:62-69, 1995; Veronese et al., J. Bioactive Compatible Polymers 12:197-207, 1997; U.S. Pat. No. 5,672,662; U.S. Pat. No. 5,932,462; U.S. Pat. No. 6,495,659; U.S. Pat. No. 6,737,505; U.S. Pat. No. 4,002,531; U.S. Pat. No. 4,179,337; U.S. Pat. No. 5,122,614; U.S. Pat. No. 5,324,844; U.S. Pat. No. 5,446,090; U.S. Pat. No. 5,612,460; U.S. Pat. No. 5,643,575; U.S. Pat. No. 5,766,581; U.S. Pat. No. 5,795,569; U.S. Pat. No. 5,808,096; U.S. Pat. No. 5,900,461; U.S. Pat. No. 5,919,455; U.S. Pat. No. 5,985,263; U.S. Pat. No. 5,990,237; U.S. Pat. No. 6,113,906; U.S. Pat. No. 6,214,966; U.S. Pat. No. 6,258,351; U.S. Pat. No. 6,340,742; U.S. Pat. No. 6,413,507; U.S. Pat. No. 6,420,339; U.S. Pat. No. 6,437,025; U.S. Pat. No. 6,448,369; U.S. Pat. No. 6,461,802; U.S. Pat. No. 6,828,401; U.S. Pat. No. 6,858,736; U.S. 2001/0021763; U.S. 2001/0044526; U.S. 2001/0046481; U.S. 2002/0052430; U.S. 2002/0072573; U.S. 2002/0156047; U.S. 2003/0114647; U.S. 2003/0143596; U.S. 2003/0158333; U.S. 2003/0220447; U.S. 2004/0013637; US 2004/0235734; U.S. 2005/0114037; U.S. 2005/0171328; U.S. 2005/0209416; EP 1064951; EP 0822199; WO 01076640; WO 05000360; WO 0002017; WO 0249673; WO 9428024; and WO 0187925).

A PEG moiety can be attached to the N-terminal amino acid, a cysteine residue (either native or non-native) or other thiol group, a lysine, or other reactive native or non-native amino acids in the protein's primary sequence.

b. Other Modifications

Organophosphorus bioscavengers used in the provided compositions, combinations and methods can be modified by fusion or attachment to proteins, such as immunoglobulins or immunoglobulin domains, albumins, transferrins, and transferrin receptor proteins, to increase stability and serum half-life. The OP bioscavenger can be fused to the N-terminus or C-terminus of the protein. In one example, the protein is fused to the C-terminus of the OP bioscavenger. In another example, the protein is fused to the N-terminus of the organophosphorus bioscavenger. Typically, the organophosphorus bioscavenger joined to the fusion protein by a linker peptide containing 1-50 amino acids, typically at least 6 amino acids, such as 1; 2, 3, 4, 5, 6, 7, 8, 9, 10, 11, 12, 13, 14, 15, 20, 25, 30, 35, 40, 45 or 50 amino acids.

For example, an immunoglobulin or immunoglobulin domain for fusion or attachment to an organophosphorus bioscavenger herein includes, but is not limited to, an immunoglobulin (Ig) domain, such as an Fc region, heavy chain constant domain of IgG1, IgG2, IgG3 or IgG4, including a C_{H}1, hinge, C_{H}2, C_{H}3 and C_{H}4, or light chain constant domain C_{L} (see, U.S. Pat. Pub. No. 2009/0249503).

An albumin for fusion or attachment to an organophosphorus bioscavenger herein includes, but is not limited to albumins, including human serum albumin (HSA) or HSA polypeptides, to form a BChE-HSA fusion protein (see, U.S. Pat. Pub. No. 2006/0253913)

In addition, butyrylcholinesterases used herein can be modified by attachment to a domain that results in expression of stable tetrameric forms of BChE, such as a proline-rich attachment domain (PRAD) (see, Bon et al., (1997) J Biol Chem 272:3016-3021 and Krejci et al., (1997) J Biol Chem 272:22840-22847). As such, any butyrylcholinesterase in the provided compositions, combinations and methods can contain a PRAD sequence that contains at least six amino acids followed by a string of at least 10 proline residues.

### D. Hyaluronan-Degrading Enzymes

Provided herein are compositions and combinations containing a hyaluronan-degrading enzyme and an OP bioscavenger. Hyaluronan-degrading enzymes act to degrade hyaluronan by cleaving hyaluronan polymers. Hyaluronan is an essential component of the extracellular matrix and a major constituent of the interstitial barrier. By catalyzing the hydrolysis of hyaluronan, hyaluronan-degrading enzymes lower the viscosity of hyaluronan, thereby increasing tissue permeability and increasing the absorption rate of fluids administered parenterally. As such, hyaluronan-degrading enzymes, such as hyaluronidases, have been used, for example, as spreading or dispersing agents in conjunction with other agents, drugs and proteins to enhance their dispersion and delivery. Hyaluronan-degrading enzymes also are used as an adjuvant to increase the absorption and dispersion of other injected drugs, for hypodermoclysis (subcutaneous fluid administration), and as an adjunct in subcutaneous urography for improving resorption of radiopaque agents. Hyaluronan-degrading enzymes, for example, hyaluronidase can be used in applications of ophthalmic procedures, for example, peribulbar and sub-Tenon's block in local anesthesia prior to ophthalmic surgery. Hyaluronidase also can be use in other therapeutic and cosmetic uses, for example, by promoting akinesia in cosmetic surgery, such as blepharoplasties and face lifts.

In particular, hyaluronan-degrading enzymes act to degrade hyaluronan by cleaving hyaluronan polymers, which are composed of repeating disaccharides units, D-glucuronic acid (GlcA) and N-acetyl-D-glucosamine (GlcNAc), linked together via alternating β-1→4 and β-1→3 glycosidic bonds. Hyaluronan chains can reach about 25,000 disaccharide repeats or more in length and polymers of hyaluronan can range in size from about 5,000 to 20,000,000 Da in vivo. Accordingly, hyaluronan-degrading enzymes for the compositions, combinations and methods provided include any enzyme having the ability to catalyze the cleavage of a hyaluronan disaccharide chain or polymer. In some examples the hyaluronan-degrading enzyme cleaves the β-glycosidic bond in the hyaluronan chain or polymer. In other examples, the hyaluronan-degrading enzyme catalyze the cleavage of the β-1→3 glycosidic bond in the hyaluronan chain or polymer.

Various forms of hyaluronan-degrading enzymes, including hyaluronidases have been prepared and approved for therapeutic use in subjects, including humans. For example, animal-derived hyaluronidase preparations include Vitrase® (ISTA Pharmaceuticals), a purified ovine testicular hyaluronidase, Amphadase® (Amphastar Pharmaceuticals), a bovine testicular hyaluronidase and Hydase™ (Prima Pharm Inc.), a bovine testicular hyaluronidase. It is understood that any animal-derived hyaluronidase preparation can be used in the compositions, combinations and methods provided herein (see, e.g., U.S. Pat. Nos. 2,488,564, 2,488,565, 2,676,139, 2,795,529, 2,806,815, 2,808,362, 5,747,027 and 5,827,721 and International PCT Publication No. WO2005/118799). Hylenex® (Halozyme Therapeutics) is a human recombinant hyaluronidase produced by genetically engineered Chinese Hamster Ovary (CHO) cells containing nucleic acid encoding soluble forms of PH20, designated rHuPH20 (see, e.g. U.S. Publication No. US 2004/0268425 and U.S. Pat. No. 7,767,429).

Exemplary hyaluronan-degrading enzymes for use in the compositions, combinations and methods provided herein include hyaluronidases, as well as other enzymes such as chondrotinases and lyases that have the ability to cleave hyaluronan. Further, hyaluronan-degrading enzymes also include soluble forms thereof that can be expressed and secreted from cells. As described below, hyaluronan-degrading enzymes exist in membrane-bound or soluble forms that are secreted from cells. For purposes herein, soluble hyaluronan-degrading enzymes are provided for use in the compositions, combinations and methods herein. Thus, where hyaluronan-degrading enzymes include a glycosylphosphatidylinositol (GPI) anchor attachment signal sequence and/or are otherwise membrane-anchored or insoluble, such hyaluronan-degrading enzymes can be provided in soluble form by truncation or deletion of the GPI anchor attachment signal sequence to render the enzyme secreted and soluble. Thus, hyaluronan-degrading enzymes include truncated variants, e.g. truncated to remove all or a portion of a GPI anchor. Exemplary of such soluble hyaluronidases. 20 are soluble PH20 hyaluronides, such as any set forth in U.S. Pat. No. 7,767,429; U.S. Publication Nos. US 2004/0268425 or US 2010/0143457.

Hyaluronan-degrading enzymes provided herein also include variants of any hyaluronan-degrading enzyme, such as any hyaluronidase or soluble hyaluronidase, for example a PH20, that is known to one of skill in the art or described herein. A variant can include an allelic or species variant or other variant. For example, a hyaluronan-degrading enzyme can contain one or more variations in its primary sequence, such as amino acid substitutions, additions and/or deletions. A variant of a hyaluronan-degrading enzyme generally exhibits at least or about 60%, 70%, 80%, 90%, 91%, 92%, 93%, 94%, 95%, 96%, 97%, 98%, 99% or more sequence identity compared to the hyaluronan-degrading enzyme not containing the variation. Any variation can be included in the hyaluronan-degrading enzyme for the purposes herein, provided the enzyme retains hyaluronidase activity, such as at least or about 5%, 10%, 15%, 20%, 25%, 30%, 35%, 40%, 45%, 50%, 55%, 60%, 65%, 70%, 75%, 80%, 85%, 90%, 95% or more of the activity of a hyaluronan-degrading enzyme not containing the variation (as measured by in vitro and/or in vivo assays well known in the art and described herein). For example, exemplary of hyaluronan-degrading enzymes are any set forth in any of SEQ ID NOS:1, 2, 4-9, 47, 48, 150-170, 183-189 and 302-313, including mature forms thereof (lacking the signal sequence) or any that exhibit at least or about 60%, 70%, 80%, 90%, 91%, 92%, 93%, 94%, 95%, 96%, 97%, 98%, 99% or more sequence identity to any of SEQ ID NOS:1, 2, 4-9, 47, 48, 150-170, 183-189 and 302-313 or a mature form thereof lacking the signal sequence.

A non-limiting description of exemplary hyaluronan-degrading enzymes, such as hyaluronidase enzymes or soluble hyaluronidase enzymes, for example PH20, for use in the combinations, compositions and methods provided herein are described below. Such hyaluronan-degrading enzymes include those that are modified, for example, by conjugation to a polymer or other moieity.

1. Hyaluronidases

Hyaluronidases are members of a large family of hyaluronan-degrading enzymes. There are three general classes of hyaluronidases: mammalian-type hyaluronidases, bacterial hyaluronidases and hyaluronidases from leeches, other parasites and crustaceans. Such enzymes can be used in the compositions, combinations and methods provided herein.

a. Mammalian-Type Hyaluronidases

Mammalian-type hyaluronidases (EC 3.2.1.35) are endo-β-N-acetylhexosaminidases that hydrolyze the β-1→4 glycosidic bond of hyaluronan into various oligosaccharide lengths such as tetrasaccharides and hexasaccharides. These enzymes have both hydrolytic and transglycosidase activities, and can degrade hyaluronan and chondroitin sulfates (CS), generally C4-S and C6-S. Hyaluronidases of this type include, but are not limited to, hyaluronidases from cows (bovine) (SEQ ID NOS:10, 11, 64, 306 and 307, and nucleic acid molecules set forth in SEQ ID NOS:190-192), sheep (Ovis aries) (SEQ ID NO: 26, 27, 63 and 65, nucleic acid molecules set forth in SEQ ID NOS:66 and 193-194), yellow jacket wasp (SEQ ID NOS:12 and 13), honey bee (SEQ ID NO:14), white-face hornet (SEQ ID NO:15), paper wasp (SEQ ID NO:16), mouse (SEQ ID NOS:17-19, 32, 308), pig (SEQ ID NOS:20-21), rat (SEQ ID NOS:22-24, 31, 309), rabbit (SEQ ID NO:25, 310), orangutan (SEQ ID NO:28), cynomolgus monkey (SEQ ID NO:29, 305), guinea pig (SEQ ID NO:30, 311), chimpanzee (SEQ ID NO:101, 302, 303), rhesus monkey (SEQ ID NO:102, 304), fox (SEQ ID NO:312 and 313), and human hyaluronidases (SEQ ID NOS:1-2, 36-39). The above hyaluronidases include PH20 hyaluronidases. Also, BH55 hyaluronidase is of this type as described in U.S. Pat. Nos. 5,747,027 and 5,827,721. Exemplary of hyaluronidases in the compositions, combinations and methods provided herein are soluble hyaluronidases that are soluble forms of any of the above hyaluronidase, and that can be secreted from cells.

Mammalian hyaluronidases can be further subdivided into those that are neutral active, predominantly found in testes extracts, and acid active, predominantly found in organs such as the liver. Exemplary neutral active hyaluronidases include PH20, including but not limited to, PH20 derived from different species such as ovine (SEQ ID NOS:27, 63 and 65), bovine (SEQ ID NO:11 and 64) and human (SEQ ID NO:1). Human PH20 (also known as SPAM1 or sperm surface protein PH20), is generally attached to the plasma membrane via a glycosylphosphatidyl inositol (GPI) anchor. It is naturally involved in sperm-egg adhesion and aids penetration by sperm of the layer of cumulus cells by digesting hyaluronic acid.

Besides human PH20 (also termed SPAM1), five hyaluronidase-like genes have been identified in the human genome, HYAL1, HYAL2, HYAL3, HYAL4 and HYALP1. HYALP1 is a pseudogene, and HYAL3 (SEQ ID NO:38) has not been shown to possess enzyme activity toward any known substrates. HYAL4 (precursor polypeptide set forth in SEQ ID NO:39) is a chondroitinase and exhibits little activity towards hyaluronan. HYAL1 (precursor polypeptide set forth in SEQ ID NO:36) is the prototypical acid-active enzyme and PH20 (precursor polypeptide set forth in SEQ ID NO:1) is the prototypical neutral-active enzyme. Acid-active hyaluronidases, such as HYAL1 and HYAL2 (precursor polypeptide set forth in SEQ ID NO:37) generally lack catalytic activity at neutral pH (i.e. pH 7). For example, HYAL1 has little catalytic activity in vitro over pH 4.5 (Frost et al. (1997) Anal. Biochem. 251:263-269). HYAL2 is an acid-active enzyme with a very low specific activity in vitro. The hyaluronidase-like enzymes also can be characterized by those which are generally attached to the plasma membrane via a glycosylphosphatidyl inositol (GPI) anchor such as human HYAL2 and human PH20 (Danilkovitch-Miagkova et al. (2003) Proc Natl Acad Sci USA 100(8):4580-5), and those which are generally soluble such as human HYAL1 (Frost et al. (1997) Biochem Biophys Res Commun. 236(1):10-5).

PH20

PH20, like other mammalian hyaluronidases, is an endo-β-N-acetyl-hexosaminidase that hydrolyzes the 131-4 glycosidic bond of hyaluronic acid into various oligosaccharide lengths such as tetrasaccharides and hexasaccharides. It has both hydrolytic and transglycosidase activities and can degrade hyaluronic acid and chondroitin sulfates, such as C4-S and C6-S. PH20 is naturally involved in sperm-egg adhesion and aids penetration by sperm of the layer of cumulus cells by digesting hyaluronic acid. PH20 is located on the sperm surface, and in the lysosome-derived acrosome, where it is bound to the inner acrosomal membrane. Plasma membrane PH20 has hyaluronidase activity only at neutral pH, while inner acrosomal membrane PH20 has activity at both neutral and acid pH. In addition to being a hyaluronidase, PH20 also appears to be a receptor for HA-induced cell signaling, and a receptor for the zona pellucida surrounding the oocyte.

Exemplary PH20 proteins include, but are not limited to, human (precursor polypeptide set forth in SEQ ID NO:1, mature polypeptide set forth in SEQ ID NO: 2), chimpanzee (SEQ ID NO:101, 302, 303), Rhesus monkey (SEQ ID NO:102, 304) bovine (SEQ ID NOS: 11, 64, 306, 307), rabbit (SEQ ID NO: 25, 310), ovine PH20 (SEQ ID NOS: 27, 63 and 65), Cynomolgus monkey (SEQ ID NO: 29, 305), guinea pig (SEQ ID NO: 30, 311), rat (SEQ ID NO: 31, 309), mouse (SEQ ID NO: 32, 308) and fox (SEQ ID NO:312 and 313) PH20 polypeptides.

Bovine PH20 is a 553 amino acid precursor polypeptide (SEQ ID NO:11). Alignment of bovine PH20 with the human PH20 shows only weak homology, with multiple gaps existing from amino acid 470 through to the respective carboxy termini due to the absence of a GPI anchor in the bovine polypeptide (see e.g., Frost GI (2007) Expert Opin. Drug. Deliv. 4: 427-440). In fact, clear GPI anchors are not predicted in many other PH20 species besides humans. Thus, PH20 polypeptides produced from ovine and bovine naturally exist as soluble forms. Though bovine PH20 exists very loosely attached to the plasma membrane, it is not anchored via a phospholipase sensitive anchor (Lalancette et al. (2001) Biol Reprod. 65(2):628-36). This unique feature of bovine hyaluronidase has permitted the use of the soluble bovine testes hyaluronidase enzyme as an extract for clinical use (Wydase®, Hyalase®).

The human PH20 mRNA transcript is normally translated to generate a 509 amino acid precursor polypeptide (SEQ ID NO:1) containing a 35 amino acid signal sequence at the N-terminus (amino acid residue positions 1-35) and a 19 amino acid glycosylphosphatidylinositol (GPI) anchor attachment signal sequence at the C-terminus (amino acid residue positions 491-509). The mature PH20 therefore, is a 474 amino acid polypeptide set forth in SEQ ID NO:2. Following transport of the precursor polypeptide to the ER and removal of the signal peptide, the C-terminal GPI-attachment signal peptide is cleaved to facilitate covalent attachment of a GPI anchor to the newly-formed C-terminal amino acid at the amino acid position corresponding to position 490 of the precursor polypeptide set forth in SEQ ID NO:1. Thus, a 474 amino acid GPI-anchored mature polypeptide with an amino acid sequence set forth in SEQ ID NO:2 is produced.

Human PH20 exhibits hyaluronidase activity at neutral and acid pH. In one aspect, human PH20 is the prototypical neutral-active hyaluronidase that is generally locked to the plasma membrane via a GPI anchor. In another aspect, PH20 is expressed on the inner acrosomal membrane where it has hyaluronidase activity at neutral and acid pH. It appears that PH20 contains two catalytic sites at distinct regions of the polypeptide: the Peptide 1 and Peptide 3 regions (Chem et al., (2001) Matrix Biology 20:515-525). Evidence indicates that the Peptide 1 region of PH20, which corresponds to amino acid positions 107-137 of the mature polypeptide set forth in SEQ ID NO:2 and positions 142-172 of the precursor polypeptide set forth in SEQ ID NO:1, is required for enzyme activity at neutral pH. Amino acids at positions 111 and 113 (corresponding to the mature PH20 polypeptide set forth in SEQ ID NO:2) within this region appear to be important for activity, as mutagenesis by amino acid replacement results in PH20 polypeptides with 3% hyaluronidase activity or undetectable hyaluronidase activity, respectively, compared to the wild-type PH20 (Arming et al., (1997) Eur. J. Biochem. 247:810-814).

The Peptide 3 region, which corresponds to amino acid positions 242-262 of the mature polypeptide set forth in SEQ ID NO:2, and positions 277-297 of the precursor polypeptide set forth in SEQ ID NO: 1, appears to be important for enzyme activity at acidic pH. Within this region, amino acids at positions 249 and 252 of the mature PH20 polypeptide appear to be essential for activity, and mutagenesis of either one results in a polypeptide essentially devoid of activity (Arming et al., (1997) Eur. J. Biochem. 247:810-814).

In addition to the catalytic sites, PH20 also contains a hyaluronan-binding site. Experimental evidence indicate that this site is located in the Peptide 2 region, which corresponds to amino acid positions 205-235 of the precursor polypeptide set forth in SEQ ID NO: 1 and positions 170-200 of the mature polypeptide set forth in SEQ ID NO:2. This region is highly conserved among hyaluronidases and is similar to the heparin binding motif. Mutation of the arginine residue at position 176 (corresponding to the mature PH20 polypeptide set forth in SEQ ID NO:2) to a glycine results in a polypeptide with only about 1% of the hyaluronidase activity of the wild type polypeptide (Arming et al., (1997) Eur. J. Biochem. 247:810-814).

There are seven potential glycosylation sites, including N-linked glycosylation sites, in human PH20 at N82, N166, N235, N254, N368, N393, S490 of the polypeptide exemplified in SEQ ID NO: 1. Because amino acids 36 to 464 of SEQ ID NO:1 appear to contain the minimally active human PH20 hyaluronidase domain, the glycosylation site S490 is not required for proper hyaluronidase activity. There are six disulfide bonds in human PH20. Two disulfide bonds between the cysteine residues C60 and C351 and between C224 and C238 of the polypeptide exemplified in SEQ ID NO: 1 (corresponding to residues C25 and C316, and C189 and C203 of the mature polypeptide set forth in SEQ ID NO:2, respectively). A further four disulfide bonds are formed between the cysteine residues C376 and C387; between C381 and C435; between C437 and C443; and between C458 and C464 of the polypeptide exemplified in SEQ ID NO: 1 (corresponding to residues C341 and C352; between C346 and C400; between C402 and C408; and between C423 and C429 of the mature polypeptide set forth in SEQ ID NO:2, respectively).

b. Bacterial Hyaluronidases

Bacterial hyaluronidases (EC 4.2.2.1 or EC 4.2.99.1) degrade hyaluronan and, to various extents, chondroitin sulfates and dermatan sulfates. Hyaluronan lyases isolated from bacteria differ from hyaluronidases (from other sources, e.g., hyaluronoglucosaminidases, EC 3.2.1.35) by their mode of action. They are endo-β-N-acetylhexosaminidases that catalyze an elimination reaction, rather than hydrolysis, of the β1→4-glycosidic linkage between N-acetyl-beta-D-glucosamine and D-glucuronic acid residues in hyaluronan, yielding 3-(4-deoxy-β-D-gluc-4-enuronosyl)-N-acetyl-D-glucosamine tetra- and hexasaccharides, and disaccharide end products. The reaction results in the formation of oligosaccharides with unsaturated hexuronic acid residues at their nonreducing ends.

Exemplary hyaluronidases from bacteria for use in the compositions, combinations and methods provided include, but are not limited to, hyaluronan-degrading enzymes in microorganisms, including strains of Arthrobacter, Bdellovibrio, Clostridium, Micrococcus, Streptococcus, Peptococcus, Propionibacterium, Bacteroides, and Streptomyces. Particular examples of such strains and enzymes include, but are not limited to Arthrobacter sp. strain FB24 (SEQ ID NO:67), Bdellovibrio bacteriovorus (SEQ ID NO:68), Propionibacterium acnes (SEQ ID NO:69), Streptococcus agalactiae ((SEQ ID NO:70); 18RS21 (SEQ ID NO:71); serotype Ia (SEQ ID NO:72); serotype 111 (SEQ ID NO:73), Staphylococcus aureus (strain COL (SEQ ID NO:74); strain MRSA252 (SEQ ID NOS:75 and 76); strain MSSA476 (SEQ ID NO:77); strain NCTC 8325 (SEQ ID NO:78); strain bovine RF122 (SEQ ID NOS:79 and 80); strain USA300 (SEQ ID NO:81), Streptococcus pneumoniae ((SEQ ID NO:82); strain ATCC BAA-255/R6 (SEQ ID NO:83); serotype 2, strain D39/NCTC 7466 (SEQ ID NO:84), Streptococcus pyogenes (serotype (SEQ ID NO:85); serotype M2, strain MGAS10270 (SEQ ID NO:86); serotype M4, strain MGAS10750 (SEQ ID NO:87); serotype M6 (SEQ ID NO:88); serotype M12, strain MGAS2096 (SEQ ID NOS:89 and 90); serotype M12, strain MGAS9429 (SEQ ID NO:91); serotype M28 (SEQ ID NO:92); Streptococcus suis (SEQ ID NOS:93-95); Vibrio fischeri (strain ATCC 700601/ES114 (SEQ ID NO:96)), and the Streptomyces hyaluronolyticus hyaluronidase enzyme, which is specific for hyaluronic acid and does not cleave chondroitin or chondroitin sulfate (Ohya, T. and Kaneko, Y. (1970) Biochim. Biophys. Acta 198:607).

c. Hyaluronidases from Leeches, Other Parasites and Crustaceans

Hyaluronidases from leeches, other parasites, and crustaceans (EC 3.2.1.36) are endo-β-glucuronidases that generate tetra- and hexasaccharide end-products. These enzymes catalyze hydrolysis of 1→3-linkages between β-D-glucuronate and N-acetyl-D-glucosamine residues in hyaluronate. Exemplary hyaluronidases from leeches include, but are not limited to, hyaluronidase from Hirudimidae (e.g., Hirudo medicinalis), Erpobdellidae (e.g., Nephelopsis obscura and Erpobdella punctata,), Glossiphoniidae (e.g., Desserobdella picta, Helobdella stagnalis, Glossiphonia complanata, Placobdella ornata and Theromyzon sp.) and Haemopidae (Haemopis marmorata) (Hovingh et al. (1999) Comp Biochem Physiol B Biochem Mol. Biol. 124(3):319-26). An exemplary hyaluronidase from bacteria that has the same mechanism of action as the leech hyaluronidase is that from the cyanobacteria, Synechococcus sp. (strain RCC307, SEQ ID NO:97).

2. Other Hyaluronan-Degrading Enzymes

In addition to the hyaluronidase family, other hyaluronan-degrading enzymes can be used in the compositions, combinations and methods provided. For example, enzymes, including particular chondroitinases and lyases, that have the ability to cleave hyaluronan can be employed. Exemplary chondroitinases that can degrade hyaluronan include, but are not limited to, chondroitin ABC lyase (also known as chondroitinase ABC), chondroitin AC lyase (also known as chondroitin sulfate lyase or chondroitin sulfate eliminase) and chondroitin C lyase. Methods for production and purification of such enzymes for use in the compositions, combinations, and methods provided are known in the art (e.g., U.S. Pat. No. 6,054,569; Yamagata, et al. (1968) J. Biol. Chem. 243(7):1523-1535; Yang et al. (1985) J. Biol. Chem. 160(30):1849-1857).

Chondroitin ABC lyase contains two enzymes, chondroitin-sulfate-ABC endolyase (EC 4.2.2.20) and chondroitin-sulfate-ABC exolyase (EC 4.2.2.21) (Hamai et al. (1997) J Biol. Chem. 272(14):9123-30), which degrade a variety of glycosaminoglycans of the chondroitin-sulfate- and dermatan-sulfate type. Chondroitin sulfate, chondroitin-sulfate proteoglycan and dermatan sulfate are the preferred substrates for chondroitin-sulfate-ABC endolyase, but the enzyme also can act on hyaluronan at a lower rate. Chondroitin-sulfate-ABC endolyase degrades a variety of glycosaminoglycans of the chondroitin-sulfate- and dermatan-sulfate type, producing a mixture of Δ4-unsaturated oligosaccharides of different sizes that are ultimately degraded to Δ4-unsaturated tetra- and disaccharides. Chondroitin-sulfate-ABC exolyase has the same substrate specificity but removes disaccharide residues from the non-reducing ends of both polymeric chondroitin sulfates and their oligosaccharide fragments produced by chondroitin-sulfate-ABC endolyase (Hamai, A. et al. (1997) J. Biol. Chem. 272:9123-9130). Exemplary chondroitin-sulfate-ABC endolyases and chondroitin-sulfate-ABC exolyases include, but are not limited to, those from Proteus vulgaris and Flavobacterium heparinum (the Proteus vulgaris chondroitin-sulfate-ABC endolyase is set forth in SEQ ID NO: 98 (Sato et al. (1994) Appl. Microbiol. Biotechnol. 41(1):39-46).

Chondroitin AC lyase (EC 4.2.2.5) is active on chondroitin sulfates A and C, chondroitin and hyaluronic acid, but is not active on dermatan sulfate (chondroitin sulfate B). Exemplary (chondroitin AC lyase) from the bacteria include, but are not limited to, those from Flavobacterium heparinum and Victivallis vadensis, set forth in SEQ ID NOS:99 and 100, respectively, and Arthrobacter aurescens (Tkalec et al. (2000) Applied and Environmental Microbiology 66(1):29-35; Ernst et al. (1995) Critical Reviews in Biochemistry and Molecular Biology 30(5):387-444).

Chondroitinase C (Chondroitin C lyase) cleaves chondroitin sulfate C producing tetrasaccharide plus an unsaturated 6-sulfated disaccharide (delta Di-6S). It also cleaves hyaluronic acid producing unsaturated non-sulfated disaccharide (delta Di-OS). Exemplary chondroitinase C enzymes from the bacteria include, but are not limited to, those from Streptococcus and Flavobacterium (Hibi et al. (1989) FEMS-Microbiol-Lett. 48(2):121-4; Michelacci et al. (1976) J. Biol. Chem. 251:1154-8; Tsuda et al. (1999) Eur. J. Biochem. 262:127-133)

3. Soluble Hyaluronan-Degrading Enzymes

Provided in the compositions, combinations, uses and methods herein are soluble hyaluronan-degrading enzymes, including soluble hyaluronidases. Soluble hyaluronan-degrading enzymes include any hyaluronan-degrading enzymes that are secreted from cells (e.g. CHO cells) upon expression and exist in soluble form. Such enzymes include, but are not limited to, soluble hyaluronidases, including non-human soluble hyaluronidases, including non-human animal soluble hyaluronidases, bacterial soluble hyaluronidases and human hyaluronidases, Hyal 1, bovine PH20 and ovine PH20, allelic variants thereof and other variants thereof. For example, included among soluble hyaluronan-degrading enzymes are any hyaluronan-degrading enzymes that have been modified to be soluble. For example, hyaluronan-degrading enzymes that contain a GPI anchor can be made soluble by truncation of and removal of all or a portion of the GPI anchor attachment signal sequence. In one example, the human hyaluronidase PH20, which is normally membrane anchored via a GPI anchor, can be made soluble by truncation of and removal of all or a portion of the GPI anchor attachment signal sequence at the C-terminus.

Soluble hyaluronan-degrading enzymes also include neutral active and acid active hyaluronidases. Depending on factors, such as, but not limited to, the desired level of activity of the enzyme following administration and/or site of administration, neutral active and acid active hyaluronidases can be selected. In a particular example, the hyaluronan-degrading enzyme for use in the compositions, combinations and methods herein is a soluble neutral active hyaluronidase.

Exemplary of a soluble hyaluronidase is PH20 from any species, such as any set forth in any of SEQ ID NOS: 1, 2, 11, 25, 27, 29-32, 63-65, 101-102 and 199-210, or truncated forms thereof lacking all or a portion of the C-terminal GPI anchor attachment signal sequence, so long as the hyaluronidase is soluble (secreted upon expression) and retains hyaluronidase activity. Also included among soluble hyaluronidases are allelic variants or other variants of any of SEQ ID NOS:1, 2, 11, 25, 27, 29-32, 63-65, 101-102 and 199-210, or truncated forms thereof. Allelic variants and other variants are known to one of skill in the art, and include polypeptides having 60%, 70%, 80%, 90%, 91%, 92%, 93%, 94%, 95%, 96%., 97%, 98%, 99% or more sequence identity to any of SEQ ID NOS: 1, 2, 11, 25, 27, 29-32, 63-65, 101-102 and 199-210, or truncated forms thereof. Amino acid variants include conservative and non-conservative mutations. It is understood that residues that are important or otherwise required for the activity of a hyaluronidase, such as any described above or known to skill in the art, are generally invariant and cannot be changed. These include, for example, active site residues. Thus, for example, amino acid residues 111, 113 and 176 (corresponding to residues in the mature PH20 polypeptide set forth in SEQ ID NO:2) of a human PH20 polypeptide, or soluble form thereof, are generally invariant and are not altered. Other residues that confer glycosylation and formation of disulfide bonds required for proper folding also can be invariant.

In some instances, the soluble hyaluronan-degrading enzyme is normally GPI-anchored (such as, for example, human PH20) and is rendered soluble by truncation at the C-terminus. Such truncation can remove all of the GPI anchor attachment signal sequence, or can remove only some of the GPI anchor attachment signal sequence. The resulting polypeptide, however, is soluble. In instances where the soluble hyaluronan-degrading enzyme retains a portion of the GPI anchor attachment signal sequence, 1, 2, 3, 4, 5, 6, 7 or more amino acid residues in the GPI-anchor attachment signal sequence can be retained, provided the polypeptide is soluble. Polypeptides containing one or more amino acids of the GPI anchor are termed extended soluble hyaluronan-degrading enzymes. One of skill in the art can determine whether a polypeptide is GPI-anchored using methods well known in the art. Such methods include, but are not limited to, using known algorithms to predict the presence and location of the GPI-anchor attachment signal sequence and co-site, and performing solubility analyses before and after digestion with phosphatidylinositol-specific phospholipase C (PI-PLC) or D (PI-PLD).

Extended soluble hyaluronan-degrading enzymes can be produced by making C-terminal truncations to any naturally GPI-anchored hyaluronan-degrading enzyme such that the resulting polypeptide is soluble and contains one or more amino acid residues from the GPI-anchor attachment signal sequence (see, e.g., U.S. Patent Publication No. US20100143457). Exemplary extended soluble hyaluronan-degrading enzymes that are C-terminally truncated but retain a portion of the GPI anchor attachment signal sequence include, but are not limited to, extended soluble PH20 (esPH20) polypeptides of primate origin, such as, for example, human and chimpanzee esPH20 polypeptides. For example, the esPH20 polypeptides can be made by C-terminal truncation of any of the mature or precursor polypeptides set forth in SEQ ID NOS:1, 2 or 101, or allelic or other variation thereof, including active fragment thereof, wherein the resulting polypeptide is soluble and retains one or more amino acid residues from the GPI-anchor attachment signal sequence. Allelic variants and other variants are known to one of skill in the art, and include polypeptides having 60%, 70%, 80%, 90%, 91%, 92%, 93%, 94%, 95% or more sequence identity to any of SEQ ID NOS: 1 or 2. The esPH20 polypeptides provided herein can be C-terminally truncated by 1, 2, 3, 4, 5, 6, 7, 8, 9, 10 or more amino acids compared to the wild type polypeptide, such as a polypeptide with a sequence set forth in SEQ ID NOS: 1, 2 or 101, provided the resulting esPH20 polypeptide is soluble and retains 1 or more amino acid residues from the GPI-anchor attachment signal sequence.

Typically, for use in the compositions, combinations and methods herein, a soluble human hylauronan degrading enzyme, such as a soluble human PH20, is used. Although hylauronan degrading enzymes, such as PH20, from other animals can be utilized, such preparations are potentially immunogenic, since they are animal proteins. For example, a significant proportion of patients demonstrate prior sensitization secondary to ingested foods, and since these are animal proteins, all patients have a risk of subsequent sensitization. Thus, non-human preparations may not be suitable for chronic use. If non-human preparations are desired, it is contemplated herein that such polypeptides can be prepared to have reduced immunogenicity. Such modifications are within the level of one of skill in the art and can include, for example, removal and/or replacement of one or more antigenic epitopes on the molecule.

Hyaluronan-degrading enzymes, including hyaluronidases (e.g., PH20), used in the methods herein can be recombinantly produced or can be purified or partially-purified from natural sources, such as, for example, from testes extracts. Methods for production of recombinant proteins, including recombinant hyaluronan-degrading enzymes, are provided elsewhere herein and are well known in the art.

a. Soluble Human PH20

Exemplary of a soluble hyaluronidase is soluble human PH20, Soluble forms of recombinant human PH20 have been produced and can be used in the compositions, combinations and methods described herein. The production of such soluble forms of PH20 is described in U.S. Patent Publication Nos. US20040268425, US20050260186, US20060104968, US20100143457 and International PCT Publication No. WO2009111066: For example, soluble PH20 polypeptides, include C-terminally truncated variant polypeptides that include a sequence of amino acids in SEQ ID NO:1 or 2, or have at least 91%, 92%, 93%, 94%, 95%, 95%, 97%, 98% sequence identity to a sequence of amino acids included in SEQ ID NO:1 or 2, retain hyaluronidase activity and are soluble. Included among these polypeptides are soluble PH20 polypeptides that completely lack all or a portion of the GPI-anchor attachment signal sequence.

Also included are extended soluble PH20 (esPH20) polypeptides that contain at least one amino acid of the GPI anchor. Thus, instead of having a GPI-anchor covalently attached to the C-terminus of the protein in the ER and being anchored to the extracellular leaflet of the plasma membrane, these polypeptides are secreted and are soluble. C-terminally truncated PH20 polypeptides can be C-terminally truncated by 1, 2, 3, 4, 5, 6, 7, 8, 9, 10, 11, 12, 13, 14, 15, 16, 17, 18, 19, 20, 25, 30, 35, 40, 45, 50, 55, 60 or more amino acids compared to the full length wild type polypeptide, such as a full length wild type polypeptide with a sequence set forth in SEQ ID NOS:1 or 2, or allelic or species variants or other variants thereof.

For example, soluble forms include, but are not limited to, C-terminal truncated polypeptides of human PH20 set forth in SEQ ID NO:1 having a C-terminal amino acid residue 467, 468, 469, 470, 471, 472, 473, 474, 475, 476, 477, 478, 479, 480, 481, 482 and 483, 484, 485, 486, 487, 488, 489, 490, 491, 492, 493, 494, 495, 496, 497, 498, 499 or 500 of the sequence of amino acids set forth in SEQ ID NO:1, or polypeptides that exhibit at least 85% identity thereto. Soluble forms of human PH20 generally include those that contain amino acids 36-464 set forth in SEQ ID NO:1. For example, when expressed in mammalian cells, the 35 amino acid N-terminal signal sequence is cleaved during processing, and the mature form of the protein is secreted. Thus, the mature soluble polypeptides contain amino acids 36 to 467, 468, 469, 470, 471, 472, 473, 474, 475, 476, 477, 478, 479, 480, 481, 482 and 483 of SEQ ID NO:1. Table 3A provides non-limiting examples of exemplary C-terminally truncated PH20 polypeptides, including C-terminally truncated soluble PH20 polypeptides. In Table 3A below, the length (in amino acids) of the precursor and mature polypeptides, and the sequence identifier (SEQ ID NO) in which exemplary amino acid sequences of the precursor and mature polypeptides of the C-terminally truncated PH20 proteins are set forth, are provided. The wild-type PH20 polypeptide also is included in Table 3A for comparison. In particular, exemplary of soluble hyaluronidases are soluble human PH20 polypeptides that are 442, 443, 444, 445, 446 or 447 amino acids in length, such as set forth in any of SEQ ID NOS: 4-9, or allelic or species variants or other variants thereof.

TABLE 3A
Exemplary C-terminally truncated PH20 polypeptides

| | Precursor | Precursor | Mature | Mature |
|---|---|---|---|---|
| Polypeptide | (amino acids) | SEQ ID NO | (amino acids) | SEQ ID NO |
| wildtype | 509 | 1 | 474 | 2 |
| SPAM1-SILF | 500 | 139 | 465 | 183 |
| SPAM-VSIL | 499 | 106 | 464 | 150 |
| SPAM1-IVSI | 498 | 140 | 463 | 184 |
| SPAM1-FIVS | 497 | 107 | 462 | 151 |
| SPAM1-MFIV | 496 | 141 | 461 | 185 |
| SPAM1-TMFI | 495 | 108 | 460 | 152 |
| SPAM1-ATMF | 494 | 142 | 459 | 186 |
| SPAM1-SATM | 493 | 109 | 458 | 153 |
| SPAM1-LS AT | 492 | 143 | 457 | 187 |
| SPAM1-TLSA | 491 | 110 | 456 | 154 |
| SPAM1-PSTL | 489 | 111 | 454 | 155 |
| SPAM1-SPST | 488 | 144 | 453 | 188 |
| SPAM1-STLS | 490 | 112 | 455 | 156 |
| SPAM1-ASPS | 487 | 113 | 452 | 157 |
| SPAM1-NASP | 486 | 145 | 451 | 189 |
| SPAM1-YNAS | 485 | 114 | 450 | 158 |
| SPAM1-FYNA | 484 | 115 | 449 | 159 |
| SPAM1-IFYN | 483 | 46 | 448 | 48 |
| SPAM1-QIFY | 482 | 3 | 447 | 4 |
| SPAM1-PQIF | 481 | 45 | 446 | 5 |
| SPAM1-EPQI | 480 | 44 | 445 | 6 |
| SPAM1-EEPQ | 479 | 43 | 444 | 7 |
| SPAM1-TEEP | 478 | 42 | 443 | 8 |
| SPAM1-ETEE | 477 | 41 | 442 | 9 |
| SPAM1 -METE | 476 | 116 | 441 | 160 |
| SPAM1-PMET | 475 | 117 | 440 | 161 |
| SPAM1-PPME | 474 | 118 | 439 | 162 |
| SPAM1-KPPM | 473 | 119 | 438 | 163 |
| SPAM1-LKPP | 472 | 120 | 437 | 164 |
| SPAM1-FLKP | 471 | 121 | 436 | 165 |
| SPAM1-AFLK | 470 | 122 | 435 | 166 |
| SPAM1-DAFL | 469 | 123 | 434 | 167 |
| SPAM1-IDAF | 468 | 124 | 433 | 168 |
| SPAM1-CIDA | 467 | 40 | 432 | 47 |
| SPAM1-VCID | 466 | 125 | 431 | 169 |
| SPAM1-GVCI | 465 | 126 | 430 | 170 |

Generally soluble forms of PH20 are produced using protein expression systems that facilitate correct N-glycosylation to ensure the polypeptide retains activity, since glycosylation is important for the catalytic activity and stability of hyaluronidases. Such cells include, for example Chinese Hamster Ovary (CHO) cells (e.g. DG44 CHO cells). Other C-terminally truncated PH20 polypeptides, including precursor and mature forms, are set forth in any of SEQ ID NOS: 103-105, 127-138, 146-149 and 171-182.

b. rHuPH20

Recombinant soluble forms of human PH20 have been generated and can be used in the compositions, combinations and methods provided herein. The generation of such soluble forms of recombinant human PH20 are described, for example, in U.S. Patent Publication Nos. US20040268425; US 20050260186; US20060104968; US20100143457; and International PCT Publication No. WO2009111066. Exemplary of such polypeptides are those generated by expression of a nucleic acid molecule encoding amino acids 1-482 (set forth in SEQ ID NO:3). Such an exemplary nucleic acid molecule is set forth in SEQ ID NO:49. Post translational processing removes the 35 amino acid signal sequence, leaving a 447 amino acid soluble recombinant human PH20 (SEQ ID NO:4). As produced in the culture medium there is heterogeneity at the C-terminus such that the product, designated rHuPH20, includes a mixture of species that can include any one or more of SEQ ID NOS. 4-9 in various abundance. Typically, rHuPH20 is produced in cells that facilitate correct N-glycosylation to retain activity, such as CHO cells (e.g. DG44 CHO cells). The specific activity of rHuPH20 is about 120,000 U/mg.

4. Glycosylation of Hyaluronan-Degrading Enzymes

Glycosylation, including N- and O-linked glycosylation, of some hyaluronan-degrading enzymes, including hyaluronidases, can be important for their catalytic activity and stability. While altering the type of glycan modifying a glycoprotein can have dramatic affects on a protein's antigenicity, structural folding, solubility, and stability, most enzymes are not thought to require glycosylation for optimal enzyme activity. For some hyaluronidases, removal of N-linked glycosylation can result in near complete inactivation of the hyaluronidase activity. Thus, for such hyaluronidases, the presence of N-linked glycans is critical for generating an active enzyme.

N-linked oligosaccharides fall into several major types (oligomannose, complex, hybrid, sulfated), all of which have (Man)₃-GlcNAc-GlcNAc- cores attached via the amide nitrogen of Asn residues that fall within -Asn-Xaa-Thr/Ser-sequences (where Xaa is not Pro). Glycosylation at an -Asn-Xaa-Cys- site has been reported for coagulation protein C. In some instances, a hyaluronan-degrading enzyme, such as a hyaluronidase, can contain both N-glycosidic and O-glycosidic linkages. For example, PH20 has O-linked oligosaccharides as well as N-linked oligosaccharides. There are seven potential glycosylation sites, including N-linked glycosylation sites, at N82, N166, N235, N254, N368, N393, S490 of human PH20 exemplified in SEQ ID NO: 1. Amino acid residues N82, N166 and N254 are occupied by complex type glycans whereas amino acid residues N368 and N393 are occupied by high mannose type glycans. Amino acid residue N235 is occupied by approximately 80% high mannose type glycans and 20% complex type glycans. As noted above, glycosylation at S490 is not required for hyaluronidase activity.

In some examples, the hyaluronan-degrading enzymes for use in the compositions, combinations and/or methods provided are glycosylated at one or all of the glycosylation sites. For example, for human PH20, or a soluble form thereof, 2, 3, 4, 5, or 6 of the N-glycosylation sites corresponding to amino acids N82, N166, N235, N254, N368, and N393 of SEQ ID NO: 1 are glycosylated. In some examples the hyaluronan-degrading enzymes are glycosylated at one or more native glycosylation sites. In other examples, the hyaluronan-degrading enzymes are modified at one or more non-native glycosylation sites to confer glycosylation of the polypeptide at one or more additional site. In such examples, attachment of additional sugar moieties can enhance the pharmacokinetic properties of the molecule, such as improved half-life and/or improved activity.

In other examples, the hyaluronan-degrading enzymes for use in the compositions, combinations and/or methods provided herein are partially deglycosylated (or N-partially glycosylated polypeptides). For example, partially deglycosylated soluble PH20 polypeptides that retain all or a portion of the hyaluronidase activity of a fully glycosylated hyaluronidase can be used in the compositions, combinations and/or methods provided herein. Exemplary partially deglycosylated hyalurodinases include soluble forms of a partially deglycosylated PH20 polypeptides from any species, such as any set forth in any of SEQ ID NOS: 1, 2, 11, 25, 27, 29-32, 63, 65, and 101-102, or allelic variants, truncated variants, or other variants thereof. Such variants are known to one of skill in the art, and include polypeptides having 60%, 70%, 80%, 90%, 91%, 92%, 93%, 94%, 95% or more sequence identity to any of SEQ ID NOS: 1, 2, 11, 25, 27, 29-32, 63, 65, and 101-102, or truncated forms thereof. The partially deglycosylated hyaluronidases provided herein also include hybrid, fusion and chimeric partially deglycosylated hyaluronidases, and partially deglycosylated hyaluronidase conjugates.

Glycosidases, or glycoside hydrolases, are enzymes that catalyze the hydrolysis of the glycosidic linkage to generate two smaller sugars. The major types of N-glycans in vertebrates include high mannose glycans, hybrid glycans and complex glycans. There are several glycosidases that result in only partial protein deglycosylation, including: EndoF1, which cleaves high mannose and hybrid type glycans; EndoF2, which cleaves biantennary complex type glycans; EndoF3, which cleaves biantennary and more branched complex glycans; and EndoH, which cleaves high mannose and hybrid type glycans. Treatment of a hyaluronan-degrading enzyme, such as a soluble hyaluronidase, such as a soluble PH20, with one or all of these glycosidases can result in only partial deglycosylation and, therefore, retention of hyaluronidase activity.

Partially deglycosylated hyaluronan-degrading enzymes, such as partially deglycosylated soluble hyaluronidases, can be produced by digestion with one or more glycosidases, generally a glycosidase that does not remove all N-glycans but only partially deglycosylates the protein. For example, treatment of PH20 (e.g. a recombinant PH20 designated rHuPH20) with one or all of the above glycosidases (e.g. EndoF1, EndoF2 and/or EndoF3) results in partial deglycosylation. These partially deglycosylated PH20 polypeptides can exhibit hyaluronidase enzymatic activity that is comparable to the fully glycosylated polypeptides. In contrast, treatment of PH20 with PNGaseF, a glycosidase that cleaves all N-glycans, results in complete removal of all N-glycans and thereby renders PH20 enzymatically inactive. Thus, although all N-linked glycosylation sites (such as, for example, those at amino acids N82, N166, N235, N254, N368, and N393 of human PH20, exemplified in SEQ ID NO: 1) can be glycosylated, treatment with one or more glycosidases can render the extent of glycosylation reduced compared to a hyaluronidase that is not digested with one or more glycosidases.

The partially deglycosylated hyaluronan-degrading enzymes, including partially deglycosylated soluble PH20 polypeptides, can have 10%, 20%, 30%, 40%, 50%, 60%, 70% or 80% of the level of glycosylation of a fully glycosylated polypeptide. In one example, 1, 2, 3, 4, 5 or 6 of the N-glycosylation sites corresponding to amino acids N82, N166, N235, N254, N368, and N393 of SEQ ID NO:1 are partially deglycosylated, such that they no longer contain high mannose or complex type glycans, but rather contain at least an N-acetylglucosamine moiety. In some examples, 1, 2 or 3 of the N-glycosylation sites corresponding to amino acids N82, N166 and N254 of SEQ ID NO:1 are deglycosylated, that is, they do not contain a sugar moiety. In other examples, 3, 4, 5, or 6 of the N-glycosylation sites corresponding to amino acids N82, N166, N235, N254, N368, and N393 of SEQ ID NO:1 are glycosylated. Glycosylated amino acid residues minimally contain an N-acetylglucosamine moiety. Typically, the partially deglyclosylated hyaluronan-degrading enzymes, including partially deglycosylated soluble PH20 polypeptides, exhibit hyaluronidase activity that is 10%, 20%, 30%, 40%, 50%, 60%, 70%, 80%, 90%, 100%, 110%, 120%, 130%, 140%, 150%, 200%, 300%, 400%, 500%, 1000% or more of the hyaluronidase activity exhibited by the fully glycosylated polypeptide.

5. Modified (Polymer-Conjugated) Hyaluronan-Degrading Enzymes

In one example, the provided compositions and combinations contain hyaluronan-degrading enzymes, in particular soluble hyaluronidases, that have been modified by conjugation to one or more polymeric molecules (polymer), typically to increase the half-life of the hyaluronan-degrading enzyme, for example, to promote prolonged/sustained treatment effects in a subject.

Covalent or other stable attachment (conjugation) of polymeric molecules, such as polyethylene glycol (PEGylation moiety (PEG)), to the hyaluronan-degrading enzymes, such as hyaluronidases, impart beneficial properties to the resulting hyaluronan-degrading enzyme-polymer composition. Such properties include improved biocompatibility, extension of protein (and enzymatic activity) half-life in the blood, cells and/or in other tissues within a subject, effective shielding of the protein from proteases and hydrolysis, improved biodistribution, enhanced pharmacokinetics and/or pharmacodynamics, and increased water solubility.

Exemplary polymers that can be conjugated to the hyaluronan-degrading enzyme, such as the hyaluronidase, include natural and synthetic homopolymers, such as polyols (i.e. poly-OH), polyamines (i.e. poly-NH₂) and polycarboxyl acids (i.e. poly-COOH), and further heteropolymers i.e. polymers comprising one or more different coupling groups e.g. a hydroxyl groups and amine groups. Examples of suitable polymeric molecules include polymeric molecules selected from among polyalkylene oxides (PAO), such as polyalkylene glycols (PAG), including polyethylene glycols (PEG), methoxypolyethylene glycols (mPEG) and polypropylene glycols, PEG-glycidyl ethers (Epox-PEG), PEG-oxycarbonylimidazole (CDI-PEG), branched polyethylene glycols (PEGs), polyvinyl alcohol (PVA), polycarboxylates, polyvinylpyrrolidone, poly-D,L-amino acids, polyethylene-co-maleic acid anhydride, polystyrene-co-maleic acid anhydride, dextrans including carboxymethyl-dextrans, heparin, homologous albumin, celluloses, including methylcellulose, carboxymethylcellulose, ethylcellulose, hydroxyethylcellulose carboxyethylcellulose and hydroxypropylcellulose, hydrolysates of chitosan, starches such as hydroxyethyl-starches and hydroxypropyl-starches, glycogen, agaroses and derivatives thereof, guar gum, pullulan, inulin, xanthan gum, carrageenan, pectin, alginic acid hydrolysates and bio-polymers.

Typically, the polymers are polyalkylene oxides (PAO), such as polyethylene oxides, such as PEG, typically mPEG, which, in comparison to polysaccharides such as dextran and pullulan, have few reactive groups capable of cross-linking. Typically, the polymers are non-toxic polymeric molecules such as (m)polyethylene glycol (mPEG) which can be covalently conjugated to the hyaluronan-degrading enzyme, such as the hyaluronidase (e.g. to attachment groups on the protein's surface) using a relatively simple chemistry.

PEGylation of therapeutics has been reported to increase resistance to proteolysis, increase plasma half-life, and decrease antigenicity and immunogenicity. Examples of PEGylation methodologies are known in the art (see for example, Lu and Felix, Int. J. Peptide Protein Res., 43:127-138, 1994; Lu and Felix, Peptide Res., 6:140-6, 1993; Felix et al., Int. J. Peptide Res., 46:253-64, 1995; Benhar et al., J. Biol. Chem., 269: 13398-404, 1994; Brumeanu et al., J Immunol., 154:3088-95, 1995; see also, Caliceti et al. (2003) Adv. Drug Deliv. Rev. 55(10):1261-77 and Molineux (2003) Pharmacotherapy 23 (8 Pt 2):3S-8S). PEGylation also can be used in the delivery of nucleic acid molecules in vivo. For example, PEGylation of adenovirus can increase stability and gene transfer (see, e.g., Cheng et al. (2003) Pharm. Res. 20(9):1444-51).

Suitable polymeric molecules for attachment to the hyaluronan-degrading enzymes, including hyaluronidases, include, but are not limited to, polyethylene glycol (PEG) and PEG derivatives such as methoxy-polyethylene glycols (mPEG), PEG-glycidyl ethers (Epox-PEG), PEG-oxycarbonylimidazole (CDI-PEG), branched PEGs, and polyethylene oxide (PEO) (see e.g. Roberts et al., Advanced Drug Delivery Review (2002) 54: 459-476; Harris and Zalipsky, S (eds.) “Poly(ethylene glycol), Chemistry and Biological Applications” ACS Symposium Series 680, 1997; Mehvar et al., J. Pharm. Pharmaceut. Sci., 3(1):125-136, 2000; Harris, (2003) Nature Reviews Drug Discovery 2:214-221; and Tsubery, (2004) J Biol. Chem. 279(37):38118-24). The polymeric molecule can be of a molecular weight typically ranging from about 3 kDa to about 60 kDa. In some embodiments the polymeric molecule that is conjugated to a protein, such as rHuPH20, has a molecular weight of 5, 10, 15, 20, 25, 30, 35, 40, 45, 50, 55, 60 or more than 60 kDa.

PEGylated Soluble Hyaluronan-Degrading Enzymes

The hyaluronan-degrading enzyme used in the methods, compositions and combinations herein can be a PEGylated hyaluronan-degrading enzyme, such as a PEGylated soluble hyaluronan-degrading enzyme. In one example, it is a PEGylated soluble hyaluronidase, e.g. PEGylated rHuPH20. Various methods of modifying polypeptides by covalently attaching (conjugating) a PEG or PEG derivative (i.e. “PEGylation”) are known in the art (see e.g., U.S. 2006/0104968; U.S. Pat. No. 5,672,662; U.S. Pat. No. 6,737,505; and U.S. 2004/0235734). Techniques for PEGylation include, but are not limited to, specialized linkers and coupling chemistries (see e.g., Roberts, Adv. Drug Deliv. Rev. 54:459-476, 2002), attachment of multiple PEG moieties to a single conjugation site (such as via use of branched PEGs; see e.g., Guiotto et al., Bioorg. Med. Chem. Lett. 12:177-180, 2002), site-specific PEGylation and/or mono-PEGylation (see e.g., Chapman et al., Nature Biotech. 17:780-783, 1999), and site-directed enzymatic PEGylation (see e.g., Sato, Adv. Drug Deliv. Rev., 54:487-504, 2002). Methods and techniques described in the art can produce proteins having 1, 2, 3, 4, 5, 6, 7, 8, 9, 10 or more than 10 PEGs or PEG derivatives attached to a single protein molecule (see e.g., U.S. 2006/0104968).

Numerous reagents for PEGylation have been described in the art. Such reagents include, but are not limited to, N-hydroxysuccinimidyl (NHS) activated PEG, succinimidyl mPEG, mPEG₂-N-hydroxysuccinimide, mPEG succinimidyl alpha-methylbutanoate, mPEG succinimidyl propionate, mPEG succinimidyl butanoate, mPEG carboxymethyl 3-hydroxybutanoic acid succinimidyl ester, homobifunctional PEG-succinimidyl propionate, homobifunctional PEG propionaldehyde, homobifunctional PEG butyraldehyde, PEG maleimide, PEG hydrazide, p-nitrophenyl-carbonate PEG, mPEG-benzotriazole carbonate, propionaldehyde PEG, mPEG butryaldehyde, branched mPEG₂ butyraldehyde, mPEG acetyl, mPEG piperidone, mPEG methylketone, mPEG “linkerless” maleimide, mPEG vinyl sulfone, mPEG thiol, mPEG orthopyridylthioester, mPEG orthopyridyl disulfide, Fmoc-PEG-NHS, Boc-PEG-NHS, vinylsulfone PEG-NHS, acrylate PEG-NHS, fluorescein PEG-NHS, and biotin PEG-NHS (see e.g., Monfardini et al., Bioconjugate Chem. 6:62-69, 1995; Veronese et al., J. Bioactive Compatible Polymers 12:197-207, 1997; U.S. Pat. No. 5,672,662; U.S. Pat. No. 5,932,462; U.S. Pat. No. 6,495,659; U.S. Pat. No. 6,737,505; U.S. Pat. No. 4,002,531; U.S. Pat. No. 4,179,337; U.S. Pat. No. 5,122,614; U.S. Pat. No. 5,324,844; U.S. Pat. No. 5,446,090; U.S. Pat. No. 5,612,460; U.S. Pat. No. 5,643,575; U.S. Pat. No. 5,766,581; U.S. Pat. No. 5,795,569; U.S. Pat. No. 5,808,096; U.S. Pat. No. 5,900,461; U.S. Pat. No. 5,919,455; U.S. Pat. No. 5,985,263; U.S. Pat. No. 5,990,237; U.S. Pat. No. 6,113,906; U.S. Pat. No. 6,214,966; U.S. Pat. No. 6,258,351; U.S. Pat. No. 6,340,742; U.S. Pat. No. 6,413,507; U.S. Pat. No. 6,420,339; U.S. Pat. No. 6,437,025; U.S. Pat. No. 6,448,369; U.S. Pat. No. 6,461,802; U.S. Pat. No. 6,828,401; U.S. Pat. No. 6,858,736; U.S. 2001/0021763; U.S. 2001/0044526; U.S. 2001/0046481; U.S. 2002/0052430; U.S. 2002/0072573; U.S. 2002/0156047; U.S. 2003/0114647; U.S. 2003/0143596; U.S. 2003/0158333; U.S. 2003/0220447; U.S. 2004/0013637; US 2004/0235734; U.S. 2005/0114037; U.S. 2005/0171328; U.S. 2005/0209416; EP 1064951; EP 0822199; WO 0176640; WO 05000360; WO 0002017; WO 0249673; WO 9428024; and WO 0187925).

E. Pharmaceutical Compositions and Formulations, Dosage and Administration

Provided herein are compositions and combinations containing an organophosphorus bioscavenger and a hyaluronan-degrading enzyme. The compositions and combinations can be formulated into suitable pharmaceutical preparations, such as solutions and suspensions. Typically, the OP bioscavengers and hyaluronan-degrading enzymes are formulated into pharmaceutical compositions using techniques and procedures well known in the art (see e.g., Ansel Introduction to Pharmaceutical Dosage Forms, Fourth Edition, 1985, 126). The OP bioscavenger can be co-formulated or co-administered with the hyaluronan-degrading enzyme to treat organophosphorus poisoning. When co-administered, the hyaluronan-degrading enzyme can be administered prior to, simultaneously with or subsequent to the organophosphorus bioscavenger. Also provided herein are pharmaceutical compositions containing one or more additional agents used to treat organophosphorus poisoning. Exemplary of such agents are carbamates, anti-muscarinics, ChE-reactivators and anti-convulsives (see Section I below). The provided compositions and combinations of an organophosphorus bioscavenger and hyaluronan-degrading enzyme can be co-formulated or co-administered with pharmaceutical compositions of such second agents.

1. Dosages and Formulations for Administration

In the compositions, combinations and methods provided herein, the organophosphorus bioscavenger and hyaluronan-degrading enzyme are administered in an amount to prevent, ameliorate or reduce both organophosphorus poisoning and side effects or adverse effects of organophosphorus poisoning. The compositions and combinations can be used in methods of treatment and uses for counteracting the effects of poisoning caused by OP compounds and nerve agents, including in prophylactic treatments. Pharmaceutical compositions of organophosphorus bioscavengers and hyaluronan-degrading enzymes can be administered by any suitable route of administration, including but not limited to orally, intravenously (IV), subcutaneously, intramuscularly, intra-tumorally, intradermally, topically, transdermally, rectally or sub-epidermally. Other routes of administration are contemplated, such as any route known to those of skill in the art.

The most suitable route in any given case depends on a variety of factors, such as the nature, progress and severity of the OP poisoning for which the composition is used. Typically, for purposes herein, the organophosphorus bioscavengers and hyaluronan-degrading enzymes are administered parenterally. These include, for example, subcutaneous and intramuscular routes of administration. Thus, in one example, local administration can be achieved by injection, such as from a syringe or other article of manufacture containing an injection device such as a needle. Other modes of administration also are contemplated. Pharmaceutical compositions can be formulated in dosage forms appropriate for each route of administration.

The organophosphorus bioscavenger can be administered sequentially, at the same time or in the same composition as the hyaluronan-degrading enzyme. Compositions also can be administered with other biologically active second agents or treatments, such as carbamates, anti-muscarinics and ChE-reactivators, either sequentially, intermittently, at the same time or in the same composition. Administration also can include controlled release systems including controlled release formulations and device controlled release, such as by means of a pump.

Further, the concentration of the pharmaceutically active agent(s) can be adjusted so that administration provides an effective amount to produce the desired pharmacological effect. The exact dose depends on the age, weight and condition of the patient or animal as is known in the art. The unit-dose parenteral preparations are packaged in an ampoule, a vial or a syringe with a needle. The volume of liquid solution or reconstituted powder preparation, containing the pharmaceutically active agent(s), is a function of severity of the disease and the particular article of manufacture chosen for package. All preparations for parenteral administration must be sterile, as is known and practiced in the art.

Also, it is understood that the precise dosage and duration of treatment is a function of the organophosphorus poisoning and can be determined empirically using known testing protocols or by extrapolation from in vivo or in vitro test data. It is to be noted that concentrations and dosage values also can vary with the severity of the poisoning. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or use of compositions and combinations containing them. The compositions can be administered by the minute, hourly, daily, weekly, monthly, yearly or once. Generally, dosage regimens are chosen to limit toxicity. It should be noted that the attending physician would know how to and when to terminate, interrupt or adjust therapy to lower dosage due to toxicity, or bone marrow, liver or kidney or other tissue dysfunctions. Conversely, the attending physician would also know how to and when to adjust treatment to higher levels if the clinical response is not adequate (precluding toxic side effects).

The pharmaceutical compositions can be formulated in dosage forms appropriate for each route of administration. Pharmaceutically and therapeutically active agents and derivatives thereof are typically formulated and administered in unit dosage forms or multiple dosage forms. Each unit dose contains a predetermined quantity of therapeutically active agent sufficient to produce the desired therapeutic effect, in association with the required pharmaceutical carrier, vehicle or diluent. Examples of unit dose forms include ampoules and syringes and individually packaged tablets or capsules. Unit dose forms can be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, a multiple dose form is a multiple of unit doses that are not segregated in packaging. Generally, dosage forms or compositions containing active ingredient in the range of 0.005% to 100% with the balance made up from non-toxic carrier can be prepared. The therapeutic agent(s) can be formulated as pharmaceutical compositions for single or multiple dosage use.

Pharmaceutically acceptable compositions are prepared in view of approvals for a regulatory agency or other agency prepared in accordance with generally recognized pharmacopeia for use in animals and in humans. Compositions can take the form of solutions, suspensions, emulsion, tablets, pills, capsules, powders, and sustained release formulations. A composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, and other such agents. The formulation should suit the mode of administration.

Pharmaceutical compositions can include carriers such as a diluent, adjuvant, excipient, or vehicle with which an enzyme is administered. Examples of suitable pharmaceutical carriers are described in “Remington's Pharmaceutical Sciences” by E. W. Martin. Such compositions will contain a therapeutically effective amount of the compound, generally in purified form, together with a suitable amount of carrier so as to provide the form for proper administration to the patient. Such pharmaceutical carriers can be sterile liquids, such as water and oils, including those of petroleum, animal, vegetable or synthetic origin, such as peanut oil, soybean oil, mineral oil, and sesame oil. Water is a typical carrier when the pharmaceutical composition is administered intravenously. Saline solutions and aqueous dextrose and glycerol solutions also can be employed as liquid carriers, particularly for injectable solutions. Compositions can contain along with an active ingredient: a diluent such as lactose, sucrose, dicalcium phosphate, or carboxymethylcellulose; a lubricant, such as magnesium stearate, calcium stearate and talc; and a binder such as starch, natural gums, such as gum acaciagelatin, glucose, molasses, polyvinylpyrrolidine, celluloses and derivatives thereof, povidone, crospovidones and other such binders known to those of skill in the art. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, and ethanol. A composition, if desired, also can contain minor amounts of wetting or emulsifying agents, or pH buffering agents, for example, acetate, sodium citrate, cyclodextrin derivatives, sorbitan monolaurate, triethanolamine sodium acetate, triethanolamine oleate, and other such agents.

Formulations are provided for administration to humans and animals in unit dosage forms, such as tablets, capsules, pills, powders, granules, sterile parenteral solutions or suspensions, and oral solutions or suspensions, and oil water emulsions containing suitable quantities of the compounds or pharmaceutically acceptable derivatives thereof. Pharmaceutically and therapeutically active compounds and derivatives thereof are typically formulated and administered in unit dosage forms or multiple dosage forms. Each unit dose contains a predetermined quantity of therapeutically active compound sufficient to produce the desired therapeutic effect, in association with the required pharmaceutical carrier, vehicle or diluent. Examples of unit dose forms include ampoules and syringes and individually packaged tablets or capsules. Unit dose forms can be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses that are not segregated in packaging. Generally, dosage forms or compositions containing active ingredient in the range of 0.005% to 100% with the balance made up from non-toxic carrier can be prepared. The compositions and combinations provided herein of a hyaluronan-degrading enzyme and organophosphorus bioscavenger can be provided as a liquid or lyophilized formulation. Lyophilized formulations are ideal for long term storage of the combinations and compositions provided herein.

In one example, pharmaceutical preparation can be in liquid form, for example, solutions, syrups or suspensions. If provided in liquid form, the pharmaceutical preparation of OP bioscavenger (e.g. rBChE) and/or a hyaluronan-degrading enzyme, for example, can be provided as a concentrated preparation to be diluted to a therapeutically effective concentration. The hyaluronan-degrading enzyme and OP bioscavenger (e.g. rBCheE) co-formulation can be prepared and stored together or can be mixed together immediately prior to administration. Alternatively, the hyaluronan-degrading enzyme and OP bioscavenger are prepared as separate formulations, and are administered simultaneously, intermittently or sequentially. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid).

In another example, pharmaceutical preparations can be presented in lyophilized form for reconstitution with water or other suitable vehicle before use. For example, the pharmaceutical preparations of an OP bioscavenger (e.g. rBChE) and/or a hyaluronan-degrading enzyme can be reconstituted with a solution, generally a buffer or liquid solution.

Administration methods can be employed to decrease the exposure of organophosphorus bioscavengers and/or hyaluronan-degrading enzymes to degradative processes, such as proteolytic degradation and immunological intervention via antigenic and immunogenic responses. Examples of such methods include local administration at the site of treatment. PEGylation of therapeutics has been reported to increase resistance to proteolysis, increase plasma half-life, and decrease antigenicity and immunogenicity. Examples of PEGylation methodologies are known in the art (see for example, Lu and Felix, Int. J. Peptide Protein Res., 43: 127-138, 1994; Lu and Felix, Peptide Res., 6:140-6, 1993; Felix et al., Int. J. Peptide Res., 46: 253-64, 1995; Benhar et al., J. Biol. Chem., 269: 13398-404, 1994; Brumeanu et al., J Immunol., 154: 3088-95, 1995; see also, Caliceti et al. (2003) Adv. Drug Deliv. Rev. 55(10):1261-77 and Molineux (2003) Pharmacotherapy 23 (8 Pt 2):3S-8S). PEGylation also can be used in the delivery of nucleic acid molecules in vivo. For example, PEGylation of adenovirus can increase stability and gene transfer (see, e.g., Cheng et al. (2003) Pharm. Res. 20(9): 1444-51).

a. Organophosphorus Bioscavengers

A selected organophosphorus bioscavenger, for example, a cholinesterase, e.g. rBChE, is included in an amount sufficient that exhibits a therapeutically useful effect. The composition containing the OP bioscavenger, hyaluronan-degrading enzyme, or both, can include a pharmaceutically acceptable carrier. A therapeutically effective concentration can be determined empirically by testing the compounds in known in vivo or in vitro systems, such as the assays provided herein. The concentration of a selected organophosphorus bioscavenger, for example rBChE, in the composition depends on absorption, inactivation and excretion rates, the physicochemical characteristics, the dosage schedule, and amount administered as well as other factors known to those of skill in the art. For example, it is understood that the precise dosage and duration of treatment is a function of the subject being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope thereof.

The amount of a selected organophosphorus bioscavenger, for example, rBChE, to be administered in the compositions and combinations provided herein for the treatment of organophosphorus poisoning, including pesticide poisoning or nerve agent poisoning, can be determined by standard clinical techniques. In addition, in vitro assays and animal models can be employed to help identify optimal dosage ranges. The precise dosage, which can be determined empirically, can depend on the particular OP bioscavenger, the organophosphorus compound and its method of administration, and the severity of the OP poisoning. OP bioscavengers in the compositions and combinations provided herein are formulated in an amount in a range between or between about 0.5 μg to 2000 mg, such as 1 μg to 1000 mg, for example, between or between about 1 μg to 100 mg, 1 μg to 1 mg, 1 mg to 1000 mg, 1 mg to 500 mg, 1 mg to 100 mg, 50 mg to 1000 mg, 250 mg to 1000 mg, or 250 mg to 750 mg, such as, for example, between or between about 1 μg to 100 mg, 1 μg to 50 mg, 1 μg to 25 mg, 1 μg to 10 mg, 1 μg to 1 mg, 1 μg to 500 μg, 1 μg to 250 μg, 1 kg to 100 kg, 1 μg to 50 μg, 50 μg to 100 mg, 50 μg to 25 mg, 50 μg to 10 mg, 50 μg to 1 mg, 50 μg to 500 μg, 50 μg to 250 μg, 50 μg to 100 μg, 100 μg to 100 mg, 100 μg to 50 mg, 100 μg to 25 mg, 100 μg to 10 mg, 100 μg to 1 mg, 100 μg to 500 μg, 100 μg to 250 μg, 250 μg to 100 mg, 250 μg to 50 mg, 250 μg to 25 mg, 250 μg to 10 mg, 250 μg to 1 mg, 250 μg to 500 μg, 500 μg to 100 mg, 500 μg to 50 mg, 500 μg to 25 mg, 500 μg to 10 mg, 500 μg to 1 mg, 1 mg to 500 mg, 1 mg to 250 mg, 1 mg to 150 mg, 1 mg to 100 mg, 1 mg to 75 mg, 1 mg to 50 mg, 25 mg to 500 mg, 25 mg to 250 mg, 25 mg to 150 mg, 25 mg to 100 mg, 25 mg to 75 mg, 25 mg to 50 mg, 50 mg to 750 mg, 50 mg to 500 mg, 50 mg to 250 mg, 50 mg to 150 mg, 50 mg to 100 mg, 100 mg to 1000 mg, 100 mg to 750 mg, 100 mg to 500 mg, 100 mg to 250, 250 mg to 1000 mg, 250 mg to 750 mg, 250 mg to 500 mg, 500 mg to 1000 mg, 500 mg to 750 mg, or can be at or about or at least 1 μg, 10 μg, 50 μg, 100 μg, 250 μg, 500 μg, 750 μg, 1 mg, 5 mg, 10 mg, 15 mg, 20 mg, 25 mg, 30 mg, 35 mg, 40 mg, 45 mg, 50 mg, 60 mg, 70 mg, 80 mg, 90 mg, 100 mg, 150 mg, 200 mg, 250 mg, 300 mg, 340 mg, 350 mg, 400 mg, 500 mg, 600 mg, 700 mg, 750 mg, 800 mg, 900 mg or 1000 mg or more. The particular amount depends on the organophosphorus bioscavenger. Higher dosage amounts can be provided for stoichiometric bioscavengers whereas lower doses can be provided for catalytic bioscavengers. The amount can be for direct administration. The amount in the composition can be for single dosage administration or multiple dosage administration.

For example, a catalytic bioscavenger in the compositions and combinations provided herein can be formulated in an amount from between or between about 1 μg to 100 mg, for example, between or between about 1 μg to 50 mg, 1 μg to 1 mg, 1 μg to 500 μg, 250 μg to 1 mg, 500 μg to 1 mg, 500 μg to 10 mg, 500 μg to 50 mg, 1 mg to 100 mg, such as, for example, between or between about 1 μg to 100 mg, 1 μg to 50 mg, 1 μg to 25 mg, 1 μg to 10 mg, 1 μg to 1 mg, 1 μg to 500 μg, 1 μg to 250 μg, 1 μg to 100 μg, 1 μg to 50 μg, 50 μg to 100 mg, 50 μg to 25 mg, 50 μg to 10 mg, 50 μg to 1 mg, 50 μg to 500 μg, 50 μg to 250 μg, 50 μg to 100 μg, 100 μg to 100 mg, 100 μg to 50 mg, 100 μg to 25 mg, 100 μg to 10 mg, 100 μg to 1 mg, 100 μg to 500 μg, 100 μg to 250 μg, 250 μg to 100 mg, 250 μg to 50 mg, 250 μg to 25 mg, 250 μg to 10 mg, 250 μg to 1 mg, 250 μg to 500 μg, 500 μg to 100 mg, 500 μg to 50 mg, 500 μg to 25 mg, 500 μg to 10 mg, 500 μg to 1 mg, or can be at or about or at least 1 μg, 10 μg, 50 μg, 100 μg, 250 μg, 500 μg, 750 μg, 1 mg, 5 mg, 10 mg, 15 mg, 20 mg, 25 mg, 30 mg, 35 mg, 40 mg, 45 mg, 50 mg, 60 mg, 70 mg, 80 mg, 90 mg or 100 mg. The amount in the composition can be for single dosage administration or multiple dosage administration. The amount can be for direct administration.

In another example, a stoichiometric bioscavenger in the compositions and combinations provided herein can be formulated in a dosage amount from between or between about 1 mg to 1000 mg, for example, between or between about 1 mg to 1000 mg, 1 mg to 500 mg, 1 mg to 100 mg, 50 mg to 1000 mg, 250 mg to 1000 mg, or 250 mg to 750 mg, such as, for example, between or between about 1 mg to 500 mg, 1 mg to 250 mg, 1 mg to 150 mg, 1 mg to 100 mg, 1 mg to 75 mg, 1 mg to 50 mg, 25 mg to 500 mg, 25 mg to 250 mg, 25 mg to 150 mg, 25 mg to 100 mg, 25 mg to 75 mg, 25 mg to 50 mg, 50 mg to 750 mg, 50 mg to 500 mg, 50 mg to 250 mg, 50 mg to 150 mg, 50 mg to 100 mg, 100 mg to 1000 mg, 100 mg to 750 mg, 100 mg to 500 mg, 100 mg to 250, 250 mg to 1000 mg, 250 mg to 750 mg, 250 mg to 500 mg, 500 mg to 1000 mg, 500 mg to 750 mg, or can be at or about or at least 1 mg, 5 mg, 10 mg, 15 mg, 20 mg, 25 mg, 30 mg, 35 mg, 40 mg, 45 mg, 50 mg, 60 mg, 70 mg, 80 mg, 90 mg, 100 mg, 150 mg, 200 mg, 250 mg, 300 mg, 340 mg, 350 mg, 400 mg, 500 mg, 600 mg, 700 mg, 750 mg, 800 mg, 900 mg or 1000 mg or more. The amount in the composition can be for single dosage administration or multiple dosage administration. The amount can be for direct administration.

The particular dosage and formulation thereof depends upon the indication and individual. If necessary dosages can be empirically determined. Typically any of the compositions above are formulated in a volume of 0.1-100 mL, particularly, 0.1-10 mL, 0.1-5 mL, 0.1-3 mL, 0.1-1 mL, 1-10 mL, 3-5 mL, 3-10 mL, 5-10 mL, 1-50 mL, 10-50 mL, 10-30 mL, 1-20 mL, 1-5 mL, 1-3 mL, 2 to 8 mL, 4 to 7 mL or 4 to 5 mL volumes following reconstitution, such as by addition of an appropriate buffer. Typically, such dosage amounts are from at or about 50 mg to 1000 mg, generally 250 mg to 750 mg, in a 0.1 to 10 mL final volume. Hence, the concentration of organophosphorus bioscavenger present in a composition can be between 1 μg/mL to 10,000 mg/mL, such as from or from about 1 μg/mL to 1000 μg/mL, 1 to 500 μg/mL, 1 to 250 μg/mL, 1 to 100 μg/mL, 50 to 1000 μg/mL, 50 to 750 μg/mL, 50 to 500 μg/mL, 50 to 250 μg/mL, 100 to 1000 μg/mL, 100 to 500 mg/mL, 100 to 250 μg/mL, 250 to 1000 μg/mL, 250 to 750 μg/mL, 250 to 500 μg/mL, 500 to 1000 μg/mL, 0.5 to 50 mg/mL, 0.5 to 10 mg/mL, 0.5 to 1 mg/mL, 1 to 100 mg/mL, 1 to 50 mg/mL, 1 to 25 mg/mL, 1 to 1000 mg/mL, 1 to 500 mg/mL, 1 to 250 mg/mL, 10 to 500 mg/mL, 10 to 250 mg/mL, 10 to 150 mg/mL, 10 to 100 mg/mL, 10 to 50 mg/mL, 50 to 1500 mg/mL, 50 to 1000 mg/mL, 50 to 750 mg/mL, 50 to 500 mg/mL, 50 to 250 mg/mL, 100 to 1500 mg/mL, 100 to 1000 mg/mL, 100 to 750 mg/mL, 100 to 500 mg/mL, 100 to 7500 mg/mL, 100 to 5000 mg/mL, 100 to 2500 mg/mL, 500 to 10000 mg/mL, 500 to 7500 mg/mL, 500 to 5000 mg/mL, 500 to 2500 mg/mL, or 500 to 1000 mg/mL. An organophosphorus bioscavenger, for example rBChE, can be administered at once, or can be divided into a number of smaller doses to be administered at intervals of time. Selected OP bioscavengers, for example rBChE, can be administered in one or more doses over the course of a treatment time for example over several minutes, hours, days, weeks, or months. In some cases, continuous administration is useful. It is understood that the precise dosage and course of administration depends on the particular subject and methods.

b. Hyaluronan-Degrading Enzymes

A selected hyaluronan-degrading enzyme, for example, a hyaluronidase, e.g. rHuPH20, is included in an amount sufficient that exhibits a therapeutically useful effect. The composition containing the OP bioscavenger, hyaluronan-degrading enzyme, or both, can include a pharmaceutically acceptable carrier. A therapeutically effective concentration can be determined empirically by testing the compounds in known in vivo or in vitro systems, such as the assays provided herein.

The amount of a selected hyaluronan-degrading enzyme, for example, rHuPH20, to be administered in the compositions and combinations provided herein for the treatment of organophosphorus poisoning, including pesticide poisoning or nerve agent poisoning, can be determined by standard clinical techniques. In addition, the therapeutically effective concentration can be determined empirically by testing the polypeptides in known in vitro and in vivo systems such as by using the assays provided herein or known in the art (see e.g., Taliani et al. (1996) Anal. Biochem., 240: 60-67; Filocamo et al. (1997) J Virology, 71: 1417-1427; Sudo et al. (1996) Antiviral Res. 32: 9-18; Bouffard et al. (1995) Virology, 209:52-59; Bianchi et al. (1996) Anal. Biochem., 237: 239-244; Hamatake et al. (1996) Intervirology 39:249-258; Steinkuhler et al. (1998) Biochem., 37:8899-8905; D'Souza et al. (1995) J Gen. Virol., 76:1729-1736; Takeshita et al. (1997) Anal. Biochem., 247:242-246; see also e.g, Shimizu et al. (1994) J. Virol. 68:8406-8408; Mizutani et al. (1996) J. Virol. 70:7219-7223; Mizutani et al. (1996) Biochem. Biophys. Res. Commun., 227:822-826; Lu et al. (1996) Proc. Natl. Acad. Sci. (USA), 93:1412-1417; Hahm et al., (1996) Virology, 226:318-326; Ito et al. (1996) J. Gen. Virol., 77:1043-1054; Mizutani et al. (1995) Biochem. Biophys. Res. Commun., 212:906-911; Cho et al. (1997) J. Virol. Meth. 65:201-207 and then extrapolated therefrom for dosages for humans.

For example, a hyaluronan-degrading enzyme in a composition or combination provided herein can be formulated at an amount between or between about 0.5 μg to 1000 mg, such as 100 μg to 1 mg, 1 mg to 20 mg or 1 mg to 100 mg, for example, 100 μg to 5 mg, 0.5 μg to 1450 μg, 1 μg to 1000 μg, 5 μg to 1250 μg, 10 μg to 750 μg, 50 μg to 500 μg, 0.5 μg to 500 μg, 500 μg to 1450 μg, 1 mg to 500 mg, 1 mg to 250 mg, 1 mg to 100 mg, 1 mg to 50 mg, 10 mg to 500 mg, 10 mg to 250 mg, 10 mg to 100 mg, 10 mg to 50 mg, 25 mg to 500 mg, 25 mg to 250 mg, 25 mg to 100 mg, 25 mg to 50 mg, 50 mg to 1000 mg, 50 mg to 500 mg, 50 mg to 250 mg, 50 mg to 100 mg, 100 mg to 1000 mg, 100 mg to 500 mg, 100 mg to 250 mg, 250 mg to 1000 mg, 250 mg to 500 mg, or 500 mg to 1000 mg, or is at least or least about or is 10 μg, 50 μg, 100 μs, 200 μg, 300 μg, 400 μg, 500 μg, 600 μg, 700 μg, 800 μg, 900 μg, 1 mg, 5 mg, 10 mg, 25 mg, 50 mg, 100 mg, 150 mg, 250 mg, 500 mg or 1000 mg or more. The amount in the composition can be for single dosage administration or multiple dosage administration. The amount can be for direct administration.

For example, an exemplary rHuPH20 preparation exhibits a specific activity of 120,000 Units/mg, while a PEGylated form of rHuPH20 exhibits a specific activity of between at or about 18,000 and at or about 45,000 U/mg, such as about or at least 32,000 Units/mg.

Hence, in examples herein, a hyaluronan-degrading enzyme in a composition or combination provided herein can be formulated in a Unit amount. For example, a hyaluronan-degrading enzyme in a composition or combination provided herein can be formulated at an amount between or between about 10 to 50,000,000 Units, 10 to 40,000,000 Units, 10 to 36,000,000 Units, 10 to 12,000,000 Units, 10 to 1,200,000 Units, 10 to 1,000,000 Units, 10 to 500,000 Units, 100 to 100,000 Units, 500 to 50,000 Units, 1000 to 10,000 Units, 5000 to 7500 Units, 5000 Units to 50,000 Units, 1,000 to 10,000 Units, 10,000 Units to 6,000,000 Units, 10,000 U to 150,000 U, 10,000 U to 100,000 U, 10,000 U to 50,000 U, 50,000 U to 200,000 U, 50,000 U to 150,000 U, 50,000 U to 100,000 U, 10,000 U to 1,000,000 U, 50,000 U to 1,000,000 U, 500,000 U to 6,000,000 U, 500,000 U to 4,000,000 U, 500,000 U to 2,000,000 U, 500,000 U to 1,000,000 U, 1,000,000 U to 6,000,000 U, 1,000,000 U to 5,000,000 U, 1,000,000 U to 4,000,000 U, 1,000,000 U to 3,000,000 U, 1,000,000 U to 2,000,000 U, 2,000,000 U to 6,000,000 U, 2,000,000 U to 5,000,000 U, 2,000,000 U to 4,000,000 U, 2,000,000 U to 3,000,000 U, 3,000,000 U to 6,000,000 U, 4,000,000 U to 6,000,000 U, 5,000,000 U to 6,000,000 U, such as at least or least about or 1 Unit (U), 10 U, 50 U, 100 U, 500 U, 1,000 U, 5,000 U, 10,000 U, 20,000 U, 30,000 U, 40,000 U, 50,000 U, 60,000 U, 70,000 U, 80,000 U, 90,000 U, 100,000 U, 110,000 U, 120,000 U, 130,000 U, 140,000 U, 150,000 U, 160,000 U, 170,000 U, 180,000 U, 190,000 U, 200,000 U, 300,000 U, 400,000 U, 500,000 U, 600,000 U, 700,000 U, 800,000 U, 900,000 U, 1,000,000 U, 1,500,000 U, 2,000,000 U, 2,500,000 U, 3,000,000 U, 3,500,000 U, 4,000,000 U, 5,000,000 U, 6,000,000 U or more. The composition can be formulated for direct administration. The amount in the composition can be for single dosage administration or multiple dosage administration.

In some examples, dosages can be provided as a ratio to organophosphorus bioscavenger administered. For example, the hyaluronan-degrading enzyme can be administered at a ratio of hyaluronan-degrading enzyme:organophosphorus bioscavenger from between or between about 1:100 to 1:3,000, for example, at or about 1:100, 1:200, 1:300, 1:400, 1:500, 1:600, 1:700, 1:1:800, 1:900 1:1,000, 1:1,100, 1:1,200, 1:1,300, 1:1,400, 1:1,500, 1:1,600, 1:1,700, 1:1,800, 1:1,900, 1:2,000, 1:2,100, 1:2,200, 1:2,300, 1:2,400, 1:2,500, 1:2,600, 1:2,700, 1:2,800, 1:2,900 or 1:3,000.

Typically any of the compostions above are formulated in an amount to administer for indications described herein in a volume of 0.1-100 mL, particularly, 0.1-10 mL, 0.1-5 mL, 0.1-3 mL, 0.1-1 mL, 1-10 mL, 3-5 mL, 3-10 mL, 5-10 mL, 1-50 mL, 10-50 mL, 10-30 mL, 1-20 mL, 1-5 mL or 1-3 mL volumes. The hyaluronan-degrading enzyme (e.g. a hyaluronidase) can be provided in a composition at a concentration of from or from about 10 U/mL to 100,000 U/mL, 1000 U/mL to 50,000 U/mL, 5,000 U/mL to 20,000 U/mL, 10 U/mL to 10,000 U/mL, 10 U/mL to 5000 U/mL, or 50 U/mL to 15,000 U/mL, for example from or from about 10 U/mL to 500 U/mL, 50 U/mL to 1000 U/mL, 1000 U/mL to 15,000 U/mL, 100 U/mL to 5,000 U/mL, 500 U/mL to 5,000 U/mL, 100 U/mL to 750 U/mL, or 100 U/mL to 400 U/mL, for example at least or at least about or about or 50 U/mL, 100 U/mL, 150 U/mL, 200 U/mL, 400 U/mL, 500 U/mL, 1000 U/mL, 2000 Units/mL, 3000 U/mL, 4000 U/mL, 5000 U/mL, 6000 U/mL, 7000 U/mL, 8000 U/mL, 9000 U/mL, 10,000 U/mL, 11,000 U/mL, 12,000 U/mL, or 12,800 U/mL, or more. Generally the hyaluronan-degrading enzyme (e.g. a hyaluronidase) is present in a composition in a concentration that is at least or at least about or about 10 U/mL, 50 U/mL, 100 U/mL, 150 U/mL, 200 U/mL, 400 U/mL or 500 U/mL or can be provided in a more concentrated form, for example at least or at least about or about 1000 U/mL, 1500 Units/mL, 2000 U/mL, 4000 U/mL or 5000 U/mL for use directly or for dilution to the effective concentration prior to use.

For example, a hyaluronan-degrading enzyme in a composition for administration can be present in a concentration between or between about 0.5 μg to 5000 mg/mL, such as 0.5 mg/mL to 500 mg/mL, for example, between 0.5 mg/mL to 250 mg/mL, 0.5 mg/mL to 100 mg/mL, 0.5 mg/mL to 50 mg/mL, 0.5 mg/mL to 10 mg/mL, 1 mg/mL to 500 mg/mL, 1 mg/mL to 250 mg/mL, 1 mg/mL to 100 mg/mL, 1 mg/mL to 50 mg/mL, 50 mg/mL to 500 mg/mL, 50 mg/mL to 250 mg/mL, 50 mg/mL to 100 mg/mL, 100 mg/mL to 500 mg/mL, 100 mg/mL to 250 mg/mL or 250 mg/mL to 500 mg/mL, or is at least or least about or is 0.5 mg/mL, 1 mg/mL, 10 mg/mL, 20 mg/mL, 30 mg/mL, 40 mg/mL, 50 mg/mL, 60 mg/mL, 70 mg/mL, 80 mg/mL, 90 mg/mL, 100 mg/mL, 150 mg/mL, 200 mg/mL, 250 mg/mL, 300 mg/mL, 400 mg/mL or 500 mg/mL. The composition can be for single dosage administration or multiple dosage administration.

A hyaluronan-degrading enzyme, for example a PH20 (e.g. rHuPH20), can be administered at once, or can be divided into a number of smaller doses to be administered at intervals of time. The hyaluronan-degrading enzyme can be administered in one or more doses over the course of a treatment time for example over several minutes, hours, days, weeks, or months. In some cases, continuous administration is useful. It is understood that the precise dosage and course of administration depends on the particular subject and methods.

For practice of the methods described herein, the hyaluronan-degrading enzyme in a composition or combination provided herein can be administered at an amount between or between about 1 Units/kg to 800,00 Units/kg, 10 Units/kg to 10,000 Units/kg, 100 Units/kg to 10,000 Units/kg, for example, between 1 Unit/kg to 1000 Units/kg, 1 Units/kg to 500 Units/kg or 10 Units/kg to 50 Units/kg, 100 U/kg to 5,000 U/kg, 100 U/kg to 2,500 U/kg, 100 U/kg to 1000 U/kg, 100 U/kg to 500 U/kg, 500 U/kg to 10,000 U/kg, 500 U/kg to 5,000 U/kg, 500 U/kg to 1000 U/kg, 750 U/kg to 5,000 U/kg, 750 U/kg to 2,500 U/kg, 1000 U/kg to 10,000 U/kg, 1000 U/kg to 5,000 U/kg, 1000 to 2,500 U/kg, 1000 U/kg to 2,000 U/kg, 1,500 U/kg to 5,000 U/kg, 1,500 U/kg to 2,500 U/kg, or 1,500 U/kg to 2,000 U/kg, or at least or at least about or 1 U/kg, 10 U/kg, 100 U/kg, 250 U/kg, 500 U/kg, 1000 U/kg, 1,250 U/kg, 1,500 U/kg, 1,750 U/kg, 2,000 U/kg, 2,100 U/kg, 2,200 U/kg, 2,300 U/kg, 2,400 U/kg, 2,500 U/kg, 3,000 U/kg, 4,000 U/kg, 5,000 U/kg, 7,500 U/kg or 1000 U/kg or more.

2. Injectables, Solutions and Emulsions

Parenteral administration, generally characterized by injection, either subcutaneously or intramuscularly is contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. The pharmaceutical compositions also may contain other minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow-release or sustained-release system, such that a constant level of dosage is maintained (see, e.g., U.S. Pat. No. 3,710,795) is also contemplated herein. The percentage of active compound contained in such parenteral compositions is highly dependent on the specific nature thereof, as well as the activity of the compound and the needs of the subject.

Parenteral administration of the compositions generally includes sub-epidermal routes of administration such as subcutaneous and intramuscular administrations. If desired, intravenous administration also is contemplated. Injectables are designed for local and systemic administration. For purposes herein, local administration is desired for direct administration to the affected interstitium. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous. If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers, which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcellulose, hydroxypropyl methylcellulose and polyvinylpyrrolidone. Emulsifying agents include Polysorbate 80 (TWEEN 80). A sequestering or chelating agent of metal ions includes EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art. The unit-dose parenteral preparations are packaged in an ampoule, a vial or a syringe with a needle. The volume of liquid solution or reconstituted powder preparation, containing the pharmaceutically active compound, is a function of the disease to be treated and the particular article of manufacture chosen for package. For example, for the treatment of organophosphorus poisoning, it is contemplated that for parenteral injection the injected volume is or is about 0.5 to 10 milliliters. All preparations for parenteral administration must be sterile, as is known and practiced in the art.

Lyophilized Powders

Of interest herein are lyophilized powders, which can be reconstituted for administration as solutions, emulsions and other mixtures. They may also be reconstituted and formulated as solids or gels.

The sterile, lyophilized powder is prepared by dissolving a compound of inactive enzyme in a buffer solution. The buffer solution may contain an excipient which improves the stability or other pharmacological component of the powder or reconstituted solution, prepared from the powder. Subsequent sterile filtration of the solution followed by lyophilization under standard conditions known to those of skill in the art provides the desired formulation. Briefly, the lyophilized powder is prepared by dissolving an excipient, such as dextrose, sorbitol, fructose, corn syrup, xylitol, glycerin, glucose, sucrose or other suitable agent, in a suitable buffer, such as citrate, sodium or potassium phosphate or other such buffer known to those of skill in the art. Then, a selected enzyme is added to the resulting mixture, and stirred until it dissolves. The resulting mixture is sterile filtered or treated to remove particulates and to insure sterility, and apportioned into vials for lyophilization. Each vial will contain a single dosage (1-1000 mg, generally 250-750 mg, such as 500-750 mg) or multiple dosages of the compound. The lyophilized powder can be stored under appropriate conditions, such as at about 4° C. to room temperature.

Reconstitution of this lyophilized powder with a buffer solution provides a formulation for use in parenteral administration. The solution chosen for reconstitution can be any buffer. For reconstitution about 0.1 to 10 mL, preferably 0.5 to 10 mL, more preferably 0.5 to 5 mL of buffer or other suitable carrier is added. The precise amount depends upon the indication treated and selected compound. Such amount can be empirically determined.

4. Compositions for Other Routes of Administration

Depending upon the condition treated other routes of administration, such as topical application, transdermal patches, oral and rectal administration are also contemplated herein. For example, pharmaceutical dosage forms for rectal administration are rectal suppositories, capsules and tablets for systemic effect. Rectal suppositories include solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The typical weight of a rectal suppository is about 2 to 3 gm. Tablets and capsules for rectal administration are manufactured using the same pharmaceutically acceptable substance and by the same methods as for formulations for oral administration.

Formulations suitable for rectal administration can be provided as unit dose suppositories. These can be prepared by admixing the active compound with one or more conventional solid carriers, for example, cocoa butter, and then shaping the resulting mixture.

For oral administration, pharmaceutical compositions can take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well-known in the art.

Formulations suitable for buccal (sublingual) administration include, for example, lozenges containing the active compound in a flavored base, usually sucrose and acacia or tragacanth; and pastilles containing the compound in an inert base such as gelatin and glycerin or sucrose and acacia.

Pharmaceutical compositions also can be administered by controlled release formulations and/or delivery devices (see, e.g., in U.S. Pat. Nos. 3,536,809; 3,598,123; 3,630,200; 3,845,770; 3,847,770; 3,916,899; 4,008,719; 4,687,610; 4,769,027; 5,059,595; 5,073,543; 5,120,548; 5,354,566; 5,591,767; 5,639,476; 5,674,533 and 5,733,566).

F. Methods of Producing Nucleic Acids Encoding an Organophosphorus Bioscavenger or Hyaluronan-Degrading Enzyme and Polypeptides Thereof

Polypeptides of an organophosphorus bioscavenger, such as butyrylcholinesterase, or hyaluronan-degrading enzyme, such as a soluble hyaluronidase, set forth herein, can be obtained by methods well known in the art for protein purification and recombinant protein expression. Any method known to those of skill in the art for identification of nucleic acids that encode desired genes can be used. Any method available in the art can be used to obtain a full length (i.e., encompassing the entire coding region) cDNA or genomic DNA clone encoding a hyaluronan-degrading enzyme, such as from a cell or tissue source. Modified or variant organophosphorus bioscavengers and hyaluronan-degrading enzymes, can be engineered from a wildtype polypeptide, such as by site-directed mutagenesis.

Polypeptides can be cloned or isolated using any available methods known in the art for cloning and isolating nucleic acid molecules. Such methods include PCR amplification of nucleic acids and screening of libraries, including nucleic acid hybridization screening, antibody-based screening and activity-based screening. Methods for amplification of nucleic acids can be used to isolate nucleic acid molecules encoding a desired polypeptide, including for example, polymerase chain reaction (PCR) methods. A nucleic acid containing material can be used as a starting material from which a desired polypeptide-encoding nucleic acid molecule can be isolated. For example, DNA and mRNA preparations, cell extracts, tissue extracts, fluid samples (e.g. blood, serum, saliva), samples from healthy and/or diseased subjects can be used in amplification methods. Nucleic acid libraries also can be used as a source of starting material. Primers can be designed to amplify a desired polypeptide. For example, primers can be designed based on expressed sequences from which a desired polypeptide is generated. Primers can be designed based on back-translation of a polypeptide amino acid sequence. Nucleic acid molecules generated by amplification can be sequenced and confirmed to encode a desired polypeptide.

Additional nucleotide sequences can be joined to a polypeptide-encoding nucleic acid molecule, including linker sequences containing restriction endonuclease sites for the purpose of cloning the synthetic gene into a vector, for example, a protein expression vector or a vector designed for the amplification of the core protein coding DNA sequences. Furthermore, additional nucleotide sequences specifying functional DNA elements can be operatively linked to a polypeptide-encoding nucleic acid molecule. Examples of such sequences include, but are not limited to, promoter sequences designed to facilitate intracellular protein expression, and secretion sequences, for example heterologous signal sequences, designed to facilitate protein secretion. Such sequences are known to those of skill in the art. Additional nucleotide residues sequences such as sequences of bases specifying protein binding regions also can be linked to enzyme-encoding nucleic acid molecules. Such regions include, but are not limited to, sequences of residues that facilitate or encode proteins that facilitate uptake of an enzyme into specific target cells, or otherwise alter pharmacokinetics of a product of a synthetic gene. For example, enzymes can be linked to PEG moieties.

In addition, tags or other moieties can be added, for example, to aid in detection or affinity purification of the polypeptide. For example, additional nucleotide residues sequences such as sequences of bases specifying an epitope tag or other detectable marker also can be linked to enzyme-encoding nucleic acid molecules. Exemplary of such sequences include nucleic acid sequences encoding a His tag (e.g., 6×His, HHHHHH; SEQ ID NO:54) or Flag Tag (DYKDDDDK; SEQ ID NO:55).

The identified and isolated nucleic acids can then be inserted into an appropriate cloning vector. A large number of vector-host systems known in the art can be used. Possible vectors include, but are not limited to, plasmids or modified viruses, but the vector system must be compatible with the host cell used. Such vectors include, but are not limited to, bacteriophages such as lambda derivatives, or plasmids such as pCMV4, pBR322 or pUC plasmid derivatives or the Bluescript vector (Stratagene, La Jolla, Calif.). Other expression vectors include the HZ24 expression vector exemplified herein. The insertion into a cloning vector can, for example, be accomplished by ligating the DNA fragment into a cloning vector which has complementary cohesive termini. Insertion can be effected using TOPO cloning vectors (Invitrogen, Carlsbad, Calif.). If the complementary restriction sites used to fragment the DNA are not present in the cloning vector, the ends of the DNA molecules can be enzymatically modified. Alternatively, any site desired can be produced by ligating nucleotide sequences (linkers) onto the DNA termini; these ligated linkers can contain specific chemically synthesized oligonucleotides encoding restriction endonuclease recognition sequences. In an alternative method, the cleaved vector and protein gene can be modified by homopolymeric tailing. Recombinant molecules can be introduced into host cells via, for example, transformation, transfection, infection, electroporation and sonoporation, so that many copies of the gene sequence are generated.

In specific embodiments, transformation of host cells with recombinant DNA molecules that incorporate the isolated protein gene, cDNA, or synthesized DNA sequence enables generation of multiple copies of the gene. Thus, the gene can be obtained in large quantities by growing transformants, isolating the recombinant DNA molecules from the transformants and, when necessary, retrieving the inserted gene from the isolated recombinant DNA.

1. Vectors and Cells

For recombinant expression of one or more of the desired proteins, such as any organophosphorus bioscavenger or hyaluronan-degrading enzyme polypeptide described herein, the nucleic acid containing all or a portion of the nucleotide sequence encoding the protein can be inserted into an appropriate expression vector, i.e., a vector that contains the necessary elements for the transcription and translation of the inserted protein coding sequence. The necessary transcriptional and translational signals also can be supplied by the native promoter for enzyme genes, and/or their flanking regions.

Also provided are vectors that contain a nucleic acid encoding the enzyme. Cells containing the vectors also are provided. The cells include eukaryotic and prokaryotic cells, and the vectors are any suitable for use therein.

Prokaryotic and eukaryotic cells, including endothelial cells, containing the vectors are provided. Such cells include bacterial cells, yeast cells, fungal cells, Archea, plant cells, insect cells and animal cells. The cells are used to produce a protein thereof by growing the above-described cells under conditions whereby the encoded protein is expressed by the cell, and recovering the expressed protein. For purposes herein, for example, the enzyme can be secreted into the medium.

Provided are vectors that contain a sequence of nucleotides that encodes the organophosphorus bioscavenger or hyaluronan-degrading enzyme polypeptide, in some examples a butyrylcholinesterase or soluble hyaluronidase polypeptide, coupled to the native or heterologous signal sequence, as well as multiple copies thereof. The vectors can be selected for expression of the enzyme protein in the cell or such that the enzyme protein is expressed as a secreted protein.

A variety of host-vector systems can be used to express the protein coding sequence. These include but are not limited to mammalian cell systems infected with virus (e.g. vaccinia virus, adenovirus and other viruses); insect cell systems infected with virus (e.g. baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system used, any one of a number of suitable transcription and translation elements can be used.

Any methods known to those of skill in the art for the insertion of DNA fragments into a vector can be used to construct expression vectors containing a Chimeric gene containing appropriate transcriptional/translational control signals and protein coding sequences. These methods can include in vitro recombinant DNA and synthetic techniques and in vivo recombinants (genetic recombination). Expression of nucleic acid sequences encoding protein, or domains, derivatives, fragments or homologs thereof, can be regulated by a second nucleic acid sequence so that the genes or fragments thereof are expressed in a host transformed with the recombinant DNA molecule(s). For example, expression of the proteins can be controlled by any promoter/enhancer known in the art. In a specific embodiment, the promoter is not native to the genes for a desired protein. Promoters which can be used include but are not limited to the SV40 early promoter (Bernoist and Chambon, Nature 290:304-310 (1981)), the promoter contained in the 3′ long terminal repeat of Rous sarcoma virus (Yamamoto et al. Cell 22:787-797 (1980)), the herpes thymidine kinase promoter (Wagner et al., Proc. Natl. Acad. Sci. USA 78:1441-1445 (1981)), the regulatory sequences of the metallothionein gene (Brinster et al., Nature 296:39-42 (1982)); prokaryotic expression vector promoters such as the β-lactamase promoter (Jay et al., (1981) Proc. Natl. Acad. Sci. USA 78:5543) or the tac promoter (DeBoer et al., Proc. Natl. Acad. Sci. USA 80:21-25 (1983)); see also “Useful Proteins from Recombinant Bacteria”: in Scientific American 242:79-94 (1980); plant expression vector promoters such as the nopaline synthetase promoter (Herrera-Estrella et al., Nature 303:209-213 (1984)) or the cauliflower mosaic virus ³⁵S RNA promoter (Garder et al., Nucleic Acids Res. 9:2871 (1981)), and the promoter of the photosynthetic enzyme ribulose bisphosphate carboxylase (Herrera-Estrella et al., Nature 310:115-120 (1984)); promoter elements from yeast and other fungi such as the Gal4 promoter, the alcohol dehydrogenase promoter, the phosphoglycerol kinase promoter, the alkaline phosphatase promoter, and the following animal transcriptional control regions that exhibit tissue specificity and have been used in transgenic animals: elastase I gene control region which is active in pancreatic acinar cells (Swift et al., Cell 38:639-646 (1984); Ornitz et al., Cold Spring Harbor Symp. Quant. Biol. 50:399-409 (1986); MacDonald, Hepatology 7:425-515 (1987)); insulin gene control region which is active in pancreatic beta cells (Hanahan et al., Nature 315:115-122 (1985)), immunoglobulin gene control region which is active in lymphoid cells (Grosschedl et al., Cell 38:647-658 (1984); Adams et al., Nature 318:533-538 (1985); Alexander et al., Mol. Cell Biol. 7:1436-1444 (1987)), mouse mammary tumor virus control region which is active in testicular, breast, lymphoid and mast cells (Leder et al., Cell 45:485-495 (1986)), albumin gene control region which is active in liver (Pinkert et al., Genes and Devel. 1:268-276 (1987)), alpha-fetoprotein gene control region which is active in liver (Krumlauf et al., Mol. Cell Biol. 5:1639-1648 (1985); Hammer et al., Science 235:53-58 1987)), alpha-1 antitrypsin gene control region which is active in liver (Kelsey et al., Genes and Devel. 1:161-171 (1987)), beta globin gene control region which is active in myeloid cells (Magram et al., Nature 315:338-340 (1985); Kollias et al., Cell 46:89-94 (1986)), myelin basic protein gene control region which is active in oligodendrocyte cells of the brain (Readhead et al., Cell 48:703-712 (1987)), myosin light chain-2 gene control region which is active in skeletal muscle (Shani, Nature 314:283-286 (1985)), and gonadotrophic releasing hormone gene control region which is active in gonadotrophs of the hypothalamus (Mason et al., Science 234:1372-1378 (1986)).

In a specific embodiment, a vector is used that contains a promoter operably linked to nucleic acids encoding a desired protein, or a domain, fragment, derivative or homolog, thereof, one or more origins of replication, and optionally, one or more selectable markers (e.g., an antibiotic resistance gene). Exemplary plasmid vectors for transformation of E. coli cells, include, for example, the pQE expression vectors (available from Qiagen, Valencia, Calif.; see also literature published by Qiagen describing the system). pQE vectors have a phage T5 promoter (recognized by E. coli RNA polymerase) and a double lac operator repression module to provide tightly regulated, high-level expression of recombinant proteins in E. coli, a synthetic ribosomal binding site (RBS II) for efficient translation, a 6×His tag coding sequence, t₀ and T1 transcriptional terminators, ColE1 origin of replication, and a beta-lactamase gene for conferring ampicillin resistance. The pQE vectors enable placement of a 6×His tag at either the N- or C-terminus of the recombinant protein. Such plasmids include pQE 32, pQE 30, and pQE 31 which provide multiple cloning sites for all three reading frames and provide for the expression of N-terminally 6×His-tagged proteins. Other exemplary plasmid vectors for transformation of E. coli cells, include, for example, the pET expression vectors (see, U.S. Pat. No. 4,952,496; available from Novagen, Madison, Wis.; see, also literature published by Novagen describing the system). Such plasmids include pET 11a, which contains the T7lac promoter, T7 terminator, the inducible E. coli lac operator, and the lac repressor gene; pET 12a-c, which contains the T7 promoter, T7 terminator, and the E. coli ompT secretion signal; and pET 15b and pET19b (Novagen, Madison, Wis.), which contain a His-Tag™ leader sequence for use in purification with a His column and a thrombin cleavage site that permits cleavage following purification over the column, the T7-lac promoter region and the T7 terminator.

Exemplary of a vector for mammalian cell expression, in particular of a hyaluronan-degrading enzyme, is the HZ24 expression vector. The HZ24 expression vector was derived from the pCI vector backbone (Promega). It contains DNA encoding the Beta-lactamase resistance gene (AmpR), an F1 origin of replication, a Cytomegalovirus immediate-early enhancer/promoter region (CMV), and an SV40 late polyadenylation signal (SV40). The expression vector also has an internal ribosome entry site (IRES) from the ECMV virus (Clontech) and the mouse dihydrofolate reductase (DHFR) gene.

2. Expression

Organophosphorus bioscavengers and hyaluronan-degrading enzyme polypeptides, including butyrylcholinesterases and soluble hyaluronidase polypeptides, can be produced by any method known to those of skill in the art including in vivo and in vitro methods. Desired proteins can be expressed in any organism suitable to produce the required amounts and forms of the proteins, such as for example, needed for administration and treatment. Expression hosts include prokaryotic and eukaryotic organisms such as E. coli, yeast, plants, insect cells, mammalian cells, including human cell lines and transgenic animals. Expression hosts can differ in their protein production levels as well as the types of post-translational modifications that are present on the expressed proteins. The choice of expression host can be made based on these and other factors, such as regulatory and safety considerations, production costs and the need and methods for purification.

Many expression vectors are available and known to those of skill in the art and can be used for expression of proteins. The choice of expression vector will be influenced by the choice of host expression system. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector.

Organophosphorus bioscavengers and hyaluronan-degrading enzyme polypeptides, such as butyrylcholinesterases and soluble hyaluronidase polypeptides, also can be utilized or expressed as protein fusions. For example, an enzyme fusion can be generated to add additional functionality to an enzyme. Examples of enzyme fusion proteins include, but are not limited to, fusions of a signal sequence, a tag such as for localization, e.g. a his₆ tag or a myc tag, or a tag for purification, for example, a GST fusion, and a sequence for directing protein secretion and/or membrane association.

a. Prokaryotic Cells

Prokaryotes, especially E. coli, provide a system for producing large amounts of proteins. Transformation of E. coli is simple and rapid technique well known to those of skill in the art. Expression vectors for E. coli can contain inducible promoters, such promoters are useful for inducing high levels of protein expression and for expressing proteins that exhibit some toxicity to the host cells. Examples of inducible promoters include the lac promoter, the trp promoter, the hybrid tac promoter, the T7 and SP6 RNA promoters and the temperature regulated XPL promoter.

Proteins, such as any provided herein, can be expressed in the cytoplasmic environment of E. coli. The cytoplasm is a reducing environment and for some molecules, this can result in the formation of insoluble inclusion bodies. Reducing agents such as dithiothreitol and β-mercaptoethanol and denaturants, such as guanidine-HCl and urea can be used to resolubilize the proteins. An alternative approach is the expression of proteins in the periplasmic space of bacteria which provides an oxidizing environment and chaperonin-like and disulfide isomerases and can lead to the production of soluble protein. Typically, a leader sequence is fused to the protein to be expressed which directs the protein to the periplasm. The leader is then removed by signal peptidases inside the periplasm. Examples of periplasmic-targeting leader sequences include the pelB leader from the pectate lyase gene and the leader derived from the alkaline phosphatase gene. In some cases, periplasmic expression allows leakage of the expressed protein into the culture medium. The secretion of proteins allows quick and simple purification from the culture supernatant. Proteins that are not secreted can be obtained from the periplasm by osmotic lysis. Similar to cytoplasmic expression, in some cases proteins can become insoluble and denaturants and reducing agents can be used to facilitate solubilization and refolding. Temperature of induction and growth also can influence expression levels and solubility, typically temperatures between 25° C. and 37° C. are used. Typically, bacteria produce aglycosylated proteins. Thus, if proteins require glycosylation for function, glycosylation can be added in vitro after purification from host cells.

b. Yeast Cells

Yeasts such as Saccharomyces cerevisae, Schizosaccharomyces pombe, Yarrowia lipolytica, Kluyveromyces lactis and Pichia pastoris are well known yeast expression hosts that can be used for production of proteins, such as any described herein. Yeast can be transformed with episomal replicating vectors or by stable chromosomal integration by homologous recombination. Typically, inducible promoters are used to regulate gene expression. Examples of such promoters include GAL1, GAL7 and GAL5 and metallothionein promoters, such as CUP1, AOX1 or other Pichia or other yeast promoter. Expression vectors often include a selectable marker such as LEU2, TRP1, HIS3 and URA3 for selection and maintenance of the transformed DNA. Proteins expressed in yeast are often soluble. Co-expression with chaperonins such as Bip and protein disulfide isomerase can improve expression levels and solubility. Additionally, proteins expressed in yeast can be directed for secretion using secretion signal peptide fusions such as the yeast mating type alpha-factor secretion signal from Saccharomyces cerevisae and fusions with yeast cell surface proteins such as the Aga2p mating adhesion receptor or the Arxula adeninivorans glucoamylase. A protease cleavage site such as for the Kex-2 protease, can be engineered to remove the fused sequences from the expressed polypeptides as they exit the secretion pathway. Yeast also is capable of glycosylation at Asn-X-Ser/Thr motifs.

c. Insect Cells

Insect cells, particularly using baculovirus expression, are useful for expressing polypeptides such as organophosphorus bioscavengers and hyaluronidase polypeptides. Insect cells express high levels of protein and are capable of most of the post-translational modifications used by higher eukaryotes. Baculovirus have a restrictive host range which improves the safety and reduces regulatory concerns of eukaryotic expression. Typical expression vectors use a promoter for high level expression such as the polyhedrin promoter of baculovirus. Commonly used baculovirus systems include the baculoviruses such as Autographa californica nuclear polyhedrosis virus (AcNPV), and the Bombyx mori nuclear polyhedrosis virus (BmNPV) and an insect cell line such as Sf9 derived from Spodoptera frugiperda, Pseudaletia unipuncta (A7S) and Danaus plexippus (DpN1). For high-level expression, the nucleotide sequence of the molecule to be expressed is fused immediately downstream of the polyhedrin initiation codon of the virus. Mammalian secretion signals are accurately processed in insect cells and can be used to secrete the expressed protein into the culture medium. In addition, the cell lines Pseudaletia unipuncta (A7S) and Danaus plexippus (DpN1) produce proteins with glycosylation patterns similar to mammalian cell systems.

An alternative expression system in insect cells is the use of stably transformed cells. Cell lines such as the Schneider 2 (S2) and Kc cells (Drosophila melanogaster) and C7 cells (Aedes albopictus) can be used for expression. The Drosophila metallothionein promoter can be used to induce high levels of expression in the presence of heavy metal induction with cadmium or copper. Expression vectors are typically maintained by the use of selectable markers such as neomycin and hygromycin.

d. Mammalian Cells

Mammalian expression systems can be used to express proteins including organophosphorus bioscavengers and hyaluronan-degrading enzyme polypeptides, such as soluble hyaluronidase polypeptides. Expression constructs can be transferred to mammalian cells by viral infection such as adenovirus or by direct DNA transfer such as liposomes, calcium phosphate, DEAE-dextran and by physical means such as electroporation and microinjection. Expression vectors for mammalian cells typically include an mRNA cap site, a TATA box, a translational initiation sequence (Kozak consensus sequence) and polyadenylation elements. IRES elements also can be added to permit bicistronic expression with another gene, such as a selectable marker. Such vectors often include transcriptional promoter-enhancers for high-level expression, for example the SV40 promoter-enhancer, the human cytomegalovirus (CMV) promoter and the long terminal repeat of Rous sarcoma virus (RSV). These promoter-enhancers are active in many cell types. Tissue and cell-type promoters and enhancer regions also can be used for expression. Exemplary promoter/enhancer regions include, but are not limited to, those from genes such as elastase I, insulin, immunoglobulin, mouse mammary tumor virus, albumin, alpha fetoprotein, alpha 1 antitrypsin, beta globin, myelin basic protein, myosin light chain 2, and gonadotropic releasing hormone gene control. Selectable markers can be used to select for and maintain cells with the expression construct. Examples of selectable marker genes include, but are not limited to, hygromycin B phosphotransferase, adenosine deaminase, xanthine-guanine phosphoribosyl transferase, aminoglycoside phosphotransferase, dihydrofolate reductase (DHFR) and thymidine kinase. For example, expression can be performed in the presence of methotrexate to select for only those cells expressing the DHFR gene. Fusion with cell surface signaling molecules such as TCR-ζ and Fc_{ε}RI-γ can direct expression of the proteins in an active state on the cell surface.

Many cell lines are available for mammalian expression including mouse, rat human, monkey, chicken and hamster cells. Exemplary cell lines include but are not limited to CHO, Balb/3T3, HeLa, MT2, mouse NS0 (nonsecreting) and other myeloma cell lines, hybridoma and heterohybridoma cell lines, lymphocytes, fibroblasts, Sp2/0, COS, NIH3T3, HEK293, 293S, 2B8, and HKB cells. Cell lines also are available adapted to serum-free media which facilitates purification of secreted proteins from the cell culture media. Examples include CHO-S cells (Invitrogen, Carlsbad, Calif., cat #11619-012) and the serum free EBNA-1 cell line (Pham et al., (2003) Biotechnol. Bioeng. 84:332-342). Cell lines also are available that are adapted to grow in special media optimized for maximal expression. For example, DG44 CHO cells are adapted to grow in suspension culture in a chemically defined, animal product-free medium.

i. Generation of Transgenic Animals

Protocols for the generation of non-human transgenic mammals are well established in the art (see, for example, Transgenesis Techniques Murphy, et al., Eds., Human Press, Totowa, N.J. (1993); Genetic Engineering of Animals A. Puhler, Ed. VCH Verlagsgesellschaft, Weinheim, N.Y. (1993); and Transgenic Animals in Agriculture Murray, et al., eds. Oxford University Press). For example, efficient protocols are available for the production of transgenic mice (Manipulating the Mouse Embryo 2nd Edition Hogan, et al. Cold Spring Harbor Press (1994) and Mouse Genetics and Transgenics: A Practical Approach. Jackson and Abbott, eds. Oxford University Press (2000)), transgenic cows (U.S. Pat. No. 5,633,076), transgenic pigs (U.S. Pat. No. 6,271,436), and transgenic goats (U.S. Pat. No. 5,907,080). Non-limiting examples of such protocols are summarized below.

Transgenic animals can be generated using stably transfected host cells derived from in vitro transfection. Where said host cells are pluripotent or totipotent, such cells can be used in morula aggregation or blastocyst injection protocols to generate chimeric animals. Preferred pluripotent/totipotent stably transfected host cells include primoridal germ cells, embryonic stem cells, and embryonal carcinoma cells. In a morula aggregation protocol, stably transfected host cells are aggregated with non-transgenic morula-stage embryos. In a blastocyst injection protocol, stably transfected host cells are introduced into the blastocoelic cavity of a non-transgenic blastocyst-stage embryo. The aggregated or injected embryos are then transferred to a pseudopregnant recipient female for gestation and birth of chimeras. Chimeric animals in which the transgenic host cells have contibuted to the germ line can be used in breeding schemes to generate non-chimeric offspring which are wholly transgenic.

In an alternative protocol, such stably transfected host cells can be used as nucleus donors for nuclear transfer into recipient oocytes (as per Wilmut, et al. (1997) Nature 385: 810-813). For nuclear transfer, the stably transfected host cells need not be pluripotent or totipotent. Thus, for example, stably transfected fetal fibroblasts can be used (e.g., Cibelli, et al. (1998) Science 280: 1256-8 and Keefer, et al. (2001) Biology of Reproduction 64:849-856). The recipient oocytes are preferrably enucleated prior to transfer. Following nuclear transfer, the oocyte is transferred to a pseudopregnant recipient female for gestation and birth. Such offspring will be wholly transgenic (that is, not chimeric).

In another alternative protocol, transgenic animals are generated by direct introduction of expression construct DNA into a recipient oocyte, zygote, or embryo. Such direct introduction can be achieved by pronuclear microinjection (Wang et al. (2002) Molecular Reproduction and Development 63:437-443), cytoplasmic microinjection (Page et al. (1995) Transgenic Res 4(6):353-360), retroviral infection (e.g., Lebkowski et al. (1988) Mol Cell Biol 8(10):3988-3996), or electroporation (“Molecular Cloning: A Laboratory Manual. Second Edition” by Sambrook, et al. Cold Spring Harbor Laboratory: 1989).

For microinjection and electroporation protocols, the introduced DNA should contain linear expression construct DNA, free of vector sequences, as prepared from the expression constructs of the invention. Following DNA introduction and any necessary in vitro culture, the oocyte, zygote, or embryo is transferred to a pseudopregnant recipient female for gestation and birth. Such offspring can or can not be chimeric, depending on the timing and efficiency of transgene integration. For example, if a single cell of a two-cell stage embryo is microinjected, the resultant animal will most likely be chimeric.

Transgenic animals containing two or more independent transgenes can be made by introducing two or more different expression constructs into host cells using any of the above described methods. The presence of the transgene in the genomic DNA of an animal, tissue, or cell of interest, as well as transgene copy number, can be confirmed by techniques well known in the art, including hybridization and PCR techniques.

Some of the transgensis protocols result in the production of chimeric animals. Chimeric animals in which the transgenic host cells have contributed to the tissue-type wherein the promoter of the expression construct is active (e.g., mammary gland for WAP promoter) can be used to characterize or isolate recombinant proteins. For example, where the transgenic host cells have contibuted to the germ line, chimeras can be used in breeding schemes to generate non-chimeric offspring which are wholly transgenic.

Wholly transgenic offspring, whether generated directly by a transgensis protocol or by breeding of chimeric animals, can be used for breeding purposes to maintain the transgenic line and to characterize or isolate recombinant proteins. Where transgene expression is driven by a urinary endothelium-specific promoter, urine of transgenic animals can be collected for purification and characterization of recombinant enzymes. Where transgene expression is driven by a mammary gland-specific promoter, lactation of the transgenic animals can be induced or maintained, where the resultant milk can be collected for purification and characterization of recombinant enzymes. For female transgenics, lactation can be induced by pregnancy or by administration of hormones. For male transgenics, lactation may be induced by administration of hormones (Ebert et al. (1994) Biotechnology 12:699-702). Lactation is maintained by continued collection of milk from a lactating transgenic.

e. Plants

Transgenic plant cells and plants can be used to express proteins such as any described herein. Expression constructs are typically transferred to plants using direct DNA transfer such as microprojectile bombardment and PEG-mediated transfer into protoplasts, and with agrobacterium-mediated transformation. Expression vectors can include promoter and enhancer sequences, transcriptional termination elements and translational control elements. Expression vectors and transformation techniques are usually divided between dicot hosts, such as Arabidopsis and tobacco, and monocot hosts, such as corn and rice. Examples of plant promoters used for expression include the cauliflower mosaic virus promoter, the nopaline synthetase promoter, the ribose bisphosphate carboxylase promoter and the ubiquitin and UBQ3 promoters. Selectable markers such as hygromycin, phosphomannose isomerase and neomycin phosphotransferase are often used to facilitate selection and maintenance of transformed cells. Transformed plant cells can be maintained in culture as cells, aggregates (callus tissue) or regenerated into whole plants. Transgenic plant cells also can include algae engineered to produce hyaluronidase polypeptides. Because plants have different glycosylation patterns than mammalian cells, this can influence the choice of protein produced in these hosts.

3. Purification Techniques

Method for purification of polypeptides, including organophosphorus bioscavengers (e.g. butyrylcholinesterases) and hyaluronan-degrading enzyme polypeptides (e.g. soluble hyaluronidase polypeptides) or other proteins, from host cells will depend on the chosen host cells and expression systems. For secreted molecules, proteins are generally purified from the culture media after removing the cells. For intracellular expression, cells can be lysed and the proteins purified from the extract. When transgenic organisms such as transgenic plants and animals are used for expression, tissues or organs can be used as starting material to make a lysed cell extract. Additionally, transgenic animal production can include the production of polypeptides in milk or eggs, which can be collected, and if necessary, the proteins can be extracted and further purified using standard methods in the art.

Proteins, such as soluble hyaluronidase polypeptides, can be purified using standard protein purification techniques known in the art including but not limited to, SDS-PAGE, size fraction and size exclusion chromatography, ammonium sulfate precipitation and ionic exchange chromatography, such as anion exchange chromatography. Affinity purification techniques also can be utilized to improve the efficiency and purity of the preparations. For example, antibodies, receptors and other molecules that bind hyaluronidase enzymes can be used in affinity purification. Expression constructs also can be engineered to add an affinity tag to a protein such as a myc epitope, GST fusion or His₆ and affinity purified with myc antibody, glutathione resin and Ni-resin, respectively. Purity can be assessed by any method known in the art including gel electrophoresis and staining and spectrophotometric techniques. Purified rHuPH20 compositions, as provided herein, typically have a specific activity of at least 70,000 to 100,000 Units/mg, for example, about 120,000 Units/mg. The specific activity can vary upon modification, such as with a polymer.

4. PEGylation

Polyethylene glycol (PEG) has been widely used in biomaterials, biotechnology and medicine primarily because PEG is a biocompatible, nontoxic, water-soluble polymer that is typically nonimmunogenic (Zhao and Harris, ACS Symposium Series 680: 458-72, 1997). In the area of drug delivery, PEG derivatives have been widely used in covalent attachment (i.e., “PEGylation”) to proteins to reduce immunogenicity, proteolysis and kidney clearance and to enhance solubility (Zalipsky, Adv. Drug Del. Rev. 16:157-82, 1995). Similarly, PEG has been attached to low molecular weight, relatively hydrophobic drugs to enhance solubility, reduce toxicity and alter biodistribution. Typically, PEGylated drugs are injected as solutions.

A closely related application is synthesis of crosslinked degradable PEG networks or formulations for use in drug delivery since much of the same chemistry used in design of degradable, soluble drug carriers can also be used in design of degradable gels (Sawhney et al., Macromolecules 26: 581-87, 1993). It also is known that intermacromolecular complexes can be formed by mixing solutions of two complementary polymers. Such complexes are generally stabilized by electrostatic interactions (polyanion-polycation) and/or hydrogen bonds (polyacid-polybase) between the polymers involved, and/or by hydrophobic interactions between the polymers in an aqueous surrounding (Krupers et al., Eur. Polym J. 32:785-790, 1996). For example, mixing solutions of polyacrylic acid (PAAc) and polyethylene oxide (PEO) under the proper conditions results in the formation of complexes based mostly on hydrogen bonding. Dissociation of these complexes at physiologic conditions has been used for delivery of free drugs (i.e., non-PEGylated). In addition, complexes of complementary polymers have been formed from both homopolymers and copolymers.

Numerous reagents for PEGylation have been described in the art. Such reagents include, but are not limited to, N-hydroxysuccinimidyl (NHS) activated PEG, succinimidyl mPEG, mPEG₂-N-hydroxysuccinimide, mPEG succinimidyl alpha-methylbutanoate, mPEG succinimidyl propionate, mPEG succinimidyl butanoate, mPEG carboxymethyl 3-hydroxybutanoic acid succinimidyl ester, homobifunctional PEG-succinimidyl propionate, homobifunctional PEG propionaldehyde, homobifunctional PEG butyraldehyde, PEG maleimide, PEG hydrazide, p-nitrophenyl-carbonate PEG, mPEG-benzotriazole carbonate, propionaldehyde PEG, mPEG butryaldehyde, branched mPEG₂ butyraldehyde, mPEG acetyl, mPEG piperidone, mPEG methylketone, mPEG “linkerless” maleimide, mPEG vinyl sulfone, mPEG thiol, mPEG orthopyridylthioester, mPEG orthopyridyl disulfide, Fmoc-PEG-NHS, Boc-PEG-NHS, vinylsulfone PEG-NHS, acrylate PEG-NHS, fluorescein PEG-NHS, and biotin PEG-NHS (see e.g., Monfardini et al., Bioconjugate Chem. 6:62-69, 1995; Veronese et al., J. Bioactive Compatible Polymers 12:197-207, 1997; U.S. Pat. No. 5,672,662; U.S. Pat. No. 5,932,462; U.S. Pat. No. 6,495,659; U.S. Pat. No. 6,737,505; U.S. Pat. No. 4,002,531; U.S. Pat. No. 4,179,337; U.S. Pat. No. 5,122,614; U.S. Pat. No. 5,324,844; U.S. Pat. No. 5,446,090; U.S. Pat. No. 5,612,460; U.S. Pat. No. 5,643,575; U.S. Pat. No. 5,766,581; U.S. Pat. No. 5,795,569; U.S. Pat. No. 5,808,096; U.S. Pat. No. 5,900,461; U.S. Pat. No. 5,919,455; U.S. Pat. No. 5,985,263; U.S. Pat. No. 5,990,237; U.S. Pat. No. 6,113,906; U.S. Pat. No. 6,214,966; U.S. Pat. No. 6,258,351; U.S. Pat. No. 6,340,742; U.S. Pat. No. 6,413,507; U.S. Pat. No. 6,420,339; U.S. Pat. No. 6,437,025; U.S. Pat. No. 6,448,369; U.S. Pat. No. 6,461,802; U.S. Pat. No. 6,828,401; U.S. Pat. No. 6,858,736; U.S. 2001/0021763; U.S. 2001/0044526; U.S. 2001/0046481; U.S. 2002/0052430; U.S. 2002/0072573; U.S. 2002/0156047; U.S. 2003/0114647; U.S. 2003/0143596; U.S. 2003/0158333; U.S. 2003/0220447; U.S. 2004/0013637; US 2004/0235734; U.S. 2005/0114037; U.S. 2005/0171328; U.S. 2005/0209416; EP 1064951; EP 0822199; WO 0176640; WO 05000360; WO 0002017; WO 0249673; WO 9428024; and WO 0187925).

In one example, the polyethylene glycol has a molecular weight ranging from about 3 kD to about 50 kD, and typically from about 5 kD to about 30 kD. Covalent attachment of the PEG to the drug (known as “PEGylation”) can be accomplished by known chemical synthesis techniques. For example, the PEGylation of protein can be accomplished by reacting NHS-activated PEG with the protein under suitable reaction conditions.

While numerous reactions have been described for PEGylation, those that are most generally applicable confer directionality, utilize mild reaction conditions, and do not necessitate extensive downstream processing to remove toxic catalysts or bi-products. For instance, monomethoxy PEG (mPEG) has only one reactive terminal hydroxyl, and thus its use limits some of the heterogeneity of the resulting PEG-protein product mixture. Activation of the hydroxyl group at the end of the polymer opposite to the terminal methoxy group is generally necessary to accomplish efficient protein PEGylation, with the aim being to make the derivatised PEG more susceptible to nucleophilic attack. The attacking nucleophile is usually the epsilon-amino group of a lysyl residue, but other amines also can react (e.g. the N-terminal alpha-amine or the ring amines of histidine) if local conditions are favorable. A more directed attachment is possible in proteins containing a single lysine or cysteine. The latter residue can be targeted by PEG-maleimide for thiol-specific modification. Alternatively, PEG hydrazide can be reacted with a periodate oxidized hyaluronan-degrading enzyme and reduced in the presence of NaCNBH₃. More specifically, PEGylated CMP sugars can be reacted with a hyaluronan-degrading enzyme in the presence of appropriate glycosyl-transferases. One technique is the “PEGylation” technique where a number of polymeric molecules are coupled to the polypeptide in question. When using this technique the immune system has difficulties in recognizing the epitopes on the polypeptide's surface responsible for the formation of antibodies, thereby reducing the immune response. For polypeptides introduced directly into the circulatory system of the human body to give a particular physiological effect (i.e. pharmaceuticals) the typical potential immune response is an IgG and/or IgM response, while polypeptides which are inhaled through the respiratory system (i.e. industrial polypeptide) potentially can cause an IgE response (i.e. allergic response). One of the theories explaining the reduced immune response is that the polymeric molecule(s) shield(s) epitope(s) on the surface of the polypeptide responsible for the immune response leading to antibody formation. Another theory or at least a partial factor is that the heavier the conjugate is, the more reduced immune response is obtained.

Typically, to make the PEGylated organophosphorus bioscavengers and hyaluronan-degrading enzymes provided herein, PEG moieties are conjugated, via covalent attachment, to the polypeptides. Techniques for PEGylation include, but are not limited to, specialized linkers and coupling chemistries (see e.g., Roberts, Adv. Drug Deliv. Rev. 54:459-476, 2002), attachment of multiple PEG moieties to a single conjugation site (such as via use of branched PEGs; see e.g., Guiotto et al., Bioorg. Med. Chem. Lett. 12:177-180, 2002), site-specific PEGylation and/or mono-PEGylation (see e.g., Chapman et al., Nature Biotech. 17:780-783, 1999), and site-directed enzymatic PEGylation (see e.g., Sato, Adv. Drug Deliv. Rev., 54:487-504, 2002). Methods and techniques described in the art can produce proteins having 1, 2, 3, 4, 5, 6, 7, 8, 9, 10 or more than 10 PEGs or PEG derivatives attached to a single protein molecule (see e.g., U.S. 2006/0104968).

As an exemplary illustration of the PEGylation of an illustrative method for making PEGylated hyaluronan-degrading enzymes, such as PEGylated hyaluronidases, PEG aldehydes, succinimides and carbonates have each been applied to conjugate PEG moieties, typically succinimidyl PEGs, to rHuPH20. For example, rHuPH20 has been conjugated with exemplary succinimidyl monoPEG (mPEG) reagents including mPEG-Succinimidyl Propionates (mPEG-SPA), mPEG-Succinimidyl Butanoates (mPEG-SBA), and (for attaching “branched” PEGs) mPEG2-N-Hydroxylsuccinimide. These PEGylated succinimidyl esters contain different length carbon backbones between the PEG group and the activated cross-linker, and either a single or branched PEG group. These differences can be used, for example, to provide for different reaction kinetics and to potentially restrict sites available for PEG attachment to rHuPH20 during the conjugation process.

Succinimidyl PEGs (as above) comprising either linear or branched PEGs can be conjugated to rHuPH20. PEGs can used to generate rHuPH20s reproducibly containing molecules having, on the average, between about three to six or three to six PEG molecules per hyaluronidase. Such PEGylated rHuPH20 compositions can be readily purified to yield compositions having specific activities of approximately 25,000 or 30,000 Unit/mg protein hyaluronidase activity, and being substantially free of non-PEGylated rHuPH20 (less than 5% non-PEGylated).

Using various PEG reagents, exemplary versions of organophosphorus bioscavengers and hyaluronan-degrading enzymes, in particular soluble human recombinant hyaluronidases (e.g. rHuPH20), can be prepared, for example, using mPEG-SBA (30 kD), mPEG-SMB (30 kD), and branched versions based on mPEG2-NHS (40 kD) and mPEG2-NHS (60 kD). PEGylated versions of rHuPH20 have been generated using NHS chemistries, as well as carbonates, and aldehydes, using each of the following reagents: mPEG2-NHS-40K branched, mPEG-NHS-10K branched, mPEG-NHS-20K branched, mPEG2-NHS-60K branched; mPEG-SBA-5K, mPEG-SBA-20K, mPEG-SBA-30K; mPEG-SMB-20K, mPEG-SMB-30K; mPEG-butyrldehyde; mPEG-SPA-20K, mPEG-SPA-30K; and PEG-NHS-5K-biotin. PEGylated hyaluronidases have also been prepared using PEG reagents available from Dowpharma, a division of Dow Chemical Corporation; including hyaluronidases PEGylated with Dowpharma's p-nitrophenyl-carbonate PEG (30 kDa) and with propionaldehyde PEG (30 kDa).

In one example, the PEGylation includes conjugation of mPEG-SBA, for example, mPEG-SBA-30K (having a molecular weight of about 30 kDa) or another succinimidyl esters of PEG butanoic acid derivative, to a soluble hyaluronidase. Succinimidyl esters of PEG butanoic acid derivatives, such as mPEG-SBA-30K readily couple to amino groups of proteins. For example, covalent conjugation of m-PEG-SBA-30K and to the exemplary hyaluronan-degrading enzyme preparation designated rHuPH20 (which is approximately 60 KDa in size) provides stable amide bonds between rHuPH20 and mPEG, as shown in Scheme 1, below.

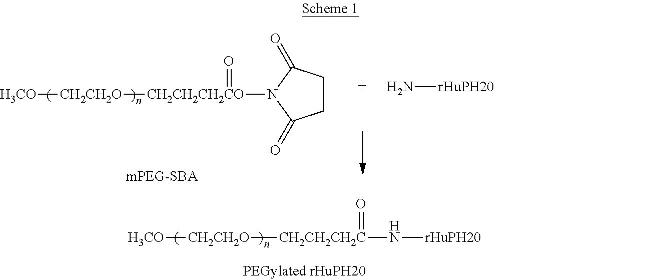

With respect to PEGylation of a hyaluronan-degrading enzyme, typically, the mPEG-SBA-30K or other PEG is added to the hyaluronan-degrading enzyme, in some instances a hyaluronidase, at a PEG:polypeptide molar ratio of 10:1 in a suitable buffer, e.g. 130 mM NaCl/10 mM HEPES at pH 6.8 or 70 mM phosphate buffer, pH 7, followed by sterilization, e.g. sterile filtration, and continued conjugation, for example, with stirring, overnight at 4° C. in a cold room. In one example, the conjugated PEG-hyaluronan-degrading enzyme is concentrated and buffer-exchanged.

Other methods of coupling succinimidyl esters of PEG butanoic acid derivatives, such as mPEG-SBA-30K are known in the art (see e.g., U.S. Pat. No. 5,672,662; U.S. Pat. No. 6,737,505; and U.S. 2004/0235734). For example, a polypeptide, such as an organophosphorus bioscavenger or hyaluronan-degrading enzyme (e.g. a hyaluronidase), can be coupled to an NHS activated PEG derivative by reaction in a borate buffer (0.1 M, pH 8.0) for one hour at 4° C. The resulting PEGylated protein can be purified by ultrafiltration. Alternatively, PEGylation of a bovine alkaline phosphatase can be accomplished by mixing the phosphatase with mPEG-SBA in a buffer containing 0.2 M sodium phosphate and 0.5 M NaCl (pH 7.5) at 4° C. for 30 minutes. Unreacted PEG can be removed by ultrafiltration. Another method reacts polypeptide with mPEG-SBA in deionized water to which triethylamine is added to raise the pH to 7.2-9. The resulting mixture is stirred at room temperature for several hours to complete the PEGylation.

Methods for PEGylation of organophosphorus bioscavengers and hyaluronan-degrading polypeptides, including, for example, animal-derived hyaluronidases and bacterial hyaluronan-degrading enzymes, are known to one of skill in the art. See, for example, European Patent No. EP 0400472, which describes the PEGylation of bovine testes hyaluorindase and chondroitin ABC lyase. Also, U.S. Publication No. 2006014968 describes PEGylation of a human hyaluronidase derived from human PH20. For example, the PEGylated hyaluronan-degrading enzyme generally contains at least 3 PEG moieties per molecule. For example, the hyaluronan-degrading enzyme can have a PEG to protein molar ratio between 5:1 and 9:1, for example, 7:1. U.S. Publication No. 20090208480 also describes the PEGylation of butyrylcholinesterase.

### G. Methods of Assessing Activity

Provided herein are methods of assessing the activity of an organophosphorus bioscavenger in compositions or combinations with a hyaluronan-degrading enzyme to assess one or more activities of either or both components. For example, inactivating activity, hydrolytic activity and/or binding activity of an OP bioscavenger can be assessed in in vitro or in vivo assays. In another example, hyaluronidase activity of a hyaluronan-degrading enzyme can be assessed in an in vitro or in vivo assay. For methods of preventing or treating of organophosphorus poisoning, the OP bioscavenger exhibits inactivating activity, hydrolytic activity and/or binding activity and the hyaluronan-degrading enzyme exhibits hyaluronidase activity.

1. Organophosphorus Bioscavenger Activity

a. Enzymatic Activity

Various methods for assessing the enzymatic activity of OP bioscavengers, such as a cholinesterase, are described in the art (see, for example, Lockridge and La Du, J Biol Chem (1978) 253:361-366; Lockridge, et al. Biochemistry (1997) 36:786-795; Platteborze and Broomfield, Biotechnol. Appl. Biochem. (2000) 31:226-229; and Blong, et al. Biochem J (1997) 327:747-757). For example, a sample containing a cholinesterase can be tested for the presence of enzymatically active AChE or BChE by using the activity assay of Ellman (Ellman, et al. Biochem Pharmacol (1961) 7:88). In some cases kits or reagents are available from commercial or other publicly available sources. For example, Example 5 describes a Cholinesterase BTC kit for measuring butyrylcholinesterase activity. The sample can be, for example, a solution prepared in vitro containing a recombinant cholinesterase or can be a sample, such as a plasma sample, obtained from a subject. In some examples, the sample, such as a plasma sample, is obtained from a subject treated with recombinant cholinesterase.

In an exemplary assay, levels of AChE or BChE activity can be estimated by mixing a sample, such a plasma sample, with the cholinesterase substrate, such as butyrylthiocholine (BTCh) or acetylthiocholine (ATCh), and the photometric reagent, 5,5′-Dithiobis(2-nitrobenzoic acid) (DTNB; Ellman's Reagent), which quantifies thiols in the sample. The cholinesterase hydrolyzes the BTCh or ATCh to release thiocholine which reacts with the DNTB, cleaving the disulfide bond to give 2-nitro-5-thiobenzoate (NTB⁻), which ionizes to the NTB²⁻ dianion in water at neutral and alkaline pH. The NTB²⁻ ion has a yellow color and can be quantified by measuring absorption of the sample at 405 nM. Such assays can be performed in multiwell format, such as a microtiter plate. In other exemplary assays, levels of AChE or BChE activity can be estimated by staining non-denaturing 4-30% polyacrylamide gradient gels with 2 mM echothiophate iodide as substrate (as described in Lockridge, et al. Biochemistry (1997) 36:786-795). or 2 mM butyrylthiocholine as substrate (see Karnovsky and Roots, J Histochem Cytochem (1964) 12:219).

An additional exemplary assay for assessing cholinesterase activity is the Walter Reed Army Institute of Research Whole Blood cholinesterase assay (WRAIR WB, see U.S. Pat. No. 6,746,850). WRAIR can measure cholinesterase activity in whole blood samples (e.g. blood from a finger prick) and involves calculating the concentration of active AChE and BChE by measuring the hydrolysis of three substrates with known hydrolysis rates by AChE and BChE (e.g. acetylthiocholine iodide (ATC), propionylthiocholine iodide (PTC) and butyrylthiocholine iodide) in the presence of 4,4″-dithidiopyridine (DTP), the indicator for the hydrolyzed thiocholine (UV absorbance at 324). Because AChE and BChE enzymes possess different affinities for the different substrates the precise concentration of each active enzyme in the sample can be calculated. Thus, the concentrations of active plasma BChE and red blood cell AChE can be measure together in this assay.

Using these methods, the catalytic properties of an OP bioscavenger, such as a recombinant AChE or BChE protein, including Kₘ, Vₘₐₓ, and k_{cat} values, can be determined using butyrylthiocholine or acetylthiocholine as substrates. Similar methodologies can be used with other OP bioscavengers and are known to one of those skilled in the art or can be empirically determined from the description herein. Other methodologies known in the art can also be used to assess ChE function, including electrometry, spectrophotometry, chromatography, ELISA and radiometric methodologies.

The activity of the enzyme can be calculated in unit measures. For example, for butyrylcholinesterase, one unit of enzyme activity is defined as the amount required to hydrolyze 1 mmol substrate per minute with 720 units equivalent to 1 mg purified human plasma BChE (Duysen et al. (2002) J Pharmacol Exp Ther 302:751-758).

In order to normalize or correct the measured level of exogenous OP bioscavenger from the corresponding endogenous cholinesterase present in an animal plasma or sample, a baseline measurement can be made by assessing cholinesterase activity in plasma prior to dosage or administration with an OP bioscavenger. The baseline corrected plasma concentration is the concentration as measured at a time after administration that includes subtraction of the predose plasma concentration. Hence, this permits normalization of the measured concentrations to remove the endogenous component concentration. Where the baseline endogenous cholinesterase activity is not known, one of skill in the art can generally assign a baseline activity based on height, weight, age, health status and other factors known in the art.

The OP bioscavenger plasma concentration can be determined by the measured activity of bioscavenger in the plasma as described above. Typically, using an enzymatic assay as described above, the activity is set forth as Units (U) OP Bioscavenger activity per mL of plasma (U/mL). These values can be converted to μg/mL using the specific activity of the particular OP bioscavenger. For example, as described in the examples, PEG-rBChE has a specific activity of about or about between 500 to 800 U/mg. The level circulating OP bioscavenger in the blood can be assessed over time after it is administered by obtaining a sample of plasma and assessing the activity and plasma concentration of the OP bioscavenger in each sample normalized to baseline.

b. Endogenous Cholinesterase Activity

The ability of an organophosphorus bioscavenger in combination with a hyaluronan degrading enzyme to prevent or treat the symptoms of organophosphorus poisoning can be assessed by measuring endogenous cholinesterase activity. Acetylcholinesterase (AChE) is an endogenous target of organophosphorus compounds, thereby resulting in cholinergic toxicity. The symptoms of cholinergic toxicity as described in detail elsewhere herein derive from cholinesterase inhibition. Thus, assays to measure endogenous cholinesterase activity also can be employed to assess the activity of the administered OP bioscavenger, such as acetylcholinesterase or butyrylcholinesterase.

Generally, cholinergic toxicity and organophosphorus poisoning can occur when circulating levels of ChE are less than 20% of normal or when the circulating ChE is completely bound. Hence, endogenous cholinesterase activity can be assessed as a measure of cholinergic toxicity. An organophosphorus bioscavenger prevents or reduces cholinergic toxicity if in the presence of the OP bioscavenger endogenous cholinesterase activity or levels are above 20% or more of the normal or baseline endogenous activity or levels, such as above 25%, 30%, 40%, 50%, 60%, 70%, 80%, 90% of the baseline or normal levels.

The endogenous activity of ChE can be assessed as described above acetylthiocholine (ATCh) substrate and a photometric reagent. In other examples, human AChE also can be assessed by immunoblotting with antibodies specific to human AChE. For example, an antibody raised against either the common domain unique to human or mouse AChE (N19 and E-19, respectively can be used (available from Santa Cruz Biotechnology, Inc., Santa Cruz, Calif.; see also Evron et al. (2007) The FASEB J., 21:2961).

It is within the level of one of skill in the art to distinguish endogenous AChE from exogenous OP bioscavenger that is administered. Also, generally, prior to treatment with an OP bioscavenger, the baseline or normal values of AChE are determined. Then, after administration with an OP bioscavenger, the endogenous AChE levels can be monitored over time.

c. Methods to Assess Therapeutic Efficacy

Therapeutic effectiveness of a treatment with an organophosphorus bioscavenger, such as AChE or BChE, in combination with a hyaluronan degrading enzyme for the treatment or prevention of organophosphorus poisoning can be assessed in animal models, such as rodents or primates (see for example as in Raveh, et al. Toxicol. Applied Pharm. (1997) 145:43-53; Broomfield, et al. J Pharmacol Exp Ther (1991) 259:633-638; Brandeis, et al. Pharmacol Biochem Behav (1993) 46:889-896; Ashani, et al Biochem Pharmacol (1991) 41:37-41; and Rosenberg, et al. Life Sciences (2002) 72:125-134). Exemplary methods for assessing therapeutic efficacy provided herein can be employed to determine therapeutic amounts for administration of an organophosphorus bioscavenger in combination with a hyaluronan degrading enzyme to a human subject for the treatment or prevention of organophosphorus poisoning. Further, the methods can be employed for assessing the efficacy of therapy of a human subject.

Exemplary methods to assess the therapeutic effectiveness of an organophosphorus bioscavenger in combination with a hyaluronan degrading enzyme include, but are not limited to, assessing cholinesterase activity in the subject, assessing symptoms of organophosphorus poisoning, and assessing the presence of metabolites of hydrolysis of the organophosphorus compound. In these methods, the effect of the hyaluronan degrading enzyme on the therapeutic activity of the organophosphorus bioscavenger can be assessed by performing the method in the presence or absence of the hyaluronan degrading enzyme.

i. Assessing Cholinesterase Activity

In exemplary methods, cholinesterase activity as described herein can be measured in a subject in the presence or absence of the organophosphorus bioscavenger in combination with a hyaluronan degrading enzyme and compared to cholinesterase activity following exposure to the organophosphorus agent. In one example, the change in the level of cholinesterase activity in a subject following exposure to an organophosphorus compound can be assessed and compared to the cholinesterase activity in the subject treated with an organophosphorus bioscavenger in combination with a hyaluronan degrading enzyme. For assessing prophylactic activity, the organophosphorus bioscavenger in combination with a hyaluronan degrading enzyme can be administered prior to administration of the organophosphorus agent. For assessing therapeutic activity following exposure, the organophosphorus bioscavenger in combination with a hyaluronan degrading enzyme can be administered following administration of the organophosphorus agent. Following exposure of the subject to the organophosphorus agent, it is expected that the endogenous cholinesterase activity in the subject will decrease. The organophosphorus bioscavenger in combination with a hyaluronan degrading enzyme is determined to be therapeutically effective if it is able to prevent or reduce the decrease in cholinesterase activity in the presence of the organophosphorus agent.

In these methods, the effect of the hyaluronan degrading enzyme on the therapeutic activity of the organophosphorus bioscavenger can be assessed by performing the method in the presence or absence of the hyaluronan degrading enzyme and comparing the effects on cholinesterase activity in the assay.

ii. Assessing Symptoms of Organophosphorus Poisoning

In exemplary methods, one or more symptoms of organophosphorus poisoning can be assessed in a subject following exposure to the organophosphorus agent and compared to prophylactic or post-exposure treatment with the organophosphorus bioscavenger in combination with a hyaluronan degrading enzyme. For example, the ability of treatment with an organophosphorus bioscavenger in combination with a hyaluronan degrading enzyme to prevent, reduce or eliminate one or more symptoms of organophosphorus poisoning, such as but not limited to miosis, blurred vision, dark vision, headache, nausea, dizziness, vomiting, hypersecretion (e.g. sweating, salivation, lacrimation, and rhinorrhea), abdominal cramps, diarrhea, urinary incontinence, muscle twitching/fasciculations, paralysis, pallor, muscle weakness, tremors, convulsions, incoordination, diaphoresis, bronchospasm, bronchorrhea, tightness in chest, wheezing, productive cough, pulmonary edema, bradycardia, sinus arrest, tachycardia, hypertension, toxic myocardiopathy, mydriasis, ataxia, anxiety, restlessness, choreiform movement, loss of consciousness, respiratory depression, fatigue, seizures, and psychiatric symptoms (e.g. depression, memory loss, confusion, toxic psychosis), can be assessed.

In exemplary animal models of organophosphorus poisoning, the ability of treatment with an organophosphorus bioscavenger in combination with a hyaluronan degrading enzyme to prevent reduce or eliminate the adverse effects of cognitive and locomotive impairment can be assessed. There are a variety of tests for cognitive function, including learning and memory testing that can be performed in animal models, such as rats or mice (see, for example, United States Patent Publication No. 2010/0010097). Learning and/or memory tests include, for example, Inhibitory Avoidance Test, contextual fear conditioning, visual delay non-match to sample, spatial delay non-match to sample, visual discrimination, Barnes circular maze, Morris water maze, radial arm maze tests, Ray Auditory-Visual Learning Test, the Wechsler Logical Memory Test, and the Providence Recognition Memory Test. Additional behavioral test include, but are not limited to postural reflex testing, incline plane test, forepaw grip test and beam walking test (such tests are well known in the art; a description of exemplary tests can also be found in, for example, Abou-Donia et al. Toxicological Sciences 66:148-158 (2002))

In exemplary tests, animals' retention of the learned behavior can be determined, for example, after at least about 1, 2, 4, 6, 8, 12, 14, 16, 18, 20, 22, 24 or more hours after completion of the learning phase to determine whether treatment with a combination provided herein of a hyaluronan degrading enzyme and an organophosphorus bioscavenger can alleviate or inhibit the effects of an organophosphorus compound on memory consolidation prior to or following treatment with the compound. In some examples, prior to testing, the animals can be pretreated with a combination provided herein of a hyaluronan degrading enzyme and an organophosphorus bioscavenger followed by treatment with the organophosphorus compound. Control animals can include, for example, no treatment, treatment with the either the hyaluronan degrading enzyme or the organophosphorus bioscavenger, alone or following treatment with the organophosphorus compound. In some examples, the animals are treated with the organophosphorus compound prior to the administration of the combination of a hyaluronan degrading enzyme and an organophosphorus bioscavenger.

An exemplary maze testing embodiment is the water maze working memory test. In general, the method utilizes an apparatus which has a circular water tank. The water in the tank is made cloudy by the addition of milk powder. A clear plexiglass platform, supported by a movable stand rest on the bottom of the tank, is submerged just below the water surface. Normally, a swimming rat cannot perceive the location of the platform but it may recall it from a previous experience and training, unless it suffers from some memory impairment. The time taken to locate the platform is measured and referred to as the latency. During the experiment, all orientational cues such as ceiling lights, remain unchanged. Longer latencies are generally observed with rats with some impairment to their memory.

An exemplary Inhibitory Avoidance Test utilizes an apparatus that has a lit chamber that can be separated from a dark chamber by a sliding door. At training, the animal is placed in the lit chamber for some period of time, and the door is opened. The animal moves to the dark chamber after a short delay—the step-through latency—which is recorded. Upon entry into the dark chamber, the door is shut closed and a foot shock is delivered. Retention of the experience is determined after various time intervals, e.g., 24 or 48 hours, by repeating the test and recording the latency. The protocol is one of many variants of the passive avoidance procedures (for review, see Rush (1988) Behav. Neural. Biol. 50:255).

iii. Assessing the Metabolites Organophosphorus Compound Hydrolysis

For organophosphorus bioscavengers that hydrolyze organophosphorus compounds, the products of hydrolysis also can be assessed. For example, following treatment of a subject with an organophosphorus bioscavenger and exposure to the organophosphorus compound, samples obtained from the subject, such as urine or plasma samples, and assayed for an increase in the metabolites. Methods of assaying include but are not limited to gas chromatography (GC) using an electron detector (ECD), a nitrogen/phosphorous detector (NPD), a flame photometric detector (FPD) in phosphorous mode, or a mass spectroscopy detector (MS). In some instances an antibody that is specific to the metabolite can be employed for immunodetection.

d. Exemplary Method for Prophylactic Efficacy

In an exemplary method, the prophylactic efficacy of an organophosphorus bioscavenger, such as a pegylated recombinant butyrylcholinesterase (PEG-rBChE) alone or in combination with a hyaluronan degrading enzyme, such as recombinant human PH20 (rHuPH20), is evaluated in Hartley guinea pigs exposed to an organophosphorus compound such as a nerve agent, such as Soman or VX. PEG rBChE is administered by intramuscular injection followed by administration of the nerve agent. Animals are observed 6 hrs. post-challenge for cholinergic toxicity and tested at 30 and 48 hrs in a balance beam test and at 1 wk. in the Morris water maze. Therapeutic efficiency can be compared to therapy with other agents for organophosphorus poisoning, such as Atropine, 2-PAM, Diazepam, or to combination therapy with such agents. The pharmacokinetics of an organophosphorus bioscavenger, for example, rBChE (Protexia®, PEG-RBChE), when administered alone or in combination with a hyaluronan-degrading enzyme (e.g. rHuPH20) by intramuscular injection or subcutaneous injection to minipigs are then determined. The results can be compared with intravenous injection of PEG-rBChE alone as a single dose. For pharmacokinetic analysis, serial blood samples are collected from the minipigs by venipuncture of the anterior vena cava following the single dose at the following nominal times: predose (immediately prior to dosing). Plasma samples are analyzed for PEG-rBChE plasma concentration levels using a qualified enzymatic activity assay. Pharmacokinetic sampling are performed to determine maximum “peak” concentration (Cₘₐₓ), Tₘₐₓ (value associated with the observed Cₘₐₓ), elimination rate constant (λ_{z}), terminal elimination phase half-life (T_{1/2}), AUC, bioavailability, Clearance, volume of distribution (Vz), mean residence time (MRT).

e. Pharmacokinetics and Tolerability

Pharmacokinetic and tolerability studies can be performed using animal models or can be performed during clinical studies with patients. Animal models include, but are not limited to, mice, rats, rabbits, dogs, guinea pigs and non-human primate models, such as cynomolgus monkeys or rhesus macaques. In some instances, pharmacokinetic and tolerability studies are performed using healthy animals. In other examples, the studies are performed using animal models of organophosphorus poisoning.

The pharmacokinetics of an administered organophosphorus bioscavenger, such as an acetylcholinesterase or butyrylcholinesterase, can be assessed by measuring such parameters as the maximum (peak) plasma concentration of the organophosphorus bioscavenger (Cₘₐₓ), the peak time (i.e. when maximum plasma concentration of the organophosphorus bioscavenger occurs; Tₘₐₓ), the minimum plasma concentration (i.e. the minimum plasma concentration of the organophosphorus bioscavenger between doses; Cₘᵢₙ), the elimination half-life (T_{1/2}) and area under the curve (i.e. the area under the curve generated by plotting time versus plasma of the organophosphorus bioscavenger concentration; AUC), following administration. The absolute bioavailability of the administered of the organophosphorus bioscavenger is determined by comparing the area under the curve of the organophosphorus bioscavenger following subcutaneous delivery (AUC_{sc}) with the AUC of the organophosphorus bioscavenger following intravenous delivery (AUCᵢᵥ). Absolute bioavailability (F), can be calculated using the formula: F=([AUC]_{sc}×dose_{sc})/([AUC]ᵢᵥ×doseᵢᵥ). The concentration of the organophosphorus bioscavenger in the plasma following administration can be measured using any method known in the art suitable for assessing concentrations of an organophosphorus bioscavenger in samples of blood. Exemplary methods include, but are not limited to, ELISA. Peak blood level of the cholinesterase may be determined following intramuscular injection of AChE or BChE with or without the hyaluronan degrading enzyme (see, e.g., Raveh, et al. Biochem Pharmacol (1993) 45(12):2465).

A range of doses and different dosing frequencies can be administered in the pharmacokinetic studies to assess the effect of increasing or decreasing concentrations of the organophosphorus bioscavenger and a hyaluronan degrading enzyme in the dose. Pharmacokinetic properties of the administered organophosphorus bioscavenger, such as bioavailability, also can be assessed with or without co-administration of the hyaluronan degrading enzyme. The pharmacokinetic studies can be performed to assess therapeutic concentrations of the organophosphorus bioscavenger attained in circulation when administered with the hyaluronan degrading enzyme and maintenance of therapeutic concentration of the organophosphorus bioscavenger over time.

Studies to assess safety and tolerability also are known in the art and can be used herein. Following administration of an organophosphorus bioscavenger, with or without co-administration of a hyaluronan degrading enzyme, the development of any adverse reactions can be monitored. Adverse reactions can include, but are not limited to, injection site reactions, such as edema or swelling, headache, fever, fatigue, chills, flushing, dizziness, urticaria, wheezing or chest tightness, nausea, vomiting, rigors, back pain, chest pain, muscle cramps, seizures or convulsions, changes in blood pressure and anaphylactic or severe hypersensitivity responses. Typically, a range of doses and different dosing frequencies are administered in the safety and tolerability studies to assess the effect of increasing or decreasing concentrations of the organophosphorus bioscavenger and/or hyaluronan degrading enzyme in the dose.

2. Assays to Assess Hyaluronan Activity

Assays to assess hyaluronan activity can be performed separately or in conjugation with those mentioned above to assess the bioscavenging ability of an organophosphorus bioscavenger in combination with a hyaluronan degrading enzyme. Such assays can include, but are not limited to, measuring amounts of hyaluronan in tissue or soluble hyaluronan in plasma, measurements of hyaluronan catabolites in blood or urine, measurements of hyaluronidase activity in plasma, or measurements of interstitial fluid pressure, vascular volume or water content in tumors. Other assays such as measurements of pharmacokinetics, methods for which are well known to those of skill in the art, can be used to assess the pharmacokinetic parameters of hyaluronan administration

The activity of a hyaluronan degrading enzyme can be assessed using methods well known in the art. For example, the USP XXII assay for hyaluronidase determines activity indirectly by measuring the amount of undegraded hyaluronic acid, or hyaluronan, (HA) substrate remaining after the enzyme is allowed to react with the HA for 30 min at 37° C. (USP XXII-NF XVII (1990) 644-645 United States Pharmacopeia Convention, Inc, Rockville, Md.). A Hyaluronidase Reference Standard (USP) or National Formulary (NF) Standard Hyaluronidase solution can be used in an assay to ascertain the activity, in units, of any hyaluronidase. In one example, activity is measured using a microturbididy assay. This is based on the formation of an insoluble precipitate when hyaluronic acid binds with serum albumin. The activity is measured by incubating hyaluronidase or a sample containing hyaluronidase, for example blood or plasma, with sodium hyaluronate (hyaluronic acid) for a set period of time (e.g. 10 minutes) and then precipitating the undigested sodium hyaluronate with the addition of acidified serum albumin. The turbidity of the resulting sample is measured at 640 nm after an additional development period. The decrease in turbidity resulting from hyaluronidase activity on the sodium hyaluronate substrate is a measure of hyaluronidase enzymatic activity.

In another example, hyaluronidase activity is measured using a microliter assay in which residual biotinylated hyaluronic acid is measured following incubation with hyaluronidase or a sample containing hyaluronidase, for example, blood or plasma (see e.g. Frost and Stern (1997) Anal. Biochem. 251:263-269, U.S. Patent Publication No. 20050260186). The free carboxyl groups on the glucuronic acid residues of hyaluronic acid are biotinylated, and the biotinylated hyaluronic acid substrate is covalently coupled to a microtiter plate. Following incubation with hyaluronidase, the residual biotinylated hyaluronic acid substrate is detected using an avidin-peroxidase reaction, and compared to that obtained following reaction with hyaluronidase standards of known activity. Other assays to measure hyaluronidase activity also are known in the art and can be used in the methods herein (see e.g. Delpech et al., (1995) Anal. Biochem. 229:35-41; Takahashi et al., (2003) Anal. Biochem. 322:257-263).

The ability of an active hyaluronan degrading enzyme to act as a spreading or diffusing agent also can be assessed. For example, trypan blue dye can be injected, such as subcutaneously or intradermally, with or without a hyaluronan degrading enzyme into the lateral skin on each side of nude mice. The dye area is then measured, such as with a microcaliper, to determine the ability of the hyaluronan degrading enzyme to act as a spreading agent (see e.g. U.S. Published Patent No. 20060104968).

The above assays can be performed using a hyaluronan degrading enzyme in the presence or absence of an organophsphate bioscavenging agent or using the blood or plasma of a patient or animal treated with a hyuronan degrading enzyme with or without an organophsphate bioscavenging agent.

### H. Therapeutic and Prophylactic Uses

Provided herein are methods and uses of a hyaluronan degrading enzyme in combination with an organophosphorus bioscavenger for the prevention and treatment of the symptoms and adverse effects induced by exposure to organophosphorus compounds, including organophosphorus pesticides and nerve agents. Such agents are efficiently absorbed by inhalation, ingestion, and skin penetration and are able to cause toxicity through inhibition of the acetylcholinesterase neurotransmitter. The compositions provided herein of a hyaluronan degrading enzyme in combination with an organophosphorus bioscavenger can be administered for the prevention and treatment of adverse effects of organophosphorus poisoning, including, but not limited to, adverse effects on the muscarinic, nicotinic and central nervous systems. For example, the compositions provided herein of a hyaluronan degrading enzyme in combination with an organophosphorus bioscavenger can be employed for the prevention or treatment of symptoms of organophosphorus poisoning, including but not limited to miosis, blurred vision, dark vision, headache, nausea, dizziness, vomiting, hypersecretion (e.g. sweating, salivation, lacrimation, and rhinorrhea), abdominal cramps, diarrhea, urinary incontinence, muscle twitching/fasciculations, paralysis, pallor, muscle weakness, tremors, convulsions, incoordination, diaphoresis, bronchospasm, bronchorrhea, tightness in chest, wheezing, productive cough, pulmonary edema, bradycardia; sinus arrest, tachycardia, hypertension, toxic myocardiopathy, mydriasis, ataxia, anxiety, restlessness, choreiform movement, loss of consciousness, respiratory depression, fatigue, seizures, and psychiatric symptoms (e.g. depression, memory loss, confusion, toxic psychosis). It is within the level of one of skill in the art to assess whether administration of the compositions provided herein prevent or treat such side effects.

Any OP bioscavenger provided herein above can be used in the compositions or combinations herein in methods or uses for preventing or treating organophosphate poisoning and associated symptoms. For example, the organophosphorus bioscavenger for use in the prophylactic and treatment methods provided herein can be selected from any organophosphorus bioscavenger provided herein or known in the art, including, but not limited to proteins that bind to or hydrolyze organophosphorus compounds, including but not limited to cholinesterases, such as acetylcholinesterase (AChE) and butyrylcholinesterase (BChE), paraoxonases (e.g. PON), organophosphate hydrolases, such as parathion hydrolase, sarinase, phosphotriesterase, and prolidase, aryldialkylphosphatases, diisopropylfluorophosphatases (e.g. DFPase), organophosphorus acid anhydrase. organophosphate acid anhydrolases (e.g. OPAA), and variants thereof, such as allelic or species variants and derivatives thereof.

The compositions provided herein of a hyaluronan degrading enzyme in combination with an organophosphorus bioscavenger can be administered for the prevention and treatment of adverse effects caused by organophosphorus nerve agents including, but not limited to, a C-series nerve agent, a V-series nerve agent or other organophosphorus nerve agents. Exemplary nerve agents include, but are not limited to tabun (GA), methyl parathion, sarin (GB), soman (GD), cyclosarin (GF), GV, EA-3148, VE, VG, VM, VR, VX, diisopropylfluorophosphate (DFP), and PB. In particular examples, the compositions provided herein are used to prevent or treat one or more symptoms of exposure to an organophosphorus nerve agent that is sarin, tabun, or VX.

The compositions and combinations provided herein of a hyaluronan degrading enzyme in combination with an organophosphorus bioscavenger can be administered for the prevention and treatment adverse effects caused by organophosphorus pesticide agents including, but not limited to, acephate (Orthene), azinphos-methyl (Gusathion, Guthion), bensulide (Betasan, Lecscosan), bornyl (Swat), bromophos (Nexion), bromophos-ethyl (Nexagan), cadusafos (Apache, Ebufos, Rugby), carabophenothion (Trithion), chlorethoxyfos (Fortress), chlorfenvinphos (Apachlor, Birlane), chlormephos (Dotan), chlorphoxim (Bathion-C), chlorpyrifos (Brodan, Dursban, Lorsban), chlorpyrifos-methyl, chlorthiophos (Celathion), coumaphos (Asuntol, Co-Ral), crytoxyphos (Ciodrin, Cypona), crufomate (Rulene), cyanophenphos (Surecide), cyanophos (Cyanox), cythioate (Cyflee, Proban), DEF (De-Green, E-Z Off D), demeton (Systox), demeton-5-methyl (Duratox, Metasystoxl), dialifor (Torak), diazinon, dichlorofenthion (VC-13 Nemacide), dichlorvos (DDVP, Vapona), dicrotophos (Bidrin), diisopropyl fluorophosphate, dimefos (Hanane, Pestox XIV), dimethoate (Cygon, DeFend), dioxathion (Delnav), disulfoton (Disyston), ditalimfos, edifenphos, endothion, EPBP (S-Seven), EPN, ethion (Ethanox), ethoprop (Mocap), ethyl parathion (E605, Parathion, thiophos), etrimfos (Ekamet), famphur (Bash, Bo-Ana, Famfos), fenamiphos (Nemacur), fenitrothion (Accothion, Agrothion, Sumithion), •fenophosphon (Agritoxn trichloronate), fensulfothion (Dasanit), fenthion (Baytex, Entex, Tiguvon), fonofos (Dyfonate, N-2790), formothion (Anthio), fosthietan (Nem-A-Tak), fosthiazate, heptenophos (Hostaquick), hiometon (Ekatin), hosalone (Zolone), IBP (Kitazin), iodofenphos (Nuvanol-N), isazofos (Brace, Miral, Triumph), isofenphos (Amaze, Oftanol), isoxathion (E-48, Karphos), leptophos (Phosvel), malathion (Cythion), mephosfolan (Cytrolane), merphos (Easy off-D, Folex), methamidophos (Monitor), methidathion (Supracide, Ultracide), methyl parathion (E 601, Penncap-M), methyl trithion, mevinphos (Duraphos, Phosdrin), mipafox (Isopestox, Pestox XV), monocrotophos (Azodrin), naled (Dibrom), omethioate, oxydemeton-methyl (Metasystox-R), oxydeprofos (Metasystox-S), parathion, parathion-methyl, phencapton (G 28029), phenthoate (dimephenthoate, Phenthoate), phorate (Rampart, Thimet), phosalone (Azofene, Zolone), phosfolarr (Cylan, Cyolane), phosmet (Imidan, Prolate), phosphamidon (Dimecron), phostebupirim (Aztec), phoxim (Baythion), pirimiphos-ethyl (Primicid), pirimiphos-methyl (Actellic), profenofos (Curacron), propetamphos (Safrotin), propyl thiopyrophosphate (Aspon), prothoate (Fac), pyrazophos (Afugan, Curamil), pyridaphenthion (Ofunack), quinalphos (Bayrusil), ronnel (Fenchlorphos, Korlan), schradan (OMPA), sulfotep (Bladafum, Dithione, Thiotepp), sulprofos (Bolstar, Helothion), temephos (Abate, Abathion), terbufos (Contraven, Counter), tetrachlorvinphos (Gardona, Rabon), tetraethyl pyrophosphate (TEPP), triazophos (Hostathion), tribufos, and trichlorfon (Dipterex, Dylox, Neguvon, Proxol). In particular examples, the compositions provided herein are used to prevent or treat one or more symptoms of exposure to an organophosphorus pesticide that is dichlorvos, diazinon, chlorpyrifos, ethionmethyl parathion, parathion, malathion, or azinphos-methyl

Subjects for treatment with compositions of a hyaluronan degrading enzyme in combination with an organophosphorus bioscavenger include mammals, including humans, who are exposed to or risk exposure to an organophosphorus agent. Exemplary subjects for treatment include, for example, persons exposed to nerve agents, military personnel, agricultural workers and other persons who handle or come in contact with organophosphorus compounds. Factors, such as, but not limited to, the risk of exposure of exposure or toxicity or the particular organophosphorus agent can be considered for selecting subjects or prophylactic or post-exposure treatment.

Some organophosphorus agents, including but not limited to V-series nerve agents, are persistent agents, meaning that these agents do not degrade or wash away easily and can therefore remain on clothes and other surfaces for long periods. Accordingly, the compositions containing an organophosphorus bioscavenger and a hyaluronan degrading enzyme provided herein can be employed to treat clothing or surfaces contaminated with the organophosphorus agent.

1. Prophylactic Treatment and Prevention

Methods for the treatment or prevention of organophosphorus poisoning include prophylactic treatment prior to exposure to the organophosphorus agent. In such methods, the subject is pretreated with a therapeutically effective amount of a composition of an organophosphorus bioscavenger in combination with a hyaluronan degrading enzyme sufficient to reduce or prevent the occurrence of one or more symptoms of organophosphorus poisoning. Such methods include treatment of a subject about at least or 1, 2, 3, 4, 5, 6, 7, 8, 9, 10, 12, 14, 16, 18, 20, 24, 30, 36, 42, 48 or more hours prior to exposure to the organophosphorus agent.

The amount of an organophosphorus bioscavenger administered in combination with a hyaluronan degrading enzyme to a subject is sufficient to maintain endogenous cholinesterase activity at a level that does not fall below a level to contribute to cholinergic toxicity and concomitant adverse side effects of exposure to the organophosphorus agent (i.e. cholinergic toxicity). Assays to assess endogenous cholinesterase activity are described above. Typically as described above, the threshold level of a bioscavenger for protection is an amount that maintains the endogenous cholinesterase activity above 20% of baseline activity, such as above 30%, 40%, 50%, 60%, 70%, 80%, 90% or more above baseline activity. In some examples, the amount of bioscavenger is such that the level of endogenous cholinesterase activity is sufficient to prevent any signs of cholinergic toxicity. In some examples, the amount or dosage amount of bioscavenger is selected such that the level of endogenous cholinesterase activity is sufficient to reduce signs of cholinergic toxicity by at least 10%, 15%, 20%, 25%, 30%, 35%, 45%, 50%, 55%, 60%, 65%, 75%, 80%, 90%, 95%, 96%, 97%, 98%, 99%, or more.

Hence, for prophylactic use, the amount of organophosphorus bioscavenger administered in combination with a hyaluronan degrading enzyme to a subject prior to exposure to an organophosphorus agent is sufficient to maintain at least about 20%, 25%, 30%, 35%, 45%, 50%, 55%, 60%, 65%, 75%, 80%, 85%, 90%, 95% or more of the baseline endogenous cholinesterase activity in the subject following exposure to the organophosphorus agent for several hours or days. Generally, the amount of an organophosphorus bioscavenger administered in combination with a hyaluronan degrading enzyme is sufficient to maintain endogenous cholinesterase activity to at least about 20%, at least about 25%, at least about 30%, at least about 35%, or at least about 40% of the baseline cholinesterase activity in the subject. In a particular example, the amount of an organophosphorus bioscavenger administered in combination with a hyaluronan degrading enzyme is sufficient to maintain endogenous cholinesterase activity to at least about 30% of the baseline cholinesterase activity in the subject. Typically, the cholinesterase activity is maintained at the above recited levels over a desired or predetermined time after administration of the bioscavenger and/or exposure to a nerve agent. For example, cholinesterase activity is maintained at at least about 30% of baseline cholinesterase activity for at least 24 hours, 2 days, 3, 4, 5, 6, 7, 8, 9, 10, 11, 12, 13, 14, 15, 16, 17, 18, 19, 20 or more days, and generally at least for 10 days.

For example, the amount of OP bioscavenger, such as a butyrylcholinesterase, is administered in an amount that maintains at least 15 μg/mL, such as at least 20 μg/mL, 21 μg/mL, 22 μg/mL 23 μg/mL, 24 μg/mL, 25 μg/mL, 26 μg/mL, 27 μg/mL, 28 μg/mL, 29 μg/mL, 30 μg/mL, 40 μg/mL, 50 μg/mL, 60 μg/mL, 70 μg/mL, 80 μg/mL, 90 μg/mL, 100 μg/mL of the OP bioscavenger in plasma within 24 hours of administration and that last for at least 10 days at that level or higher. Generally, the amount of organophosphorus bioscavenger administered in combination with a hyaluronan degrading enzyme that is sufficient to prevent cholinergic toxicity following exposure to the organophosphorus agent or treat organophosphorus poisoning is less than the amount of the organophosphorus bioscavenger required in the absence of the hyaluronan degrading enzyme. Such dosages and amounts are described elsewhere herein and can be empirically determined based on the particular OP bioscavenger, organophosphorus compound (e.g. nerve agent), route of administration, the subject to be treated and/or other parameters that can influence the precise dosage.

Exemplary amounts that are administered for treatment of organophosphate poisoning include, for example, 50 mg to 1000 mg of OP bioscavenger, such as 100 mg to 800 mg, 200 mg to 750 mg, and in particular at least 500 μg or at least 750 mg OP bioscavenger. For example, dosages of an OP bioscavenger that can be administered include, 0.5 mg/kg to 20 mg/kg, such as 1 mg/kg to 10 mg/kg, 2 mg/kg to 8 mg/kg or 4 mg/kg to 6 mg/kg.

Dosages of co-administered hyaluronan-degrading enzymes also are described above and elsewhere herein and include, for example, administering at least or least about or 1 Unit (U), 10 U, 100 U, 500 U, 1000 U, 5,000 U, 10,000 U, 20,000 U, 30,000 U, 40,000 U, 50,000 U, 60,000 U, 70,000 U, 80,000 U, 90,000 U, 100,000 U, 110,000 U, 120,000 U, 130,000 U, 140,000 U, 150,000 U, 160,000 U, 170,000 U, 180,000 U, 190,000 U, 200,000 U, 300,000 U, 400,000 U, 500,000 U; 600,000 U; 700,000 U; 800,000 U; 900,000 U; 1,000,000 U; 1,500,000 U; 2,000,000 U; 2,500,000 U; 3,000,000 U; 3,500,000 U; 4,000,000 U; 5,000,000 U; 6,000,000 U or more, per single dosage. Generally, a hyaluronan-degrading enzyme is administered to a subject in an amount that is between or about between 0.01 μg/kg to 25 mg/kg, such as 0.0005 mg/kg (0.5 μg/kg) to 25 mg/kg, 0.5 μg/kg to 10 mg/kg (320,000 U/kg), 0.02 mg/kg to 1.5 mg/kg, 0.01 μg/kg to 15 μg/kg, 0.05 μg/kg to 10 μg/kg, 0.75 μg/kg to 7.5 μg/kg or 1.0 μg/kg to 3.0 μg/kg. For example, a hyaluronan-degrading enzyme can be administered at or about 1 Unit/kg to 800,000 Units/kg, 10 to 100,000 Units/kg, 1 Unit/kg to 1000 Units/kg, 1 Units/kg to 500 Units/kg or 10 Units/kg to 50 Units/kg of the mass of the subject to whom it is administered.

In particular examples, the OP bioscavenger and hyaluronan-degrading enzyme are administered by percutaneous administration, such as by subcutaneous or intramuscular administration. For example, as found herein, intramuscular administration is associated with increased absorption and bioavailability of the OP bioscavenger when administered in combination with a hyaluronan-degrading enzyme.

In the prophylactic methods herein for preventing organophosphate poisoning provided herein, the OP bioscavenger is administered prior to exposure with the organosphosphate compound (e.g. nerve agent). For example, the OP bioscavenger is administered at least 1 hour, 2 hour, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 26 hours, 28 hours, 30 hours, 32 hours, 34 hours, 36 hours, 42 hours, or 48 hours prior to exposure with the organophosphate compound (e.g. nerve agent). Hence, generally, the amount of an organophosphorus bioscavenger administered is sufficient to provide protection from organophosphorus poisoning within 4, 6, 8, 10, 12, 14, 16, 18, 20, 24, 26, 28, 30, 32, 34, 36, 42, or 48 hours after administration of the organophosphorus bioscavenger. Generally, for prophylactic use, the amount of an organophosphorus bioscavenger administered in combination with a hyaluronan degrading enzyme is sufficient to provide protection from organophosphorus poisoning within at least about 24 hours after administration of the organophosphorus bioscavenger.

The hyaluronan-degrading enzyme is co-formulated or co-administered with the OP bioscavenger. When the hyaluronan-degrading enzyme is co-administered with the OP bioscavenger, it is administered prior to, intermittently, simultaneously or subsequently. For example, the hyaluronan-degrading enzyme can be administered at least 1 minute, 10 minutes, 30 minutes, 1 hour, 2 hours, 3 hours, 4 hours, 5 hours, 6 hours, 12 hours or 24 hours prior to administering the OP bioscavenger. In other examples, the hyaluronan-degrading enzyme is administered within 1 minutes, 10 minutes, 30 minutes, 1 hour, 2 hours, 3 hours, 4 hours, 5 hours, 6 hours, 12 hours or 24 hours after administering the OP bioscavenger.

In the methods herein, administration of an OP bioscavenger can be repeated for a cycle of administration over weeks, months or years. The particular cycle of administration can depend on the potential exposure of a subject to an organophosphorus compound or poisoning agent. The particular cycle also can depend on the length of organophosphorus poisoning of the particular compound. The frequency of administration of a composition or combination containing an OP bioscavenger can be at least every or every 2 days, 3 days, 4 days, 5 days, 6 days, 7 days, 8 days, 9 days, 10 days, 11 days, 12 days, 13 days, 14 days, 3 weeks or one month. For example, the OP bioscavenger and hyaluronan-degrading enzyme are administered at least every 10 days.

2. Treatment after Exposure

Methods for the treatment or prevention of organophosphorus poisoning include treatment of a subject that has been exposed to an organophosphorus agent, including a subject that exhibits one or more symptoms of organophosphorus poisoning. In such methods, the subject is treated with a therapeutically effective amount of a composition of a hyaluronan degrading enzyme in combination with an organophosphorus bioscavenger sufficient to reduce or eliminate one or more symptoms of organophosphorus poisoning. Such methods include treatment of a subject about at least or 1, 5, 10, 20, 30 or more minutes, or about or at least 1, 2, 3, 4, 5, 6, 7, 8, 9, 10, 12, 14, 16, 18, 20, 22, 24, 26, 28, 30, 32, 34, 36 or more hours following exposure to the organophosphorus agent.

For treatment following exposure to the organophosphorus agent, the amount of organophosphorus bioscavenger administered in combination with a hyaluronan degrading enzyme to a subject following exposure to an organophosphorus agent is sufficient to restore endogenous cholinesterase activity in the subject to at least about 20%, 25%, 30%, 35%, 45%, 50%, 55%, 60%, 65%, 75%, 80%, 85%, 90%, 95% or more of the baseline cholinesterase activity in the subject. Generally, the amount of an organophosphorus bioscavenger administered in combination with a hyaluronan degrading enzyme is sufficient to restore endogenous cholinesterase activity to at least about 20%, at least about 25%, at least about 30%, at least about 35%, or at least about 40% of the baseline cholinesterase activity in the subject. In a particular example, the amount of an organophosphorus bioscavenger administered in combination with a hyaluronan degrading enzyme is sufficient to restore endogenous cholinesterase activity to at least about 30% of the baseline cholinesterase activity in the subject.

In such methods, the amount of organophosphorus bioscavenger administered in combination with a hyaluronan degrading enzyme that is sufficient to reduce or eliminate one or more symptoms of organophosphorus poisoning is less than the amount of the organophosphorus bioscavenger required in the absence of the hyaluronan degrading enzyme. Such dosages and amounts are described elsewhere herein and can be empirically determined based on the particular OP bioscavenger, organophosphorus compound (e.g. nerve agent), route of administration, the subject to be treated and/or other parameters that can influence the precise dosage.

Exemplary amounts that are administered to reduce or eliminate one or more symptoms of organophosphorus poisoning include, for example, 50 mg to 1000 mg of OP bioscavenger, such as 100 mg to 800 mg, 200 mg to 750 mg, and in particular at least 500 mg or at least 750 mg OP bioscavenger. For example, dosages of an OP bioscavenger that can be administered include, 0.5 mg/kg to 20 mg/kg, such as 1 mg/kg to 10 mg/kg, 2 mg/kg to 8 mg/kg or 4 mg/kg to 6 mg/kg.

Dosages of co-administered hyaluronan-degrading enzymes also are described above and elsewhere herein and include, for example, administering at least or least about or 1 Unit (U), 10 U, 100 U, 500 U, 1000 U, 5,000 U, 10,000 U, 20,000 U, 30,000 U, 40,000 U, 50,000 U, 60,000 U, 70,000 U, 80,000 U, 90,000 U, 100,000 U, 110,000 U, 120,000 U, 130,000 U, 140,000 U, 150,000 U, 160,000 U, 170,000 U, 180,000 U, 190,000 U, 200,000 U, 300,000 U, 400,000 U, 500,000 U; 600,000 U; 700,000 U; 800,000 U; 900,000 U; 1,000,000 U; 1,500,000 U; 2,000,000 U; 2,500,000 U; 3,000,000 U; 3,500,000 U; 4,000,000 U; 5,000,000 U; 6,000,000 U or more, per single dosage. Generally, a hyaluronan-degrading enzyme is administered to a subject in an amount that is between or about between 0.01 μg/kg to 25 mg/kg, such as 0.0005 mg/kg (0.5 μg/kg) to 25 mg/kg, 0.5 μg/kg to 10 mg/kg (320,000 U/kg), 0.02 mg/kg to 1.5 mg/kg, 0.01 μg/kg to 15 μg/kg, 0.05 μg/kg to 10 μg/kg, 0.75 μg/kg to 7.5 μg/kg or 1.0 mg/kg to 3.0 μg/kg. For example, a hyaluronan-degrading enzyme can be administered at or about 1 Unit/kg to 800,000 Units/kg, 10 to 100,000 Units/kg, 1 Unit/kg to 1000 Units/kg, 1 Units/kg to 500 Units/kg or 10 Units/kg to 50 Units/kg of the mass of the subject to whom it is administered.

### I. Combination Therapies

The compositions and combinations of a hyaluronan degrading enzyme in combination with an organophosphorus bioscavenger can be administered in a combination treatment for the prevention or treatment of organophosphorus poisoning. For example, compositions of a hyaluronan degrading enzyme in combination with an organophosphorus bioscavenger can be further co-formulated or co-administered together with, prior to, intermittently with, or subsequent to, other therapeutic or pharmacologic agents or treatments, such as procedures, for example, for the prevention or treatment of one or more effects or symptoms of organophosphorus poisoning. Such agents include, but are not limited to, small molecule compounds, biologics, supportive care, including oxygen therapy, and combinations thereof. Such other agents and treatments that are available for the treatment of organophosphorus poisoning, including all those exemplified herein, are known to one of skill in the art or can be empirically determined.

A preparation of a second agent or agents or treatment or treatments can be administered at once, or can be divided into a number of smaller doses to be administered at intervals of time. Selected agent/treatment preparations can be administered in one or more doses over the course of a treatment time for example over several hours, days, weeks, or months. In some cases, continuous administration is useful. It is understood that the precise dosage and course of administration depends on the indication and patient's tolerability. Generally, dosing regimes for second agents/treatments herein are known to one of skill in the art.

In one example, a composition of a hyaluronan degrading enzyme in combination with an organophosphorus bioscavenger is administered as part of a combination therapy, by administering the composition and a second agent for the prevention or treatment of organophosphorus poisoning. In one example, the composition of a hyaluronan degrading enzyme in combination with an organophosphorus bioscavenger and second agent or treatment can be co-formulated and administered together. In another example, the composition of a hyaluronan degrading enzyme in combination with an organophosphorus bioscavenger is administered subsequently, intermittently or simultaneously with the second agent or treatment preparation.

Exemplary treatments for organophosphorus poisoning involve administration, such as by intravenous or intramuscular injection, of drugs that antagonize the effects of elevated acetylcholine levels, restore normal acetylcholinesterase activity, and treat one or more symptoms of organophosphorus poisoning. Exemplary drugs for the treatment of organophosphorus poisoning include, but are not limited to, carbamates, anti-muscarinics, and cholinesterase (ChE)-reactivators, such as monopyridium and bispyridium oximes. Exemplary drugs for the treatment of delirium associated with organophosphorus poisoning include benzodiazepines, such as diazepam. Exemplary carbamates include, for example, pyridostigmine. Exemplary anti-muscarinics include, for example, atropine. Exemplary oximes include, for example, pralidoxime chloride (pyridinium-2-aldoxime, 2-PAM, Protopam) trimedoxime (TMB-4), obidoxime (LuH-6, Toxogonin) and asoxime (HI-6). Additional treatments for treatment of organophosphorus poisoning include, but are not limited to, gastric lavage for immediate treatment of ingested organophosphorus agents, magnesium sulphate, sodium bicarbonate, glycopyrrolate and alpha-adrenergic receptor agonists.

### J. Articles of Manufacture and Kits

Pharmaceutical compositions of organophosphorus bioscavengers and hyaluronan-degrading enzymes, for example butyrylcholinesterases and hyaluronidases, or nucleic acids encoding organophosphorus bioscavengers and hyaluronan-degrading enzymes, or a derivative or variant thereof can be packaged as articles of manufacture containing packaging material, a pharmaceutical composition which is effective for treating the disease or disorder, and a label that indicates that a selected organophosphorus bioscavenger or nucleic acid molecule is to be used for treating the disease or disorder. Instructions for use can be provided. For example, instructions can be provided that specify that the organophosphorus bioscavenger is to be reconstituted with the accompanying liquid buffer or solution immediately before administration. Instructions also can be provided to specify the timing of administration, the route of administration, the particular dosage or amount to be administered and other instructions related to the components and their administration. Combinations of an organophosphorus bioscavenger, for example BChE, or derivative or variant thereof and a hyaluronan-degrading enzyme, e.g., rHuPH20, or derivative or variant thereof, also can be packaged in an article of manufacture.

The articles of manufacture provided herein contain packaging materials. Packaging materials for use in packaging pharmaceutical products are well known to those of skill in the art. See, for example, U.S. Pat. Nos. 5,323,907, 5,052,558 and 5,033,252, each of which is incorporated herein in its entirety. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. The articles of manufacture can include a needle or other injection device so as to facilitate administration (e.g. sub-epidermal administration) for local injection purposes. A wide array of formulations of the compounds and compositions provided herein are contemplated as are a variety of treatments for organophosphorus poisoning.

The choice of package depends on the organophosphorus bioscavenger and hyaluronan-degrading enzyme (if included therewith), and whether such compositions will be packaged together or separately. In general, the packaging is non-reactive with the compositions contained therein such that the organophosphorus bioscavenger retains its activity. In one example, the organophosphorus bioscavenger can be packaged in lyophilized form with a buffer or diluent for reconstitution. The buffer or diluent can be stored separately and provide the hyaluronan-degrading enzyme, or can be provided in a form capable of providing the hyaluronan-degrading enzyme when desired. For example, instructions can be provided to add the hyaluronan-degrading enzyme to the buffer or diluent before use.

In other examples, the organophosphorus bioscavenger is packaged in the same container as the hyaluronan-degrading enzyme, such that the composition can be administered by the user at will. In one example, the organophosphorus bioscavenger and hyaluronan-degrading enzyme can be packaged together in lyophilized form with a buffer or diluent for reconstitution. The buffer or diluent can be stored separately for use when desired.

In other examples, the organophosphorus bioscavenger is packaged in a container with the hyaluronan-degrading enzyme such that the composition can be administered by the user at will. Generally, examples of such containers include those that have an enclosed, defined space that contains the organophosphorus bioscavenger, and a separate enclosed, defined space containing the hyaluronan-degrading enzyme such that the two spaces are separated by a readily removable membrane which, upon removal, permits the components to mix. Any container or other article of manufacture is contemplated, so long as the organophosphorus bioscavenger is separated from the hyaluronan-degrading enzyme. Exposure of the hyaluronan-degrading enzyme to the organophosphorus bioscavenger is prior to use. For example, the physical separation means are those that are readily removed by the user, to permit mixing. For example, an article of manufacture can contain an organophosphorus bioscavenger in one compartment and an hyaluronan-degrading enzyme in an adjacent compartment. The compartments are separated by a dividing member, such as a membrane, that, upon compression of the article or manufacture ruptures permitting separated components to mix. For suitable embodiments see e.g., containers described in U.S. Pat. Nos. 3,539,794 and 5,171,081.

Following are some examples of the packaging requirements of various end uses of organophosphorus bioscavengers. These are offered as examples only and in no way are intended as limiting.

1. Single Chamber Apparatus

Among the simplest embodiments herein, are those in which the apparatus contains a single chamber or container and, if needed, ejection means. Single chamber housings or containers include any item in which an organophosphorus bioscavenger is included in the container. The organophosphorus bioscavenger is housed in the vessel in liquid phase or as a powder or other paste or other convenient composition. The vessel or liquid can be stored at any temperature such that the organophosphorus bioscavenger is stable. An organophosphorus bioscavenger can be reconstituted with an appropriate liquid diluent or buffer containing the hyaluronan-degrading enzyme or the hyaluronan-degrading enzyme can be administered separately at the site of administration. Kits containing the item and the hyaluronan-degrading enzyme also are provided.

2. Dual Chamber Apparatus

An example of an apparatus contemplated for use herein is a dual chamber container. In general, this apparatus has two chambers or compartments thereby maintaining the organophosphorus bioscavenger from the hyaluronan-degrading enzyme until use is desired. The apparatus can include a mixing chamber to permit mixing of the components prior to dispensing from the apparatus. Alternatively, mixing can occur by ejection of the hyaluronan-degrading enzyme from one chamber into a second chamber containing the organophosphorus bioscavenger. For example, the organophosphorus bioscavenger can be provided in lyophilized form, and reconstitution can be achieved by ejection of the hyaluronan-degrading enzyme from a first chamber into the second chamber containing the lyophilized enzyme.

In one embodiment, a dual chamber apparatus employs a mechanical pump mechanism in its operation. In such an example, the dispensing apparatus maintains the components in separate chambers. A pump mechanism is operated to withdraw the contents from each chamber and into a mixing chamber, or from one chamber into the second chamber. Upon mixing, the mixed composition is activated by reaction of the components in the chambers. The pump mechanism can be manually operated, for example, by a plunger. Exemplary of such dual chamber apparatus include dual chamber syringes (see e.g., U.S. Pat. Nos. 6,972,005, 6,692,468, 5,971,953, 4,529,403, 4,202,314, 4,214,584, 4,983,164, 5,788,670, 5,395,326; and Intl. Patent Publication Nos. WO2007006030, WO2001047584).

Another embodiment of a dual chamber fluid dispensing apparatus contemplated for use herein takes the form of a compressible bottle or tube or other similar device. The device has two compartments within it that keep the components separated. The cap of the device can serve as a mixing chamber, a mixing chamber can be positioned between the two chambers and the cap, or mixing can be achieved within one of the chambers. The components are forced by compression from the separate compartments into the mixing chamber. They are then dispensed from the mixing chamber. For example, the mixed contents can be removed from the device by attaching a plunger/syringe apparatus to the dispensing end and withdrawing the contents therethrough. Such devices are known in the art (see e.g., Intl. Pat. Publication No. WO1994015848).

3. Kits

Selected organophosphorus bioscavengers, e.g., BChE, and hyaluronan-degrading enzymes, e.g., rHuPH20, and/or articles of manufacture thereof also can be provided as kits. Kits can include a pharmaceutical composition described herein and an item for administration provided as an article of manufacture. For example a selected organophosphorus bioscavenger can be supplied with a device for administration, such as a syringe, an inhaler, a dosage cup, a dropper, or an applicator. The compositions can be contained in the item for administration or can be provided separately to be added later. Generally, kits contain an item with an organophosphorus bioscavenger and/or a hyaluronan-degrading enzyme. The kit can, optionally, include instructions for application including dosages, dosing regimens, instructions for using the hyaluronan-degrading enzyme, and instructions for modes of administration. Kits also can include a pharmaceutical composition described herein and an item for diagnosis. For example, such kits can include an item for measuring the concentration, amount or activity of the selected OP bioscavenger in a subject.

### K. Examples

### Example 1

### Generation of a Soluble rHuPH20-Expressing Cell Line

The HZ24 plasmid (set forth in SEQ ID NO:52) was used to transfect Chinese Hamster Ovary (CHO cells) (see e.g. U.S. Pat. Nos. 7,767,429 and 7,781,607 and U.S. Publication No. 2006-0104968). The HZ24 plasmid vector for expression of soluble rHuPH20 contains a pCI vector backbone (Promega), DNA encoding amino acids 1-482 of human PH20 hyaluronidase (SEQ ID NO:49), an internal ribosomal entry site (IRES) from the ECMV virus (Clontech), and the mouse dihydrofolate reductase (DHFR) gene. The pCI vector backbone also includes DNA encoding the Beta-lactamase resistance gene (AmpR), an f1 origin of replication, a Cytomegalovirus immediate-early enhancer/promoter region (CMV), a chimeric intron, and an SV40 late polyadenylation signal (SV40). The DNA encoding the soluble rHuPH20 construct contains an NheI site and a Kozak consensus sequence prior to the DNA encoding the methionine at amino acid position 1 of the native 35 amino acid signal sequence of human PH20, and a stop codon following the DNA encoding the tyrosine corresponding to amino acid position 482 of the human PH20 hyaluronidase set forth in SEQ ID NO: 1, followed by a BamHI restriction site. The construct pCI-PH20-IRES-DHFR-SV40pa (HZ24), therefore, results in a single mRNA species driven by the CMV promoter that encodes amino acids 1-482 of human PH20 (set forth in SEQ ID NO:3) and amino acids 1-186 of mouse dihydrofolate reductase (set forth in SEQ ID NO:53), separated by the internal ribosomal entry site (IRES).

Non-transfected DG44 CHO cells growing in GIBCO Modified CD-CHO media for DHFR(−) cells, supplemented with 4 mM Glutamine and 18 mL/L Plurionic F68/L (Gibco), were seeded at 0.5×10⁶ cells/mL in a shaker flask in preparation for transfection. Cells were grown at 37° C. in 5% CO₂ in a humidified incubator, shaking at 120 rpm. Exponentially growing non-transfected DG44 CHO cells were tested for viability prior to transfection.

Sixty million viable cells of the non-transfected DG44 CHO cell culture were pelleted and resuspended to a density of 2×10⁷ cells in 0.7 mL of 2× transfection buffer (2×HeBS: 40 mM Hepes, pH 7.0, 274 mM NaCl, 10 mM KCl, 1.4 mM Na₂HPO₄, 12 mM dextrose). To each aliquot of resuspended cells, 0.09 mL (250 μg) of the linear HZ24 plasmid (linearized by overnight digestion with Cla I (New England Biolabs) was added, and the cell/DNA solutions were transferred into 0.4 cm gap BTX (Gentronics) electroporation cuvettes at room temperature. A negative control electroporation was performed with no plasmid DNA mixed with the cells. The cell/plasmid mixes were electroporated with a capacitor discharge of 330 V and 960 μF or at 350 V and 960 μF.

The cells were removed from the cuvettes after electroporation and transferred into 5 mL of Modified CD-CHO media for DHFR(−) cells, supplemented with 4 mM Glutamine and 18 mL/L Plurionic F68/L (Gibco), and allowed to grow in a well of a 6-well tissue culture plate without selection for 2 days at 37° C. in 5% CO₂ in a humidified incubator.

Two days post-electroporation, 0.5 mL of tissue culture media was removed from each well and tested for the presence of hyaluronidase activity, using a standard microturbidity assay.

TABLE 4
Initial Hyaluronidase Activity of HZ24 Transfected DG44 CHO
cells at 40 hours post-transfection

| | Dilution | Activity (Units/mL) |
|---|---|---|
| Transfection 1 330 V | 1 to 10 | 0.25 |
| Transfection 2 350 V | 1 to 10 | 0.52 |
| Negative Control | 1 to 10 | 0.015 |

Cells from Transfection 2 (350V) were collected from the tissue culture well, counted and diluted to 1×10⁴ to 2×10⁴ viable cells per mL. A 0.1 mL aliquot of the cell suspension was transferred to each well of five, 96 well round bottom tissue culture plates. One hundred microliters of CD-CHO media (GIBCO) containing 4 mM GlutaMAX™-1 supplement (GIBCO™, Invitrogen Corporation) and without hypoxanthine and thymidine supplements were added to the wells containing cells (final volume 0.2 mL).

Ten clones were identified from the 5 plates grown without methotrexate.

TABLE 5
Hyaluronidase activity of identified clones

| Plate/Well ID | Relative Hyaluronidase |
|---|---|
| 1C3 | 261 |
| 2C2 | 261 |
| 3D3 | 261 |
| 3E5 | 243 |
| 3C6 | 174 |
| 2G8 | 103 |
| 1B9 | 304 |
| 2D9 | 273 |
| 4D10 | 302 |

Six HZ24 clones were expanded in culture and transferred into shaker flasks as single cell suspensions. Clones 3D3, 3E5, 2G8, 2D9, 1E11, and 4D10 were plated into 96-well round bottom tissue culture plates using a two-dimensional infinite dilution strategy in which cells were diluted 1:2 down the plate, and 1:3 across the plate, starting at 5000 cells in the top left hand well. Diluted clones were grown in a background of 500 non-transfected DG44 CHO cells per well, to provide necessary growth factors for the initial days in culture. Ten plates were made per subclone, with 5 plates containing 50 nM methotrexate and 5 plates without methotrexate.

Clone 3D3 produced 24 visual subclones (13 from the no methotrexate treatment, and 11 from the 50 nM methotrexate treatment. Significant hyaluronidase activity was measured in the supernatants from 8 of the 24 subclones (>50 Units/mL), and these 8 subclones were expanded into T-25 tissue culture flasks. Clones isolated from the methotrexate treatment protocol were expanded in the presence of 50 nM methotrexate: Clone 3D35M was further expanded in 500 nM methotrexate giving rise to clones producing in excess of 1,000 Units/mL in shaker flasks (clone 3D35M; or Gen1 3D35M). A master cell bank (MCB) of the 3D35M cells was then prepared.

### Example 2

### Production Gen2 Cells Containing Soluble human PH20 (rHuPH20)

The Gen1 3D35M cell line described in Example 1 was adapted to higher methotrexate levels to produce generation 2 (Gen2) clones. 3D35M cells were seeded from established methotrexate-containing cultures into CD CHO medium containing 4 mM GlutaMAX-1™ and 1.0 μM methotrexate. The cells were adapted to a higher methotrexate level by growing and passaging them 9 times over a period of 46 days in a 37° C., 7% CO₂ humidified incubator. The amplified population of cells was cloned out by limiting dilution in 96-well tissue culture plates containing medium with 2.0 μM methotrexate. After approximately 4 weeks, clones were identified and clone 3E10B was selected for expansion. 3E10B cells were grown in CD CHO medium containing 4 mM GlutaMAX-1™ and 2.0 μM methotrexate for 20 passages. A master cell bank (MCB) of the 3E10B cell line was created and frozen and used for subsequent studies.

Amplification of the cell line continued by culturing 3E10B cells in CD CHO medium containing 4 mM GlutaMAX-1™ and 4.0 μM methotrexate. After the 12^{th} passage, cells were frozen in vials as a research cell bank (RCB). One vial of the RCB was thawed and cultured in medium containing 8.0 μM methotrexate. After 5 days, the methotrexate concentration in the medium was increased to 16.0 μM, then 20.0 μM 18 days later. Cells from the 8^{th} passage in medium containing 20.0 μM methotrexate were cloned out by limiting dilution in 96-well tissue culture plates containing CD CHO medium containing 4 mM GlutaMAX-1™ and 20.0 μM methotrexate. Clones were identified 5-6 weeks later and clone 2B2 was selected for expansion in medium containing 20.0 μM methotrexate. After the 11th passage, 2B2 cells were frozen in vials as a research cell bank (RCB).

The resultant 2B2 cells are dihydrofolate reductase deficient (dhfr-) DG44 CHO cells that express soluble recombinant human PH20 (rHuPH20). The soluble PH20 is present in 2B2 cells at a copy number of approximately 206 copies/cell. Southern blot analysis of Spe I-, Xba I- and BamH I/Hind III-digested genomic 2B2 cell DNA using a rHuPH20-specific probe revealed the following restriction digest profile: one major hybridizing band of −7.7 kb and four minor hybridizing bands (˜13.9, ˜6.6, ˜5.7 and ˜4.6 kb) with DNA digested with Spe I; one major hybridizing band of ˜5.0 kb and two minor hybridizing bands (˜13.9 and ˜6.5 kb) with DNA digested with Xba I; and one single hybridizing band of ˜1.4 kb observed using 2B2 DNA digested with BamH I/Hind III. Sequence analysis of the mRNA transcript indicated that the derived cDNA (SEQ ID NO:49) was identical to the reference sequence (SEQ ID NO:56) except for one base pair difference at position 1131, which was observed to be a thymidine (T) instead of the expected cytosine (C). This is a silent mutation, with no effect on the amino acid sequence.

### Example 3

### A. Production of Gen2 Soluble rHuPH20 in 300 L Bioreactor Cell Culture

A vial of HZ24-2B2 was thawed and expanded from shaker flasks through 36 L spinner flasks in CD-CHO media (Invitrogen, Carlsbad, Calif.) supplemented with 20 μM methotrexate and GlutaMAX-1™ (Invitrogen). Briefly, the vial of cells was thawed in a 37° C. water bath, media was added and the cells were centrifuged. The cells were re-suspended in a 125 mL shake flask with 20 mL of fresh media and placed in a 37° C., 7% CO₂ incubator. The cells were expanded up to 40 mL in the 125 mL shake flask. When the cell density reached greater than 1.5×10⁶ cells/mL, the culture was expanded into a 125 mL spinner flask in a 100 mL culture volume. The flask was incubated at 37° C., 7% CO₂. When the cell density reached greater than 1.5×10⁶ cells/mL, the culture was expanded into a 250 mL spinner flask in 200 mL culture volume, and the flask was incubated at 37° C., 7% CO₂. When the cell density reached greater than 1.5×10⁶ cells/mL, the culture was expanded into a 1 L spinner flask in 800 mL culture volume and incubated at 37° C., 7% CO₂. When the cell density reached greater than 1.5×10⁶ cells/mL the culture was expanded into a 6 L spinner flask in 5000 mL culture volume and incubated at 37° C., 7% CO₂. When the cell density reached greater than 1.5×10⁶ cells/mL the culture was expanded into a 36 L spinner flask in 32 L culture volume and incubated at 37° C., 7% CO₂.

A 400 L reactor was sterilized and 230 mL of CD-CHO media was added. Before use, the reactor was checked for contamination. Approximately 30 L cells were transferred from the 36 L spinner flasks to the 400 L bioreactor (Braun) at an inoculation density of 4.0×10⁵ viable cells per mL and a total volume of 260 L. Parameters were temperature setpoint, 37° C.; Impeller Speed 40-55 RPM; Vessel Pressure: 3 psi; Air Sparge 0.5-1.5 L/Min.; Air Overlay: 3 L/min. The reactor was sampled daily for cell counts, pH verification, media analysis, protein production and retention. Also, during the run nutrient feeds were added. At 120 hrs (day 5), 10.4 L of Feed #1 Medium (4×CD-CHO+33 g/L Glucose+160 mL/L Glutamax-1™+83 mL/L Yeastolate+33 mg/L rHuInsulin) was added. At 168 hours (day 7), 10.8 L of Feed #2 (2×CD-CHO+33 g/L Glucose+80 mL/L Glutamax-1™+167 mL/L Yeastolate+0.92 g/L Sodium Butyrate) was added, and culture temperature was changed to 36.5° C. At 216 hours (day 9), 10.8 L of Feed #3 (1× CD-CHO+50 g/L Glucose+50 mL/L Glutamax-1™+250 mL/L Yeastolate+1.80 g/L Sodium Butyrate) was added, and culture temperature was changed to 36° C. At 264 hours (day 11), 10.8 L of Feed #4 (1× CD-CHO+33 g/L Glucose+33 mL/L Glutamax-1™+250 mL/L Yeastolate+0.92 g/L Sodium Butyrate) was added, and culture temperature was changed to 35.5° C. The addition of the feed media was observed to dramatically enhance the production of soluble rHuPH20 in the final stages of production. The reactor was harvested at 14 or 15 days or when the viability of the cells dropped below 40%. The process resulted in a final productivity of 17,000 Units per mL with a maximal cell density of 12 millioncells/mL. At harvest, the culture was sampled for mycoplasma, bioburden, endotoxin and virus in vitro and in vivo, by Transmission Electron Microscopy (TEM) and enzyme activity.

The culture was pumped by a peristaltic pump through four Millistak filtration system modules (Millipore) in parallel, each containing a layer of diatomaceous earth graded to 4-8 μm and a layer of diatomaceous earth graded to 1.4-1.1 μm, followed by a cellulose membrane, then through a second single Millistak filtration system (Millipore) containing a layer of diatomaceous earth graded to 0.4-0.11 μm and a layer of diatomaceous earth graded to <0.1 μm, followed by a cellulose membrane, and then through a 0.22 μm final filter into a sterile single use flexible bag with a 350 L capacity. The harvested cell culture fluid was supplemented with 10 mM EDTA and 10 mM Tris to a pH of 7.5. The culture was concentrated 10× with a tangential flow filtration (TFF) apparatus using four Sartoslice TFF 30 kDa molecular weight cut-off (MWCO) polyether sulfone (PES) filter (Sartorious), followed by a 10× buffer exchange with 10 mM Tris, 20 mM Na₂SO₄, pH 7.5 into a 0.22 μm final filter into a 50 L sterile storage bag.

The concentrated, diafiltered harvest was inactivated for virus. Prior to viral inactivation, a solution of 10% Triton® X-100, 3% tri(n-butyl)phosphate (TNBP) was prepared. The concentrated, diafiltered harvest was exposed to 1% Triton® X-100, 0.3% TNBP for 1 hour in a 36 L glass reaction vessel immediately prior to purification on the Q column.

B. Purification of Gen2 Soluble rHuPH20

A Q Sepharose (Pharmacia) ion exchange column (9 L resin, H=29 cm, D=20 cm) was prepared. Wash samples were collected for a determination of pH, conductivity and endotoxin (LAL) assay. The column was equilibrated with 5 column volumes of 10 mM Tris, 20 mM Na₂SO₄, pH 7.5. Following viral inactivation, the concentrated, diafiltered harvest was loaded onto the Q column at a flow rate of 100 cm/hr. The column was washed with 5 column volumes of 10 mM Tris, 20 mM Na₂SO₄, pH 7.5 and 10 mM Hepes, 50 mM NaCl, pH7.0. The protein was eluted with 10 mM Hepes, 400 mM NaCl, pH 7.0 into a 0.22 μm final filter into sterile bag. The eluate sample was tested for bioburden, protein concentration and hyaluronidase activity. A₂₈₀ absorbance readings were taken at the beginning and end of the exchange.

Phenyl-Sepharose (Pharmacia) hydrophobic interaction chromatography was next performed. A Phenyl-Speharose (PS) column (19-21 L resin, H=29 cm, D=30 cm) was prepared. The wash was collected and sampled for pH, conductivity and endotoxin (LAL assay). The column was equilibrated with 5 column volumes of 5 mM potassium phosphate, 0.5 M ammonium sulfate, 0.1 mM CaCl₂, pH 7.0. The protein eluate from the Q sepharose column was supplemented with 2M ammonium sulfate, 1 M potassium phosphate and 1 M CaCl₂ stock solutions to yield final concentrations of 5 mM, 0.5 M and 0.1 mM, respectively. The protein was loaded onto the PS column at a flow rate of 100 cm/hr and the column flow thru collected. The column was washed with 5 mM potassium phosphate, 0.5 M ammonium sulfate and 0.1 mM CaCl₂ pH 7.0 at 100 cm/hr and the wash was added to the collected flow thru. Combined with the column wash, the flow through was passed through a 0.22 μm final filter into a sterile bag. The flow through was sampled for bioburden, protein concentration and enzyme activity.

An aminophenyl boronate column (Prometics) was prepared. The wash was collected and sampled for pH, conductivity and endotoxin (LAL assay). The column was equilibrated with 5 column volumes of 5 mM potassium phosphate, 0.5 M ammonium sulfate. The PS flow through containing purified protein was loaded onto the aminophenyl boronate column at a flow rate of 100 cm/hr. The column was washed with 5 mM potassium phosphate, 0.5 M ammonium sulfate, pH 7.0. The column was washed with 20 mM bicine, 0.5 M ammonium sulfate, pH 9.0. The column was washed with 20 mM bicine, 100 mM sodium chloride, pH 9.0. The protein was eluted with 50 mM Hepes, 100 mM NaCl, pH 6.9 and passed through a sterile filter into a sterile bag. The eluted sample was tested for bioburden, protein concentration and enzyme activity.

The hydroxyapatite (HAP) column (Biorad) was prepared. The wash was collected and tested for pH, conductivity and endotoxin (LAL assay). The column was equilibrated with 5 mM potassium phosphate, 100 mM NaCl, 0.1 mM CaCl₂, pH 7.0. The aminophenyl boronate purified protein was supplemented to final concentrations of 5 mM potassium phosphate and 0.1 mM CaCl₂ and loaded onto the HAP column at a flow rate of 100 cm/hr. The column was washed with 5 mM potassium phosphate, pH 7, 100 mM NaCl, 0.1 mM CaCl₂. The column was next washed with 10 mM potassium phosphate, pH 7, 100 mM NaCl, 0.1 mM CaCl₂. The protein was eluted with 70 mM potassium phosphate, pH 7.0 and passed through a 0.22 μm sterile filter into a sterile bag. The eluted sample was tested for bioburden, protein concentration and enzyme activity.

The HAP purified protein was then passed through a viral removal filter. The sterilized Viosart filter (Sartorius) was first prepared by washing with 2 L of 70 mM potassium phosphate, pH 7.0. Before use, the filtered buffer was sampled for pH and conductivity. The HAP purified protein was pumped via a peristaltic pump through the 20 nM viral removal filter. The filtered protein in 70 mM potassium phosphate, pH 7.0 was passed through a 0.22 μm final filter into a sterile bag. The viral filtered sample was tested for protein concentration, enzyme activity, oligosaccharide, monosaccharide and sialic acid profiling. The sample also was tested for process related impurities.

The protein in the filtrate was then concentrated to 10 mg/mL using a 10 kD molecular weight cut off (MWCO) Sartocon Slice tangential flow filtration (TFF) system (Sartorius). The filter was first prepared by washing with 10 mM histidine, 130 mM NaCl, pH 6.0 and the permeate was sampled for pH and conductivity. Following concentration, the concentrated protein was sampled and tested for protein concentration and enzyme activity. A 6× buffer exchange was performed on the concentrated protein into the final buffer: 10 mM histidine, 130 mM NaCl, pH 6.0. Following buffer exchange, the concentrated protein was passed though a 0.22 μm filter into a 20 L sterile storage bag. The protein was sampled and tested for protein concentration, enzyme activity, free sulfydryl groups, oligosaccharide profiling and osmolarity.

The sterile filtered bulk protein was then asceptically dispensed at 20 mL into 30 mL sterile Teflon vials (Nalgene). The vials were then flash frozen and stored at −20±5° C.

### Example 4

### Production of rBChE

In this example, recombinant butyrylcholinesterase (rBChE) was produced in the mammary gland of transgenic animals, including mice and goats. In short, a DNA expression vector bCN-BchE was developed that contained a 2.4-kb dimerized chicken β-globin gene insulator; a 6.7-kb goat (3-casein gene promoter fragment, including the signal sequence in exon 2; a 1.7-kb human butyrylcholinesterase (HuBChE) cDNA clone [ATCC Catalog No. 65726, Manassas, Va.; SEQ ID NO:208)]; and a 6.1-kb fragment containing the β-casein coding and 3′ noncoding regions (see U.S. Pat. Publ. No. 20040016005 and Huang et al., (2007) Proc Natl Acad Sci USA 104:1360e-13608). The plasmid backbone of bCN-BChE was removed by NotI digestion and the 16.9-kb rBChE transgene fragment was gel-purified and microinjected into the pronuclei of in vitro-produced zygotes to generate transgenic mice and goats. rBChE was expressed in the transgenic animals and purified.

a. Plasmid Construction and Preparation of the Transgene DNA Expression Cassette

All DNA cloning was performed using E. coli Stb12™ competent cells (Invitrogen, Burlington, ON, Canada). Primers for sequencing and PCR were synthesized by Sigma-Genosys (Oakville, ON, Canada). PCR was performed using Ready-To-Go PCR beads (GE Healthcare Life Sciences, Baie d'Urfe, QC, Canada) or a High Fidelity PCR kit (Roche Diagnostics, Laval, QC, Canada).

The goat b-casein promoter (SEQ ID NO:195), including the 5′ untranscribed region up to exon 2 of the b-casein gene, was amplified by PCR using genomic DNA isolated from blood of a Nigerian dwarf goat (Karatzas and Turner (1997) J Dairy Sci 80:2225-2232) with a sense primer containing the 5′ end of the promoter (Acb582; SEQ ID NO:196) and an anti-sense primer containing an XhoI site downstream of exon 2 just before the ATG codon (Acb591; SEQ ID NO:197). The resulting 6.0-kb PCR product was subcloned into the pUC18 vector (Promega, Madison, Wis.; SEQ ID NO:198) to generate pUC18/5′ bCN. The β-casein gene (SEQ ID NO:199) containing exon 7 and the 3′ end was PCR amplified from the goat genomic DNA with primers Acb583 (SEQ ID NO:200) and Acb601 (SEQ ID NO:201). The fragment was subcloned into the pUC18 vector (SEQ ID NO:198) to generate pUC18/3′ bCN.

The 4.3-kB fragment encompassing exon 7 and the 3′ end of the goat β-casein gene was then PCR amplified from pUC18/3′ bCN using primer Acb620 (SEQ ID NO:202) which introduced NotI and XhoI sites and Acb621 (SEQ ID NO:203) which introduced SalI and NotI sites. This fragment was subcloned into the pUC18 vector and designated pUC18bCNA. A 4.9-kB fragment containing the 5′ end of the β-casein promoter including sequences through exon 2 was PCR amplified from pUC18/5′ bCN using primer Acb618 (SEQ ID NO:204) which introduced a BamHI and Sad site at the 5′ end and primer Acb619 (SEQ ID NO:205) which introduced an XhoI site. The amplified product included an XhoI restriction site and was 6.1 kb. To generate the pUC18/bCN vector, the pUC18bCNA vector was cleaved with XhoI and ligated with the XhoI-digested 6.1-kb PCR product.

This new vector was then digested with NotI and BamHI and ligated to the insulator fragment derived from an upstream region of the chicken b-globin gene (Chung et al. (1997) Proc Natl Acad Sci USA 94:575-580). The insulator fragment was derived from PCR amplification of genomic DNA from a chicken with two insulator-specific primers Insulator-p1 (SEQ ID NO:206) and Insulator-p2 (SEQ ID NO:207). The PCR product was divided into two portions and cleaved with either NotI and XhoI or BamHI and SalI, then ligated together to form a 2.4-kb insulator fragment with NotI and BamHI on either end. The HuBChE cDNA was amplified by PCR from cDNA clone ATCC #65726 (Arpagaus et al. (1990) Biochemistry 29:124-131) (ATCC, Manassas, Va.; SEQ ID NO:208) with the sense primer Acb719 (SEQ ID NO:209) containing an XhoI site, goat b-casein signal sequence, a partial sequence corresponding to the mature huBChE, and the antisense primer Acb718 (SEQ ID NO:210) containing an XhoI site and partial 3′ sequence of the human BChE cDNA. The 1.7-kb PCR product was subcloned into the pGEM-T easy vector (Promega; SEQ ID NO:211), the BChE insert fully sequenced, and the plasmid was digested with XhoI to remove the BChE insert which was purified with GFX matrix (GE Healthcare Life Sciences). The purified BChE insert was ligated to XhoI-digested pUC18/bCN plasmid to generate the final bCN-BChE vector.

Transgenic mice and goats were generated with purified, NotI digested linear DNA of the same vector. Briefly, cesium chloride purified circular bCN-BChE DNA was digested with NotI, separated by electrophoresis and the bCN-BChE fragment isolated from the gel. The gel-purified bCN-BChE DNA was then mixed with cesium chloride and centrifuged at 60,000 rpm for 16 to 20 h at 20° C. in a Beckman L7 ultracentrifuge using a Ti70.1 rotor (Beckman Instruments, Fullerton, Calif.). The DNA band was removed, dialyzed against WFI water for 2 to 4 h and precipitated with ethanol. The precipitated DNA was resuspended in injection buffer (5 mM Tris pH 7.5, 0.1 mM EDTA, 10 mM NaCl) and dialyzed against the same buffer at 4° C. for 8 h, then dialyzed again against the buffer for 16 h and 8 h, respectively. Following dialysis, the DNA was quantified using a fluorometer and stored at 4° C. The DNA was diluted to a concentration of 3 mg/mL in injection buffer before use.

### B. Production of Founder and Subsequent Generation of Transgenic Animals

Transgenic mice were produced and maintained at McIntyre Transgenic Core Facility of McGill University (Montreal, QC, Canada). Animal studies were carried out in accordance with guidelines on the care and use of experimental animals from the Canadian Council of Animal Care. Transgenic mice were generated in a friend virus B-type (FVB) background strain (Charles River Laboratories, Wilmington, Mass.) (Hogan et al., (1986) Manipulating the Mouse Embryo: A Laboratory Manual (Cold Spring Harbor Lab Press, Cold Spring Harbor, N.Y.)). The bCN-BChE expression vector containing the transgene was microinjected into fertilized eggs, and 22 pups were born. At 2-3 weeks of age, tail biopsies were taken under anesthesia, and DNA was prepared according to standard procedures (see Sambrook et al., (1989) Molecular Cloning: A Laboratory Manual (Cold Spring Harbor Lab Press, Cold Spring Harbor, N.Y.)). Transgenic founder mice were bred with wild-type mice of the same strain for the production of F₁, F₂, and F₃ generations.

The production and maintenance of transgenic goats were conducted at the PharmAthene Canada Caprine Production Farm. Animal studies were carried out according to protocols approved by the Animal Care Committee of PharmAthene Canada. The production of the founder goats and subsequent generation of rBChE transgenic goats was performed as described in Wang et al., (2002) Mol Reprod Dev 63:437-443. Briefly, 3 μg/mL of the transgene DNA fragment were microinjected into in vitro produced goat zygotes. After a brief in vitro culture, the zygotes were transferred to recipient goats and pregnancies confirmed by transrectal ultrasonography. Newborn kids were separated from recipient goats at birth to prevent disease transmission. Transgenic founder goats were bred with wild-type goats of the same strain for the production of subsequent generations. Cloned copies of a selected female founder goat, 1871F, were generated by somatic cell nuclear transfer (Keefer et al., (2002) Biol Reprod 66:199-203). F₁ goats of the cloned copies of the founder goat were obtained by laparoscopic ovum pick-up-in vitro fertilization (see Baldassarre et al., (2004) Cloning Stem Cells 6:25-29). Herd expansion was performed in nontransgenic New Zealand herd goats by artificial insemination from a master semen bank established from one of the male transgenic F₁ goats, 2219M and some of its male offspring. Transgenic female goats were hormonally induced into lactation (see Cammuso et al., (2000) Anim Biotechnol 11:1-17) at 2 months of age to measure expression of rBChE in the milk before natural lactation took place at 12 months age.

rBChE transgenic animals were identified and characterized by PCR, Southern blot and FISH analysis, using standard protocols.

C. Purification of rBChE from Milk of Transgenic Goats.

All of the purification procedures were performed at 20° C.±2° C., unless otherwise noted. Milk containing rBChE at a concentration of 1-5 g per liter was filtered through a tangential flow filtration system to remove fat and caseins. More than 80% of the rBChE was recovered. The clarified milk (whey) was washed with 7 bed volumes of 10 mM phosphate buffer, pH 7.2, 1 mM EDTA, 140 mM NaCl and concentrated using a 30-kDa flat sheet cartridge. Washed whey was applied to a HQ50 ion exchange column (Applied BioSystems, Foster City, Calif.) previously equilibrated with the same buffer. The eluent (containing the rBChE) was collected and the column was subsequently washed with 10 mM phosphate buffer, pH 7.2, 1 mM EDTA, 1 M NaCl to remove any captured impurities. The HQ50 eluent was loaded onto a procainamide affinity column previously equilibrated with 10 mM phosphate buffer, pH 7.2, 1 mM EDTA, 140 mM NaCl. The column was washed with 10 bed volumes of the same equilibration buffer and the protein was eluted with 10 mM phosphate buffer, pH 7.2, 1 mM EDTA, 500 mM NaCl. The purified rBChE was filtered sterile and stored at 4° C. The purified protein was tested for BChE activity and total protein concentration was determined. The purity of the protein was assessed by SDS/PAGE with silver staining (Invitrogen) as described by the manufacturer.

D. Analysis of rBChE Expressed in the Milk of Transgenic Animals

Milk samples from transgenic animals, collected after initiation of induced or natural lactation, were analyzed for the presence of the rBChE, using nondenaturing polyacrylamide gels stained for cholinesterase activity (Karnovsky and Roots (1964) J Histochem Cytochem 12:219-221). The rBChE produced in the milk of the transgenic animals migrated as a mixture of dimer, tetramer and monomer, with dimer as the predominant form. Western blot analysis under denaturing and reducing conditions with a polyclonal anti-huBChE antibody confirmed rBChE was expressed in the milk of the transgenic animals migrating at the expected size of the protein (˜90 kDa).

### Example 5

### Butyrylcholinesterase Activity

In this example, the butyrylcholinesterase activity of rBChE was determined. Milk collected from the transgenic animals during induced or natural lactation was analyzed for BChE activity using a Cholinesterase (BTC) kit (Sigma-Aldrich) at 30° C. with 5 mM butyrylthiocholine as the substrate in a buffer (pH 7.2) or a microtiter plate modification of the previously described Ellman assay (Ellman et al., (1961) Biochem Pharmacol 7, 88-95).

Using a microplate assay, all wells, including sample wells for a standard curve and control wells, were plated in duplicate. A 40-mL milk sample, diluted in 10 mM potassium phosphate buffer (pH 7.0) containing 1 mM EDTA and 1 mg/mL BSA, was mixed at room temperature with 200 mL of reaction mix containing 1 mM butyrylthiocholine and 0.25 mM 5,5′-Dithiobis(2-nitrobenzoic acid) (DTNB) in 100 mM potassium phosphate buffer (pH 8.0). The sample plate was loaded into a Vₘₐₓ Kinetic Microplate reader (Molecular Devices, Sunnyvale, Calif.) and run for 1 min at 405 nM. The results were analyzed by the Softmax software attached to the Microplate reader. One unit of enzyme activity was defined as the amount required to hydrolyze 1 mmol substrate per minute with 720 units equivalent to 1 mg purified human plasma BChE (Duysen et al. (2002) J Pharmacol Exp Ther 302:751-758).

Due to the presence of endogenous cholinesterase activity present in naïve animal plasma which was measured in the assay for PEG-rBChE, corrections to the entire plasma concentration-time data were made in order to evaluate only the pharmacokinetic behavior of the exogenously administered agent. For each animal, the baseline (predose) plasma concentration value was subtracted from the rest of concentration-time points for that animal in order to normalize the measured concentrations and remove the endogenous component concentration. This correction was based on the assumption that the endogenous substance measured at baseline would have remained constant over the course of the study.

### Example 6

### PEGylated rBChE

In this example, rBChE was modified by PEGylation and its pharmacokinetic parameters and prophylactic efficacy against nerve agent exposure were determined.

A. PEGylation of rBChE

rBChE purified in Example 4 above was PEGylated by reaction of rBChE with PEG 20,000 using lysine linkage chemistry (see published U.S. Patent Publication No. US2011/0135623). Purified rBChE (0.5 mg/mL) was mixed quickly with PEG 20,000 at a ratio of 1:80 in 50 mM sodium phosphate, pH 8.0 and incubated at 24° C. for 2 h. The PEGylated rBChE was purified by affinity chromatography with a procainamide column as described in Example 4 above. SDS/PAGE and SEC-HPLC/light scattering revealed that one to three PEGs were attached to each rBChE molecule.

Single dose administration in male guinea pigs and subsequent compartmental data analysis of plasma concentration-time data (see Example 7 below) revealed the bioavailability of PEGylated rBChE was −46% with a plasma half-life of −44 hours as compared to −7.3% with a plasma half-life of −6.5 hours for unmodified rBChE.

### B. Safety and Prophylactic Efficacy

A study was performed to assess performance deficits in animals given PEG rBChE or vehicle control 18 hours prior to subcutaneous (SC) administration of a saline nerve agent sham. No toxicity or performance deficits were observed in any of the animals.

The prophylactic efficacy of PEG-rBChE was evaluated in Hartley guinea pigs exposed to Soman or VX. The experimental design and dosing schedule is set forth in Table 6 below. PEG rBChE was administered at a dosage of 140 mg/kg by intramuscular (1M) injection. Nerve agents VX (LD₅₀=8 μg/kg) or Soman (GD; LD₅₀=28 μg/kg) was administered at a total cumulative dose of 5.5×LD₅₀ 18 hours following administration of PEG rBChE or vehicle control. As a comparison, the effect of other nerve agent therapies also were tested, which were administered upon exposure to nerve agent. Atropine was administered at a dosage of 2 mg/kg by IM injection. Pralidoxime chloride (2PAM, Protopam) was administered at a dosage of 25 mg/kg by IM injection. Diazepam was administered at a dosage of 10 mg/kg by SC injection. Animals were observed 6 hrs. post-challenge for cholinergic toxicity and were tested at 30 and 48 hrs in a balance beam test and at 1 wk. in the Morris water maze. The study was performed at the US Army Medical Research Institute of Chemical Defense (USAMRICD). The results are set forth in Table 6 below. All animals treated with PEG-rBChE prior to nerve agent exposure survived with no signs of cholinergic toxicity. Most animals treated with conventional therapy at the time of nerve agent exposure survived but had significant signs of cholinergic toxicity as well as marked impairment in the balance beam and water maze tests.

TABLE 6
Prophylactic efficacy of PEG-rBChE in Guinea Pigs
exposed to Soman or VX

| | | | Time of | |
|---|---|---|---|---|
| SQ Nerve | Nerve | | Nerve Agent | # of Survivors |
| Agent Dose | Agent | Treatment | Exposure | (Sequelae) |
| Saline Sham | Sham | Vehicle | +18 hrs | 12/12 |
| | | control | | (Normal) |
| 5.5 X LD₅₀ | Soman | PEG-rBChE | +18 hrs | 12/12 |
| | | | | (Normal) |
| 5.5 X LD₅₀ | VX | PEG-rBChE | +18 hrs | 12/12 |
| | | | | (Normal) |
| 1.5 X LD₅₀ | Soman | Atropine/2PAM | +0 hrs | 2/4 |
| | | Diazepam | | (Marked |
| | | | | impairment) |
| 1.5 X LD₅₀ | VX | Atropine/2PAM | +0 hrs | 10/10 |
| | | Diazepam | | (Marked |
| | | | | impairment) |

### Example 7

### Single Dose Pharmacokinetic Evaluation of PEG-rBChE and rHuPH20 by Intramuscular Injection or Subcutaneous Injection in Minipigs

In this example, the pharmacokinetics of rBChE (Protexia®, PEG-RBChE) when administered alone or in combination with recombinant human hyaluronidase (rHuPH20; produced as described in Examples 1-3) by intramuscular injection or subcutaneous injection to minipigs were determined. The results were compared with intravenous injection of PEG-rBChE alone as a single dose.

### A. Experimental Methods

Male Gottingen minipigs, ˜23 wks of age, were randomized by weight (˜11-16 kg) into five study groups with 4 male minipigs in each group. Animals were anesthetized prior to dose administration. The experimental design and dosing schedule is set forth in Table 7 below. For the route of administration, intravenous (IV) injection was into the marginal ear vein, intramuscular (IM) injection was in the thigh and subcutaneous (SC) injection was into the inguinal fold. A single dose of 25 mg/kg of PEG-rBChE was administered at Day 1. rHuPH20 was co-administered with PEG-rBChE, in the same composition, to groups 3 and 5 at a dose of 11 μg/Kg. Dose volumes were calculated using the most recent body weight measurement taken prior to Day 1 for each minipig.

TABLE 7
Dosing Schedule

| Group | No. of Animals | Dose Material | Route of |
|---|---|---|---|
| No. | Males | (25 mg/kg PEG-rBChE) | Administration |
| 1 | 4 | PEG-rBChE | Intravenous |
| 2 | 4 | PEG-rBChE | Intramuscular |
| 3 | 4 | PEG-rBChE + rHuPH20 | Intramuscular |
| 4 | 4 | PEG-rBChE | Subcutaneous |
| 5 | 4 | PEG-rBChE + rHuPH20 | Subcutaneous |

### B. Pharmacokinetic Analysis

For pharmacokinetic analysis, serial blood samples were collected from all minipigs by venipuncture of the anterior vena cava following the single dose at the following nominal times: predose (immediately prior to dosing), 5, 10, 20 and 30 minutes, 1, 2, 4, 8, 12 and 16 hours and on Days 2, 3, 5, 7, 9, 11, 13 and 15 post-dose. Blood was collected into vacutainer tubes containing K2EDTA as anticoagulant. The blood sample tubes were placed in ice following collection and processed to plasma and frozen. The resultant plasma samples were then analyzed for PEG-rBChE concentrations in all samples from all minipigs. Minipigs were humanely euthanized on Day 15. Animal care and use procedures followed the USDA Animal Welfare Act and the Guide for the Care and Use of Laboratory Animals from the National Research Council.

Plasma samples were analyzed for PEG-rBChE plasma concentration levels using a qualified enzymatic activity assay as described in Example 5 above. The linear range of the assay was 50 mU/mL to 1200 mU/mL. Reported PEG-rBChE plasma concentration units were mU/mL. These values were converted to μg/mL using the specific activity constant of 644.43 U/mg. Therefore, 1 mU/mL=0.001552 μg/mL.

Baseline corrected PEG-rBChE plasma concentration-time data was determined as described in Example 6, and were used for further analysis by non-compartmental analysis (NCA) using the software program WinNonlin (WinNonlin Professional version 5.3, PharSight Corp., Mountain View, Calif.). For the non-compartmental analysis, at least 3 measurable concentrations were available in each concentration-time profile. The methods listed below were used, if applicable, in determining the pharmacokinetic parameters.

a. Determination of Cₘₐₓ

The maximum “peak” concentration (Cₘₐₓ) was obtained by visual inspection of the baseline corrected concentration-time profiles. In the event of two or more identical “peak” concentrations, the earlier value was reported to be Cₘₐₓ for purposes of accurate evaluation of Tₘₐₓ.

b. Determination of Tₘₐₓ

Tₘₐₓ was determined as the time value associated with the observed Cₘₐₓ.

c. Determination of the Elimination Rate Constant

Where feasible, the apparent elimination rate constant (λ_{z}) was determined using unweighted linear regression analysis on at least three log-transformed concentrations visually assessed to be on the linear portion of the terminal slope but not including the peak concentration. In general, objective selection of points included in the estimation of half-life required selection of those points which maximized the R² (0.9 or above) for the linear regression.

The elimination rate constant was not determined if there were less than three concentrations selected. The maximum concentration was not included in the estimation of the elimination rate constant. Furthermore, unless the terminal data point appeared to be part of a new elimination phase, or there was reason to believe that the last concentration was in error, the last measurable concentration was always included.

d. Determination of Terminal Elimination Half-Life

Where feasible, the terminal elimination phase half-life (T_{1/2}) was calculated as the ratio of logₑ2 to λ_{z} (e.g., T_{1/2}=0.693/λ_{z}).

e. Determination of AUC

i. AUC₀₋ₜ

The area under the baseline corrected concentration-time curve from time zero to the last quantifiable concentration (AUC₀₋ₜ) was estimated by a combination of linear trapezoidal method on concentrations up to Cₘₐₓ and logarithmic trapezoidal methods on concentrations after Cₘₐₓ. At least three quantifiable concentration values had to be available for calculation of an individual value of AUC.

ii. AUC_{inf}

Where feasible, the AUC extrapolated to infinity, AUC_{inf}, was calculated as the sum AUC₀₋ₜ and Cₜ/λ_{z}, where Cₜ is the predicted concentration at time t obtained from the regression analysis used to determine the elimination rate constant. At least three quantifiable concentration values had to be available for calculation of an individual value of AUC, and the elimination rate constant (λ_{z}) had to be estimated as well.

f. Determination of Bioavailability

The bioavailability was estimated as the ratio of the mean AUC₀₋ₜ for the IM or SC routes to mean AUC₀₋ₜ the from the IV route. As doses were the same for all routes of administration, no dose correction was required.

g. Determination of Clearance

Where feasible, the clearance was estimated using the relationship CL/F=Dose/AUC_{inf}. The dose used for this evaluation was the actual administered dose. Clearance could only be estimated for those minipigs that had associated values for AUC_{inf}.

h. Determination of Volume of Distribution

Where feasible, the volume of distribution (Vz) was estimated using the relationship Vz/F=Dose/(λ_{z}*AUC_{inf}). The dose used was the actual administered dose. Clearance could only be estimated for those minipigs that had associated values for AUC_{inf}.

i. Determination of Mean Residence Time

Where feasible, the mean residence time (MRT) from the time of dosing to the time of the last measurable concentration was estimated as AUMC₀₋ₜ/AUC₀₋ₜ.

### C. Results

Final pharmacokinetic analysis was conducted for minipigs in Groups 1 through 5 who had measurable concentration-time data. One blood sample was received in hemolyzed condition at the bioanalytical lab. No PEG-rBChE plasma concentrations were reported for this sample (animal #S5234558/M, Group 5, Day 1, minute).

After baseline correction (as described in Example 5), plasma concentrations were available in all minipigs until Day 15, the last day for pharmacokinetic sampling. Actual sampling times calculated from the time of dosing were used in the analysis along with the back calculated total dose based on the rounded off dose volumes in each minipig.

The terminal slopes in two out of four minipigs in Groups 1, 2, 4 and 5 by Day 8 declined rapidly, indicating nonlinear behavior. Consequently, the elimination rate constant could not be estimated robustly, and therefore only Cₘₐₓ Tₘₐₓ and AUC₀₋ₜ were calculated and reported for those profiles and all comparisons across groups were made based only on these parameters.

The 12 hr time point on Day 1 in animal # S5232971/M in Group 3, showed a sudden decrease for no obvious reason. The concentrations returned to the expected levels on the subsequent sample. There was no indication of any problem with the sample or assay. The value was included in the NCA as the impact on AUC was minor.

A summary of the mean pharmacokinetic parameters is presented in Table 8. The overall pharmacokinetic parameters are presented in Tables 9-13 below for Groups 1 through 5, respectively. Results of each pharmacokinetic parameter are summarized below.

1. Baseline Concentration of PEG-rBChE

The results show that following administration of PEG-rBChE, the baseline corrected concentrations of PEG-rBChE reached peak levels 0.67-1.03 day post-dose, then decreased from peak concentration in a mono-exponential fashion. This concentration-time profile was broadly similar across all dose groups, although there were two animals each in Groups 1, 2, 4 and 5 that by Day 8 declined rapidly, indicating nonlinear pharmacokinetic behavior. The terminal slopes in these two out of four minipigs in Groups 1, 2, 4 and 5 declined rapidly by Day 8. Prior to baseline correction and after baseline correction, the terminal phase remained essentially flat from Day 2 to the end of the evaluation period in the majority of the profiles, indicating all exogenously administered drug had been eliminated.

2. Tₘₐₓ

The median Tₘₐₓ was observed 0.67-1.03 day post-dose day in all minipigs across all dose groups. For intramuscular (IM) administration, the median Tₘₐₓ was 1.03 and 0.67 for PEG-rBChE and PEG-rBChE+rHuPH20, respectively. For subcutaneous (SC) administration, the median Tₘₐₓ values were 1.03 and 0.99, for PEG-rBChE and PEG-rBChE+rHuPH20, respectively.

3. Cₘₐₓ

The mean Cₘₐₓ increased 1.4-fold when PEG-rBChE was administered with rHuPH20 compared to PEG-rBChE administered alone for either route of administration, intramuscularly or subcutaneously. Mean Cₘₐₓ decreased by approximately 18% when administered SC compared to IM administration irrespective of the presence of rHuPH20.

Overall, the mean Cₘₐₓ was approximately 59% to 76% lower in Groups administered IM or SC either in the presence or absence of rHuPH20 when compared to intravenous administration of PEG-rBChE alone.

4. AUC

The mean AUC₀₋ₜ increased 1.1 and 1.2-fold when PEG-rBChE was administered with rHuPH20 compared to PEG-rBChE administered alone via the IM or SC routes, respectively. Mean AUC₀₋ₜ decreased by approximately 26% and 18% when administered SC compared to IM administration of PEG-rBChE alone and in the presence of rHuPH20, respectively.

The absolute bioavailability based on mean AUC₀₋ₜ across Groups administered with PEG-rBChE alone, showed a 73% and 53% bioavailability for intramuscular and subcutaneous administrations, respectively, when compared to intravenous bolus administration. The bioavailability increased to 81% and 66% when the respective administrations combined with rHuPH20.

5. Half-Life

Overall, the median half-life for PEG-rBChE was approximately 3 to 4 days across all dose groups studied.

6. Mean Residence Time (MRT)

The mean residence time was approximately 3 days across all dose groups.

### D. Summary

This study demonstrated that the bioavailability of PEG-rBChE in the first 24 hours, by either route of administration (IM or SC), was greatly increased when co-administered with rHuPH20 as compared to PEG-rBChE administered alone. The overall increase in early exposure of PEG-rBChE (alone or with rHuPH20) was greater when administered by the IM route than when administered by the SC route. Absolute bioavailability of PEG-rBChE increased across all groups, and was at least 12% better when administered with rHuPH20 as compared to administration of PEG-rBChE alone. Co-administration with rHuPH20 caused increased absorption of PEG-rBChE by either SC or IM administration in the first 24 hours.

TABLE 8
Mean (SD) Pharmacokinetic Parameters for PEG-rBChE in Minipigs

| | Dose | | Cₘₐₓ | Tₘₐₓ* | AUC₀₋ₜ |
|---|---|---|---|---|---|
| Group | (25 mg/kg) | N | (μg/mL) | (day) | (μg · day/mL) |
| 1 | IV | 4 | 778 | 0.02 | 1253 |
| | PEG-rBChE | | (127) | (0.00-0.04) | (381) |
| 2 | IM | 4 | 224 | 1.03 | 911 |
| | PEG-rBChE | | (56) | (0.50-1.04) | (90) |
| 3 | IM PEG-rBChE + | 4 | 320 | 0.67 | 1014 |
| | rHuPH20 | | (20) | (0.66-1.00) | (129) |
| 4 | SC | 4 | 184 | 1.03 | 670 |
| | PEG-rBChE | | (51) | (1.03-2.00) | (195) |
| 5 | SC PEG-rBChE + | 4 | 264 | 0.99 | 827 |
| | rHuPH20 | | (18) | (0.50-1.00) | (109) |

IV: Intravenous;
IM: Intramuscular;
SC: Subcutaneous;
*median (range)

TABLE 9
Individual and Summary Pharmacokinetic Parameters of PEG-rBChE in Minipigs in Group 1
Following Single Intravenous Injection of PEG-rBChE on Day 1

| | | | Cmax | Tmax | AUC₀₋ₜ | AUC_{inf} | CL | Vz | MRT₀₋ₜ | MRT_{inf} | T½ |
|---|---|---|---|---|---|---|---|---|---|---|---|
| Animal | Dose | Weight | (μg/mL) | (day) | (μg · day/mL) | (μg · day/mL) | (mL/day) | (mL) | (day) | (day) | (day) |
| S5233128/M | 395.85 | 15.81 | 657.294 | 0.04 | 1122.23 | ND | ND | ND | 2.24 | ND | ND |
| S5234205/M | 291.2 | 11.65 | 892.789 | 0.04 | 1821.98 | 2094.02 | 139.06 | 940.4 | 4.02 | 6.20 | 4.69 |
| S5235252/M | 327.6 | 13.09 | 679.022 | 0.01 | 1032.76 | ND | ND | ND | 2.02 | ND | ND |
| S5235342/M | 309.4 | 12.35 | 882.333 | 0.00 | 1037.01 | 1045.44 | 295.95 | 911.58 | 2.38 | 2.50 | 2.14 |
| N | — | — | 4 | 4 | 4 | 2 | 2 | 2 | 4 | 2 | 2 |
| Mean | — | — | 777.860 | 0.023 | 1253.495 | 1569.733 | 217.507 | 925.992 | 2.667 | 4.347 | 3.411 |
| SD | — | — | 127.054 | 0.021 | 381.222 | 741.459 | 110.937 | 20.381 | 0.914 | 2.614 | 1.805 |
| Min | — | — | 657.29 | 0 | 1032.76 | 1045.44 | 139.06 | 911.58 | 2.02 | 2.50 | 2.14 |
| Median | — | — | 780.68 | 0.02 | 1079.62 | 1569.73 | 217.51 | 925.99 | 2.31 | 4.35 | 3.41 |
| Max | — | — | 892.79 | 0.04 | 1821.98 | 2094.02 | 295.95 | 940.4 | 4.02 | 6.20 | 4.69 |
| CV % | — | — | 16.3 | 90.1 | 30.4 | 47.2 | 51.0 | 2.2 | 34.3 | 60.1 | 52.9 |
| Geo Mean | — | — | 770.027 | 0.014 | 1216.472 | 1479.588 | 202.869 | 925.88 | 2.568 | 3.934 | 3.163 |

ND = Not Determined

TABLE 10
Individual and Summary Pharmacokinetic Parameters of PEG-rBChE in Minipigs in Group 2
Following Single Intramuscular Injection of PEG-rBChE on Day 1

| | | | Cmax | Tmax | AUC₀₋ₜ | AUC_{inf} | CL/F | Vz/F | MRT₀₋ₜ | MRT_{inf} | T½ |
|---|---|---|---|---|---|---|---|---|---|---|---|
| Animal | Dose | Weight | (μg/mL) | (day) | (μg · day/mL) | (μg · day/mL) | (mL/day) | (mL) | (day) | (day) | (day) |
| S5232946/M | 368.55 | 14.74 | 184.303 | 1.04 | 807.36 | ND | ND | ND | 3.24 | ND | ND |
| S5233306/M | 337.61 | 13.50 | 175.500 | 0.50 | 920.18 | ND | ND | ND | 3.42 | ND | ND |
| S5235333/M | 325.78 | 13.02 | 237.334 | 1.03 | 892.51 | 933.05 | 349.16 | 1483.23 | 3.48 | 4.12 | 2.94 |
| S5235368/M | 303.03 | 12.09 | 297.218 | 1.03 | 1024.68 | 1072.17 | 282.63 | 1244.07 | 3.36 | 4.03 | 3.05 |
| N | — | — | 4 | 4 | 4 | 2 | 2 | 2 | 4 | 2 | 2 |
| Mean | — | — | 223.589 | 0.901 | 911.182 | 1002.611 | 315.894 | 1363.652 | 3.377 | 4.076 | 2.998 |
| SD | — | — | 56.173 | 0.267 | 89.610 | 98.375 | 47.04 | 169.112 | 0.104 | 0.069 | 0.075 |
| Min | — | — | 175.50 | 0.50 | 807.36 | 933.05 | 282.63 | 1244.07 | 3.24 | 4.03 | 2.94 |
| Median | — | — | 210.82 | 1.03 | 906.34 | 1002.61 | 315.89 | 1363.65 | 3.39 | 4.08 | 3 |
| Max | — | — | 297.22 | 1.04 | 1024.68 | 1072.17 | 349.16 | 1483.23 | 3.48 | 4.12 | 3.05 |
| CV % | — | — | 25.1 | 29.7 | 9.8 | 9.8 | 14.9 | 12.4 | 3.1 | 1.7 | 2.5 |
| Geo Mean | — | — | 218.555 | 0.863 | 907.894 | 1000.195 | 314.138 | 1358.399 | 3.376 | 4.075 | 2.997 |

ND: Not Determined

TABLE 11
Individual and Summary Pharmacokinetic Parameters of PEG-rBChE in Minipigs in Group 3
Following Single Intramuscular Injection of PEG-rBChE and rHuPH20 on Day 1

| | | | Cmax | Tmax | AUC₀₋ₜ | AUC_{inf} | CL/F | Vz/F | MRT₀₋ₜ | MRT_{inf} | T½ |
|---|---|---|---|---|---|---|---|---|---|---|---|
| Animal | Dose | Weight | (μg/mL) | (day) | (μg · day/mL) | (μg · day/mL) | (mL/day) | (mL) | (day) | (day) | (day) |
| S5232971/M | 316.83 | 12.69 | 341.867 | 0.67 | 859.04 | 885.52 | 357.79 | 1446.95 | 3.18 | 3.63 | 2.80 |
| S5233446/M | 379.48 | 15.20 | 321.687 | 1.00 | 1020.2 | 1037.86 | 365.64 | 1312.24 | 3.16 | 3.41 | 2.49 |
| S5234027/M | 332.94 | 13.32 | 293.423 | 0.66 | 1001.44 | 1058.31 | 314.6 | 1455.98 | 3.41 | 4.22 | 3.21 |
| S5234892/M | 294.46 | 11.79 | 322.000 | 0.66 | 1173.97 | 1252.16 | 235.16 | 1146.88 | 3.68 | 4.62 | 3.38 |
| N | — | — | 4 | 4 | 4 | 4 | 4 | 4 | 4 | 4 | 4 |
| Mean | — | — | 319.744 | 0.748 | 1013.665 | 1058.463 | 318.295 | 1340.514 | 3.357 | 3.969 | 2.97 |
| SD | — | — | 19.926 | 0.167 | 128.84 | 150.392 | 59.796 | 144.86 | 0.239 | 0.553 | 0.402 |
| Min | — | — | 293.42 | 0.66 | 859.04 | 885.52 | 235.16 | 1146.88 | 3.16 | 3.41 | 2.49 |
| Median | — | — | 321.84 | 0.67 | 1010.82 | 1048.09 | 336.19 | 1379.6 | 3.29 | 3.92 | 3.01 |
| Max | — | — | 341.87 | 1.00 | 1173.97 | 1252.16 | 365.64 | 1455.98 | 3.68 | 4.62 | 3.38 |
| CV % | — | — | 6.2 | 22.3 | 12.7 | 14.2 | 18.8 | 10.8 | 7.1 | 13.9 | 13.5 |
| Geo Mean | — | — | 319.272 | 0.736 | 1007.503 | 1050.516 | 313.652 | 1334.399 | 3.351 | 3.94 | 2.949 |

TABLE 12
Individual and Summary Pharmacokinetic Parameters of PEG-rBChE in Minipigs in Group 4
Following Single Subcutaneous Injection of PEG-rBChE on Day 1

| | | | Cmax | Tmax | AUC₀₋ₜ | AUC_{inf} | CL/F | Vz/F | MRT₀₋ₜ | MRT_{inf} | T½ |
|---|---|---|---|---|---|---|---|---|---|---|---|
| Animal | Dose | Weight | (μg/mL) | (day) | (μg · day/mL) | (μg · day/mL) | (mL/day) | (mL) | (day) | (day) | (day) |
| S5232598/M | 353.99 | 14.14 | 216.731 | 1.03 | 830.30 | 913.82 | 387.37 | 2297.36 | 4.03 | 5.48 | 4.11 |
| S5232602/M | 317.59 | 12.69 | 117.100 | 1.03 | 388.35 | 406.82 | 780.66 | 3608.04 | 3.52 | 4.20 | 3.20 |
| S5232695/M | 340.34 | 13.61 | 229.411 | 1.03 | 755.27 | ND | ND | ND | 2.79 | ND | ND |
| S5233250/M | 285.74 | 11.41 | 171.177 | 2.00 | 704.82 | ND | ND | ND | 3.44 | ND | ND |
| N | — | — | 4 | 4 | 4 | 2 | 2 | 2 | 4 | 2 | 2 |
| Mean | — | — | 183.605 | 1.27 | 669.684 | 660.319 | 584.02 | 2952.701 | 3.446 | 4.843 | 3.657 |
| SD | — | — | 50.902 | 0.486 | 194.515 | 358.501 | 278.098 | 926.79 | 0.511 | 0.903 | 0.641 |
| Min | — | — | 117.1 | 1.03 | 388.35 | 406.82 | 387.37 | 2297.36 | 2.79 | 4.2 | 3.2 |
| Median | — | — | 193.95 | 1.03 | 730.04 | 660.32 | 584.02 | 2952.7 | 3.48 | 4.84 | 3.66 |
| Max | — | — | 229.41 | 2.00 | 830.3 | 913.82 | 780.66 | 3608.04 | 4.03 | 5.48 | 4.11 |
| CV % | — | — | 27.7 | 38.3 | 29.0 | 54.3 | 47.6 | 31.4 | 14.8 | 18.6 | 17.5 |
| Geo Mean | — | — | 177.678 | 1.213 | 643.664 | 609.721 | 549.918 | 2879.057 | 3.416 | 4.801 | 3.629 |

ND: Not Determined

TABLE 13
Individual and Summary Pharmacokinetic Parameters of PEG-rBChE in Minipigs in Group 5
Following Single Subcutaneous Injection of PEG-rBChE and rHuPH20 on Day 1

| | | | Cmax | Tmax | AUC₀₋ₜ | AUC_{inf} | CL/F | Vz/F | MRT₀₋ₜ | MRT_{inf} | T½ |
|---|---|---|---|---|---|---|---|---|---|---|---|
| Animal | Dose | Weight | (μg/mL) | (day) | (μg · day/mL) | (μg · day/mL) | (mL/day) | (mL) | (day) | (day) | (day) |
| S5233489/M | 358 | 14.35 | 239.333 | 0.50 | 671.08 | 677.45 | 528.45 | 1590.81 | 2.81 | 2.94 | 2.09 |
| S5234043/M | 364.27 | 14.57 | 260.757 | 1.00 | 904.73 | 959.08 | 379.81 | 1711.01 | 3.44 | 4.29 | 3.12 |
| S5234558/M | 269.4 | 10.77 | 281.164 | 0.99 | 901.54 | ND | ND | ND | 2.85 | ND | ND |
| S5235295/M | 296.25 | 11.85 | 273.185 | 0.99 | 831.97 | ND | ND | ND | 2.92 | ND | ND |
| N | — | — | 4 | 4 | 4 | 2 | 2 | 2 | 4 | 2 | 2 |
| Mean | — | — | 263.610 | 0.871 | 827.331 | 818.264 | 454.130 | 1650.907 | 3.005 | 3.617 | 2.605 |
| SD | — | — | 18.233 | 0.246 | 109.441 | 199.143 | 105.109 | 84.992 | 0.297 | 0.958 | 0.733 |
| Min | — | — | 239.33 | 0.50 | 671.08 | 677.45 | 379.81 | 1590.81 | 2.81 | 2.94 | 2.09 |
| Median | — | — | 266.97 | 0.99 | 866.76 | 818.26 | 454.13 | 1650.91 | 2.88 | 3.62 | 2.6 |
| Max | — | — | 281.16 | 1.00 | 904.73 | 959.08 | 528.45 | 1711.01 | 3.44 | 4.29 | 3.12 |
| CV % | — | — | 6.9 | 28.3 | 13.2 | 24.3 | 23.1 | 5.1 | 9.9 | 26.5 | 28.1 |
| Geo Mean | — | — | 263.126 | 0.838 | 821.48 | 806.056 | 448.007 | 1649.813 | 2.995 | 3.553 | 2.553 |

ND = Not Determined

Since modifications will be apparent to those of skill in this art, it is intended that this invention be limited only by the scope of the appended claims.

1. A method for treating or preventing organophosphorus poisoning by administering a composition, comprising an organophosphorus (OP) bioscavenger and a hyaluronan-degrading enzyme.

2. The method of claim 1, wherein the composition is administered in a single dose.

3. The method of claim 1, wherein the composition is administered in multiple doses.

4. The method of claim 1, wherein the organophosphorus bioscavenger is an esterase, cholinesterase, paraoxonase (PON), aryldialkylphosphatase or diisopropylfluorophosphatase (DFPase).

5. The method of claim 1, wherein the organophosphorus bioscavenger is selected from among acetylcholinesterase (AChE), butyrylcholinesterase (BChE), prolidase, organophosphate acid anhydrolase (OPAA), phosphotriesterase, aryldialkylphosphatase, organophosphorus hydrolase (OPH), parathion hydrolase, diisopropylfluorophosphatase (DFPase), organophosphorus acid anhydrase, sarinase and paraoxonase (PON) and an active portion thereof or a variant thereof that exhibits at least 80% OP binding or inactivating activity.

6. The method of claim 1, wherein the organophosphorus bioscavenger has the sequence of amino acids set forth in any of SEQ ID NOS:214-256 and 258-301, an active portion thereof or a variant thereof that has at least 80% sequence identity to any of SEQ ID NOS:214-256 and 258-301.

7. The method of claim 1, wherein the organophosphorus bioscavenger is butyrylcholinesterase that has the sequence of amino acids set forth in SEQ ID NO:236, or is an active portion thereof or is a variant thereof that has at least 85% sequence identity to the sequence of amino acids set forth in SEQ ID NO:236.

8. The method of claim 1, wherein the organophosphorus bioscavenger is modified with a polymer.

9. The method of claim 8, wherein the polymer is a polyethylene glycol (PEG).

10. The method of claim 1, wherein the organophosphorus bioscavenger is linked directly or indirectly via a linker to an immunoglobulin, immunoglobulin domain, albumin, transferrin, or transferrin receptor protein.

11. The method of claim 1, wherein the hyaluronan-degrading enzyme is a hyaluronidase or a chondroitinase, or a variant or a truncated form thereof that exhibits hyaluronan-degrading activity.

12. The method of claim 1, wherein the hyaluronan-degrading enzyme is a hyaluronidase that is a PH20 or a variant or a truncated form thereof that exhibits hyaluronidase activity.

13. The method of claim 1, wherein the hyaluronan-degrading enzyme is a truncated human PH20 that consists of a sequence of amino acids set forth in SEQ ID NO:1 that contains a C-terminal truncation after amino acid position 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 of the sequence of amino acids set forth in SEQ ID NO:1, or is a variant thereof that exhibits at least 85% sequence identity to a sequence of amino acids that contains a C-terminal truncation after amino acid position 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 of the sequence of amino acids set forth in SEQ ID NO:1 and exhibits hyaluronidase activity.

14. The method of claim 13, wherein the hyaluronan-degrading enzyme has a sequence of amino acids that contains at least amino acids 36-464 of SEQ ID NO:1, or has a sequence of amino acids that has at least 85% sequence identity to a sequence of amino acids that contains at least amino acids 36-464 of SEQ ID NO:1 and exhibits hyaluronidase activity at neutral pH.

15. The method of claim 1, wherein the hyaluronan-degrading enzyme comprises the sequence of amino acids set forth in any of SEQ ID NOS:4-9 or a sequence of amino acids that exhibits at least 85% sequence identity to any of SEQ ID NOS:4-9.

16. The method of claim 1, wherein the hyaluronan-degrading enzyme is modified with a polymer.

17. The method of claim 16, wherein the polymer is a polyethylene glycol (PEG).

18. The method of claim 1, wherein the organophosphorus bioscavenger is present in the composition at a concentration of between or between about 1 to 1000 μg/mL, 0.5 to 50 mg/mL, 1 to 1000 mg/mL, 50 to 1500 mg/mL, or 100 to 750 mg/mL.

19. The method of claim 1, wherein the hyaluronan-degrading enzyme is present in the composition at a concentration between or between about 10 U/mL to 100,000 U/mL, 1000 U/mL to 50,000 U/mL, 5,000 U/mL to 20,000 U/mL or 10 U/mL to 10,000 U/mL.

20. The method of claim 1, wherein the hyaluronan-degrading enzyme is present in the composition at a concentration between or about between 10 U/mL to 5000 U/mL.

21. The method of claim 1, wherein the organophosphorus bioscavenger and hyaluronan-degrading enzyme are administered by subcutaneous administration, intramuscular administration, intralesional administration or intradermal administration.

22. The method of claim 1, wherein the organophosphorus bioscavenger and hyaluronan-degrading enzyme are administered by intramuscular administration.

23. The method of claim 1, wherein the composition is administered between at or about 6 to 48 hours, 6 to 36 hours, 6 to 24 hours, 12 to 48 hours, 12 to 36 hours, 12 to 24 hours, 24 to 48 hours, 24 to 36 hours, or at least or at least about 6, 7, 8, 9, 10, 11, 12, 13, 14, 15, 16, 17, 18, 24, 30, 36, 42 or 48 hours before exposure to the organophosphorus compound.

24. The method of claim 1, further comprising administering another pharmaceutical agent selected from among carbamates, anti-muscarinics, cholinesterase reactivators and anti-convulsives.

25. A method for treating or preventing organophosphorus poisoning by administering a combination, comprising:
administering a first composition containing an organophosphorus bioscavenger; and
administering a second composition containing a hyaluronan-degrading enzyme.

26. The method of claim 25, wherein the organophosphorus bioscavenger and hyaluronan-degrading enzyme are administered by subcutaneous administration, intramuscular administration, intralesional administration or intradermal administration.

27. The method of claim 25, wherein the organophosphorus bioscavenger and hyaluronan-degrading enzyme are administered by intramuscular administration.

28. The method of claim 25, wherein the combination is administered between at or about 6 to 48 hours, 6 to 36 hours, 6 to 24 hours, 12 to 48 hours, 12 to 36 hours, 12 to 24 hours, 24 to 48 hours, 24 to 36 hours, or at least or at least about 6, 7, 8, 9, 10, 11, 12, 13, 14, 15, 16, 17, 18, 24, 30, 36, 42 or 48 hours before exposure to the organophosphorus compound.

29. The method of claim 25, wherein the hyaluronan-degrading enzyme is a hyaluronidase that is a PH20 or a variant or a truncated form thereof that exhibits hyaluronidase activity.
